(19) United States
(12) Patent Application Publication
(10) Pub. No.: US 2024/0285808 A1
(43) Pub. Date: Aug. 29, 2024

(54) MOLECULAR PROBES FOR IN VIVO DETECTION OF ALDEHYDES

(71) Applicant: The General Hospital Corporation, Boston, MA (US)

(72) Inventors: Peter Caravan, Cambridge, MA (US); Yingying Ning, Somerville, MA (US); Hua MA, Somerville, MA (US); Sergey Shuvaev, Somerville, MA (US); Eman Akam, Somerville, MA (US)

(21) Appl. No.: 18/290,135

(22) PCT Filed: May 13, 2022
(86) PCT No.: PCT/US2022/072310

§ 371 Date: Nov. 9, 2023

Related U.S. Application Data
(60) Provisional application No. 63/188,407, filed on May 13, 2021.

(51) Int. Cl. A61K 49/10; A61P 1/16; C07D 255/02; C07D 257/02; C07D 403/12; C07D 417/12; C07D 471/08; C07F 1/08; C07F 3/06; C07F 5/00; C07F 13/00

(52) U.S. Cl. CPC A61K 49/106; A61K 49/108; A61P 1/16; C07D 255/02; C07D 257/02; C07D 403/12; C07D 417/12; C07D 471/08; C07F 1/08; C07F 3/06; C07F 5/003; C07F 13/005

(57) ABSTRACT

Disclosed herein are methods of molecular magnetic resonance (MR) imaging and positron emission tomography using extracellular probes that target extracellular allysine aldehyde and act as a noninvasive biomarker of fibrogenesis with high sensitivity and specificity in detecting fibrogenesis, for example, in rodent models and human fibrotic tissues.

### CROSS-REFERENCE TO RELATED APPLICATIONS

This application claims the benefit of U.S. Provisional Application No. 63/188,407, filed May 13, 2021, the entire disclosure of which is incorporated herein by reference.

### STATEMENT REGARDING FEDERALLY SPONSORED RESEARCH

This invention was made with government support under grant numbers H1L154125 and DK121789. The government has certain rights in the invention.

### FIELD OF THE INVENTION

The present disclosure relates to molecular probes for in vivo detection of aldehydes.

### BACKGROUND

A large number of chronic and acute diseases have a fibroproliferative component, i.e. the tissue becomes fibrotic or scarred. For example chronic liver diseases like nonalcoholic steatohepatitis, chronic kidney diseases, inflammatory bowel diseases like Crohn's disease, heart diseases like heart failure, atrial fibrillation, and myocardiac infarction, fibrotic diseases of the lung like idiopathic pulmonary fibrosis, cancers such as pancreatic ductal adenocarcinoma, scleroderma, and atherosclerosis all have a fibrotic component. Liver fibrosis plays a critical role in the evolution of most chronic liver diseases (CLD), and is characterized by a buildup of extracellular matrix, which can progress to cirrhosis, hepatocellular carcinoma, liver failure, and/or death. During the process of fibrogenesis (active fibrosis), the enzyme lysyl oxidase (LOX) and its paralogs (LOXL1, LOXL2) are upregulated. LOX oxidizes lysine residues on extracellular matrix proteins like collagens and elastins to the aldehyde containing amino acid allysine. During fibrogenesis there is a buildup of protein associated aldehydes (allysine) in the extracellular space.

### SUMMARY

Some embodiments provide a compound of Formula (I):

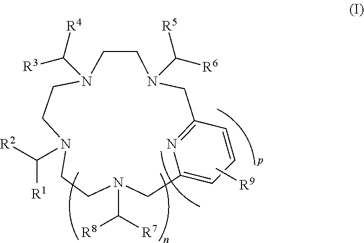

or a pharmaceutically acceptable salt thereof, wherein:

each R¹, R³, R⁵, and R⁷ are independently hydrogen or —C(═O)OH;

each R², R⁴, R⁶, and R⁸ are independently hydrogen or C₃₋₂₅ alkyl, wherein the C₃₋₂₅ alkyl is optionally substituted with 1-2 substituents independently selected from the group consisting of —NR^{A}R^{B}, —OH, halogen, C₁₋₆ alkoxy, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of halogen, —NR^{A}R^{B}, —OH, C₁₋₆ alkyl, and —C₁₋₆ alkyl-(NR^{A}R^{B}); and one or more non-adjacent carbon atoms of the C₃₋₂₅ alkyl are optionally replaced by O, N, NH, or N(CH₃);

R⁹ is H, halogen, —NR^{A}R^{B}, —OH, C₁₋₆ alkyl, or —C₁₋₆ alkyl-(NR^{A}R^{B});

each R^{A} and R^{B} are independently hydrogen or C₁₋₆ alkyl;

n is 0 or 1; and

p is 0 or 1;

wherein if n is 0 at least two of R¹, R³, and R⁵ are —C(═O)OH and if n is 1 at least two of R¹, R³, R⁵, and R⁷ are —C(═O)OH.

Some embodiments provide a composition comprising a compound of Formula (I), or a pharmaceutically acceptable salt thereof, and a pharmaceutically acceptable carrier.

Some embodiments provide a composition comprising a mixture of compounds of Formula (I), or a pharmaceutically acceptable salt thereof.

Some embodiments provide (a) a composition comprising a compound of Formula (I), or a pharmaceutically acceptable salt thereof, and a pharmaceutically acceptable carrier, or (b) a composition comprising a mixture of compounds of Formula (I), or a pharmaceutically acceptable salt thereof; wherein the composition is formulated for parenteral administration.

Some embodiments provide (a) a composition comprising a compound of Formula (I), or a pharmaceutically acceptable salt thereof, and a pharmaceutically acceptable carrier, or (b) a composition comprising a mixture of compounds of Formula (I), or a pharmaceutically acceptable salt thereof; wherein the composition is a solid formulated for dissolution in a pharmaceutically acceptable liquid medium prior to administration.

Some embodiments provide a method of magnetic resonance (MR) imaging a subject comprising:
(a) administering to a subject a compound of Formula (I) or a composition thereof as described herein; and
(b) obtaining a magnetic resonance image of the subject after a period of time.

Some embodiments provide a method of magnetic resonance (MR) imaging a subject comprising:
(a) obtaining a first magnetic resonance image of the subject;
(b) administering to a subject a compound of compound of Formula (I) or a composition thereof as described herein;
(c) obtaining a second magnetic resonance image of the subject after a period of time; and
(d) comparing the first magnetic resonance image of the subject and the second magnetic resonance image of the subject.

Some embodiments provide a method for imaging liver fibrogenesis in a subject comprising:
(a) administering to a subject a compound of Formula (I) or a composition thereof as described herein; and
(b) obtaining a magnetic resonance image of the liver of the subject after a period of time.

Some embodiments provide a method of measuring liver fibrogenesis in a subject comprising:
(a) administering to the subject a compound of Formula (I) or a composition thereof as described herein;
(b) obtaining a first magnetic resonance image of the subject after a period of time;
(c) administering to a subject a compound of Formula (I) or a composition thereof as described herein after a second period of time;
(d) obtaining a second magnetic resonance image of the subject after a period of time; and
(e) comparing the first magnetic resonance image of the subject and the second magnetic resonance image of the subject, thereby measuring liver fibrogenesis in the subject.

Some embodiments provide a method for detecting liver fibrogenesis in a subject comprising:
(a) administering to the subject a compound of Formula (I) or a composition thereof as described herein; and
(b) obtaining a magnetic resonance image of the subject after a period of time, thereby detecting the presence or absence of liver fibrogenesis in the subject.

Some embodiments provide a method of detecting liver fibrogenesis in a subject comprising:
(a) administering to the subject a compound of Formula (I) or a composition thereof as described herein;
(b) obtaining a first magnetic resonance image of the subject after a period of time;
(c) administering to a subject a Formula (I) or a composition thereof as described herein after a second period of time;
(d) obtaining a second magnetic resonance image of the subject after a period of time; and
(e) comparing the first magnetic resonance image of the subject and the second magnetic resonance image of the subject, thereby detecting the presence or absence of liver fibrogenesis in the subject.

Some embodiments provide a method for detecting liver fibrogenesis in a subject comprising obtaining a magnetic resonance image of the subject within a period of time after the subject has been administered subject a compound of Formula (I) or a composition thereof as described herein.

Some embodiments provide a method of positron emission tomography (PET) imaging a subject comprising:
(a) administering to the subject a compound of Formula (I) or a composition thereof as described herein; and
(b) obtaining a positron emission tomography image of the subject after a period of time.

Some embodiments provide a method of positron emission tomography (PET) imaging a subject comprising:
(a) obtaining a first magnetic resonance image image of the subject;
(b) administering to the subject a compound of Formula (I) or a composition thereof as described herein;
(c) obtaining a second positron emission tomography image of the subject after a period of time; and
(d) comparing the first magnetic resonance image of the subject and the second positron emission tomography image of the subject.

Some embodiments provide a compound of Formula (II)

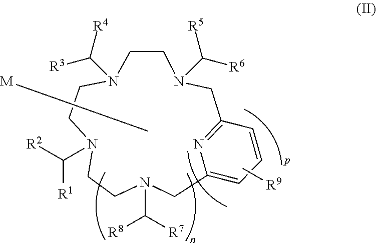

or a pharmaceutically acceptable salt thereof, wherein:

M is a metal cation;

each R¹, R³, R⁵, and R⁷ are independently hydrogen or —C(═O)OH;

each R², R⁴, R⁶, and R⁸ are independently hydrogen or C₃₋₂₅ alkyl, wherein the C₃₋₂₅ alkyl is optionally substituted with 1-2 substituents independently selected from the group consisting of —NR^{A}R^{B}, —OH, halogen, C₁₋₆ alkoxy, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of halogen, —NR^{A}R^{B}, —OH, C₁₋₆ alkyl, and —C₁₋₆ alkyl-(NR^{A}R^{B}); and one or more non-adjacent carbon atoms of the C₃₋₂₅ alkyl are optionally replaced by O, N, NH, or N(CH₃);

R⁹ is H, halogen, —NR^{A}R^{B}, —OH, C₁₋₆ alkyl, or —C₁₋₆ alkyl-(NR^{A}R^{B});

each R^{A} and R^{B} are independently hydrogen or C₁₋₆ alkyl;

n is 0 or 1; and

p is 0 or 1;

wherein if n is 0 at least two of R¹, R³, and R⁵ are —C(═O)OH and if n is 1 at least two of R¹, R³, R⁵, and R⁷ are —C(═O)OH.

### DESCRIPTION OF DRAWINGS

FIG. 1. Shows the percentage r₁ change of Gd complexes over time following incubation allysine modified bovine serum albumin, BSA-Ald.

FIG. 2. Shows the relaxivity values at 60 MHz (PBS, pH 7.40, 24 hours, 37° C.) in the presence or absence of BSA and BSA-Ald.

FIG. 3. Shows the hydrolysis of BSA-Ald bound Gd-CHyd and Gd-9 monitored by longitude relaxation at 60 MHz (PBS, pH 7.40, 37° C.).

FIG. 4. Shows the axial liver images of CCl₄ mouse imaged at pre- and 45 minutes post-injection of Gd-CHyd, Gd-10 and Gd-9 (0.1 mmol/kg i.v.).

FIG. 5. Shows the liver to muscle contrast to noise ratio (ΔCNR) of vehicle and CCl₄ mice at 45 minutes post-injection of Gd-CHyd, Gd-10 and Gd-9 (n=6/group).

FIG. 6. Shows the Gd-9 ΔCNR in CCl₄ mice before and after pretreatment with 10-fold dose of MR-silent Yb-9 (n=3).

FIG. 7. Shows the Sirius red staining, the Collagen proportional area (CPA) measured from Sirius red stained tissue, and the hydroxyproline (HYP) in vehicle and CCl₄ mice studied here (n≥15) all demonstrate that the model results in robust liver fibrosis. ***P<0.001.

FIG. 8. Shows the schematic illustration of pair-wise study for Gd-9 and Gd-10. Mice were imaged with either Gd-9 or Gd-10 and then again the next day with the other probe (0.1 mmol/kg i.v.).

FIG. 9. Shows the liver to muscle contrast to noise ratio (ΔCNR) at 45 minutes post-injection showing consistently higher ΔCNR in CCl₄ mice with Gd-9. ***P<0.001.

FIG. 10. Shows the schematic illustration of the blocking study with Yb-9.

FIG. 11. Shows axial DCE MR images of lung in pair-wise study at pre- and 25 minutes post-injection of GdCHyd or Gd-9.

FIG. 12. Shows quantification of MR signal in the lungs.

FIG. 13. Shows quantification of the gadolinium content in the left lungs of BM or naïve animals at 60 minutes post injection of Gd-9.

FIG. 14. Shows schematic illustration of the MR imaging and treatment timeline.

FIG. 15. Shows axial DCE MR images of lung at 25 minutes post-injection of Gd-9 in the mice that underwent a sham procedure (Sham), were challenged with bleomycin intratracheally 10 days (Bleo(D10)), received 11 days PBS (Vehicle(D21)) and EGCG treatment after belo injury.

FIG. 16. Shows quantification of lung to muscle contrast to noise ratio (ΔCNR) of different groups in (n=6).*P<0.05.

FIG. 17. Shows schematic illustration of the pair-wise study of Gd-CHyd and Gd-9 at the 14^{th} day post bleomycin injury.

FIG. 18. Shows lung allysine content is significantly reduced by treatment with EGCG. **p<0.01.

FIG. 19. Shows lung hydroxyproline content is also reduced. *p<0.05.

FIG. 20. Shows conversion yield of Mn complexes (25 μM) over time in the reaction with butyraldehyde (100 μM), characterized by LC-ICP.

FIG. 21. Shows relaxivity values at 60 MHz (PBS, pH 7.40, 2 hours, 37° C.) in the presence or absence of BSA and BSA-Ald.

FIG. 22. Shows axial liver images of CCl₄ and vehicle mouse imaged in pre- and 45 minutes post-injection of GdDOTA or Mn-12 (0.1 mmol/kg i.v.).

FIG. 23. Shows the change in liver to muscle contrast to noise ratio (ΔCNR) of Sham (n=4) and CCl₄ mice (n=6) at 45 minutes post-injection of GdDOTA and Mn-12. *P<0.05, **P<0.01.

FIG. 24. Shows H₂¹⁷O tranverse relaxivity in the presence of corresponding Mn²⁺ complex as a function of temperature. The peak relaxivity is indicative of the number of inner-sphere water molecules (q).

FIG. 25. Shows half life of 1 mM Mn²⁺ complexes in the transmetalation experiment with 25 mM Zn²⁺ monitored by relaxivity in 50 mM pH 6.0 MES buffer, 37° C., 1.4 T showing increased kinetic stability of Mn-12 compared to unmodified Mn-1,4-DO2A.

FIG. 26. Shows biodistribution of manganese in the absence (blank) or presence of Mn-12 and Mn-13 (0.1 mmol/kg i.v., 60 minutes post-injection) showing baseline Mn levels in liver with Mn-12 but elevated levels with Mn-13, indicating the latter is unsuitable for molecular MR of the liver because of high nonspecific signal.

FIG. 27. Shows change in liver to muscle contrast to noise ratio (ΔCNR) change over time in CCl₄ and vehicle group imaged with Mn-12 showing persistent enhancement in fibrotic liver but rapid washout in healthy liver.

FIG. 28. Shows Sirius red staining, collagen proportional area (CPA) and hydroxyproline (HYP) in vehicle and CCl₄ mice studied here demonstrating consistent fibrotic response in the livers of the CCl₄ treated mice. ***P<0.001.

FIG. 29. Shows second order rate constant (kₒₙ) for reaction of Mn-15 and Mn-17 with butyraldehyde.

FIG. 30. Shows half-life (ti/2) for hydrolysis of condensation product of Mn-15 and Mn-17 with butyraldehyde.

FIG. 31. Shows relaxivity values of Mn-15 and Mn-17 in PBS, in BSA solution, in allysine modified BSA-Ald, and bound to BSAAld.

FIG. 32. Shows axial T₁-weighted MR images of CCl₄ mice imaged at pre- and 20 minutes post-injection of Mn-15 (100 μmol/kg, i.v., liver labeled with yellow dash line).

FIG. 33. Shows change in liver-to-muscle contrast to noise ratio (ΔCNR) of vehicle (n=3) and CCl₄ (n=3) mice as a function of time following injection of Mn-15 (0.1 mmol/kg, i.v.).

FIG. 34. Shows significant difference (P=0.02) in area under the ΔCNR curve (AUC0-40) between vehicle (n=3) and CCl₄ (n=3) mice.

FIG. 35. Shows blood clearance of ⁶⁸Ga-7 in a naïve animal with distribution and elimination half-lives.

FIG. 36. Shows biodistribution of 68Ga-7 in lung, heart, liver and kidney 90 min p.i.

FIG. 37. Shows PET maximum intensity projection images of bleomycin-injured and naïve mice 55 minutes p.i.

FIG. 38. Shows axial (top) and sagittal (bottom) PET/MR images showing much higher lung signal in bleomycin-treated mice at 55 minutes p.i.

FIG. 39. Shows PET lung signal (55 min p.i.) and lung-to-heart ratio (90 min p.i.) showing significant differences between bleomycin-treated and naïve animals.

FIG. 40. Shows a schematic illustration of the development of dual Lys^{Ald} binding MRI probe for non-invasive detecting liver fibrogenesis. Chronic liver injury leads to the activation of stellate cells. In the remodeling of the extracellular matrix, closely separated Lys pairs on α1 chains of collagen telopeptide are oxidized by LOX to Lys^{Ald}. The dual hydrazine Gd³⁺ probe precisely targets these Lys^{Ald} pairs with high binding on-rate, high relaxivity upon binding and low off-rate, and results in significantly increased dynamic range and noninvasive detection by MRI.

FIG. 41. Shows chemical structures of Gd³⁺ complexes.

FIG. 42. Shows an end-to-end distance distribution obtained from molecular dynamic simulation of the O-O distance between two α1-N⁹-Lys^{Ald} residues in type-I collagen and the N—N distance in piperazino-hydrazine groups in Gd-9 and Gd-10.

FIG. 43A. Shows a plot of conversion yield versus time of corresponding Gd³⁺ (25 μmol) complexes in the presence of 100 μmol butyraldehyde. FIG. 43B. Affinity measurements of Gd³⁺ complex with butyraldehyde. Concentration-dependent binding of the probes to butyraldehyde as determined by HPLC-ICP-MS (pH 7.4 in PBS, 12 h). The corresponding dissociation constants (K_{d}) was 160, 162, 110, 110 μM for Gd-CHyd, Gd-11, Gd-9, and Gd-10 respectively.

FIG. 44. Shows time course high-performance liquid chromatography in combination with inductively coupled plasma mass spectrometry (HPLC-ICP-MS) trace of Gd-9 (25 μmol) in the presence of 100 μmol butyraldehyde in PBS at room temperature (peak at 0 and leftmost peaks at 13, 25, and 37 mins: Gd-9; rightmost peaks at 13, 25, and 37 mins: product).

FIG. 45. Shows HPLC-ICP-MS traces of reactions of corresponding Gd³⁺ complex (25 μM) with butyraldehyde (100 μM) (pH 7.4 in PBS).

FIG. 46. Shows a bar graph of relaxivity values in PBS, in PBS with BSA or BSA^{Ald} (10 mg/mL, pH 7.4, 24 h, 37° C., 1.41T). Data are mean±SD of three independent experiments.

FIG. 47. Shows a plot of percentage r₁ change of Gd complexes (0.1 mM) over time following incubation with 10 mg/mL BSA^{Ald} in PBS.

FIG. 48. Shows a plot of binding yield measurements of Gd³⁺ complex with BSA^{Ald}. Binding concentration of the probes to BSA^{Ald} (pH 7.4, PBS, 37° C., 12 h) as determined by ICP-MS following an ultrafiltration (5,000 Da cut-off) to separate the protein-bound probe from the unbound probe. Slope gives the binding yield.

FIG. 49A. Shows an HPLC-ICP-MS trace with gadolinium detection using method 23 of Gd-9 in human Plasma at 37° C. for 3 hours. FIG. 49B. Shows an HPLC-ICP-MS trace with gadolinium detection using method 23 of Gd-10 in human Plasma at 37° C. for 3 hours.

FIG. 50. Shows a plot of hydrolysis of BSA^{Ald} bound Gd-CHyd and Gd-9 monitored by longitude relaxation in PBS (pH 7.4, 37° C., 1.41T).

FIG. 51. Shows a bar graph of relaxivity of BSA^{Ald}-bound species in PBS (pH 7.4, 37° C., 1.41 T). Data are mean±SD of three independent experiments.

FIG. 52. Shows characterization of binding species using SDS-polyacrylamide gel electrophoresis (SDS-PAGE). From left to right: a, BSA; b, BSA in the presence of Gd-9; c, BSA^{Ald} in the presence of Gd-9.

FIG. 53. Schematic illustration of the relaxivity measurement in ECM.

FIG. 54. Shows a bar graph of relaxivity values in fibrotic rat liver ECM with or without addition of 100-fold excess of hydrazine (PBS, pH 7.4, 2 h, 37° C., 1.41T).

FIG. 55. Shows representative time-dependent coronal T1 weighted 3D-FLASH MR images of normal mouse before and at indicated time points after i.v. injection with 100 μmol/kg of the corresponding probe. The liver enhances slightly with first-blood pass but the signal rapidly returns to baseline after 20 min. Similar results were observed in 4 animals per probe.

FIG. 56. Schematic illustration of the animal study design of CCl₄ induced liver fibrosis. Mice were imaged after 12 weeks of oral gavage of CCl₄ with probe 1 and then again the next day with probe 2 (100 μmol/kg i.v., probes chosen randomly from Gd-DOTA, Gd-CHyd, Gd-9 and Gd-10).

FIG. 57. Shows representative images of Sirius red, LOX and Lys^{Ald} staining (scale bar: 500 μm).

FIG. 58. Shows collagen proportional area (CPA) measured from Sirius red stained tissue (n≥10, ***P<0.0001, unpaired t-test, two-tailed).

FIG. 59. Shows the percentage of LOX positive tissue measured from IHC LOX stained tissue (n 10, ***P<0.0001, unpaired t-test, two-tailed).

FIG. 60. Shows the percentage of Lys^{Ald} positive tissue measured from DNPH reactivity assay (n≥10, ***P<0.0001, unpaired t-test, two-tailed).

FIG. 61. Shows plots of the blood clearance of the probes in vehicle and CCl₄ treated mice. Determined by the change of signal intensity in T1 weighted 3D FLASH dynamic MRI in the heart (blood pool, n=6).

FIG. 62. Shows axial liver (outlined in white) MR images of CCl₄ mouse imaged before and 45 min p.i. of Gd-CHyd, Gd-10, Gd-9, and Gd-DOTA, (100 μmol/kg i.v.).

FIG. 63. Liver clearance of the probes in vehicle and CCl₄ treated mice. Determined by the change of ΔCNR in T1 weighted 3D FLASH MRI (n=6).

FIG. 64. Shows change in liver to muscle contrast to noise ratio (ΔCNR) relative to pre-injection image at 45 min p.i. showing consistently higher ΔCNR in CCl₄ mice with Gd-9 (n≥6 per group, *P=0.0433, **P=0.0084, ***P<0.0001, one-way ANOVA, post hoc comparison, two-tailed).

FIG. 65. Shows pairwise comparison of ΔCNR at 45 min for Gd-9 and Gd-10 imaged in the same mouse (100 μmol/kg i.v. injection order randomized) one day apart showing consistently higher ΔCNR in CCl₄ mice imaged with Gd-9 compared to Gd-10 (***P=0.0003, paired t-test, two-tailed).

FIG. 66. Schematic illustration of the blocking study with Yb-9.

FIG. 67. Shows plotted time course of Gd-9 ΔCNR in CCl₄ mice before and after pretreatment with 10-fold dose of MR-silent Yb-9 (n=3, **P=0.0045). All data shown as mean±SD, *P<0.05, **P<0.01, ***P<0.001.

FIG. 68. Shows liver hydroxyproline (Hyp, μg/g) in vehicle and CCl₄ treated mice (n 10, ***P<0.0001, unpaired t-test, two-tailed).

FIG. 69. Comparison of α-smooth muscle actin (α-SMA) immunoreactivity in CCl₄ and vehicle treated mice. FIG. 69A. Representative figures of α-SMA immunohistology staining in vehicle and CCl₄ treated mice (scale bar: 500 m). FIG. 69B. Percentage of α-SMA positive tissue measured from IHC stained tissue in a). n≥10, ***P<0.001, unpaired t-test, two-tailed.

FIG. 70. A diagram that shows experimental design, animal group classification, and in vivo MRI imaging. Adult C57BL/6 mice receiving standard chow served as age-matched (n=10) controls. Mice fed with choline-deficient, 1-amino acid-defined, high-fat diet (CDAHFD) for 2, 6 or 10 weeks (n=6 per group) were used to study disease progression of nonalcoholic steatohepatitis (NASH). Mice that received 10 weeks of CDAHFD subsequently either switched back to standard chow for 1 or 4 weeks (n=6 per group) were used to study treatment effect. At each time point, mice underwent Gd-9 enhanced MRI followed by sacrifice and ex vivo characterization of the liver.

FIG. 71. Shows subtraction (20 min p.i. 100 μmol Gd-9/kg-pre-injection) T1-weighted images in control and CDAHFD groups.

FIG. 72. Shows quantitative analyses of liver to muscle ΔCNR at 20 min in each group (All data shown as mean±SD. *P<0.05, **P<0.01, ***P<0.001, ns: not significant, one-way ANOVA, post hoc comparison, two-tailed).

FIG. 73A. Shows representative H&E, Sirius red, Lys^{Ald} and LOX staining of livers from control mice and CDAHFD fed mice (scale bar: 100 m). FIG. 73B. Shows quantitative analyses of fat content expressed as % lipid vacuolization in H&E-stained livers of control mice and CDAHFD fed mice (n≥4 per group, all data shown as mean±SD, **P<0.01, ***P<0.001, ns: not significant, one-way ANOVA, post hoc comparison, two-tailed).

FIG. 74A. Shows total collagen quantification assessed by liver hydroxyproline (Hyp) content as a fibrosis measure (n≥4 per group, all data shown as mean±SD, **P<0.01, ***P<0.001, ns: not significant, one-way ANOVA, post hoc comparison, two-tailed). FIG. 74B. Shows quantitative analyses of collagen proportional area (CPA) measured from Sirius red stained livers of control and CDAHFD fed mice (n≥4 per group, all data shown as mean±SD, *P<0.05, **P<0.01, ***P<0.001, ns: not significant, one-way ANOVA, post hoc comparison, two-tailed).

FIG. 75. Shows the percentage of LOX positive tissue measured from IHC LOX stained tissue (n≥4 per group, all data shown as mean±SD. *P<0.05, ***P<0.001, ns: not significant, one-way ANOVA, post hoc comparison, two-tailed).

FIG. 76A. Shows representative figures of α-smooth muscle actin (α-SMA) immunohistology staining in control and CDAHFD fed mice (scale bar: 500 m). FIG. 76B. Shows quantitative analyses of the percentage of α-SMA positive tissue measured from IHC stained tissue (n≥4 per group, all data shown as mean±SD, *P<0.05, **P<0.01, ***P<0.001, ns: not significant, one-way ANOVA, post hoc comparison, two-tailed).

FIG. 77A. Shows correlation analysis between ΔCNR at 20 min and hydroxyproline content (R=0.24, P=0.24). Each data point represents one mouse. FIG. 77B. Shows correlation analysis between ΔCNR at 20 min and the percentage of LOX positive tissue (R=0.92, P<0.0001). Each data point represents one mouse. FIG. 77C. Shows correlation analysis between ΔCNR at 20 min and the integrated intensity of Lys^{Ald} (R=0.74, P<0.0001). Each data point represents one mouse.

FIG. 78. Shows quantitative analyses of the integrated intensity of Lys^{Ald} from DNPH reactivity assay as measures of fibrogenesis (n≥4 per group, all data shown as mean±SD, *P<0.05, ***P<0.001, ns: not significant, one-way ANOVA, post hoc comparison, two-tailed).

FIG. 79. Shows correlation analysis between ΔCNR observed at 20 min after Gd-9 injection and percentage of tissue stained positive for α-SMA as a marker for tissue fibrogenesis (R=0.85, P<0.0001).

FIG. 80. Diagram shows experimental design. Bile duct ligation surgery was performed on 7-9 week old male CD rats (n=7) and the rats were imaged 10 days later before and for 30 min after 100 μmol/kg i.v. Gd-9 (n=4 for sham group). After imaging, liver was harvested and sectioned.

FIG. 81. Shows coronal MRI (greyscale) of BDL rat with pre-injection and 30 min p.i. longitudinal relaxation rate (R1) maps (color scale).

FIG. 82A. Shows the change in liver longitudinal relaxation rate (ΔR1) at 30 min p.i. induced by Gd-9 was 5-fold higher in BDL rats compared to sham rats (***P=0.0003, unpaired t-test, two-tailed). FIG. 82B. Shows the liver to muscle ΔCNR at 30 min was 4-fold higher in BDL rats compared to sham rats (**P=0.008, unpaired t-test, two-tailed).

FIG. 83. Shows that the percentage of Lys^{Ald} positive liver tissue measured from DNPH reactivity assay and LOX positive liver tissue measured from LOX IHC was both significantly higher in BDL rats compared to sham rats (n=4, ***P=0.0014, ***P=0.0006, respectively, unpaired t-test, two-tailed).

FIG. 84A. Representative H&E, Sirius red, LOX and Lys^{Ald} staining of livers from Sham and BDL rats. FIG. 84B. Quantitative analyses of collagen proportional area (CPA) measured from Sirius red stained liver in Sham and BDL rats. n=4. ***P<0.001, unpaired t-test, two-tailed. FIG. 84C. Total collagen quantification assessed by liver hydroxyproline (Hyp) content for Sham (n=4) and BDL (n=7) rats. ***P<0.001, unpaired t-test, two-tailed.

FIG. 85A. Consecutive liver slices from a BDL rat after Gd-9 enhanced MRI. Left to right: LA-ICP-MS images of gadolinium distribution in fresh harvested BDL liver, BDL liver incubated with Gd-9, BDL liver incubated with Gd-9+N₂H4, and Lys^{Ald} staining (scale bar: 500 m). FIG. 85B. Distribution map of gadolinium measured by LA-ICP-MS in the fresh harvested Sham liver at 30 min post injection of Gd-9 (scale bar: 500 m).

FIG. 86. Shows colocalization of Gd distribution and Lys^{Ald} along the line indicated by the arrow in FIG. 85A.

FIG. 87A. Shows human fibrotic/cirrhotic liver associated with NASH and normal liver specimens obtained from surgery and sectioned. FIG. 87B. Shows representative liver sections stained for Lys^{Ald} by DNPH reactivity assay to show the distribution of extracellular aldehydes (arrow marks fibrotic band). FIG. 87C. Shows images of adjacent fibrotic liver sections stained for LOX (immunofluorescence), Sirius red or incubated with Gd-9 without or with 100-fold N₂H4 and assessed by LA-ICP-MS. FIG. 87D. Shows an image of a normal human liver incubated with Gd-9 and assessed by LA-ICP-MS.

FIG. 88. Shows the detection of liver fibrogenesis in human NASH fibrotic liver specimens. Human fibrotic liver specimens from patient 2-5 were stained for Lys^{Ald}, LOX (immunofluorescence), Sirius red or incubated with Gd-9 and assessed by LA-ICP-MS (¹⁵⁷Gd).

FIG. 89. Shows the detection of liver fibrogenesis in human normal liver specimens. Human normal liver specimens from patient 6-9 were stained for Lys^{Ald}, LOX (immunofluorescence), Sirius red or incubated with Gd-9 and assessed by LA-ICP-MS (¹⁵⁷Gd).

FIG. 90A. Shows the percentage of Lys^{Ald} positive tissue for normal and fibrotic human liver measured from DNPH reactivity assay (*P=0.0232). FIG. 90B. Shows the average Gd concentration in human fibrotic and normal tissues incubated with Gd-9 and assessed by LA-ICP-MS (**P=0.0023). All data shown as mean±SD. *P<0.05, **P<0.01.

### DETAILED DESCRIPTION

Disclosed herein are methods of molecular magnetic resonance (MR) imaging and positron emission tomography using extracellular probes that target extracellular allysine aldehyde and act as a noninvasive biomarker of fibrogenesis with high sensitivity and specificity in detecting fibrogenesis, for example, in rodent models and human fibrotic tissues.

A large number of chronic and acute diseases have a fibroproliferative component, i.e. the tissue becomes fibrotic or scarred. For example chronic liver diseases like nonalcoholic steatohepatitis, chronic kidney diseases, inflammatory bowel diseases like Crohn's disease, heart diseases like heart failure, atrial fibrillation, and myocardiac infarction, fibrotic diseases of the lung like idiopathic pulmonary fibrosis, cancers such as pancreatic ductal adenocarcinoma, scleroderma, and atherosclerosis all have a fibrotic component. As an example, chronic liver disease (CLD) is caused by chronic injury from alcohol, drug abuse, viral injury, or metabolic derangements (e.g. nonalcoholic steatohepatitis, NASH). CLD accounts for approximately 2 million deaths worldwide per year with more than 2 billion people at risk. If left untreated, CLD results in scarring of the liver (fibrosis) that can further lead to cirrhosis, primary liver cancer, liver failure, and/or death.

Biopsy is the gold standard to detect and stage fibrosis but it is invasive, has sampling error, carries complication risk, and is not suited to serial monitoring. Depending on the organ and the disease, there are noninvasive methods to detect and stage fibrosis, but these all have limitations. Chiefly, these methods can only reliably detect advanced stages of fibrosis and they cannot measure disease activity, i.e. they cannot distinguish active ongoing disease (fibrogenesis) from old injury. For example in the liver, ultrasound and magnetic resonance (MR) elastography methods and some blood biomarker panels are effective in detecting advanced fibrosis, but cannot detect early onset of liver fibrosis, nor measure disease activity, i.e. fibrogenesis. Similar limitations exist in other organs, e.g. in the lung, high resolution computed tomography can detect advanced fibrosis but not measure disease activity; in the heart a MRI technique called extracellular volume mapping is used to estimate fibrosis. Without wishing to be bound by theory, sensing disease activity would provide the best guidance to reverse fibrosis and cure disease, enabling both disease detection and providing an early readout of treatment effectivity. Sensing fibrogenesis would also accelerate drug development. For instance, drug development efforts in nonacloholic steatoheptitis (NASH) have been hampered by the inability to enroll patients with early stage fibrosis, and instead recruits patients with advanced disease where drug therapies may be less effective. NASH trials also require a reduction in fibrosis stage as an endpoint, but fibrosis regression is a slow process; an early measure of treatment response would allow early termination of ineffective treatments and enable a better trial design to detect significant fibrosis reduction in promising therapies.

In tissue fibrogenesis, regardless of cause, activated myofibroblasts secrete inflammatory mediators and synthesize extracellular matrix (ECM) components. Excess accumulation of ECM and ECM crosslinking cause tissue stiffness and disrupt tissue architecture, resulting in liver dysfunction and ultimately liver failure. Collagens are the most abundant proteins in the fibrotic ECM. Noninvasive molecular imaging of collagen was explored in preclinical models and shown to be effective at staging fibrosis, but collagen imaging does not assess fibrogenesis and cannot distinguish ongoing disease from old injury.

Lysyl oxidase (LOX) and its paralogs are established markers of fibrogenesis. During liver fibrogenesis, the secretion and enzymatic activity of lysyl oxidase (LOX) and its paralogs are increased. Specifically, LOX catalyzes collagen crosslinking by preferentially oxidizing lysine amino pairs in close proximity, e.g. two Lys residues in the C-telopeptide in collagen I (both al-Lys¹⁶) and three in the N-telopeptide (two α1-Lys⁹ and one α2-Lys⁵). The oxidation products are allysine aldehyde (Lys^{Ald}) pairs that subsequently react with one F-amino group [2+1] in the receptor region of a neighboring collagen molecule to yield an intermolecular pyridinoline crosslink. Similar mechanisms were found for all collagen types and are conserved across species.

Without wishing to be bound by theory, it is believed that targeting these extracellular Lys^{Ald} or Lys^{Ald} pairs, absent in normal liver tissues, could give a highly specific and sensitive biomarker of liver fibrogenesis. During active fibrosis, Lys^{Ald} concentration is increased, but if fibrogenesis ceases, the aldehydes will be consumed by degradation. These probe design attributes should result in high MRI signal at the site of injury, and provide a non-invasive and sensitive readout for measuring the early onset of fibrogenesis across various organs and tissues.

One embodiment provides a compound of Formula (I)

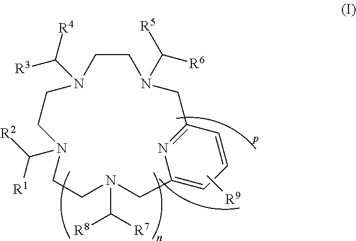

or a pharmaceutically acceptable salt thereof, wherein:

each R¹, R³, R⁵, and R⁷ are independently hydrogen or —C(═O)OH;

each R², R⁴, R⁶, and R⁸ are independently hydrogen or C₃₋₂₅ alkyl, wherein the C₃₋₂₅ alkyl is optionally substituted with 1-2 substituents independently selected from the group consisting of —NR^{A}R^{B}, —OH, halogen, C₁₋₆ alkoxy, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of halogen, —NR^{A}R^{B}, —OH, C₁0.6 alkyl, and —C₁₋₆ alkyl-(NR^{A}R^{B}); and one or more non-adjacent carbon atoms of the C₃₋₂₅ alkyl are optionally replaced by O, N, NH, or N(CH₃);

R⁹ is H, halogen, —NR^{A}R^{B}, —OH, C₁₋₆ alkyl, or —C₁₋₆ alkyl-(NR^{A}R^{B});

each R^{A} and R^{B} are independently hydrogen or C₁₋₆ alkyl;

n is 0 or 1; and

p is 0 or 1;

wherein if n is 0 at least two of R¹, R³, and R⁵ are —C(═O)OH and if n is 1 at least two of R¹, R³, R⁵, and R⁷ are —C(═O)OH.

In some embodiments, if n is 0 and p is 0, at least one of R¹, R³, and R⁵ is hydrogen.

In some embodiments, if n is 1, p is 0, R¹, R³, R⁵, and R⁷ are all —C(═O)OH, three of R², R⁴, R⁶, and R⁸ are hydrogen, and one of R², R⁴, R⁶, and R⁸ is C₃₋₂₅ alkyl, then at least one non-adjacent carbon atoms of the C₃₋₂₅ alkyl is replaced with O.

In some embodiments, if n is 1, p is 0, R¹, R³, R⁵, and R⁷ are all —C(═O)OH, three of R², R⁴, R⁶, and R⁸ are hydrogen, and one of R², R⁴, R⁶, and R⁸ is C₃₋₂₅ alkyl, then at least one non-adjacent carbon atoms of the C₃₋₂₅ alkyl is replaced with O and C₃₋₂₅ alkyl is substituted with 1 or 2 —NR^{A}R^{B}.

In some embodiments, the compound of Formula (I) is other than:

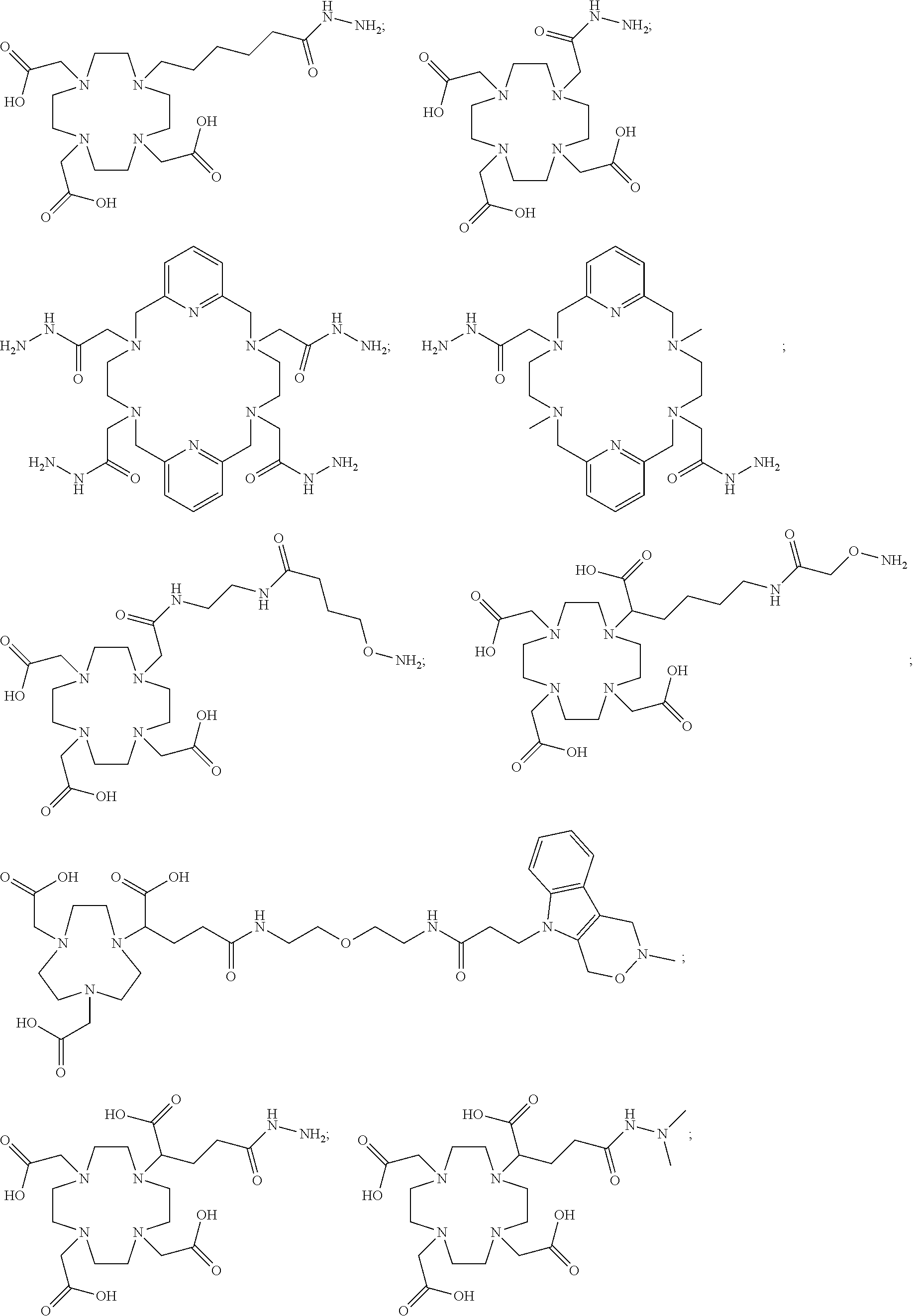

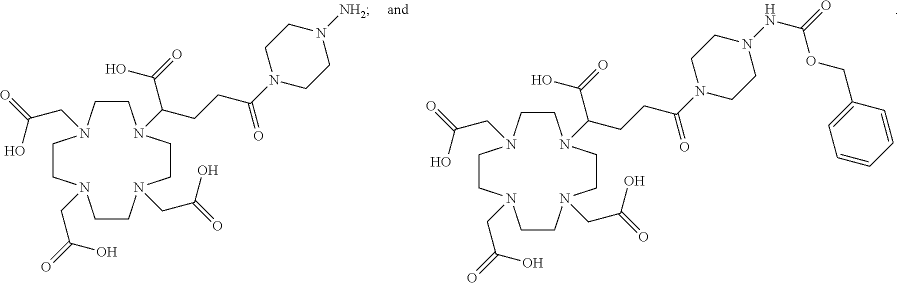 [SMILES: NN1CCN(C(=O)CCC(C(=O)O)N2CCN(CC(=O)O)CCN(CC(=O)O)CCN(CC(=O)O)CC2)CC1 ; O=C(O)CN1CCN(CC(=O)O)CCN(C(CCC(=O)N2CCN(NC(=O)OCc3ccccc3)CC2)C(=O)O)CCN(CC(=O)O)CC1]

In some embodiments, n is 0. In some embodiments, n is 1.

In some embodiments, p is 0. In some embodiments, p is 1.

In some embodiments, n is 0 and p is 0. In some embodiments, n is 0 and p is 1. In some embodiments, n is 1 and p is 1. In some embodiments, n is 1 and p is 0.

In some embodiments, R², R⁴, and R⁶ are independently selected from the group consisting of hydrogen and C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C1-6 alkyl-(NR^{A}R^{B}); and one to six non-adjacent carbon atoms of the C₃₋₂₅ alkyl are optionally replaced by O, N, NH, or N(CH₃).

In some embodiments, R² is selected from the group consisting of hydrogen and C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R⁴ is selected from the group consisting of hydrogen and C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R⁶ is selected from the group consisting of hydrogen and C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R², R⁴, and R⁶ are independently selected from the group consisting of:

Hydrogen;

C₃₋₂₅ alkyl substituted with 4-10 membered heterocyclyl substituted —NR^{A}R^{B}, wherein one non-adjacent carbon atom of the C₃₋₂₅ alkyl is replaced by NH and four non-adjacent carbon atom of the C₃₋₂₅ alkyl are replaced by O;

C₃₋₂₅ alkyl substituted with 5-10 membered heteroaryl and —NR^{A}R^{B}, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 5-10 membered heteroaryl substituted with —OH and C₁₋₆ alkyl, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with two —NR^{A}R^{B}, wherein three non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl substituted with —C₁₋₆ alkyl-(NR^{A}R^{B}), wherein two non-adjacent carbon atom of the C₃₋₂₅ alkyl are replaced by NH and three non-adjacent carbon atom of the C₃₋₂₅ alkyl are replaced by O;

C₃₋₂₅ alkyl substituted with —NR^{A}R^{B} and —OH, wherein three non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with —NR^{A}R^{B} and —OH, wherein two non-adjacent carbon atoms of the C₃₋₂₅ alkyl are replaced by NH and four non-adjacent carbon atoms of the C₃₋₂₅ alkyl are replaced by O;

C₃₋₂₅ alkyl substituted with 4-10 membered heterocyclyl substituted with —NR^{A}R^{B};

C₃₋₂₅ alkyl substituted with 4-10 membered heterocyclyl;

C₃₋₂₅ alkyl;

C₃₋₂₅ alkyl substituted with —NR^{A}R^{B}, wherein one non-adjacent carbon atom of the C₃₋₂₅ alkyl is replaced by NH;

C₃₋₂₅ alkyl substituted with —NR^{A}R^{B} and —OH, wherein two non-adjacent carbon atoms of the C₃₋₂₅ alkyl are replaced by NH;

C₃₋₂₅ alkyl substituted with —NR^{A}R^{B} and —OH, wherein one non-adjacent carbon atom of the C₃₋₂₅ alkyl is replaced by NH and one non-adjacent carbon atom of the C₃₋₂₅ alkyl is replaced by O;

C₃₋₂₅ alkyl substituted with —NR^{A}R^{B}, wherein two non-adjacent carbon atoms of the C₃₋₂₅ alkyl are replaced by NH;

C₃₋₂₅ alkyl substituted with OH, wherein one non-adjacent carbon atom of the C₃₋₂₅ alkyl is replaced by NH and one non-adjacent carbon atom of the C₃₋₂₅ alkyl is replaced by N(CH₃);

C₃₋₂₅ alkyl substituted with two —NR^{A}R^{B}, wherein one non-adjacent carbon atoms of the C₃₋₂₅ alkyl are replaced by N, three non-adjacent carbon atoms of the C₃₋₂₅ alkyl are replaced by NH, and two non-adjacent carbon atoms of the C₃₋₂₅ alkyl are replaced by O; and

C₃₋₂₅ alkyl substituted with —NR^{A}R^{B}, wherein one non-adjacent carbon atom of the C₃₋₂₅ alkyl is replaced by NH and one non-adjacent carbon atom of the C₃₋₂₅ alkyl is replaced by O.

In some embodiments, R² is C₃₋₂₅ alkyl substituted with 4-10 membered heterocyclyl substituted —NR^{A}R^{B}, wherein one non-adjacent carbon atom of the C₃₋₂₅ alkyl is replaced by NH and four non-adjacent carbon atom of the C₃₋₂₅ alkyl are replaced by O.

In some embodiments, R² is selected from the group consisting of:

Hydrogen;

C₃₋₂₅ alkyl substituted with 4-10 membered heterocyclyl substituted —NR^{A}R^{B}, wherein one non-adjacent carbon atom of the C₃₋₂₅ alkyl is replaced by NH and four non-adjacent carbon atom of the C₃₋₂₅ alkyl are replaced by O;

C₃₋₂₅ alkyl substituted with 5-10 membered heteroaryl and —NR^{A}R^{B}, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 5-10 membered heteroaryl substituted with —OH and C₁₋₆ alkyl, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with two —NR^{A}R^{B}, wherein three non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl substituted with —C₁₋₆ alkyl-(NR^{A}R^{B}), wherein two non-adjacent carbon atom of the C₃₋₂₅ alkyl are replaced by NH and three non-adjacent carbon atom of the C₃₋₂₅ alkyl are replaced by O;

C₃₋₂₅ alkyl substituted with —NR^{A}R^{B} and —OH, wherein three non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with —NR^{A}R^{B} and —OH, wherein two non-adjacent carbon atoms of the C₃₋₂₅ alkyl are replaced by NH and four non-adjacent carbon atoms of the C₃₋₂₅ alkyl are replaced by O;

C₃₋₂₅ alkyl substituted with 4-10 membered heterocyclyl substituted with —NR^{A}R^{B};

C₃₋₂₅ alkyl substituted with 4-10 membered heterocyclyl;

C₃₋₂₅ alkyl;

C₃₋₂₅ alkyl substituted with —NR^{A}R^{B}, wherein one non-adjacent carbon atom of the C₃₋₂₅ alkyl is replaced by NH;

C₃₋₂₅ alkyl substituted with —NR^{A}R^{B} and —OH, wherein two non-adjacent carbon atoms of the C₃₋₂₅ alkyl are replaced by NH;

C₃₋₂₅ alkyl substituted with —NR^{A}R^{B} and —OH, wherein one non-adjacent carbon atom of the C₃₋₂₅ alkyl is replaced by NH and one non-adjacent carbon atom of the C₃₋₂₅ alkyl is replaced by O;

C₃₋₂₅ alkyl substituted with —NR^{A}R^{B}, wherein two non-adjacent carbon atoms of the C₃₋₂₅ alkyl are replaced by NH;

C₃₋₂₅ alkyl substituted with OH, wherein one non-adjacent carbon atom of the C₃₋₂₅ alkyl is replaced by NH and one non-adjacent carbon atom of the C₃₋₂₅ alkyl is replaced by N(CH₃);

C₃₋₂₅ alkyl substituted with two —NR^{A}R^{B}, wherein one non-adjacent carbon atoms of the C₃₋₂₅ alkyl are replaced by N, three non-adjacent carbon atoms of the C₃₋₂₅ alkyl are replaced by NH, and two non-adjacent carbon atoms of the C₃₋₂₅ alkyl are replaced by O; and

C₃₋₂₅ alkyl substituted with —NR^{A}R^{B}, wherein one non-adjacent carbon atom of the C₃₋₂₅ alkyl is replaced by NH and one non-adjacent carbon atom of the C₃₋₂₅ alkyl is replaced by O.

In some embodiments, R² is C₃₋₂₅ alkyl substituted with 4-10 membered heterocyclyl substituted —NR^{A}R^{B}, wherein one non-adjacent carbon atom of the C₃₋₂₅ alkyl is replaced by NH and four non-adjacent carbon atom of the C₃₋₂₅ alkyl are replaced by O.

In some embodiments, R⁴ is selected from the group consisting of.

Hydrogen;

C₃₋₂₅ alkyl substituted with 4-10 membered heterocyclyl substituted —NR^{A}R^{B}, wherein one non-adjacent carbon atom of the C₃₋₂₅ alkyl is replaced by NH and four non-adjacent carbon atom of the C₃₋₂₅ alkyl are replaced by O;

C₃₋₂₅ alkyl substituted with 5-10 membered heteroaryl and —NR^{A}R^{B}, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 5-10 membered heteroaryl substituted with —OH and C₁₋₆ alkyl, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with two —NR^{A}R^{B}, wherein three non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl substituted with —C₁₋₆ alkyl-(NR^{A}R^{B}), wherein two non-adjacent carbon atom of the C₃₋₂₅ alkyl are replaced by NH and three non-adjacent carbon atom of the C₃₋₂₅ alkyl are replaced by O;

C₃₋₂₅ alkyl substituted with —NR^{A}R^{B} and —OH, wherein three non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with —NR^{A}R^{B} and —OH, wherein two non-adjacent carbon atoms of the C₃₋₂₅ alkyl are replaced by NH and four non-adjacent carbon atoms of the C₃₋₂₅ alkyl are replaced by O;

C₃₋₂₅ alkyl substituted with 4-10 membered heterocyclyl substituted with —NR^{A}R^{B}; C₃₋₂₅ alkyl substituted with 4-10 membered heterocyclyl;

C₃₋₂₅ alkyl;

C₃₋₂₅ alkyl substituted with —NR^{A}R^{B}, wherein one non-adjacent carbon atom of the C₃₋₂₅ alkyl is replaced by NH;

C₃₋₂₅ alkyl substituted with —NR^{A}R^{B} and —OH, wherein two non-adjacent carbon atoms of the C₃₋₂₅ alkyl are replaced by NH;

C₃₋₂₅ alkyl substituted with —NR^{A}R^{B} and —OH, wherein one non-adjacent carbon atom of the C₃₋₂₅ alkyl is replaced by NH and one non-adjacent carbon atom of the C₃₋₂₅ alkyl is replaced by O;

C₃₋₂₅ alkyl substituted with —NR^{A}R^{B}, wherein two non-adjacent carbon atoms of the C₃₋₂₅ alkyl are replaced by NH;

C₃₋₂₅ alkyl substituted with OH, wherein one non-adjacent carbon atom of the C₃₋₂₅ alkyl is replaced by NH and one non-adjacent carbon atom of the C₃₋₂₅ alkyl is replaced by N(CH₃);

C₃₋₂₅ alkyl substituted with two —NR^{A}R^{B}, wherein one non-adjacent carbon atoms of the C₃₋₂₅ alkyl are replaced by N, three non-adjacent carbon atoms of the C₃₋₂₅ alkyl are replaced by NH, and two non-adjacent carbon atoms of the C₃₋₂₅ alkyl are replaced by O; and

C₃₋₂₅ alkyl substituted with —NR^{A}R^{B}, wherein one non-adjacent carbon atom of the C₃₋₂₅ alkyl is replaced by NH and one non-adjacent carbon atom of the C₃₋₂₅ alkyl is replaced by O.

In some embodiments, R⁶ is selected from the group consisting of:

Hydrogen;

C₃₋₂₅ alkyl substituted with 4-10 membered heterocyclyl substituted —NR^{A}R^{B}, wherein one non-adjacent carbon atom of the C₃₋₂₅ alkyl is replaced by NH and four non-adjacent carbon atom of the C₃₋₂₅ alkyl are replaced by O;

C₃₋₂₅ alkyl substituted with 5-10 membered heteroaryl and —NR^{A}R^{B}, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 5-10 membered heteroaryl substituted with —OH and C₁₋₆ alkyl, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with two —NR^{A}R^{B}, wherein three non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl substituted with —C₁₋₆ alkyl-(NR^{A}R^{B}), wherein two non-adjacent carbon atom of the C₃₋₂₅ alkyl are replaced by NH and three non-adjacent carbon atom of the C₃₋₂₅ alkyl are replaced by O;

C₃₋₂₅ alkyl substituted with —NR^{A}R^{B} and —OH, wherein three non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with —NR^{A}R^{B} and —OH, wherein two non-adjacent carbon atoms of the C₃₋₂₅ alkyl are replaced by NH and four non-adjacent carbon atoms of the C₃₋₂₅ alkyl are replaced by O;

C₃₋₂₅ alkyl substituted with 4-10 membered heterocyclyl substituted with —NR^{A}R^{B};

C₃₋₂₅ alkyl substituted with 4-10 membered heterocyclyl;

C₃₋₂₅ alkyl;

C₃₋₂₅ alkyl substituted with —NR^{A}R^{B}, wherein one non-adjacent carbon atom of the C₃₋₂₅ alkyl is replaced by NH;

C₃₋₂₅ alkyl substituted with —NR^{A}R^{B} and —OH, wherein two non-adjacent carbon atoms of the C₃₋₂₅ alkyl are replaced by NH;

C₃₋₂₅ alkyl substituted with —NR^{A}R^{B} and —OH, wherein one non-adjacent carbon atom of the C₃₋₂₅ alkyl is replaced by NH and one non-adjacent carbon atom of the C₃₋₂₅ alkyl is replaced by O;

C₃₋₂₅ alkyl substituted with —NR^{A}R^{B}, wherein two non-adjacent carbon atoms of the C₃₋₂₅ alkyl are replaced by NH;

C₃₋₂₅ alkyl substituted with OH, wherein one non-adjacent carbon atom of the C₃₋₂₅ alkyl is replaced by NH and one non-adjacent carbon atom of the C₃₋₂₅ alkyl is replaced by N(CH₃);

C₃₋₂₅ alkyl substituted with two —NR^{A}R^{B}, wherein one non-adjacent carbon atoms of the C₃₋₂₅ alkyl are replaced by N, three non-adjacent carbon atoms of the C₃₋₂₅ alkyl are replaced by NH, and two non-adjacent carbon atoms of the C₃₋₂₅ alkyl are replaced by O; and

C₃₋₂₅ alkyl substituted with —NR^{A}R^{B}, wherein one non-adjacent carbon atom of the C₃₋₂₅ alkyl is replaced by NH and one non-adjacent carbon atom of the C₃₋₂₅ alkyl is replaced by O.

In some embodiments, R² is C₃₋₂₅ alkyl substituted with 4-10 membered heterocyclyl substituted —NR^{A}R^{B}, wherein one non-adjacent carbon atom of the C₃₋₂₅ alkyl is replaced by NH and four non-adjacent carbon atom of the C₃₋₂₅ alkyl are replaced by O.

In some embodiments, R² is C₃₋₂₅ alkyl substituted with 4-10 membered heterocyclyl substituted —NR^{A}R^{B}, wherein one non-adjacent carbon atom of the C₃₋₂₅ alkyl is replaced by NH and four non-adjacent carbon atom of the C₃₋₂₅ alkyl are replaced by O.

In some embodiments, R² is C₃₋₂₅ alkyl substituted with 5-10 membered heteroaryl and —NR^{A}R^{B}, wherein two non-adjacent carbon atoms of the C₃₋₂₅ alkyl are replaced by NH and three non-adjacent carbon atoms of the C₃₋₂₅ alkyl are replaced by O.

In some embodiments, R² is C₃₋₂s alkyl substituted with 5-10 membered heteroaryl substituted with —OH and C₁₋₆ alkyl, wherein two non-adjacent carbon atoms of the C₃₋₂₅ alkyl are replaced by NH and three non-adjacent carbon atoms of the C₃₋₂₅ alkyl are replaced by O.

In some embodiments, R² is C₃₋₂₅ alkyl substituted with 4-10 membered heterocyclyl, wherein two non-adjacent carbon atoms of the C₃₋₂₅ alkyl are replaced by NH and three non-adjacent carbon atoms of the C₃₋₂₅ alkyl are replaced by O.

In some embodiments, R² is C₃₋₂₅ alkyl substituted with two —NR^{A}R^{B}, wherein three non-adjacent carbon atoms of the C₃₋₂₅ alkyl are replaced by NH and three non-adjacent carbon atoms of the C₃₋₂₅ alkyl are replaced by O.

In some embodiments, R² is C₃₋₂₅ alkyl substituted with 4-10 membered heterocyclyl substituted with —C₁₋₆ alkyl-(NR^{A}R^{B}), wherein two non-adjacent carbon atom of the C₃₋₂₅ alkyl are replaced by NH and three non-adjacent carbon atom of the C₃₋₂₅ alkyl are replaced by O.

In some embodiments, R² is C₃₋₂₅ alkyl substituted with —NR^{A}R^{B} and —OH, wherein three non-adjacent carbon atoms of the C₃₋₂₅ alkyl are replaced by NH and three non-adjacent carbon atoms of the C₃₋₂₅ alkyl are replaced by O.

In some embodiments, R² is C₃₋₂₅ alkyl substituted with —NR^{A}R^{B} and —OH, wherein two non-adjacent carbon atoms of the C₃₋₂₅ alkyl are replaced by NH and four non-adjacent carbon atoms of the C₃₋₂₅ alkyl are replaced by O.

In some embodiments, R² is C₃₋₂₅ alkyl substituted with 4-10 membered heterocyclyl substituted with —NR^{A}R^{B}.

In some embodiments, R² is C₃₋₂₅ alkyl substituted with 4-10 membered heterocyclyl.

In some embodiments, R² is C₃₋₂₅ alkyl.

In some embodiments, R² is C₃₋₂₅ alkyl substituted with —NR^{A}R^{B}, wherein one non-adjacent carbon atom of the C₃₋₂₅ alkyl is replaced by NH.

In some embodiments, R² is C₃₋₂₅ alkyl substituted with —NR^{A}R^{B} and —OH, wherein two non-adjacent carbon atoms of the C₃₋₂₅ alkyl are replaced by NH.

In some embodiments, R² is C₃₋₂₅ alkyl substituted with —NR^{A}R^{B} and —OH, wherein one non-adjacent carbon atom of the C₃₋₂₅ alkyl is replaced by NH and one non-adjacent carbon atom of the C₃₋₂₅ alkyl is replaced by O.

In some embodiments, R² is C₃₋₂₅ alkyl substituted with —NR^{A}R^{B}, wherein two non-adjacent carbon atoms of the C₃₋₂₅ alkyl are replaced by NH.

In some embodiments, R² is C₃₋₂₅ alkyl substituted with OH, wherein one non-adjacent carbon atom of the C₃₋₂₅ alkyl is replaced by NH and one non-adjacent carbon atom of the C₃₋₂₅ alkyl is replaced by N(CH₃).

In some embodiments, R² is C₃₋₂₅ alkyl substituted with two —NR^{A}R^{B}, wherein one non-adjacent carbon atoms of the C₃₋₂₅ alkyl are replaced by N, three non-adjacent carbon atoms of the C₃₋₂₅ alkyl are replaced by NH, and two non-adjacent carbon atoms of the C₃₋₂₅ alkyl are replaced by O.

In some embodiments, R² is C₃₋₂₅ alkyl substituted with —NR^{A}R^{B}, wherein one non-adjacent carbon atom of the C₃₋₂₅ alkyl is replaced by NH and one non-adjacent carbon atom of the C₃₋₂₅ alkyl is replaced by O.

In some embodiments, R⁴ is C₃₋₂₅ alkyl substituted with 4-10 membered heterocyclyl substituted —NR^{A}R^{B}, wherein one non-adjacent carbon atom of the C₃₋₂₅ alkyl is replaced by NH and four non-adjacent carbon atom of the C₃₋₂₅ alkyl are replaced by O.

In some embodiments, R⁴ is C₃₋₂₅ alkyl substituted with 5-10 membered heteroaryl and —NR^{A}R^{B}, wherein two non-adjacent carbon atoms of the C₃₋₂₅ alkyl are replaced by NH and three non-adjacent carbon atoms of the C₃₋₂₅ alkyl are replaced by O.

In some embodiments, R⁴ is C₃₋₂₅ alkyl substituted with 5-10 membered heteroaryl substituted with —OH and C₁₋₆ alkyl, wherein two non-adjacent carbon atoms of the C₃₋₂₅ alkyl are replaced by NH and three non-adjacent carbon atoms of the C₃₋₂₅ alkyl are replaced by O.

In some embodiments, R⁴ is C₃₋₂₅ alkyl substituted with 4-10 membered heterocyclyl, wherein two non-adjacent carbon atoms of the C₃₋₂₅ alkyl are replaced by NH and three non-adjacent carbon atoms of the C₃₋₂₅ alkyl are replaced by O.

In some embodiments, R⁴ is C₃₋₂₅ alkyl substituted with two —NR^{A}R^{B}, wherein three non-adjacent carbon atoms of the C₃₋₂₅ alkyl are replaced by NH and three non-adjacent carbon atoms of the C₃₋₂₅ alkyl are replaced by O.

In some embodiments, R⁴ is C₃₋₂₅ alkyl substituted with 4-10 membered heterocyclyl substituted with —C₁₋₆ alkyl-(NR^{A}R^{B}), wherein two non-adjacent carbon atom of the C₃₋₂₅ alkyl are replaced by NH and three non-adjacent carbon atom of the C₃₋₂₅ alkyl are replaced by O.

In some embodiments, R⁴ is C₃₋₂₅ alkyl substituted with —NR^{A}R^{B} and —OH, wherein three non-adjacent carbon atoms of the C₃₋₂₅ alkyl are replaced by NH and three non-adjacent carbon atoms of the C₃₋₂₅ alkyl are replaced by O.

In some embodiments, R⁴ is C₃₋₂₅ alkyl substituted with —NR^{A}R^{B} and —OH, wherein two non-adjacent carbon atoms of the C₃₋₂₅ alkyl are replaced by NH and four non-adjacent carbon atoms of the C₃₋₂₅ alkyl are replaced by O.

In some embodiments, R⁴ is C₃₋₂₅ alkyl substituted with 4-10 membered heterocyclyl substituted with —NR^{A}R^{B}

In some embodiments, R⁴ is C₃₋₂₅ alkyl substituted with 4-10 membered heterocyclyl.

In some embodiments, R⁴ is C₃₋₂₅ alkyl.

In some embodiments, R⁴ is C₃₋₂₅ alkyl substituted with —NR^{A}R^{B}, wherein one non-adjacent carbon atom of the C₃₋₂₅ alkyl is replaced by NH.

In some embodiments, R⁴ is C₃₋₂₅ alkyl substituted with —NR^{A}R^{B} and —OH, wherein two non-adjacent carbon atoms of the C₃₋₂₅ alkyl are replaced by NH.

In some embodiments, R⁴ is C₃₋₂₅ alkyl substituted with —NR^{A}R^{B} and —OH, wherein one non-adjacent carbon atom of the C₃₋₂₅ alkyl is replaced by NH and one non-adjacent carbon atom of the C₃₋₂₅ alkyl is replaced by O.

In some embodiments, R⁴ is C₃₋₂₅ alkyl substituted with —NR^{A}R^{B}, wherein two non-adjacent carbon atoms of the C₃₋₂₅ alkyl are replaced by NH.

In some embodiments, R⁴ is C₃₋₂₅ alkyl substituted with OH, wherein one non-adjacent carbon atom of the C₃₋₂₅ alkyl is replaced by NH and one non-adjacent carbon atom of the C₃₋₂₅ alkyl is replaced by N(CH₃).

In some embodiments, R⁴ is C₃₋₂₅ alkyl substituted with two —NR^{A}R^{B}, wherein one non-adjacent carbon atoms of the C₃₋₂₅ alkyl are replaced by N, three non-adjacent carbon atoms of the C₃₋₂₅ alkyl are replaced by NH, and two non-adjacent carbon atoms of the C₃₋₂₅ alkyl are replaced by O.

In some embodiments, R⁴ is C₃₋₂₅ alkyl substituted with —NR^{A}R^{B}, wherein one non-adjacent carbon atom of the C₃₋₂₅ alkyl is replaced by NH and one non-adjacent carbon atom of the C₃₋₂₅ alkyl is replaced by O.

In some embodiments, R⁶ is C₃₋₂₅ alkyl substituted with 4-10 membered heterocyclyl substituted —NR^{A}R^{B}, wherein one non-adjacent carbon atom of the C₃₋₂₅ alkyl is replaced by NH and four non-adjacent carbon atom of the C₃₋₂₅ alkyl are replaced by O.

In some embodiments, R⁶ is C₃₋₂₅ alkyl substituted with 5-10 membered heteroaryl and —NR^{A}R^{B}, wherein two non-adjacent carbon atoms of the C₃₋₂₅ alkyl are replaced by NH and three non-adjacent carbon atoms of the C₃₋₂₅ alkyl are replaced by O.

In some embodiments, R⁶ is C₃₋₂₅ alkyl substituted with 5-10 membered heteroaryl substituted with —OH and C₁₋₆ alkyl, wherein two non-adjacent carbon atoms of the C₃₋₂₅ alkyl are replaced by NH and three non-adjacent carbon atoms of the C₃₋₂₅ alkyl are replaced by O.

In some embodiments, R⁶ is C₃₋₂₅ alkyl substituted with 4-10 membered heterocyclyl, wherein two non-adjacent carbon atoms of the C₃₋₂₅ alkyl are replaced by NH and three non-adjacent carbon atoms of the C₃₋₂₅ alkyl are replaced by O.

In some embodiments, R⁶ is C₃₋₂₅ alkyl substituted with two —NR^{A}R^{B}, wherein three non-adjacent carbon atoms of the C₃₋₂₅ alkyl are replaced by NH and three non-adjacent carbon atoms of the C₃₋₂₅ alkyl are replaced by O.

In some embodiments, R⁶ is C₃₋₂₅ alkyl substituted with 4-10 membered heterocyclyl substituted with —C₁₋₆ alkyl-(NR^{A}R^{B}), wherein two non-adjacent carbon atom of the C₃₋₂₅ alkyl are replaced by NH and three non-adjacent carbon atom of the C₃₋₂₅ alkyl are replaced by O.

In some embodiments, R⁶ is C₃₋₂₅ alkyl substituted with —NR^{A}R^{B} and —OH, wherein three non-adjacent carbon atoms of the C₃₋₂₅ alkyl are replaced by NH and three non-adjacent carbon atoms of the C₃₋₂₅ alkyl are replaced by O.

In some embodiments, R⁶ is C₃₋₂₅ alkyl substituted with —NR^{A}R^{B} and —OH, wherein two non-adjacent carbon atoms of the C₃₋₂₅ alkyl are replaced by NH and four non-adjacent carbon atoms of the C₃₋₂₅ alkyl are replaced by O.

In some embodiments, R⁶ is C₃₋₂₅ alkyl substituted with 4-10 membered heterocyclyl substituted with —NR^{A}R^{B}.

In some embodiments, R⁶ is C₃₋₂₅ alkyl substituted with 4-10 membered heterocyclyl.

In some embodiments, R⁶ is C₃₋₂₅ alkyl.

In some embodiments, R⁶ is C₃₋₂₅ alkyl substituted with —NR^{A}R^{B}, wherein one non-adjacent carbon atom of the C₃₋₂₅ alkyl is replaced by NH.

In some embodiments, R⁶ is C₃₋₂₅ alkyl substituted with —NR^{A}R^{B} and —OH, wherein two non-adjacent carbon atoms of the C₃₋₂₅ alkyl are replaced by NH.

In some embodiments, R⁶ is C₃₋₂₅ alkyl substituted with —NR^{A}R^{B} and —OH, wherein one non-adjacent carbon atom of the C₃₋₂₅ alkyl is replaced by NH and one non-adjacent carbon atom of the C₃₋₂₅ alkyl is replaced by O.

In some embodiments, R⁶ is C₃₋₂₅ alkyl substituted with —NR^{A}R^{B}, wherein two non-adjacent carbon atoms of the C₃₋₂₅ alkyl are replaced by NH.

In some embodiments, R⁶ is C₃₋₂₅ alkyl substituted with OH, wherein one non-adjacent carbon atom of the C₃₋₂₅ alkyl is replaced by NH and one non-adjacent carbon atom of the C₃₋₂₅ alkyl is replaced by N(CH₃).

In some embodiments, R⁶ is C₃₋₂₅ alkyl substituted with two —NR^{A}R^{B}, wherein one non-adjacent carbon atoms of the C₃₋₂₅ alkyl are replaced by N, three non-adjacent carbon atoms of the C₃₋₂₅ alkyl are replaced by NH, and two non-adjacent carbon atoms of the C₃₋₂₅ alkyl are replaced by O.

In some embodiments, R⁶ is C₃₋₂₅ alkyl substituted with —NR^{A}R^{B}, wherein one non-adjacent carbon atom of the C₃₋₂₅ alkyl is replaced by NH and one non-adjacent carbon atom of the C₃₋₂₅ alkyl is replaced by O.

In some embodiments, R², R⁴, and R⁶ are independently selected from the group consisting of:

### Hydrogen;

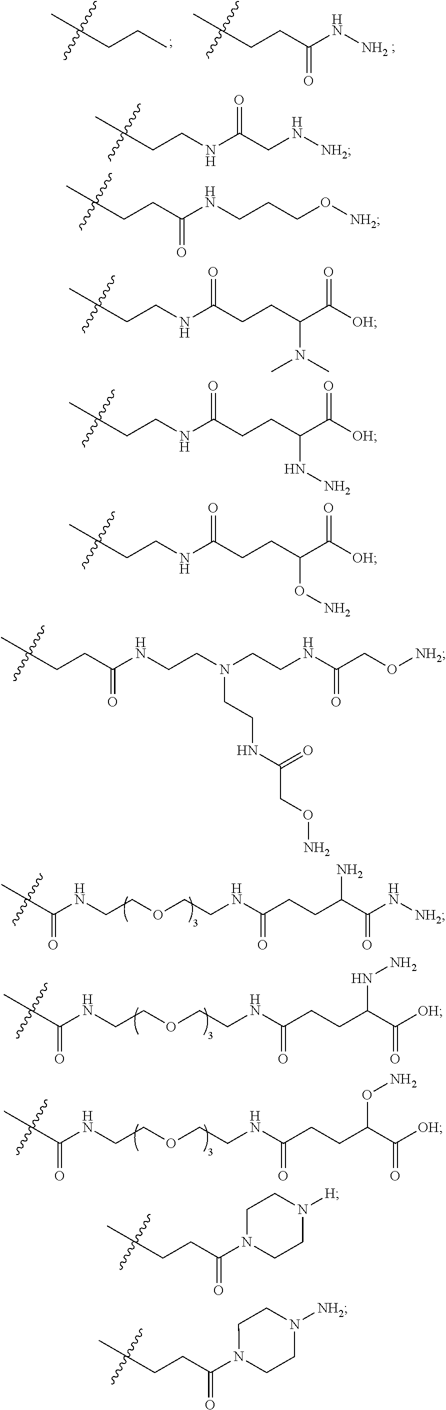

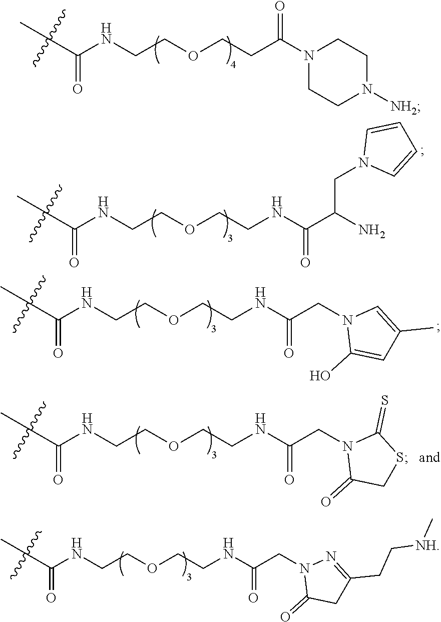

In some embodiments, R² is selected from the group consisting of Hydrogen;

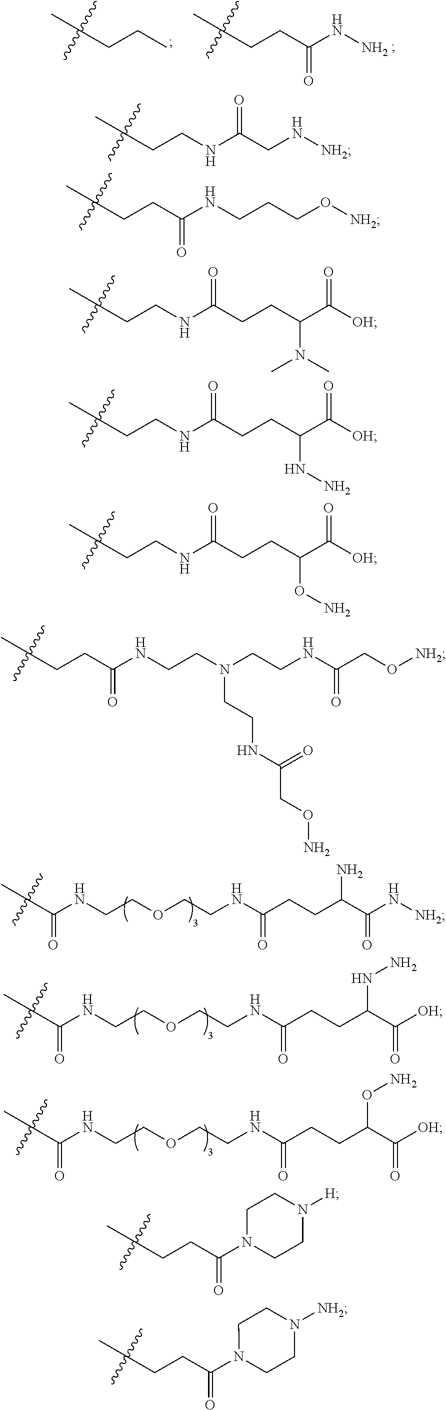

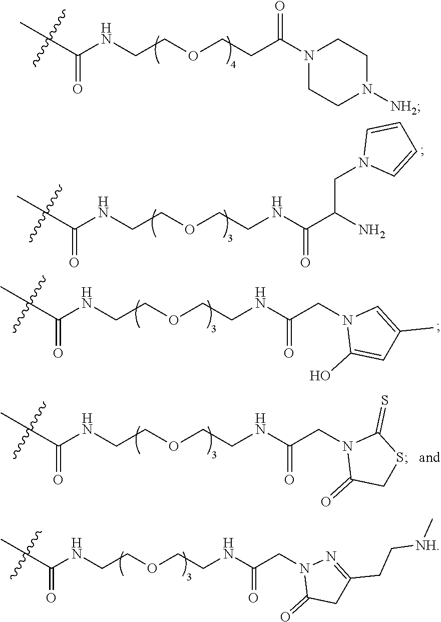

In some embodiments, R⁴ is selected from the group consisting of

### Hydrogen;

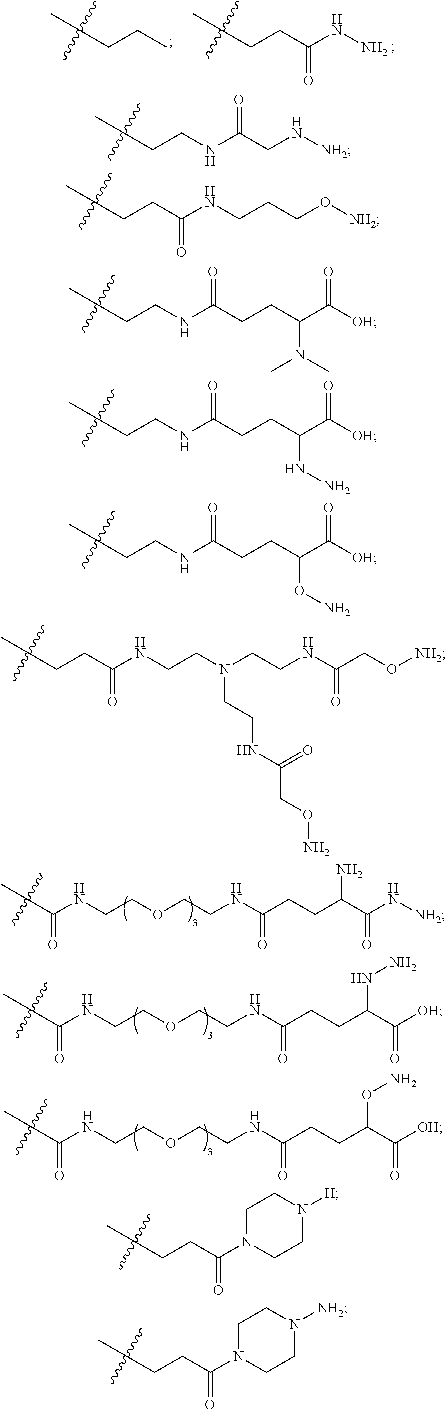

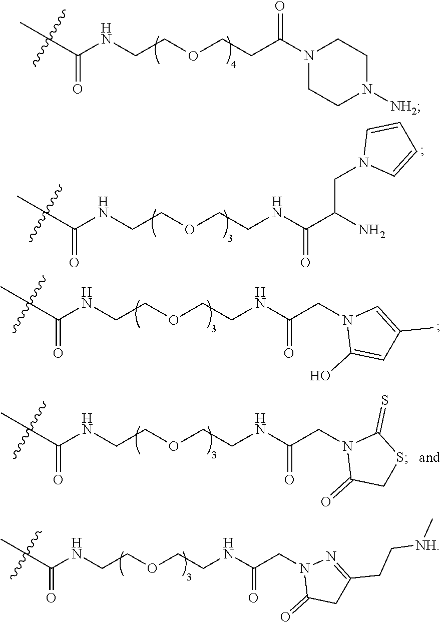

In some embodiments, R⁶ is selected from the group consisting of:

### Hydrogen;

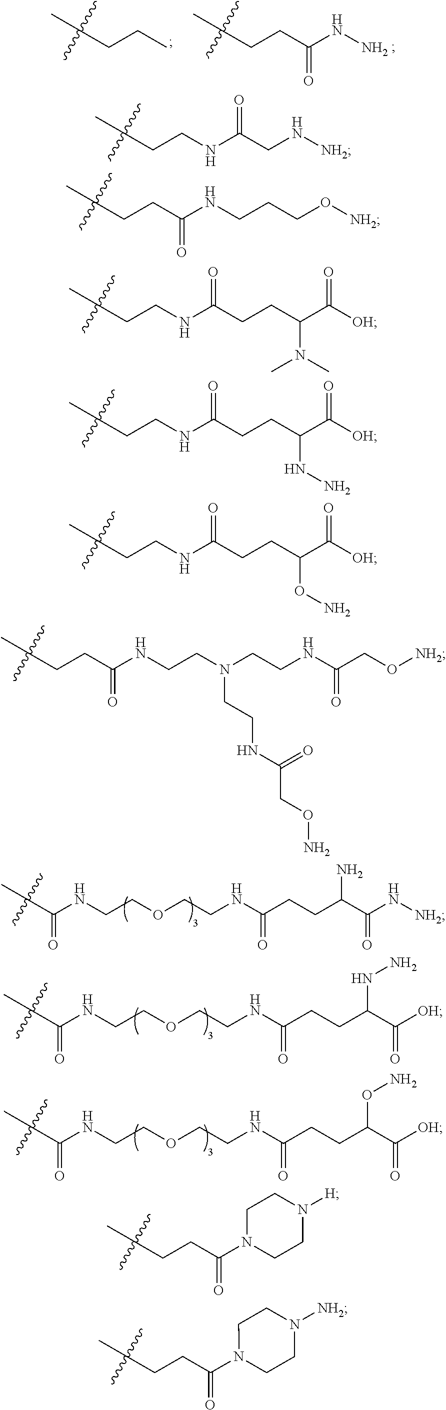

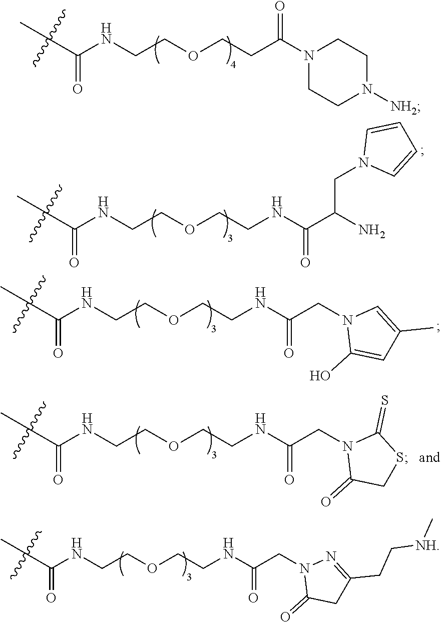

In some embodiments, R², R⁴, and R⁶ are all hydrogen. In some embodiments, R² is hydrogen. In some embodiments, R⁴ is hydrogen. In some embodiments, R⁶ is hydrogen.

In some embodiments, R², R⁴, and R⁶ are all C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R² and R⁴ are both hydrogen and R⁶ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R² and R⁴ are both hydrogen and R⁶ is selected from:

C₃₋₂₅ alkyl substituted with 4-10 membered heterocyclyl substituted —NR^{A}R^{B}, wherein one non-adjacent carbon atom of the C₃₋₂₅ alkyl is replaced by NH and four non-adjacent carbon atom of the C₃₋₂₅ alkyl are replaced by O;

C₃₋₂₅ alkyl substituted with 5-10 membered heteroaryl and —NR^{A}R^{B}, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 5-10 membered heteroaryl substituted with —OH and C₁₋₆ alkyl, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with two —NR^{A}R^{B}, wherein three non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl substituted with —C₁₋₆ alkyl-(NR^{A}R^{B}), wherein two non-adjacent carbon atom of the C₃₋₂₅ alkyl are replaced by NH and three non-adjacent carbon atom of the C₃₋₂₅ alkyl are replaced by O;

C₃₋₂₅ alkyl substituted with —NR^{A}R^{B} and —OH, wherein three non-adjacent carbon atoms of the C₃₋₂₅ alkyl are replaced by NH and three non-adjacent carbon atoms of the C₃₋₂₅ alkyl are replaced by O; and

C₃₋₂₅ alkyl substituted with —NR^{A}R^{B} and —OH, wherein two non-adjacent carbon atoms of the C₃₋₂₅ alkyl are replaced by NH and four non-adjacent carbon atoms of the C₃₋₂₅ alkyl are replaced by O.

In some embodiments, R² and R⁶ are both hydrogen and R⁴ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R² and R⁶ are both hydrogen and R⁴ is selected from:

C₃₋₂₅ alkyl substituted with —NR^{A}R^{B} and —OH, wherein two non-adjacent carbon atoms of the C₃₋₂₅ alkyl are replaced by NH;

C₃₋₂₅ alkyl substituted with —NR^{A}R^{B} and —OH, wherein one non-adjacent carbon atom of the C₃₋₂₅ alkyl is replaced by NH and one non-adjacent carbon atom of the C₃₋₂₅ alkyl is replaced by O;

C₃₋₂₅ alkyl substituted with —NR^{A}R^{B}, wherein two non-adjacent carbon atoms of the C₃₋₂₅ alkyl are replaced by NH; and

C₃₋₂₅ alkyl substituted with OH, wherein one non-adjacent carbon atom of the C₃₋₂₅ alkyl is replaced by NH and one non-adjacent carbon atom of the C₃₋₂₅ alkyl is replaced by N(CH₃).

In some embodiments, R⁶ and R⁴ are both hydrogen and R² is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R¹, R³, and R⁵ are all —C(═O)OH.

In some embodiments, R¹ and R³ are both —C(═O)OH and R⁵ is hydrogen.

In some embodiments, R¹ and R⁵ are both —C(═O)OH and R³ is hydrogen.

In some embodiments, R⁵ and R³ are both —C(═O)OH and R¹ is hydrogen.

In some embodiments, R¹ is —C(═O)OH and R² is hydrogen.

In some embodiments, R³ is —C(═O)OH and R⁴ is hydrogen.

In some embodiments, R³ is hydrogen and R⁴ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R³ is hydrogen and R⁴ is selected from: C₃₋₂₅ alkyl substituted with —NR^{A}R^{B} and —OH, wherein two non-adjacent carbon atoms of the C₃₋₂₅ alkyl are replaced by NH;

C₃₋₂₅ alkyl substituted with —NR^{A}R^{B} and —OH, wherein one non-adjacent carbon atom of the C₃₋₂₅ alkyl is replaced by NH and one non-adjacent carbon atom of the C₃₋₂₅ alkyl is replaced by O;

C₃₋₂₅ alkyl substituted with —NR^{A}R^{B}, wherein two non-adjacent carbon atoms of the C₃₋₂₅ alkyl are replaced by NH; and

C₃₋₂₅ alkyl substituted with OH, wherein one non-adjacent carbon atom of the C₃₋₂₅ alkyl is replaced by NH and one non-adjacent carbon atom of the C₃₋₂₅ alkyl is replaced by N(CH₃).

In some embodiments, R⁵ is hydrogen and R⁶ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R⁵ is hydrogen and R⁶ is selected from the group consisting of

C₃₋₂₅ alkyl substituted with 4-10 membered heterocyclyl substituted —NR^{A}R^{B}, wherein one non-adjacent carbon atom of the C₃₋₂₅ alkyl is replaced by NH and four non-adjacent carbon atom of the C₃₋₂₅ alkyl are replaced by O;

C₃₋₂₅ alkyl substituted with 5-10 membered heteroaryl and —NR^{A}R^{B}, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 5-10 membered heteroaryl substituted with —OH and C₁₋₆ alkyl, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with two —NR^{A}R^{B}, wherein three non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl substituted with —C₁₋₆ alkyl-(NR^{A}R^{B}), wherein two non-adjacent carbon atom of the C₃₋₂₅ alkyl are replaced by NH and three non-adjacent carbon atom of the C₃₋₂₅ alkyl are replaced by O;

C₃₋₂₅ alkyl substituted with —NR^{A}R^{B} and —OH, wherein three non-adjacent carbon atoms of the C₃₋₂₅ alkyl are replaced by NH and three non-adjacent carbon atoms of the C₃₋₂₅ alkyl are replaced by O; and

C₃₋₂₅ alkyl substituted with —NR^{A}R^{B} and —OH, wherein two non-adjacent carbon atoms of the C₃₋₂₅ alkyl are replaced by NH and four non-adjacent carbon atoms of the C₃₋₂₅ alkyl are replaced by O.

In some embodiments, R⁵ is —C(═O)OH and R⁶ is hydrogen.

In some embodiments, n is 1.

In some embodiments, R², R⁴, R⁶, and R⁸ are independently selected from the group consisting of hydrogen and C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R⁸ is selected from the group consisting of hydrogen and C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R², R⁴, R⁶, and R⁸ are independently selected from the group consisting of:

Hydrogen;

C₃₋₂₅ alkyl substituted with 4-10 membered heterocyclyl substituted —NR^{A}R^{B}, wherein one non-adjacent carbon atom of the C₃₋₂₅ alkyl is replaced by NH and four non-adjacent carbon atom of the C₃₋₂₅ alkyl are replaced by O;

C₃₋₂₅ alkyl substituted with 5-10 membered heteroaryl and —NR^{A}R^{B}, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 5-10 membered heteroaryl substituted with —OH and C₁₋₆ alkyl, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with two —NR^{A}R^{B}, wherein three non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl substituted with —C₁₋₆ alkyl-(NR^{A}R^{B}), wherein two non-adjacent carbon atom of the C₃₋₂₅ alkyl are replaced by NH and three non-adjacent carbon atom of the C₃₋₂₅ alkyl are replaced by O;

C₃₋₂₅ alkyl substituted with —NR^{A}R^{B} and —OH, wherein three non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with —NR^{A}R^{B} and —OH, wherein two non-adjacent carbon atoms of the C₃₋₂₅ alkyl are replaced by NH and four non-adjacent carbon atoms of the C₃₋₂₅ alkyl are replaced by O;

C₃₋₂₅ alkyl substituted with 4-10 membered heterocyclyl substituted with —NR^{A}R^{B};

C₃₋₂₅ alkyl substituted with 4-10 membered heterocyclyl;

C₃₋₂₅ alkyl;

C₃₋₂₅ alkyl substituted with —NR^{A}R^{B}, wherein one non-adjacent carbon atom of the C₃₋₂₅ alkyl is replaced by NH;

C₃₋₂₅ alkyl substituted with —NR^{A}R^{B} and —OH, wherein two non-adjacent carbon atoms of the C₃₋₂₅ alkyl are replaced by NH;

C₃₋₂₅ alkyl substituted with —NR^{A}R^{B} and —OH, wherein one non-adjacent carbon atom of the C₃₋₂₅ alkyl is replaced by NH and one non-adjacent carbon atom of the C₃₋₂₅ alkyl is replaced by O;

C₃₋₂₅ alkyl substituted with —NR^{A}R^{B}, wherein two non-adjacent carbon atoms of the C₃₋₂₅ alkyl are replaced by NH;

C₃₋₂₅ alkyl substituted with OH, wherein one non-adjacent carbon atom of the C₃₋₂₅ alkyl is replaced by NH and one non-adjacent carbon atom of the C₃₋₂₅ alkyl is replaced by N(CH₃);

C₃₋₂₅ alkyl substituted with two —NR^{A}R^{B}, wherein one non-adjacent carbon atoms of the C₃₋₂₅ alkyl are replaced by N, three non-adjacent carbon atoms of the C₃₋₂₅ alkyl are replaced by NH, and two non-adjacent carbon atoms of the C₃₋₂₅ alkyl are replaced by O; and

C₃₋₂₅ alkyl substituted with —NR^{A}R^{B}, wherein one non-adjacent carbon atom of the C₃₋₂₅ alkyl is replaced by NH and one non-adjacent carbon atom of the C₃₋₂₅ alkyl is replaced by O.

In some embodiments, R⁸ is selected from the group consisting of:

Hydrogen;

C₃₋₂₅ alkyl substituted with 4-10 membered heterocyclyl substituted —NR^{A}R^{B}, wherein one non-adjacent carbon atom of the C₃₋₂₅ alkyl is replaced by NH and four non-adjacent carbon atom of the C₃₋₂₅ alkyl are replaced by O;

C₃₋₂₅ alkyl substituted with 5-10 membered heteroaryl and —NR^{A}R^{B}, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 5-10 membered heteroaryl substituted with —OH and C₁₋₆ alkyl, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl, wherein two non-adjacent carbon atoms of the C₃₋₂s alkyl are replaced by NH and three non-adjacent carbon atoms of the C₃₋₂₅ alkyl are replaced by O;

C₃₋₂₅ alkyl substituted with two —NR^{A}R^{B}, wherein three non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl substituted with —C₁₋₆ alkyl-(NR^{A}R^{B}), wherein two non-adjacent carbon atom of the C₃₋₂₅ alkyl are replaced by NH and three non-adjacent carbon atom of the C₃₋₂₅ alkyl are replaced by O;

C₃₋₂₅ alkyl substituted with —NR^{A}R^{B} and —OH, wherein three non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with —NR^{A}R^{B} and —OH, wherein two non-adjacent carbon atoms of the C₃₋₂₅ alkyl are replaced by NH and four non-adjacent carbon atoms of the C₃₋₂₅ alkyl are replaced by O;

C₃₋₂₅ alkyl substituted with 4-10 membered heterocyclyl substituted with —NR^{A}R^{B}; C₃₋₂₅ alkyl substituted with 4-10 membered heterocyclyl;

C₃₋₂₅ alkyl;

C₃₋₂₅ alkyl substituted with —NR^{A}R^{B}, wherein one non-adjacent carbon atom of the C₃₋₂₅ alkyl is replaced by NH;

C₃₋₂₅ alkyl substituted with —NR^{A}R^{B} and —OH, wherein two non-adjacent carbon atoms of the C₃₋₂₅ alkyl are replaced by NH;

C₃₋₂₅ alkyl substituted with —NR^{A}R^{B} and —OH, wherein one non-adjacent carbon atom of the C₃₋₂₅ alkyl is replaced by NH and one non-adjacent carbon atom of the C₃₋₂₅ alkyl is replaced by O;

C₃₋₂₅ alkyl substituted with —NR^{A}R^{B}, wherein two non-adjacent carbon atoms of the C₃₋₂₅ alkyl are replaced by NH;

C₃₋₂₅ alkyl substituted with OH, wherein one non-adjacent carbon atom of the C₃₋₂₅ alkyl is replaced by NH and one non-adjacent carbon atom of the C₃₋₂₅ alkyl is replaced by N(CH₃);

C₃₋₂₅ alkyl substituted with two —NR^{A}R^{B}, wherein one non-adjacent carbon atoms of the C₃₋₂₅ alkyl are replaced by N, three non-adjacent carbon atoms of the C₃₋₂₅ alkyl are replaced by NH, and two non-adjacent carbon atoms of the C₃₋₂₅ alkyl are replaced by O; and

C₃₋₂₅ alkyl substituted with —NR^{A}R^{B}, wherein one non-adjacent carbon atom of the C₃₋₂₅ alkyl is replaced by NH and one non-adjacent carbon atom of the C₃₋₂₅ alkyl is replaced by O.

In some embodiments, R⁸ is C₃₋₂₅ alkyl substituted with 4-10 membered heterocyclyl substituted —NR^{A}R^{B}, wherein one non-adjacent carbon atom of the C₃₋₂₅ alkyl is replaced by NH and four non-adjacent carbon atom of the C₃₋₂₅ alkyl are replaced by O.

In some embodiments, R⁸ is C₃₋₂₅ alkyl substituted with 5-10 membered heteroaryl and —NR^{A}R^{B}, wherein two non-adjacent carbon atoms of the C₃₋₂₅ alkyl are replaced by NH and three non-adjacent carbon atoms of the C₃₋₂₅ alkyl are replaced by O.

In some embodiments, R⁸ is C₃₋₂₅ alkyl substituted with 5-10 membered heteroaryl substituted with —OH and C₁₋₆ alkyl, wherein two non-adjacent carbon atoms of the C₃₋₂₅ alkyl are replaced by NH and three non-adjacent carbon atoms of the C₃₋₂₅ alkyl are replaced by O.

In some embodiments, R⁸ is C₃₋₂₅ alkyl substituted with 4-10 membered heterocyclyl, wherein two non-adjacent carbon atoms of the C₃₋₂₅ alkyl are replaced by NH and three non-adjacent carbon atoms of the C₃₋₂₅ alkyl are replaced by O.

In some embodiments, R⁸ is C₃₋₂₅ alkyl substituted with two —NR^{A}R^{B}, wherein three non-adjacent carbon atoms of the C₃₋₂₅ alkyl are replaced by NH and three non-adjacent carbon atoms of the C₃₋₂₅ alkyl are replaced by O.

In some embodiments, R⁸ is C₃₋₂₅ alkyl substituted with 4-10 membered heterocyclyl substituted with —C₁₋₆ alkyl-(NR^{A}R^{B}), wherein two non-adjacent carbon atom of the C₃₋₂₅ alkyl are replaced by NH and three non-adjacent carbon atom of the C₃₋₂₅ alkyl are replaced by O.

In some embodiments, R⁸ is C₃₋₂₅ alkyl substituted with —NR^{A}R^{B} and —OH, wherein three non-adjacent carbon atoms of the C₃₋₂₅ alkyl are replaced by NH and three non-adjacent carbon atoms of the C₃₋₂₅ alkyl are replaced by O.

In some embodiments, R⁸ is C₃₋₂₅ alkyl substituted with —NR^{A}R^{B} and —OH, wherein two non-adjacent carbon atoms of the C₃₋₂₅ alkyl are replaced by NH and four non-adjacent carbon atoms of the C₃₋₂₅ alkyl are replaced by O.

In some embodiments, R⁸ is C₃₋₂₅ alkyl substituted with 4-10 membered heterocyclyl substituted with —NR^{A}R^{B}.

In some embodiments, R⁸ is C₃₋₂₅ alkyl substituted with 4-10 membered heterocyclyl.

In some embodiments, R⁸ is C₃₋₂₅ alkyl.

In some embodiments, R⁸ is C₃₋₂₅ alkyl substituted with —NR^{A}R^{B}, wherein one non-adjacent carbon atom of the C₃₋₂₅ alkyl is replaced by NH.

In some embodiments, R⁸ is C₃₋₂₅ alkyl substituted with —NR^{A}R^{B} and —OH, wherein two non-adjacent carbon atoms of the C₃₋₂₅ alkyl are replaced by NH.

In some embodiments, R⁸ is C₃₋₂₅ alkyl substituted with —NR^{A}R^{B} and —OH, wherein one non-adjacent carbon atom of the C₃₋₂₅ alkyl is replaced by NH and one non-adjacent carbon atom of the C₃₋₂₅ alkyl is replaced by O.

In some embodiments, R⁸ is C₃₋₂₅ alkyl substituted with —NR^{A}R^{B}, wherein two non-adjacent carbon atoms of the C₃₋₂₅ alkyl are replaced by NH.

In some embodiments, R⁸ is C₃₋₂₅ alkyl substituted with OH, wherein one non-adjacent carbon atom of the C₃₋₂₅ alkyl is replaced by NH and one non-adjacent carbon atom of the C₃₋₂₅ alkyl is replaced by N(CH₃).

In some embodiments, R⁸ is C₃₋₂₅ alkyl substituted with two —NR^{A}R^{B}, wherein one non-adjacent carbon atoms of the C₃₋₂₅ alkyl are replaced by N, three non-adjacent carbon atoms of the C₃₋₂₅ alkyl are replaced by NH, and two non-adjacent carbon atoms of the C₃₋₂₅ alkyl are replaced by O.

In some embodiments, R⁸ is C₃₋₂₅ alkyl substituted with —NR^{A}R^{B}, wherein one non-adjacent carbon atom of the C₃₋₂₅ alkyl is replaced by NH and one non-adjacent carbon atom of the C₃₋₂₅ alkyl is replaced by O.

In some embodiments, R², R⁴, R⁶, and R⁸ are independently selected from the group consisting of:

Hydrogen;

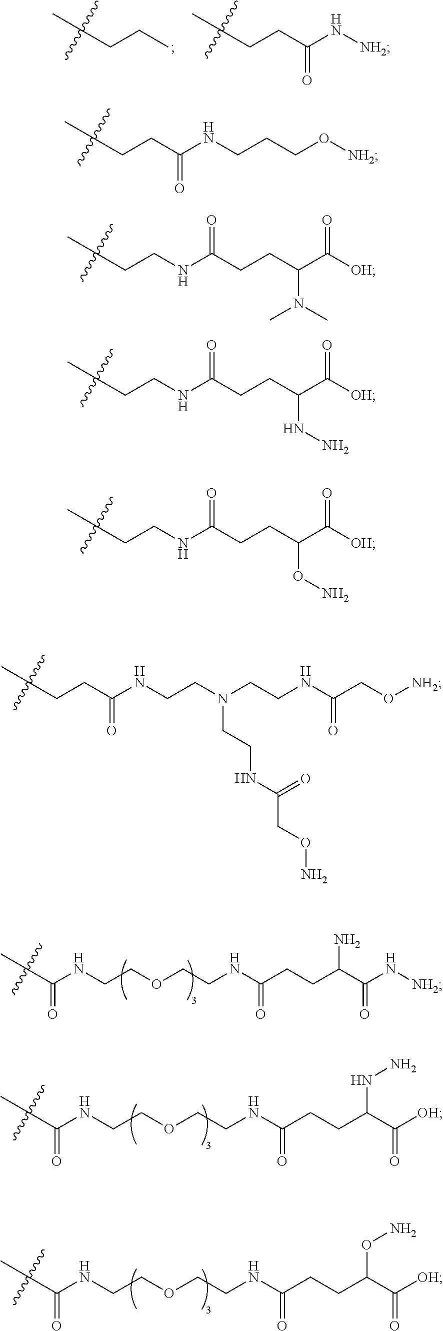

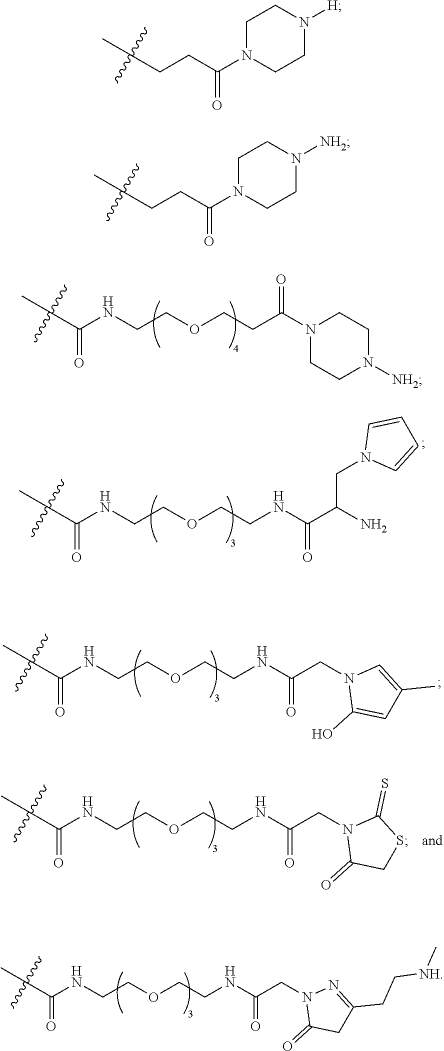

In some embodiments, R⁸ is selected from the group consisting of:

### Hydrogen;

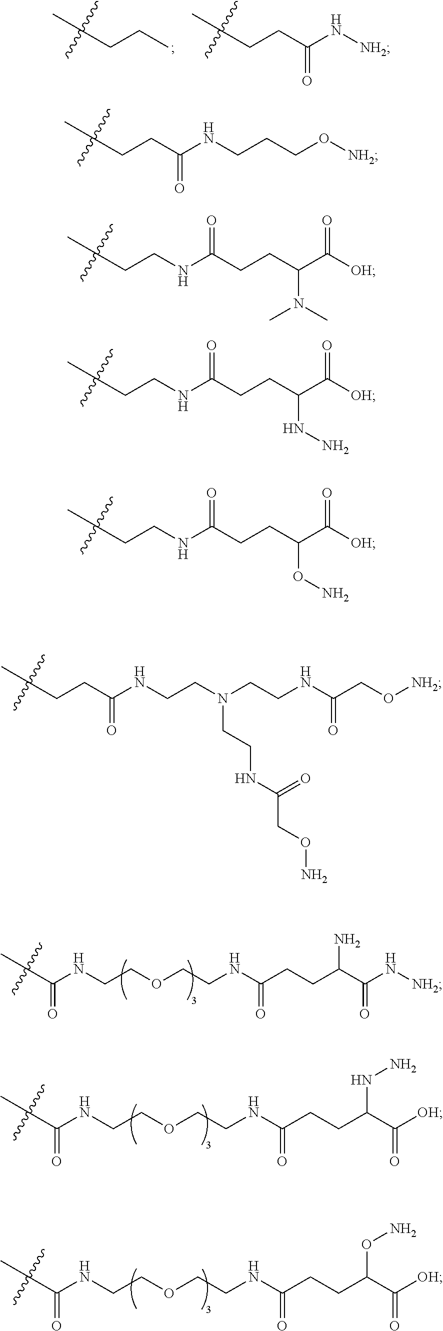

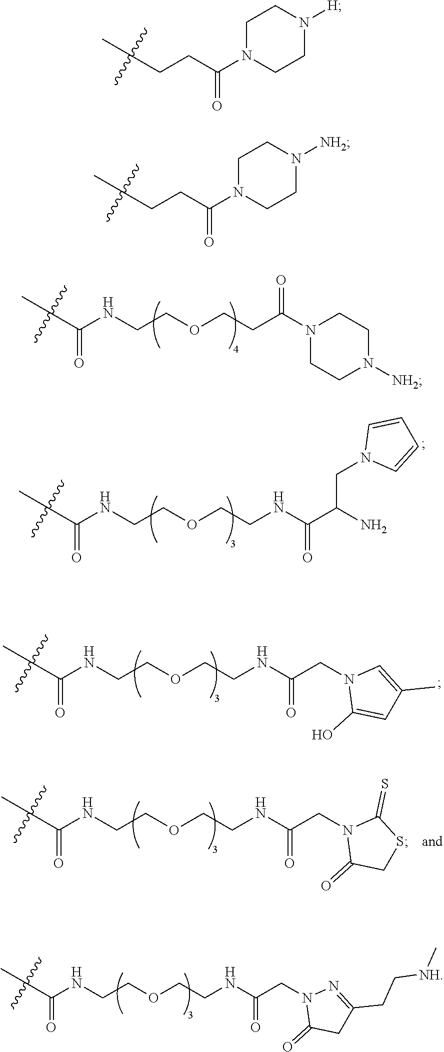

In some embodiments, R², R⁴, R⁶, and R⁸ are all hydrogen. In some embodiments R⁸ is hydrogen.

In some embodiments, R², R⁴, R⁶, and R⁸ are all C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R² and R⁴ are both hydrogen and R⁶ and R⁸ are both C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R² and R⁶ are both hydrogen and R⁴ and R are both C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R² and R⁸ are both hydrogen and R⁴ and R⁶ are both C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R⁴ and R⁶ are both hydrogen and R² and BY are both C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R⁴ and R⁸ are both hydrogen and R² and R⁶ are both C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R⁶ and R⁸ are both hydrogen and R² and R⁴ are both C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R², R⁴, and R⁶ are all hydrogen and R⁸ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R², R⁴, and R⁸ are all hydrogen and R⁶ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R², R⁶, and R⁸ are all hydrogen and R⁴ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R⁴, R⁶, and R⁸ are all hydrogen and R² is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R² and R⁴ are hydrogen and R⁶ and R⁸ are selected from: C₃₋₂₅ alkyl substituted with 4-10 membered heterocyclyl substituted with —NR^{A}R^{B};

C₃₋₂₅ alkyl substituted with 4-10 membered heterocyclyl;

C₃₋₂₅ alkyl; and

C₃₋₂₅ alkyl substituted with —NR^{A}R^{B}, wherein one non-adjacent carbon atom of the C₃₋₂₅ alkyl is replaced by NH.

In some embodiments, R⁴ and R⁸ are hydrogen and R² and R⁶ are C₃₋₂₅ alkyl substituted with 4-10 membered heterocyclyl substituted with —NR^{A}R^{B}.

In some embodiments, R², R⁴, and R⁸ are hydrogen and R⁶ is selected from:

C₃₋₂₅ alkyl substituted with two —NR^{A}R^{B}, wherein one non-adjacent carbon atoms of the C₃₋₂₅ alkyl are replaced by N, three non-adjacent carbon atoms of the C₃₋₂₅ alkyl are replaced by NH, and two non-adjacent carbon atoms of the C₃₋₂₅ alkyl are replaced by O; and

C₃₋₂₅ alkyl substituted with —NR^{A}R^{B}, wherein one non-adjacent carbon atom of the C₃₋₂₅ alkyl is replaced by NH and one non-adjacent carbon atom of the C₃₋₂₅ alkyl is replaced by O.

In some embodiments, R¹, R³, R⁵ and R⁷ are all —C(═O)OH.

In some embodiments, R¹ and R³ are both —C(═O)OH and R⁵ and R⁷ are both hydrogen.

In some embodiments, R¹ and R⁵ are both —C(═O)OH and R³ and R⁷ are both hydrogen.

In some embodiments, R¹ and R⁷ are both —C(═O)OH and R³ and R⁵ are both hydrogen.

In some embodiments, R³ and R⁵ are both —C(═O)OH and R¹ and R⁷ are both hydrogen.

In some embodiments, R³ and R⁷ are both —C(═O)OH and R¹ and R⁵ are both hydrogen.

In some embodiments, R⁵ and R⁷ are both —C(═O)OH and R¹ and R³ are both hydrogen.

In some embodiments, R¹, R³, and R⁵ are all —C(═O)OH and R⁷ is hydrogen.

In some embodiments, R¹, R³, and R⁷ are all —C(═O)OH and R⁵ is hydrogen.

In some embodiments, R¹, R⁵, and R⁷ are all —C(═O)OH and R³ is hydrogen.

In some embodiments, R³, R⁵, and R⁷ are all —C(═O)OH and R¹ is hydrogen.

In some embodiments, R¹ is —C(═O)OH and R² is hydrogen.

In some embodiments, R¹ is —C(═O)OH and R² is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R¹ is —C(═O)OH and R² is C₃₋₂₅ alkyl substituted with 4-10 membered heterocyclyl substituted with —NR^{A}R^{B}.

In some embodiments, R³ is —C(═O)OH and R⁴ is hydrogen.

In some embodiments, R⁵ is —C(═O)OH and R⁶ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R⁵ is hydrogen and R⁶ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R⁵ is —C(═O)OH and R⁶ is selected from:

C₃₋₂₅ alkyl substituted with 4-10 membered heterocyclyl substituted with —NR^{A}R^{B}.

C₃₋₂₅ alkyl substituted with two —NR^{A}R^{B}, wherein one non-adjacent carbon atoms of the C₃₋₂₅ alkyl are replaced by N, three non-adjacent carbon atoms of the C₃₋₂₅ alkyl are replaced by NH, and two non-adjacent carbon atoms of the C₃₋₂₅ alkyl are replaced by O; and

C₃₋₂₅ alkyl substituted with —NR^{A}R^{B}, wherein one non-adjacent carbon atom of the C₃₋₂₅ alkyl is replaced by NH and one non-adjacent carbon atom of the C₃₋₂₅ alkyl is replaced by O.

In some embodiments, R⁵ is hydrogen and R⁶ is selected from:

C₃₋₂₅ alkyl substituted with 4-10 membered heterocyclyl substituted with —NR^{A}R^{B}; and

C₃₋₂₅ alkyl substituted with —NR^{A}R^{B}, wherein one non-adjacent carbon atom of the C₃₋₂₅ alkyl is replaced by NH.

In some embodiments, R⁷ is —C(═O)OH and R⁸ is hydrogen.

In some embodiments, R⁷ is —C(═O)OH and R⁸ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R⁷ is hydrogen and R⁸ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

In some embodiments, R⁷ is —C(═O)OH and R⁸ is C₃₋₂₅ alkyl substituted with 4-10 membered heterocyclyl.

In some embodiments, R⁷ is hydrogen and R⁸ is selected from:

C₃₋₂₅ alkyl substituted with 4-10 membered heterocyclyl substituted with —NR^{A}R^{B};

C₃₋₂₅ alkyl; and

C₃₋₂₅ alkyl substituted with —NR^{A}R^{B}, wherein one non-adjacent carbon atom of the C₃₋₂₅ alkyl is replaced by NH.

In some embodiments, p is 0.

In some embodiments, p is 1.

In some embodiments, R⁹ is H, halogen, or —OH.

In some embodiments, R⁹ is H.

In some embodiments, R^{A} is hydrogen.

In some embodiments, R^{A} is C₁₋₆ alkyl. In some embodiments R^{A} is —CH₃.

In some embodiments, R^{B} is hydrogen.

In some embodiments, R^{B} is C₁₋₆ alkyl. In some embodiments R^{B} is —CH₃.

In some embodiments, R^{A} and R^{B} are both hydrogen.

In some embodiments, the compound of Formula (I) is a compound of Formula (IA)

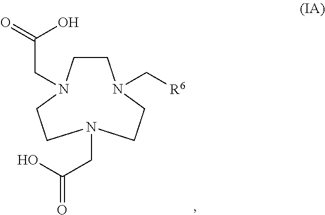

or a pharmaceutically acceptable salt thereof.

In some embodiments, R⁶ is selected from the group consisting of:

C₃₋₂₅ alkyl substituted with 4-10 membered heterocyclyl substituted —NR^{A}R^{B}, wherein one non-adjacent carbon atom of the C₃₋₂₅ alkyl is replaced by NH and four non-adjacent carbon atom of the C₃₋₂₅ alkyl are replaced by O;

C₃₋₂₅ alkyl substituted with 5-10 membered heteroaryl and —NR^{A}R^{B}, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 5-10 membered heteroaryl substituted with —OH and C₁₋₆ alkyl, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with two —NR^{A}R^{B}, wherein three non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl substituted with —C₁₋₆ alkyl-(NR^{A}R^{B}), wherein two non-adjacent carbon atom of the C₃₋₂₅ alkyl are replaced by NH and three non-adjacent carbon atom of the C₃₋₂₅ alkyl are replaced by O;

C₃₋₂₅ alkyl substituted with —NR^{A}R^{B} and —OH, wherein three non-adjacent carbon atoms of the C₃₋₂₅ alkyl are replaced by NH and three non-adjacent carbon atoms of the C₃₋₂₅ alkyl are replaced by O; and

C₃₋₂₅ alkyl substituted with —NR^{A}R^{B} and —OH, wherein two non-adjacent carbon atoms of the C₃₋₂₅ alkyl are replaced by NH and four non-adjacent carbon atoms of the C₃₋₂₅ alkyl are replaced by O.

In some embodiments,

each R¹, R³, R⁵, and R⁷ are independently hydrogen or —C(═O)OH;

each R², R⁴, R⁶, and R⁸ are independently hydrogen or C₃₋₂₅ alkyl, wherein the C₃₋₂₅ alkyl is optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃);

R⁹ is H;

each R^{A} and R^{B} are independently hydrogen or C₁₋₆ alkyl;

n is 0 or 1; and

p is 0 or 1;

wherein if n is 0 at least two of R¹, R³, and R⁵ are —C(═O)OH and if n is 1 at least two of R¹, R³, R⁵, and R⁷ are —C(═O)OH.

In some embodiments, the compound of Formula (I) is a compound of Formula (IB)

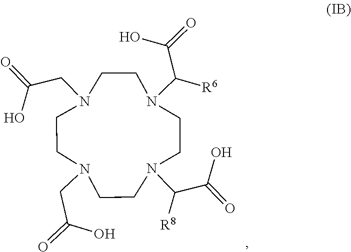

or a pharmaceutically acceptable salt thereof.

In some embodiments, R⁶ is selected from the group consisting of:

C₃₋₂₅ alkyl substituted with 4-10 membered heterocyclyl substituted with —NR^{A}R^{B}; and

C₃₋₂₅ alkyl substituted with —NR^{A}R^{B}, wherein one non-adjacent carbon atom of the C₃₋₂₅ alkyl is replaced by NH.

In some embodiments, R⁸ is selected from the group consisting of:

C₃₋₂₅ alkyl substituted with 4-10 membered heterocyclyl substituted with —NR^{A}R^{B};

C₃₋₂₅ alkyl substituted with 4-10 membered heterocyclyl;

C₃₋₂₅ alkyl; and

C₃₋₂₅ alkyl substituted with —NR^{A}R^{B}, wherein one non-adjacent carbon atom of the C₃₋₂₅ alkyl is replaced by NH.

In some embodiments, the compound of Formula (I) is a compound of Formula (IC)

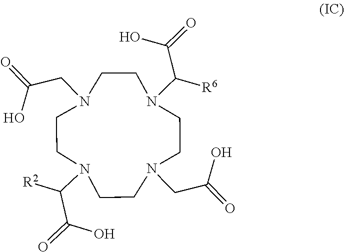

or a pharmaceutically acceptable salt thereof.

In some embodiments, R² is selected from the group consisting of:

Hydrogen; and

C₃₋₂₅ alkyl substituted with 4-10 membered heterocyclyl substituted with —NR^{A}R^{B};

In some embodiments, R⁶ is selected from the group consisting of:

C₃₋₂₅ alkyl substituted with two —NR^{A}R^{B}, wherein one non-adjacent carbon atoms of the C₃₋₂₅ alkyl are replaced by N, three non-adjacent carbon atoms of the C₃₋₂₅ alkyl are replaced by NH, and two non-adjacent carbon atoms of the C₃₋₂₅ alkyl are replaced by O; and

C₃₋₂₅ alkyl substituted with 4-10 membered heterocyclyl substituted with —NR^{A}R^{B};

In some embodiments, the compound of Formula (I) is a compound of Formula (ID)

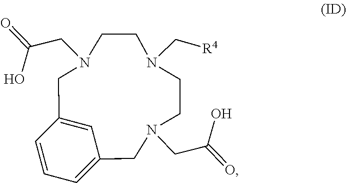

or a pharmaceutically acceptable salt thereof.

In some embodiments, R⁴ is selected form the group consisting of:

C₃₋₂₅ alkyl substituted with —NR^{A}R^{B} and —OH, wherein two non-adjacent carbon atoms of the C₃₋₂₅ alkyl are replaced by NH;

C₃₋₂₅ alkyl substituted with —NR^{A}R^{B} and —OH, wherein one non-adjacent carbon atom of the C₃₋₂₅ alkyl is replaced by NH and one non-adjacent carbon atom of the C₃₋₂₅ alkyl is replaced by O;

C₃₋₂₅ alkyl substituted with —NR^{A}R^{B}, wherein two non-adjacent carbon atoms of the C₃₋₂₅ alkyl are replaced by NH; and

C₃₋₂₅ alkyl substituted with OH, wherein one non-adjacent carbon atom of the C₃₋₂₅ alkyl is replaced by NH and one non-adjacent carbon atom of the C₃₋₂₅ alkyl is replaced by N(CH₃).

In some embodiments, the compound of Formula (I) is selected from:

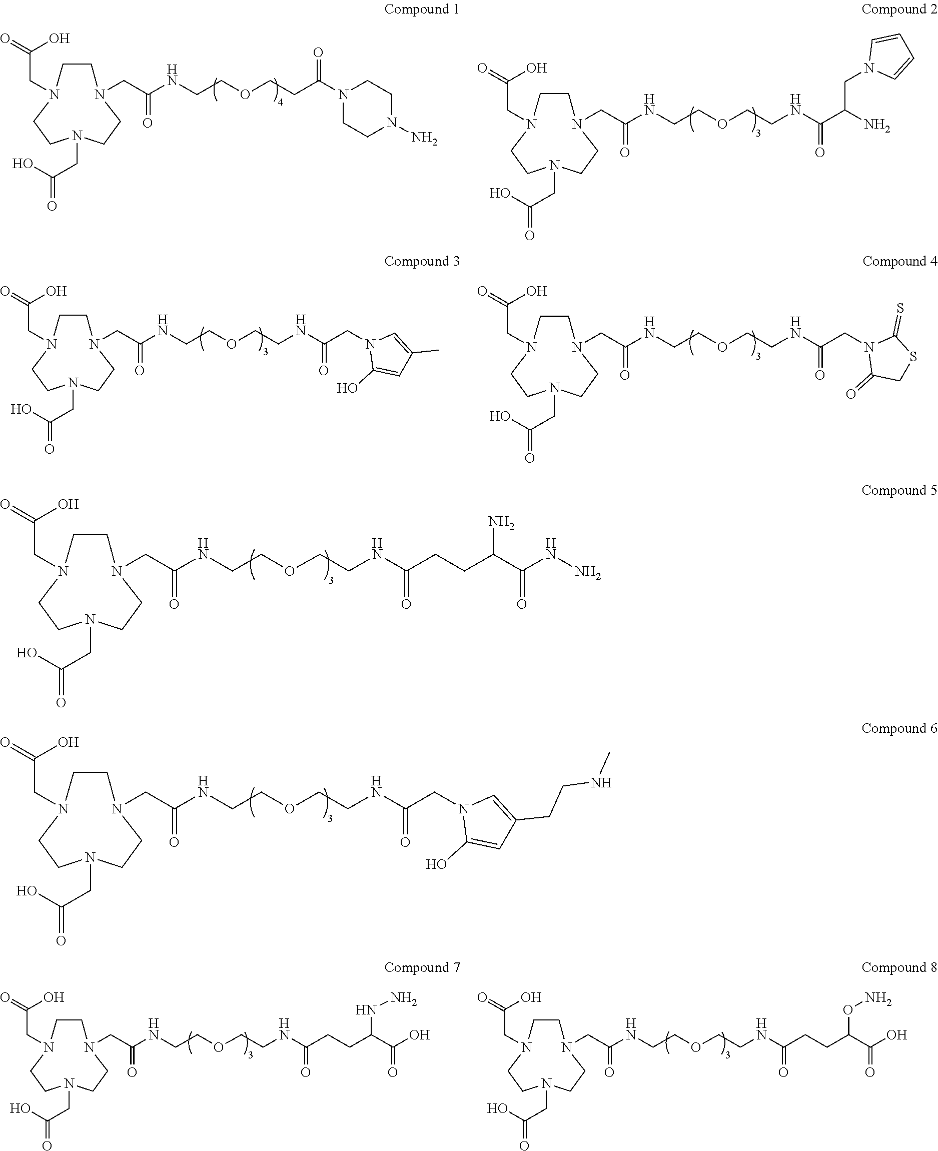

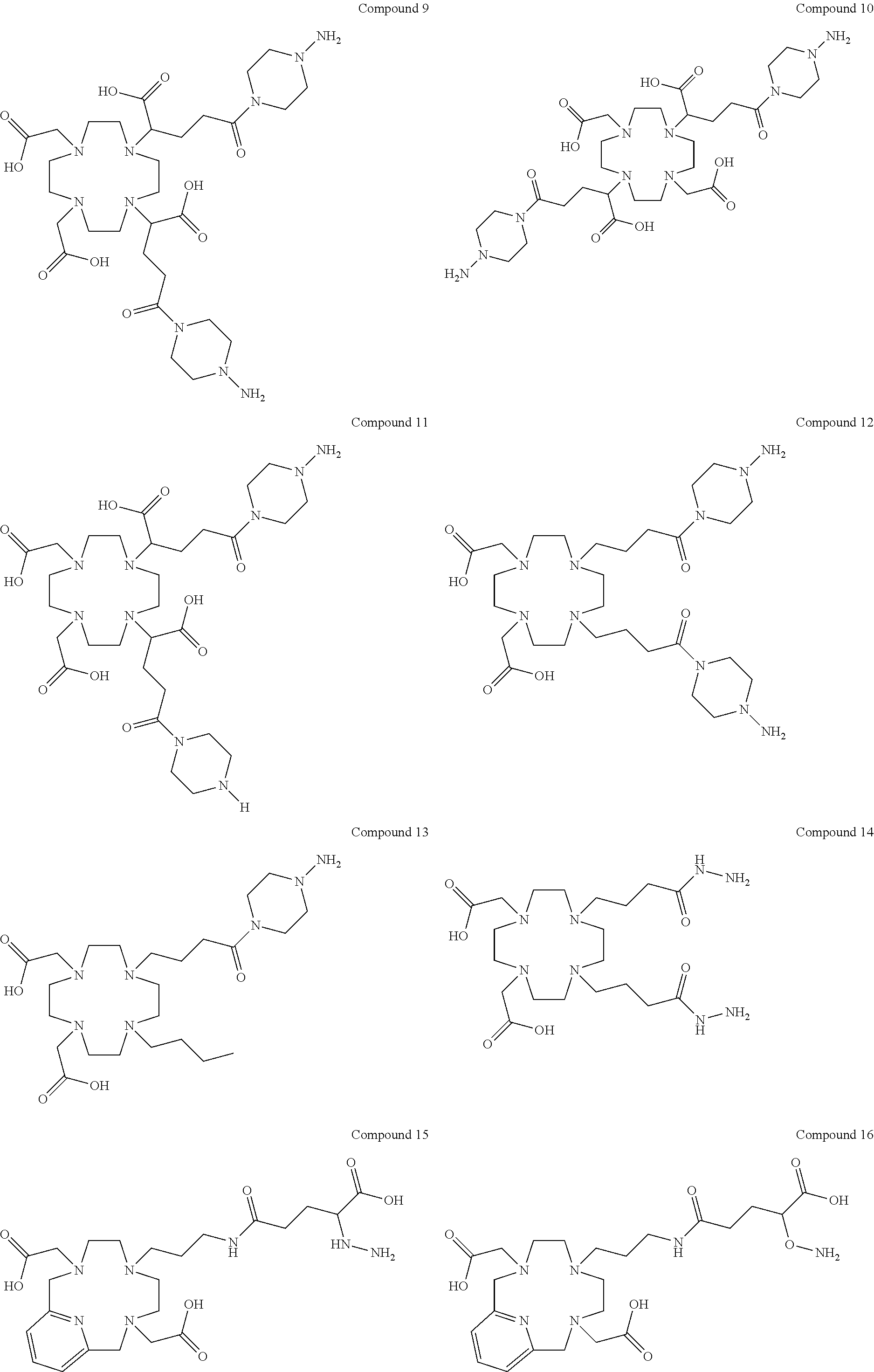

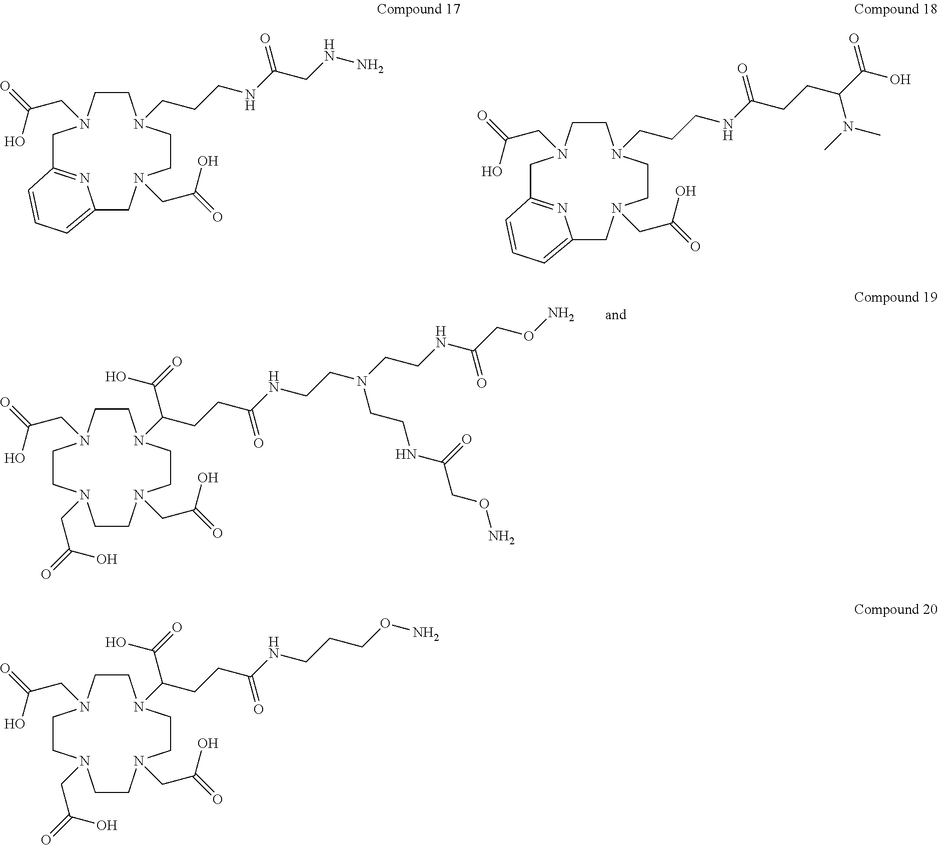 [SMILES: NNCC(=O)NCCCN1CCN(CC(=O)O)Cc2cccc(n2)CN(CC(=O)O)CC1 ; CN(C)C(CCC(=O)NCCCN1CCN(CC(=O)O)Cc2cccc(n2)CN(CC(=O)O)CC1)C(=O)O ; NOCC(=O)NCCN(CCNC(=O)CCC(C(=O)O)N1CCN(CC(=O)O)CCN(CC(=O)O)CCN(CC(=O)O)CC1)CCNC(=O)CON ; NOCCCNC(=O)CCC(C(=O)O)N1CCN(CC(=O)O)CCN(CC(=O)O)CCN(CC(=O)O)CC1]

or a pharmaceutically acceptable salt thereof.

In some embodiments, the compound further comprises a complexed metal cation. In some embodiments, the metal cation is one used in magnetic resonance imaging. In some embodiments, the metal cation is one used in positron emission tomography.

In some embodiments, the metal cation is a Zn, Ga, Gd, Cu, Yb, Mn, Tc, In, Y, or Zr cation. In some embodiments, the metal cation is Zn²⁺, Ga³⁺, Gd³⁺, Cu²⁺, Yb³⁺, or Mn²⁺. In some embodiments, the metal cation is Zn²⁺. In some embodiments, the metal cation is Ga³⁺. In some embodiments, the metal cation is Gd³⁺. In some embodiments, the metal cation is Cu²⁺. In some embodiments, the metal cation is Yb³⁺. In some embodiments, the metal cation is Mn²⁺.

In some embodiments, the metal cation is a ^{99m}Tc, ⁶⁷Ga, ⁶⁸Ga, ⁵²Mn, ⁶⁰Cu, ⁶¹Cu, ⁶²Cu, ⁶⁴Cu, ⁸⁹Zr, ⁸⁶Y, or ¹¹¹In cation. In some embodiments, the metal cation is a ⁶⁸Ga, ⁵²Mn, or ⁶⁴Cu cation. In some embodiments, the metal cation is a ^{99m}Tc cation. In some embodiments, the metal cation is a ⁶⁷Ga cation. In some embodiments, the metal cation is a ⁶⁸Ga cation. In some embodiments, the metal cation is a ²Mn cation. In some embodiments, the metal cation is a 6° C. U cation. In some embodiments, the metal cation is a 6Cu cation. In some embodiments, the metal cation is a ⁶¹Cu cation. In some embodiments, the metal cation is a ⁶²Cu cation. In some embodiments, the metal cation is a 64Cu cation. In some embodiments, the metal cation is a ⁸⁹Zr cation. In some embodiments, the metal cation is a ⁸⁶Y cation. In some embodiments, the metal cation is a ¹¹¹In cation.

In some embodiments, the compound of Formula I is

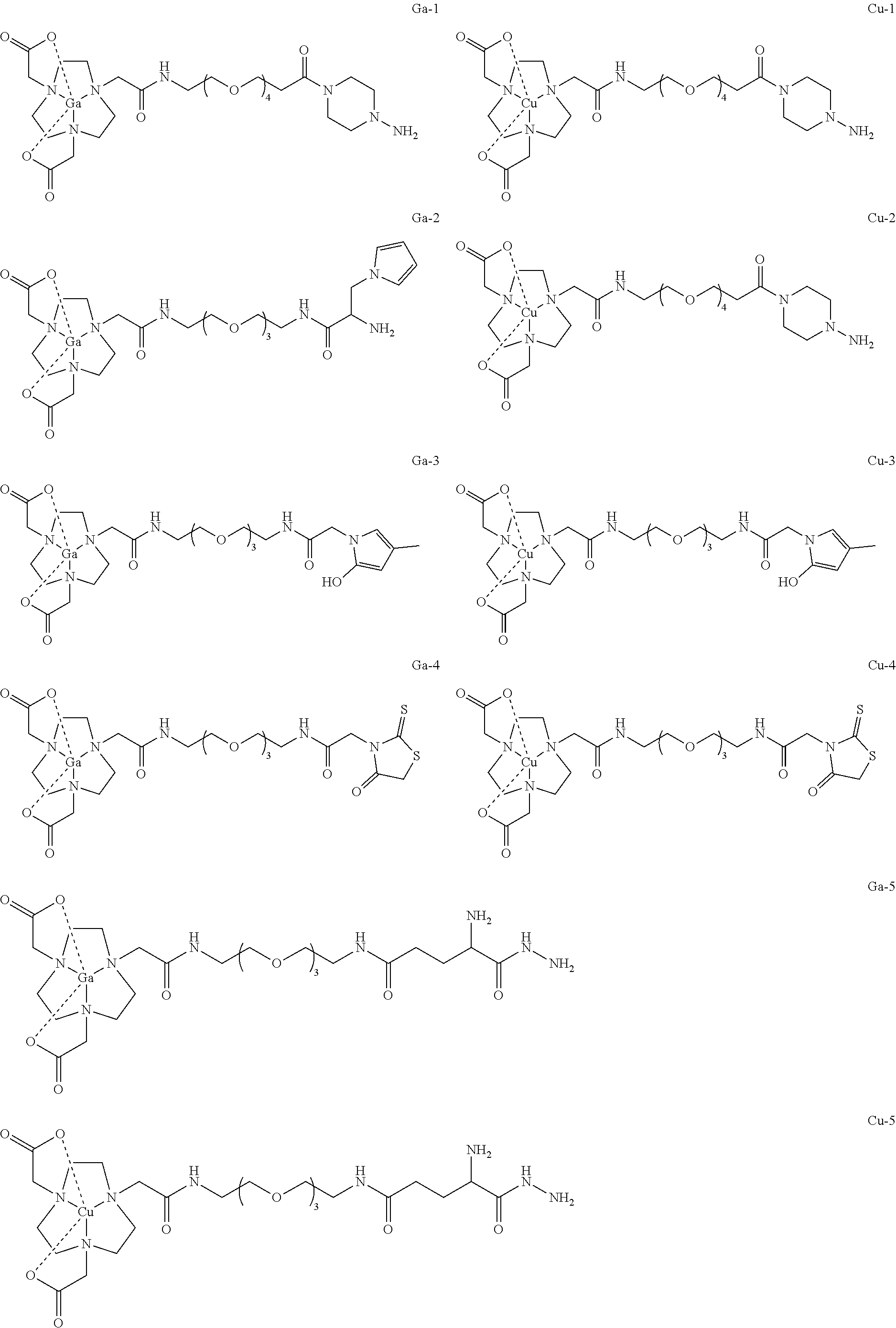

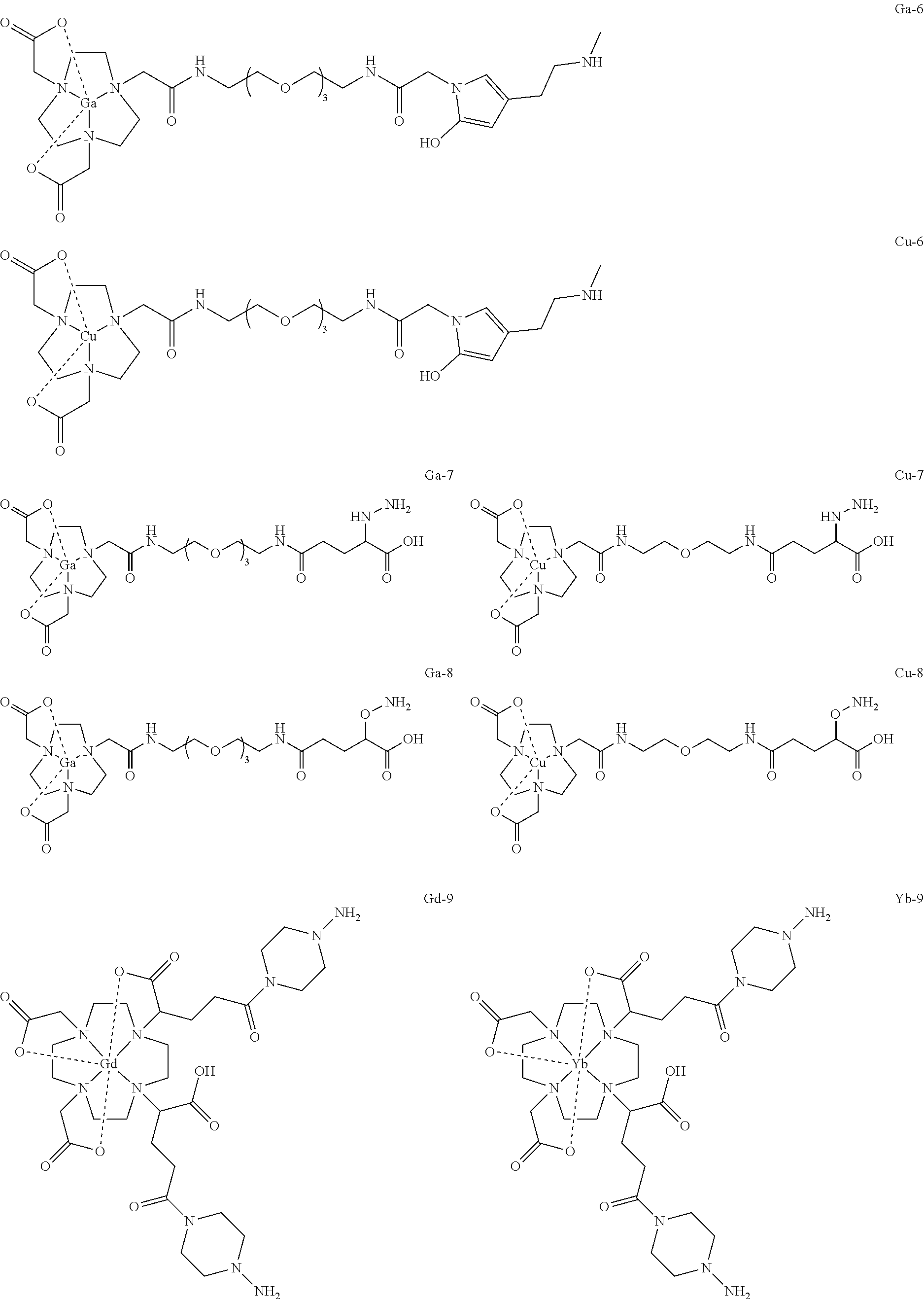

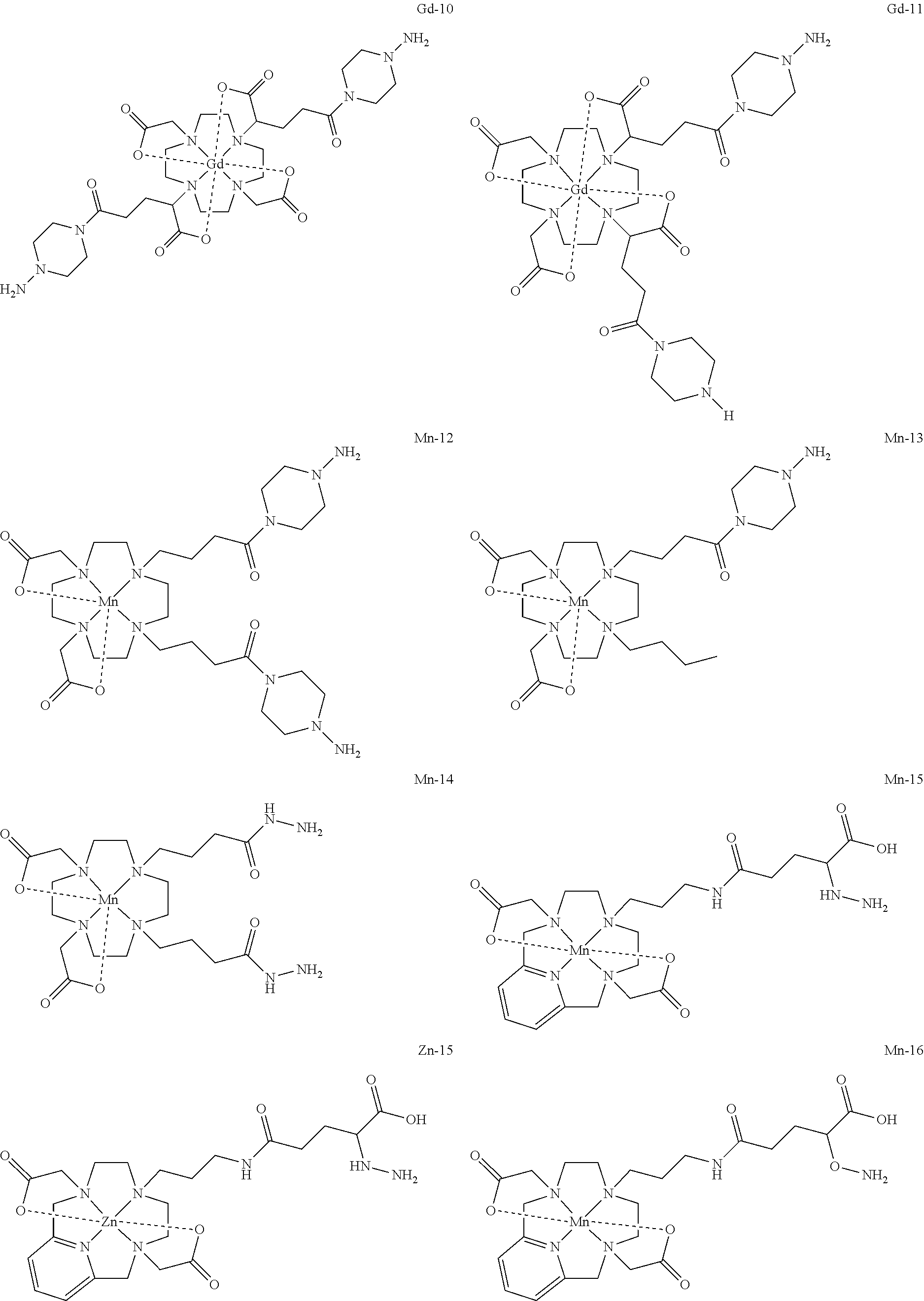

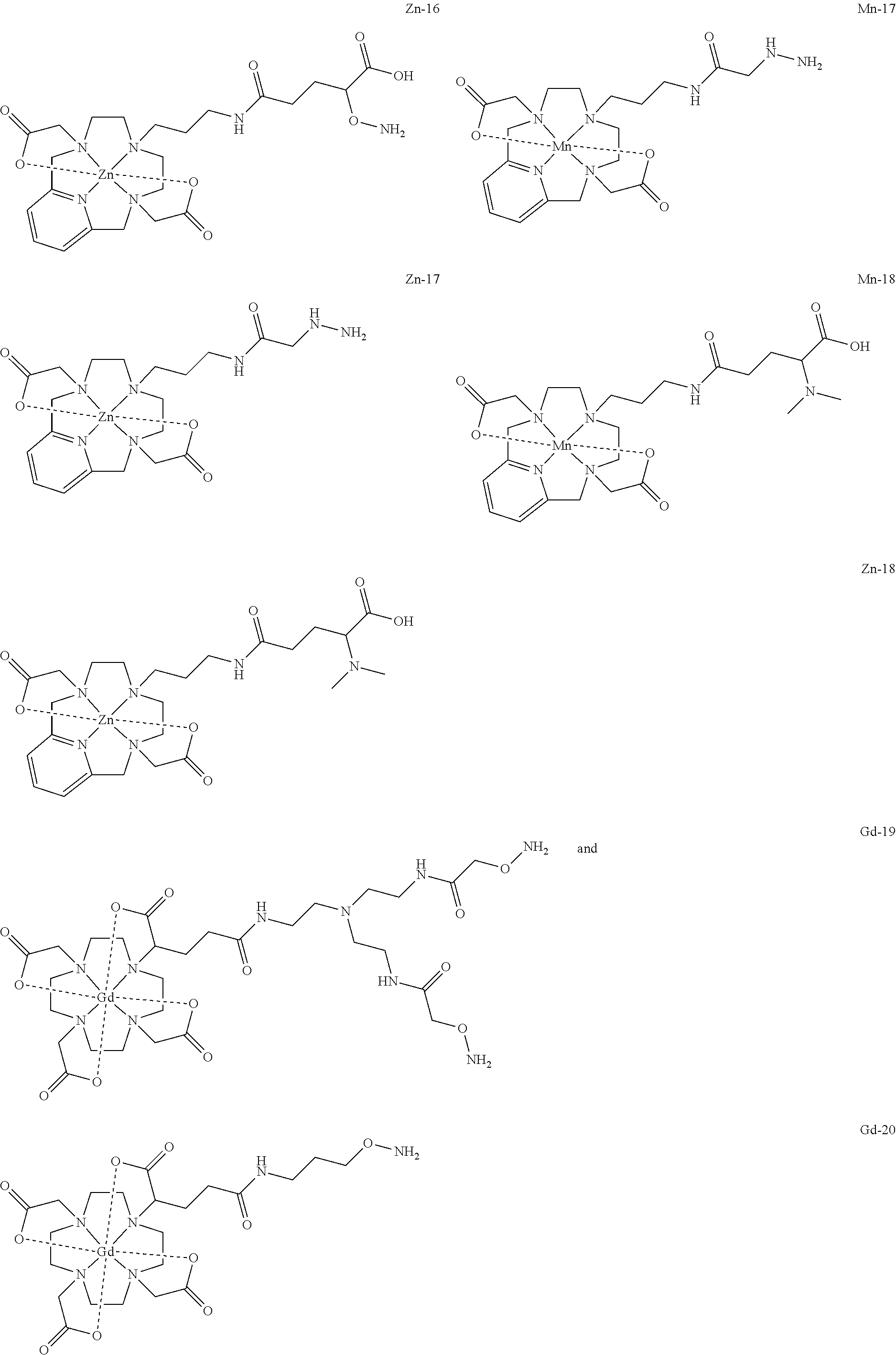

or a pharmaceutically acceptable salt thereof.

Also provided herein is a compound of Formula (II)

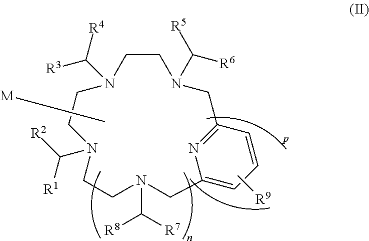

or a pharmaceutically acceptable salt thereof, wherein:

M is a metal cation;

each R¹, R³, R⁵, and R⁷ are independently hydrogen or —C(═O)OH;

each R², R⁴, R⁶, and R⁸ are independently hydrogen or C₃₋₂₅ alkyl, wherein the C₃₋₂₅ alkyl is optionally substituted with 1-2 substituents independently selected from the group consisting of —NR^{A}R^{B}, —OH, halogen, C₁₋₆ alkoxy, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of halogen, —NR^{A}R^{B}, —OH, C₁₋₆ alkyl, and —C₁₋₆ alkyl-(NR^{A}R^{B}); and one or more non-adjacent carbon atoms of the C₃₋₂₅ alkyl are optionally replaced by O, N, NH, or N(CH₃);

R⁹ is H, halogen, —NR^{A}R^{B}, —OH, C₁₋₆ alkyl, or —C₁₋₆ alkyl-(NR^{A}R^{B});

each R^{A} and R^{B} are independently hydrogen or C₁₋₆ alkyl;

n is 0 or 1; and

p is 0 or 1;

wherein if n is 0 at least two of R¹, R³, and R⁵ are —C(═O)OH and if n is 1 at least two of R¹, R³, R⁵, and R⁷ are —C(═O)OH.

In some embodiments, provided herein is a composition comprising a compound disclosed herein, or a pharmaceutically acceptable salt thereof, and a pharmaceutically acceptable carrier. In some embodiments, the composition comprises a mixture of compounds disclosed herein, or a pharmaceutically acceptable salt thereof. In some embodiments, the composition is formulated for parenteral admiration. In some embodiments, the composition is a solid formulated for dissolution in a pharmaceutically acceptable liquid medium prior to administration.

### Methods

Some embodiments provide a method for in vivo imaging of a subject comprising (a) administering to a subject a compound of any one of Formulae (I) or (II), or a mixture of any of the foregoing, or a pharmaceutical composition comprising same; and (b) obtaining an image of the subject after a period of time. Step (b) can comprises obtaining an image of an entire subject (e.g., a full body scan), imaging specific regions of the subject's body, or both.

The specific regions of the subject's body can be organs. For example, the organ can be the liver, stomach, esophagus, pancreas, lungs, kidneys, bladder, thyroid, heart, spleen, bowel or brain. In some embodiments, the organ is the liver, heart, liver and kidney.

Some embodiments provide a method of measuring fibrogenesis. Some embodiments provide a method of detecting fibrogenesis. Some embodiments provide a method of measuring bile duct ligation. Some embodiments provide a method of detecting bile duct ligation. Some embodiments provide a method of measuring bleomycin-injury. Some embodiments provide a method of detecting bleomycin-injury.

The imaging can be, for example, magnetic resonance (MR) imaging, nuclear imaging, positron emission tomography (PET) imaging, single photon emission computed tomography (SPECT) imaging, optical imaging, or optical microscopy.

Some embodiments provide a method imaging a subject comprising:
(a) administering to a subject a compound or composition disclosed herein; and
(b) obtaining an image of the subject after a period of time.

In some embodiments, the image obtained in step (b) is indicative of a disease or disorder as described herein.

Some embodiments provide a method of imaging a subject comprising:
(a) obtaining a first image of the subject;
(b) administering to a subject a compound or composition disclosed herein;
(c) obtaining a second image of the subject after a period of time; and
(d) comparing the first image of the subject and the second image of the subject.

In some embodiments, the comparing of the first image and the second image in step (d) is indicative of a disease or disorder as described herein.

### Magnetic Resonance Imaging

Some embodiments provide a method of magnetic resonance (MR) imaging a subject comprising:
(a) administering to a subject a compound or composition disclosed herein; and
(b) obtaining a magnetic resonance image of the subject after a period of time.

Some embodiments provide a method of magnetic resonance (MR) imaging a subject comprising:
(a) obtaining a first magnetic resonance image of the subject;
(b) administering to a subject a compound or composition disclosed herein;
(c) obtaining a second magnetic resonance image of the subject after a period of time; and
(d) comparing the first magnetic resonance image of the subject and the second magnetic resonance image of the subject.

In some embodiments, the comparing of the first magnetic resonance image and the second magnetic resonance image in step (d) is indicative of a disease or disorder as described herein.

Some embodiments provide a method for imaging liver fibrogenesis in a subject comprising:
(a) administering to a subject a compound or composition disclosed herein; and
(b) obtaining a magnetic resonance image of the subject after a period of time.

Some embodiments provide a method of measuring liver fibrogenesis in a subject comprising:
(a) administering to the subject a compound or composition disclosed herein;
(b) obtaining a first magnetic resonance image of the subject after a period of time;
(c) administering to a subject a compound or composition disclosed herein after a second period of time;
(d) obtaining a second magnetic resonance image of the subject after a period of time; and
(e) comparing the first magnetic resonance image of the subject and the second magnetic resonance image of the subject, thereby measuring the liver fibrogenesis in the subject.

Some embodiments provide a method for detecting liver fibrogenesis in a subject comprising:
(a) administering to a subject a compound or composition disclosed herein; and
(b) obtaining a magnetic resonance image of the subject after a period of time, thereby detecting liver fibrogenesis in the subject.

Some embodiments provide a method of detecting liver fibrogenesis in a subject comprising:
(a) administering to the subject a compound or composition disclosed herein;
(b) obtaining a first magnetic resonance image of the subject after a period of time;
(c) administering to a subject a compound or composition disclosed herein after a second period of time;
(d) obtaining a second magnetic resonance image of the subject after a period of time; and
(e) comparing the first magnetic resonance image of the subject and the second magnetic resonance image of the subject, thereby detecting the presence or absence of liver fibrogenesis in the subject.

Some embodiments provide a method for detecting liver fibrogenesis in a subject comprising obtaining a magnetic resonance image of the subject within a period of time after the subject has been administered subject a compound or composition disclosed herein.

### Positron Emission Tomography Imaging

Positron emission tomography can comprise, for example, measuring the signal in the organ of interested expressed as percent injected dose per cubic centimeter (cc) of tissue or as a standardized uptake value (SUV). In some embodiments, the signal in the organ of interest is compared to a reference tissue like muscle and the target-to-background ratio is measured.

Some embodiments provide method of positron emission tomography (PET) imaging a subject comprising:
(a) administering to a subject a compound or composition disclosed herein; and
(b) obtaining a positron emission tomography image of the subject after a period of time.

Some embodiments provide a method for imaging liver fibrogenesis in a subject comprising:
(a) administering to a subject a compound or composition disclosed herein; and
(b) obtaining a PET image of the subject after a period of time, thereby imaging liver fibrosis in the subject.

Some embodiments provide a method of measuring liver fibrogenesis in a subject comprising:
(a) administering to the subject a compound or composition disclosed herein;
(b) obtaining a first PET image of the subject after a period of time;
(c) administering to a subject a compound or composition disclosed herein after a second period of time;
(d) obtaining a second PET image of the subject after a period of time; and
(e) comparing the first PET image of the subject and the second PET image of the subject, thereby measuring liver fibrogenesis in the subject.

Some embodiments provide a method for detecting liver fibrogenesis in a subject comprising:
(a) administering to a subject a compound or composition disclosed herein; and
(b) obtaining a PET image of the subject after a period of time, thereby detecting the presence of absence of liver fibrogenesis in the subject.

Some embodiments provide a method of detecting liver fibrogenesis in a subject comprising:
(a) administering to the subject a compound or composition disclosed herein;
(b) obtaining a first PET image of the subject after a period of time;
(c) administering to a subject a compound or composition disclosed herein after a second period of time;
(d) obtaining a second PET image of the subject after a period of time; and
(e) comparing the first PET image of the subject and the second PET image of the subject, thereby detecting the presence or absence of liver fibrogenesis in the subject.

Some embodiments provide a method for detecting liver fibrogenesis in a subject comprising obtaining a PET image of the subject within a period of time after the subject has been administered subject a compound or composition disclosed herein.

### Nuclear Imaging

Some embodiments provide method of nuclear imaging a subject comprising:
(a) administering to a subject a compound or composition disclosed herein; and
(b) obtaining a nuclear image of the subject after a period of time.

Some embodiments provide method of nuclear imaging a subject comprising:
(a) obtaining a first nuclear image of the subject;
(b) administering to a subject a compound or composition disclosed herein;
(c) obtaining a second nuclear image of the subject after a period of time; and
(d) comparing the first nuclear image of the subject and the second nuclear image of the subject.

### Single Photon Emission Computed Tomography Imaging

Some embodiments provide method of single photon emission computed tomography imaging a subject comprising:
(a) administering to a subject a compound or composition disclosed herein; and
(b) obtaining a single photon emission computed tomography image of the subject after a period of time.

### Optical Imaging

Some embodiments provide method of optical imaging a subject comprising:
(a) administering to a subject a compound or composition disclosed herein; and
(b) obtaining a optical image of the subject after a period of time.

Some embodiments provide method of optical imaging a subject comprising:
(a) obtaining a first optical image of the subject;
(b) administering to a subject a compound or composition disclosed herein;
(c) obtaining a second optical image of the subject after a period of time; and
(d) comparing the first optical image of the subject and the second optical image of the subject.

### Optical Microscopy Imaging

Some embodiments provide method of optical microscopy imaging a subject comprising:
(a) administering to a subject a compound or composition disclosed herein; and
(b) obtaining a optical microscopy image of the subject after a period of time.

Some embodiments provide method of optical microscopy imaging a subject comprising:
(a) obtaining a first optical microscopy image of the subject;
(b) administering to a subject a compound or composition disclosed herein;
(c) obtaining a second optical microscopy image of the subject after a period of time; and
(d) comparing the first optical microscopy image of the subject and the second optical microscopy image of the subject.

### Definitions

The term “n-membered” where n is an integer typically describes the number of ring-forming atoms in a moiety where the number of ring-forming atoms is n. For example, piperidinyl is an example of a 6-membered heterocyclyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted with the indicated groups. The substituents are independently selected, and substitution may be at any chemically accessible position. As used herein, the term “substituted” means that a hydrogen atom is removed and replaced by the indicated substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.” Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms. The carbon atoms of a “Cₙ₋ₘ alkyl” group can be optionally substituted by one or more oxo (e.g., C(═O)).

As used herein, the term “Cₙ₋ₘ alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “halo” refers to F, Cl, Br, or I. In some embodiments, a halo is F, Cl, or Br.

As used herein, the terms “carbonyl” or “oxo”, employed alone or in combination with other terms, refers to a —C(O)— group.

As used herein, “heteroaryl” refers to a monocyclic or polycyclic (e.g., having 2, 3, or 4 fused rings) aromatic heterocycle having at least one heteroatom ring member selected from N, O, S, and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-6 monocyclic heteroaryl having 1, 2, or 3 heteroatom ring members independently selected from N, O, S, and B. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, S, and B. In some embodiments, the heteroaryl group contains 3 to 14, 4 to 14, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, pyridine, pyrimidine, pyrazine, pyridazine, pyrrole, pyrazole, azolyl, oxazole, isoxazole, thiazole, isothiazole, imidazole, furan, thiophene, triazole, tetrazole, thiadiazole, quinoline, isoquinoline, indole, benzothiophene, benzofuran, benzisoxazole, imidazo[1,2-b]thiazole, purine, triazine, thieno[3,2-b]pyridine, imidazo[1,2-a]pyridine, 1,5-naphthyridine, 1H-pyrazolo[4,3-b]pyridine, and the like.

A five-membered heteroaryl is a heteroaryl group having five ring-forming atoms wherein one or more (e.g., 1, 2, or 3) of the ring-forming atoms are independently selected from N, O, B, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine.

A six-membered heteroaryl ring is a heteroaryl with a ring having six ring-forming atoms wherein one or more (e.g., 1, 2, or 3) ring atoms are independently selected from N, O, S, and B. Exemplary six-membered ring heteroaryls are pyridyl, pyrazinyl, pyrimidinyl, triazinyl and pyridazinyl.

As used herein, “heterocyclyl” refers to monocyclic or polycyclic heterocycles having at least one non-aromatic ring (saturated or partially saturated ring), wherein one or more of the ring-forming carbon atoms of the heterocyclyl is replaced by a heteroatom selected from N, O, S, and B, and wherein the ring-forming carbon atoms and heteroatoms of a heterocyclyl group can be optionally substituted by one or more oxo or sulfide (e.g., C(O), S(O), C(S), or S(O)₂, etc). Heterocyclyl groups include monocyclic and polycyclic (e.g., having 2, 3, or 4 fused rings) systems. Included in heterocyclyl are monocyclic and polycyclic 3-14-, 4-14-, 3-10-, 4-10-, 5-10-4-7-, 5-7-, 5-6-, 5- or 6-membered heterocyclyl groups. Heterocyclyl groups can also include spirocycles and bridged rings (e.g., a 5-14 membered bridged biheterocyclyl ring having one or more ring-forming carbon atoms replaced by a heteroatom independently selected from N, O, S, and B). The heterocyclyl group can be attached through a ring-forming carbon atom or a ring-forming heteroatom. In some embodiments, the heterocyclyl group contains 0 to 3 double bonds, i.e., is partially saturated. In some embodiments, the heterocyclyl group contains 0 to 2 double bonds.

Example heterocyclyl groups include pyrrolidonyl, pyrrolidin-2-one, 1,3-isoxazolidin-2-one, pyranyl, tetrahydropyran, oxetanyl, azetidinyl, morpholinyl, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, benzazapene, azabicyclo[3.1.0]hexanyl, diazabicyclo[3.1.0]hexanyl, oxabicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxabicyclo[2.2.2]octanyl, azabicyclo[2.2.2]octanyl, azaadamantanyl, diazaadamantanyl, oxa-adamantanyl, azaspiro[3.3]heptanyl, diazaspiro[3.3]heptanyl, oxa-azaspiro[3.3]heptanyl, azaspiro[3.4]octanyl, diazaspiro[3.4]octanyl, oxa-azaspiro[3.4]octanyl, azaspiro[2.5]octanyl, diazaspiro[2.5]octanyl, azaspiro[4.4]nonanyl, diazaspiro[4.4]nonanyl, oxa-azaspiro[4.4]nonanyl, azaspiro[4.5]decanyl, diazaspiro[4.5]decanyl, diazaspiro[4.4]nonanyl, oxa-diazaspiro[4.4]nonanyl and the like. In some embodiments, the heterocyclyl group is pyrrolidonyl, pyrrolidin-2-one, 1,3-isoxazolidin-2-one, pyranyl, tetrahydropuran, oxetanyl, azetidinyl, morpholinyl, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, or azepanyl.

In some embodiments, the heterocyclyl group contains 3 to 14 ring-forming atoms, 4 to 14 ring-forming atoms, 3 to 7 ring-forming atoms, or 5 to 6 ring-forming atoms. In some embodiments, the heterocyclyl group has 1 to 4 heteroatoms, 1 to 3 heteroatoms, 1 to 2 heteroatoms or 1 heteroatom. In some embodiments, the heterocyclyl is a monocyclic 4-6 membered heterocyclyl having 1 or 2 heteroatoms independently selected from N, O, S, and B and having one or more oxidized ring members. In some embodiments, the heterocyclyl is a monocyclic or bicyclic 4-10 membered heterocyclyl having 1, 2, 3, or 4 heteroatoms independently selected from N, O, S, and B and having one or more oxidized ring members.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl or sulfonyl group. “Oxo” can also refer to an oxygen atom as a ligand to a metal atom, such as an iron atom.

“Subject” as used herein, means a human or a non-human mammal, e.g., a dog, a cat, a mouse, a rat, a cow, a sheep, a pig, a goat, a non-human primate, or a bird, e.g., a chicken, as well as any other vertebrate or invertebrate. In some embodiments, the subject is a human.

The term “about” as used in connection with a numerical value throughout the specification and the claims denotes an interval of accuracy, familiar and acceptable to a person skilled in the art. Such interval of accuracy is, for example, +10%.

Metal cations can include a metal cations with an atomic number of 21-29, 40, 42, or 57-83. For example, metal cations can include stable or unstable isotopes of metals. Metal cations can include mixtures of isotopes or a single isotope. In some embodiments, the metal cation is radioactive. In some embodiments, the metal cation is non-radioactive.

### EMBODIMENTS

Embodiment 1: A compound of Formula (I)

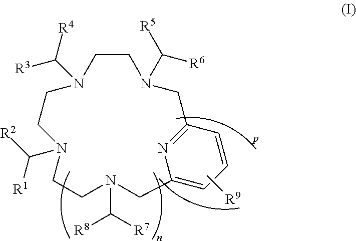

or a pharmaceutically acceptable salt thereof, wherein:

each R¹, R³, R⁵, and R⁷ are independently hydrogen or —C(═O)OH;

each R², R⁴, R⁶, and R⁸ are independently hydrogen or C₃₋₂₅ alkyl, wherein the C₃₋₂₅ alkyl is optionally substituted with 1-2 substituents independently selected from the group consisting of —NR^{A}R^{B}, —OH, halogen, C₁₋₆ alkoxy, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of halogen, —NR^{A}R^{B}, —OH, C₁₋₆ alkyl, and —C₁₋₆ alkyl-(NR^{A}R^{B}); and one or more non-adjacent carbon atoms of the C₃₋₂₅ alkyl are optionally replaced by O, N, NH, or N(CH₃);

R⁹ is H, halogen, —NR^{A}R^{B}, —OH, C₁₋₆ alkyl, or —C₁₋₆ alkyl-(NR^{A}R^{B});

each R^{A} and R^{B} are independently hydrogen or C₁₋₆ alkyl;

n is 0 or 1; and

p is 0 or 1;

wherein if n is 0 at least two of R¹, R³, and R⁵ are —C(═O)OH and if n is 1 at least two of R¹, R³, R⁵, and R⁷ are —C(═O)OH.

Embodiment 2: The compound of embodiment 1, or a pharmaceutically acceptable salt thereof, wherein n is 0.

Embodiment 3: The compound of embodiment 2, or a pharmaceutically acceptable salt thereof, wherein R², R⁴, and R⁶ are independently selected from the group consisting of hydrogen and C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 4: The compound of embodiment 2, or a pharmaceutically acceptable salt thereof, wherein R², R⁴, and R⁶ are independently selected from the group consisting of:
Hydrogen;
C₃₋₂₅ alkyl substituted with 4-10 membered heterocyclyl substituted —NR^{A}R^{B}, wherein one non-adjacent carbon atom of the C₃₋₂₅ alkyl is replaced by NH and four non-adjacent carbon atom of the C₃₋₂₅ alkyl are replaced by O;
C₃₋₂₅ alkyl substituted with 5-10 membered heteroaryl and —NR^{A}R^{B}, wherein two non-adjacent carbon atoms of the C₃₋₂₅ alkyl are replaced by NH and three non-adjacent carbon atoms of the C₃₋₂₅ alkyl are replaced by O;
C₃₋₂₅ alkyl substituted with 5-10 membered heteroaryl substituted with —OH and C₁₋₆ alkyl, wherein two non-adjacent carbon atoms of the C₃₋₂₅ alkyl are replaced by NH and three non-adjacent carbon atoms of the C₃₋₂₅ alkyl are replaced by O;
C₃₋₂₅ alkyl substituted with 4-10 membered heterocyclyl, wherein two non-adjacent carbon atoms of the C₃₋₂₅ alkyl are replaced by NH and three non-adjacent carbon atoms of the C₃₋₂₅ alkyl are replaced by O;
C₃₋₂₅ alkyl substituted with two —NR^{A}R^{B}, wherein three non-adjacent carbon atoms of the C₃₋₂₅ alkyl are replaced by NH and three non-adjacent carbon atoms of the C₃₋₂₅ alkyl are replaced by O;
C₃₋₂₅ alkyl substituted with 4-10 membered heterocyclyl substituted with —C₁₋₆ alkyl-(NR^{A}R^{B}), wherein two non-adjacent carbon atom of the C₃₋₂₅ alkyl are replaced by NH and three non-adjacent carbon atom of the C₃₋₂₅ alkyl are replaced by O;
C₃₋₂₅ alkyl substituted with —NR^{A}R^{B} and —OH, wherein three non-adjacent carbon atoms of the C₃₋₂₅ alkyl are replaced by NH and three non-adjacent carbon atoms of the C₃₋₂₅ alkyl are replaced by O;
C₃₋₂₅ alkyl substituted with —NR^{A}R^{B} and —OH, wherein two non-adjacent carbon atoms of the C₃₋₂₅ alkyl are replaced by NH and four non-adjacent carbon atoms of the C₃₋₂₅ alkyl are replaced by O;
C₃₋₂₅ alkyl substituted with 4-10 membered heterocyclyl substituted with —NR^{A}R^{B};
C₃₋₂₅ alkyl substituted with 4-10 membered heterocyclyl;
C₃₋₂₅ alkyl;
C₃₋₂₅ alkyl substituted with —NR^{A}R^{B}, wherein one non-adjacent carbon atom of the C₃₋₂₅ alkyl is replaced by NH;
C₃₋₂₅ alkyl substituted with —NR^{A}R^{B} and —OH, wherein two non-adjacent carbon atoms of the C₃₋₂₅ alkyl are replaced by NH;
C₃₋₂₅ alkyl substituted with —NR^{A}R^{B} and —OH, wherein one non-adjacent carbon atom of the C₃₋₂₅ alkyl is replaced by NH and one non-adjacent carbon atom of the C₃₋₂₅ alkyl is replaced by O;
C₃₋₂₅ alkyl substituted with —NR^{A}R^{B}, wherein two non-adjacent carbon atoms of the C₃₋₂₅ alkyl are replaced by NH;
C₃₋₂₅ alkyl substituted with OH, wherein one non-adjacent carbon atom of the C₃₋₂₅ alkyl is replaced by NH and one non-adjacent carbon atom of the C₃₋₂₅ alkyl is replaced by N(CH₃);
C₃₋₂₅ alkyl substituted with two —NR^{A}R^{B}, wherein one non-adjacent carbon atoms of the C₃₋₂₅ alkyl are replaced by N, three non-adjacent carbon atoms of the C₃₋₂₅ alkyl are replaced by NH, and two non-adjacent carbon atoms of the C₃₋₂₅ alkyl are replaced by O; and
C₃₋₂₅ alkyl substituted with —NR^{A}R^{B}, wherein one non-adjacent carbon atom of the C₃₋₂₅ alkyl is replaced by NH and one non-adjacent carbon atom of the C₃₋₂₅ alkyl is replaced by O.

Embodiment 5: The compound of embodiment 2, or a pharmaceutically acceptable salt thereof, wherein R², R⁴, and R⁶ are independently selected from the group consisting of:

Hydrogen;

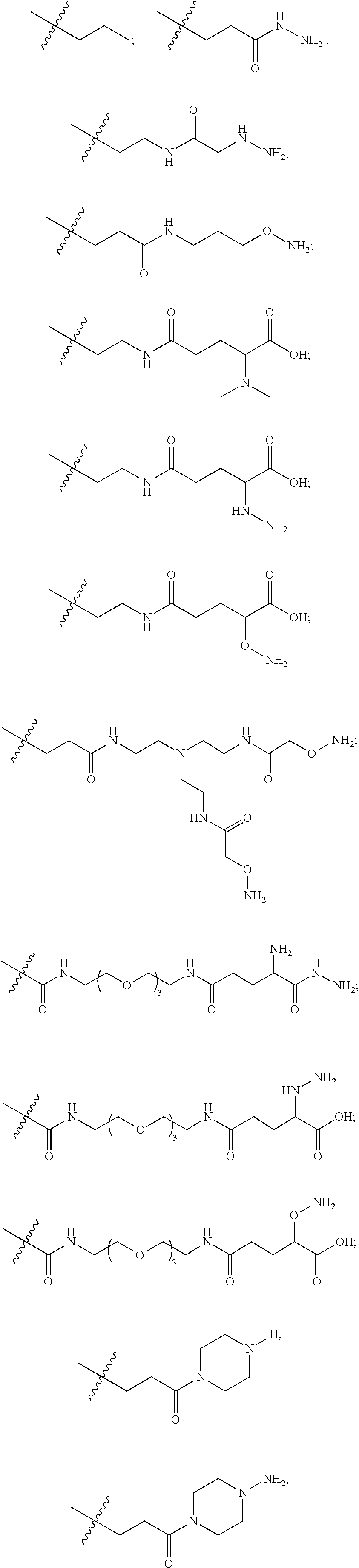

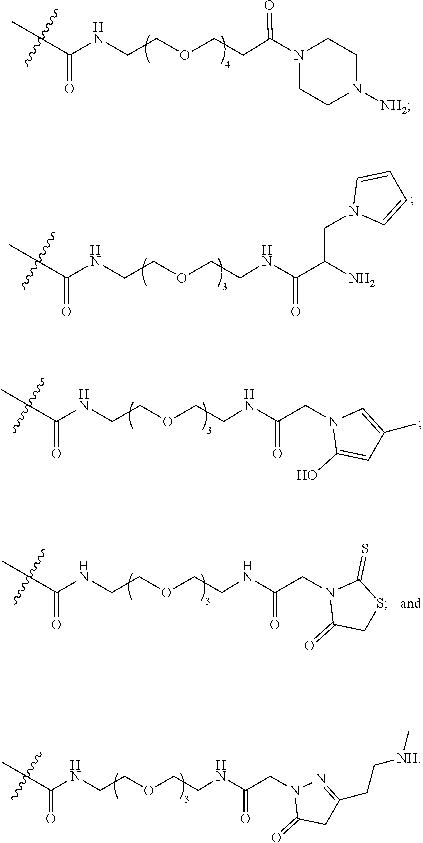

Embodiment 6: The compound of embodiment 2, or a pharmaceutically acceptable salt thereof, wherein R², R⁴, and R⁶ are all hydrogen.

Embodiment 7: The compound of embodiment 2, or a pharmaceutically acceptable salt thereof, wherein R², R⁴, and R⁶ are all C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 8: The compound of embodiment 2, or a pharmaceutically acceptable salt thereof, wherein R² and R⁴ are both hydrogen and R⁶ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 9: The compound of embodiment 2, or a pharmaceutically acceptable salt thereof, wherein R² and R⁶ are both hydrogen and R⁴ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 10: The compound of embodiment 2, or a pharmaceutically acceptable salt thereof, wherein R⁶ and R⁴ are both hydrogen and R² is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 11: The compound of any one of embodiments 2-10, or a pharmaceutically acceptable salt thereof, wherein R¹, R³, and R⁵ are all —C(═O)OH.

Embodiment 12: The compound of any one of embodiments 2-10, or a pharmaceutically acceptable salt thereof, wherein R¹ and R³ are both —C(═O)OH and R⁵ is hydrogen.

Embodiment 13: The compound of any one of embodiments 2-10, or a pharmaceutically acceptable salt thereof, wherein R¹ and R⁵ are both —C(═O)OH and R³ is hydrogen.

Embodiment 14: The compound of any one of embodiments 2-10, or a pharmaceutically acceptable salt thereof, wherein R⁵ and R³ are both —C(═O)OH and R¹ is hydrogen.

Embodiment 15: The compound of embodiment 2, or a pharmaceutically acceptable salt thereof, wherein R¹ is —C(═O)OH and R² is hydrogen.

Embodiment 16: The compound of embodiments 2 or 15, or a pharmaceutically acceptable salt thereof, wherein R³ is —C(═O)OH and R⁴ is hydrogen.

Embodiment 17: The compound of embodiment 2 or 15, or a pharmaceutically acceptable salt thereof, wherein R³ is hydrogen and R⁴ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by 0, N, NH, or N(CH).

Embodiment 18: The compound of any one of embodiments 2 and 15-17, or a pharmaceutically acceptable salt thereof, wherein R⁵ is hydrogen and R⁶ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 19: The compound of any one of embodiment 2 and 15-17, or a pharmaceutically acceptable salt thereof, wherein R⁵ is —C(═O)OH and R⁶ is hydrogen.

Embodiment 20: The compound of embodiment 1, or a pharmaceutically acceptable salt thereof, wherein n is 1.

Embodiment 21: The compound of embodiment 20, or a pharmaceutically acceptable salt thereof, wherein R², R⁴, R⁶, and R⁸ are independently selected from the group consisting of hydrogen and C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 22: The compound of embodiment 20, or a pharmaceutically acceptable salt thereof, wherein R², R⁴, R⁶, and RY are independently selected from the group consisting of:
Hydrogen;
C₃₋₂₅ alkyl substituted with 4-10 membered heterocyclyl substituted —NR^{A}R^{B}, wherein one non-adjacent carbon atom of the C₃₋₂₅ alkyl is replaced by NH and four non-adjacent carbon atom of the C₃₋₂₅ alkyl are replaced by O;
C₃₋₂₅ alkyl substituted with 5-10 membered heteroaryl and —NR^{A}R^{B}, wherein two non-adjacent carbon atoms of the C₃₋₂₅ alkyl are replaced by NH and three non-adjacent carbon atoms of the C₃₋₂₅ alkyl are replaced by O;
C₃₋₂₅ alkyl substituted with 5-10 membered heteroaryl substituted with —OH and C₁₋₆ alkyl, wherein two non-adjacent carbon atoms of the C₃₋₂₅ alkyl are replaced by NH and three non-adjacent carbon atoms of the C₃₋₂₅ alkyl are replaced by O;
C₃₋₂₅ alkyl substituted with 4-10 membered heterocyclyl, wherein two non-adjacent carbon atoms of the C₃₋₂₅ alkyl are replaced by NH and three non-adjacent carbon atoms of the C₃₋₂₅ alkyl are replaced by O;
C₃₋₂₅ alkyl substituted with two —NR^{A}R^{B}, wherein three non-adjacent carbon atoms of the C₃₋₂₅ alkyl are replaced by NH and three non-adjacent carbon atoms of the C₃₋₂₅ alkyl are replaced by O;
C₃₋₂₅ alkyl substituted with 4-10 membered heterocyclyl substituted with —C₁₋₆ alkyl-(NR^{A}R^{B}), wherein two non-adjacent carbon atom of the C₃₋₂₅ alkyl are replaced by NH and three non-adjacent carbon atom of the C₃₋₂₅ alkyl are replaced by O;
C₃₋₂₅ alkyl substituted with —NR^{A}R^{B} and —OH, wherein three non-adjacent carbon atoms of the C₃₋₂₅ alkyl are replaced by NH and three non-adjacent carbon atoms of the C₃₋₂₅ alkyl are replaced by O;
C₃₋₂₅ alkyl substituted with —NR^{A}R^{B} and —OH, wherein two non-adjacent carbon atoms of the C₃₋₂₅ alkyl are replaced by NH and four non-adjacent carbon atoms of the C₃₋₂₅ alkyl are replaced by O;
C₃₋₂₅ alkyl substituted with 4-10 membered heterocyclyl substituted with —NR^{A}R^{B};
C₃₋₂₅ alkyl substituted with 4-10 membered heterocyclyl;
C₃₋₂₅ alkyl;
C₃₋₂₅ alkyl substituted with —NR^{A}R^{B}, wherein one non-adjacent carbon atom of the C₃₋₂₅ alkyl is replaced by NH;
C₃₋₂₅ alkyl substituted with —NR^{A}R^{B} and —OH, wherein two non-adjacent carbon atoms of the C₃₋₂₅ alkyl are replaced by NH;
C₃₋₂₅ alkyl substituted with —NR^{A}R^{B} and —OH, wherein one non-adjacent carbon atom of the C₃₋₂₅ alkyl is replaced by NH and one non-adjacent carbon atom of the C₃₋₂₅ alkyl is replaced by O;
C₃₋₂₅ alkyl substituted with —NR^{A}R^{B}, wherein two non-adjacent carbon atoms of the C₃₋₂₅ alkyl are replaced by NH;
C₃₋₂₅ alkyl substituted with OH, wherein one non-adjacent carbon atom of the C₃₋₂₅ alkyl is replaced by NH and one non-adjacent carbon atom of the C₃₋₂₅ alkyl is replaced by N(CH₃);
C₃₋₂₅ alkyl substituted with two —NR^{A}R^{B}, wherein one non-adjacent carbon atoms of the C₃₋₂₅ alkyl are replaced by N, three non-adjacent carbon atoms of the C₃₋₂₅ alkyl are replaced by NH, and two non-adjacent carbon atoms of the C₃₋₂₅ alkyl are replaced by O; and
C₃₋₂₅ alkyl substituted with —NR^{A}R^{B}, wherein one non-adjacent carbon atom of the C₃₋₂₅ alkyl is replaced by NH and one non-adjacent carbon atom of the C₃₋₂₅ alkyl is replaced by O.

Embodiment 23: The compound of embodiment 20, or a pharmaceutically acceptable salt thereof, wherein R², R⁴, R⁶, and R⁸ are independently selected from the group consisting of
Hydrogen;

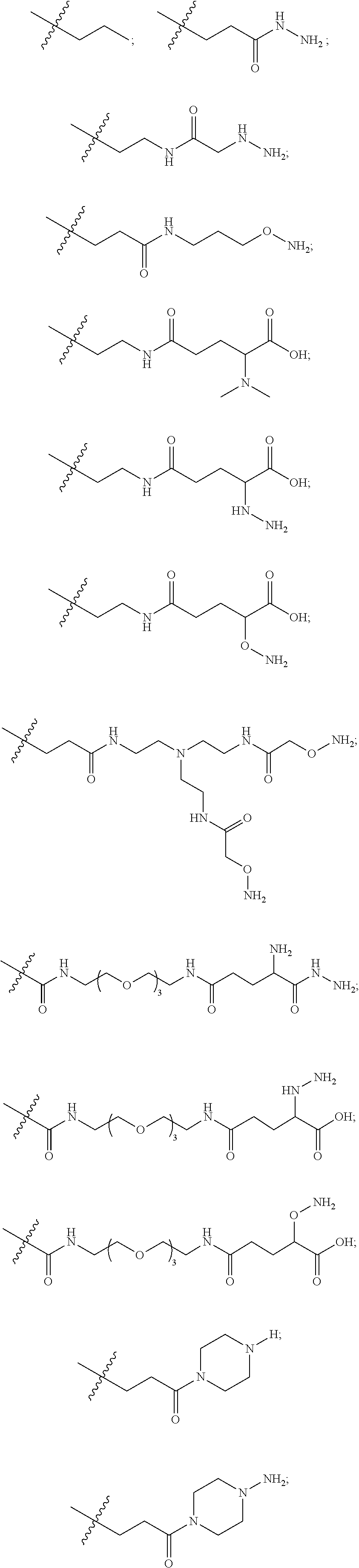

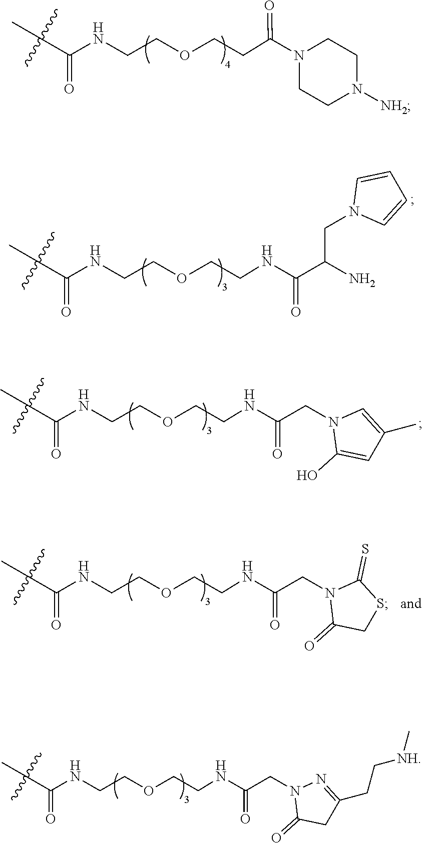

Embodiment 24: The compound of embodiment 20, or a pharmaceutically acceptable salt thereof, wherein R², R⁴, R⁶, and R⁸ are all hydrogen.

Embodiment 25: The compound of embodiment 20, or a pharmaceutically acceptable salt thereof, wherein R², R⁴, R⁶, and R⁸ are all C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 26: The compound of embodiment 20, or a pharmaceutically acceptable salt thereof, wherein R² and R⁴ are both hydrogen and R⁶ and R⁸ are both C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 27: The compound of embodiment 20, or a pharmaceutically acceptable salt thereof, wherein R² and R⁶ are both hydrogen and R⁴ and R⁸ are both C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 28: The compound of embodiment 20, or a pharmaceutically acceptable salt thereof, wherein R² and R⁸ are both hydrogen and R⁴ and R⁶ are both C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 29: The compound of embodiment 20, or a pharmaceutically acceptable salt thereof, wherein R⁴ and R⁶ are both hydrogen and R² and R⁸ are both C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 30: The compound of embodiment 20, or a pharmaceutically acceptable salt thereof, wherein R⁴ and R⁸ are both hydrogen and R² and R⁶ are both C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 31: The compound of embodiment 20, or a pharmaceutically acceptable salt thereof, wherein R⁶ and R⁸ are both hydrogen and R² and R⁴ are both C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 32: The compound of embodiment 20, or a pharmaceutically acceptable salt thereof, wherein R², R⁴, and R⁶ are all hydrogen and R⁸ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by 0, N, NH, or N(CH₃).

Embodiment 33: The compound of embodiment 20, or a pharmaceutically acceptable salt thereof, wherein R², R⁴, and R⁸ are all hydrogen and R⁶ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 34: The compound of embodiment 20, or a pharmaceutically acceptable salt thereof, wherein R², R⁶, and R⁸ are all hydrogen and R⁴ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 35: The compound of embodiment 20, or a pharmaceutically acceptable salt thereof, wherein R⁴, R⁶, and R⁸ are all hydrogen and R² is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 36: The compound of any one of embodiments 20-35, or a pharmaceutically acceptable salt thereof, wherein R¹, R³, R⁵ and R⁷ are all —C(═O)OH.

Embodiment 37: The compound of any one of embodiments 20-35, or a pharmaceutically acceptable salt thereof, wherein R¹ and R³ are both —C(═O)OH and R⁵ and R⁷ are both hydrogen.

Embodiment 38: The compound of any one of embodiments 20-35, or a pharmaceutically acceptable salt thereof, wherein R¹ and R⁵ are both —C(═O)OH and R³ and R⁷ are both hydrogen.

Embodiment 39: The compound of any one of embodiments 20-35, or a pharmaceutically acceptable salt thereof, wherein R¹ and R⁷ are both —C(═O)OH and R³ and R⁵ are both hydrogen.

Embodiment 40: The compound of any one of embodiments 20-35, or a pharmaceutically acceptable salt thereof, wherein R³ and R⁵ are both —C(═O)OH and R¹ and R⁷ are both hydrogen.

Embodiment 41: The compound of any one of embodiments 20-35, or a pharmaceutically acceptable salt thereof, wherein R³ and R⁷ are both —C(═O)OH and R¹ and R⁵ are both hydrogen.

Embodiment 42: The compound of any one of embodiments 20-35, or a pharmaceutically acceptable salt thereof, wherein R⁵ and R⁷ are both —C(═O)OH and R¹ and R³ are both hydrogen.

Embodiment 43: The compound of any one of embodiments 20-35, or a pharmaceutically acceptable salt thereof, wherein R¹, R³, and R⁵ are all —C(═O)OH and R⁷ is hydrogen.

Embodiment 44: The compound of any one of embodiments 20-35, or a pharmaceutically acceptable salt thereof, wherein R¹, R³, and R⁷ are all —C(═O)OH and R⁵ is hydrogen.

Embodiment 45: The compound of any one of embodiments 20-35, or a pharmaceutically acceptable salt thereof, wherein R¹, R⁵, and R⁷ are all —C(═O)OH and R³ is hydrogen.

Embodiment 46: The compound of any one of embodiments 20-35, or a pharmaceutically acceptable salt thereof, wherein R³, R⁵, and R⁷ are all —C(═O)OH and R¹ is hydrogen.

Embodiment 47: The compound of embodiment 20, or a pharmaceutically acceptable salt thereof, wherein R¹ is —C(═O)OH and R² is hydrogen.

Embodiment 48: The compound of embodiment 20, or a pharmaceutically acceptable salt thereof, wherein R¹ is —C(═O)OH and R² is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 49: The compound of any one of embodiments 20, 47, and 48, or a pharmaceutically acceptable salt thereof, wherein R³ is —C(═O)OH and R⁴ is hydrogen.

Embodiment 50: The compound of any one of embodiments 20 and 47-49, or a pharmaceutically acceptable salt thereof, wherein R⁵ is —C(═O)OH and R⁶ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 51: The compound of any one of embodiments 20 and 47-49, or a pharmaceutically acceptable salt thereof, wherein R⁵ is hydrogen and R⁶ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 52: The compound of any one of embodiments 20 and 47-51, or a pharmaceutically acceptable salt thereof, wherein R⁷ is —C(═O)OH and R⁸ is hydrogen.

Embodiment 53: The compound of any one of embodiments 20 and 47-51, or a pharmaceutically acceptable salt thereof, wherein R⁷ is —C(═O)OH and R⁸ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

Embodiment 54: The compound of any one of embodiments 20 and 47-51, or a pharmaceutically acceptable salt thereof, wherein R⁷ is hydrogen and R⁸ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by 0, N, NH, or N(CH₃).

Embodiment 55: The compound of any one of embodiments 1-54, or a pharmaceutically acceptable salt thereof, wherein p is 1.

Embodiment 56: The compound of any one of embodiments 1-55, or a pharmaceutically acceptable salt thereof, wherein R⁹ is H, halogen, or —OH.

Embodiment 57: The compound of any one of embodiments 1-55, or a pharmaceutically acceptable salt thereof, wherein R⁹ is H.

Embodiment 58: The compound of any one of embodiments 1-54, or a pharmaceutically acceptable salt thereof, wherein p is 0.

Embodiment 59: The compound of any one of embodiments 1-58, or a pharmaceutically acceptable salt thereof, wherein R^{A} is hydrogen.

Embodiment 60: The compound of any one of embodiments 1-58, or a pharmaceutically acceptable salt thereof, wherein R^{A} is C₁₋₆ alkyl.

Embodiment 61: The compound of any one of embodiments 1-60, or a pharmaceutically acceptable salt thereof, wherein R^{B} is hydrogen.

Embodiment 62: The compound of any one of embodiments 1-60, or a pharmaceutically acceptable salt thereof, wherein R^{B} is C₁₋₆ alkyl.

Embodiment 63: The compound of any one of embodiments 1-58, or a pharmaceutically acceptable salt thereof, wherein R^{A} and R^{B} are both hydrogen.

Embodiment 64: The compound of embodiment 1, wherein the compound of Formula (I) is a compound of Formula (IA)

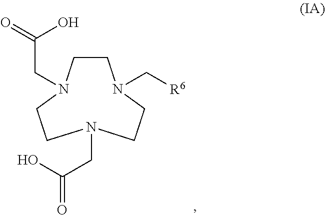

or a pharmaceutically acceptable salt thereof.

Embodiment 65: The compound of embodiment 64, or a pharmaceutically acceptable salt thereof, wherein R⁶ is selected from the group consisting of:

C₃₋₂₅ alkyl substituted with 4-10 membered heterocyclyl substituted —NR^{A}R^{B}, wherein one non-adjacent carbon atom of the C₃₋₂₅ alkyl is replaced by NH and four non-adjacent carbon atom of the C₃₋₂₅ alkyl are replaced by O;

C₃₋₂₅ alkyl substituted with 5-10 membered heteroaryl and —NR^{A}R^{B}, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 5-10 membered heteroaryl substituted with —OH and C₁0.6 alkyl, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl, wherein two non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with two —NR^{A}R^{B}, wherein three non-adjacent carbon atoms of the C₃₋₂₅ alkyl are replaced by NH and three non-adjacent carbon atoms of the C₃₋₂₅ alkyl are replaced by O;

C₃₋₂₅ alkyl substituted with 4-10 membered heterocyclyl substituted with —C₁₋₆ alkyl-(NR^{A}R^{B}), wherein two non-adjacent carbon atom of the C₃₋₂₅ alkyl are replaced by NH and three non-adjacent carbon atom of the C₃₋₂₅ alkyl are replaced by O;

C₃₋₂₅ alkyl substituted with —NR^{A}R^{B} and —OH, wherein three non-adjacent carbon atoms of the C₃₋₂₅ alkyl are replaced by NH and three non-adjacent carbon atoms of the C₃₋₂₅ alkyl are replaced by O; and

C₃₋₂₅ alkyl substituted with —NR^{A}R^{B} and —OH, wherein two non-adjacent carbon atoms of the C₃₋₂₅ alkyl are replaced by NH and four non-adjacent carbon atoms of the C₃₋₂₅ alkyl are replaced by O.

Embodiment 66: The compound of embodiment 1, wherein the compound of Formula (I) is a compound of Formula (IB)

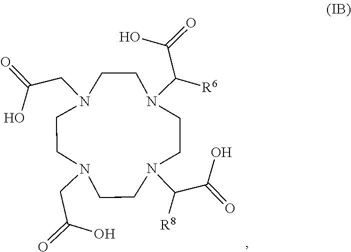

or a pharmaceutically acceptable salt thereof.

Embodiment 67: The compound of embodiment 66, or a pharmaceutically acceptable salt thereof, wherein R⁶ is selected from the group consisting of:

C₃₋₂₅ alkyl substituted with 4-10 membered heterocyclyl substituted with —NR^{A}R^{B}; and

C₃₋₂₅ alkyl substituted with —NR^{A}R^{B}, wherein one non-adjacent carbon atom of the C₃₋₂₅ alkyl is replaced by NH.

Embodiment 68: The compound of embodiment 66 or 67, or a pharmaceutically acceptable salt thereof, wherein R⁸ is selected from the group consisting of:

C₃₋₂₅ alkyl substituted with 4-10 membered heterocyclyl substituted with —NR^{A}R^{B};

C₃₋₂₅ alkyl substituted with 4-10 membered heterocyclyl;

C₃₋₂₅ alkyl; and

C₃₋₂₅ alkyl substituted with —NR^{A}R^{B}, wherein one non-adjacent carbon atom of the C₃₋₂₅ alkyl is replaced by NH.

Embodiment 69: The compound of embodiment 1, wherein the compound of Formula (I) is a compound of Formula (IC)

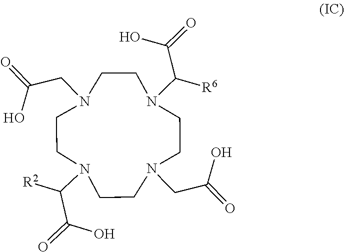

or a pharmaceutically acceptable salt thereof.

Embodiment 70: The compound of embodiment 69, or a pharmaceutically acceptable salt thereof, wherein R² is selected from the group consisting of:

Hydrogen; and

C₃₋₂₅ alkyl substituted with 4-10 membered heterocyclyl substituted with —NR^{A}R^{B};

Embodiment 71: The compound of embodiment 69 or 70, or a pharmaceutically acceptable salt thereof, wherein R⁶ is selected from the group consisting of:

C₃₋₂₅ alkyl substituted with two —NR^{A}R^{B}, wherein one non-adjacent carbon atoms of the C₃₋₂₅ alkyl are replaced by N, three non-adjacent carbon atoms of the C₃₋₂₅ alkyl are replaced by NH, and two non-adjacent carbon atoms of the C₃₋₂₅ alkyl are replaced by O; and

C₃₋₂₅ alkyl substituted with 4-10 membered heterocyclyl substituted with —NR^{A}R^{B};

Embodiment 72: The compound of embodiment 1, wherein the compound of Formula (I) is a compound of Formula (ID)

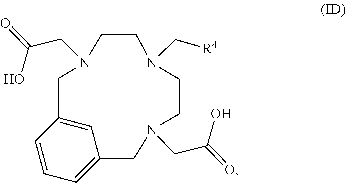

or a pharmaceutically acceptable salt thereof.

Embodiment 73: The compound of embodiment 72, or a pharmaceutically acceptable salt thereof, wherein R⁴ is selected form the group consisting of:

C₃₋₂₅ alkyl substituted with —NR^{A}R^{B} and —OH, wherein two non-adjacent carbon atoms of the C₃₋₂₅ alkyl are replaced by NH;

C₃₋₂₅ alkyl substituted with —NR^{A}R^{B} and —OH, wherein one non-adjacent carbon atom of the C₃₋₂₅ alkyl is replaced by NH and one non-adjacent carbon atom of the C₃₋₂₅ alkyl is replaced by O;

C₃₋₂₅ alkyl substituted with —NR^{A}R^{B}, wherein two non-adjacent carbon atoms of the C₃₋₂₅ alkyl are replaced by NH; and

C₃₋₂₅ alkyl substituted with OH, wherein one non-adjacent carbon atom of the C₃₋₂₅ alkyl is replaced by NH and one non-adjacent carbon atom of the C₃₋₂₅ alkyl is replaced by N(CH₃).

Embodiment 74: The compound of embodiment 1, wherein the compound of Formula (I) is selected from:

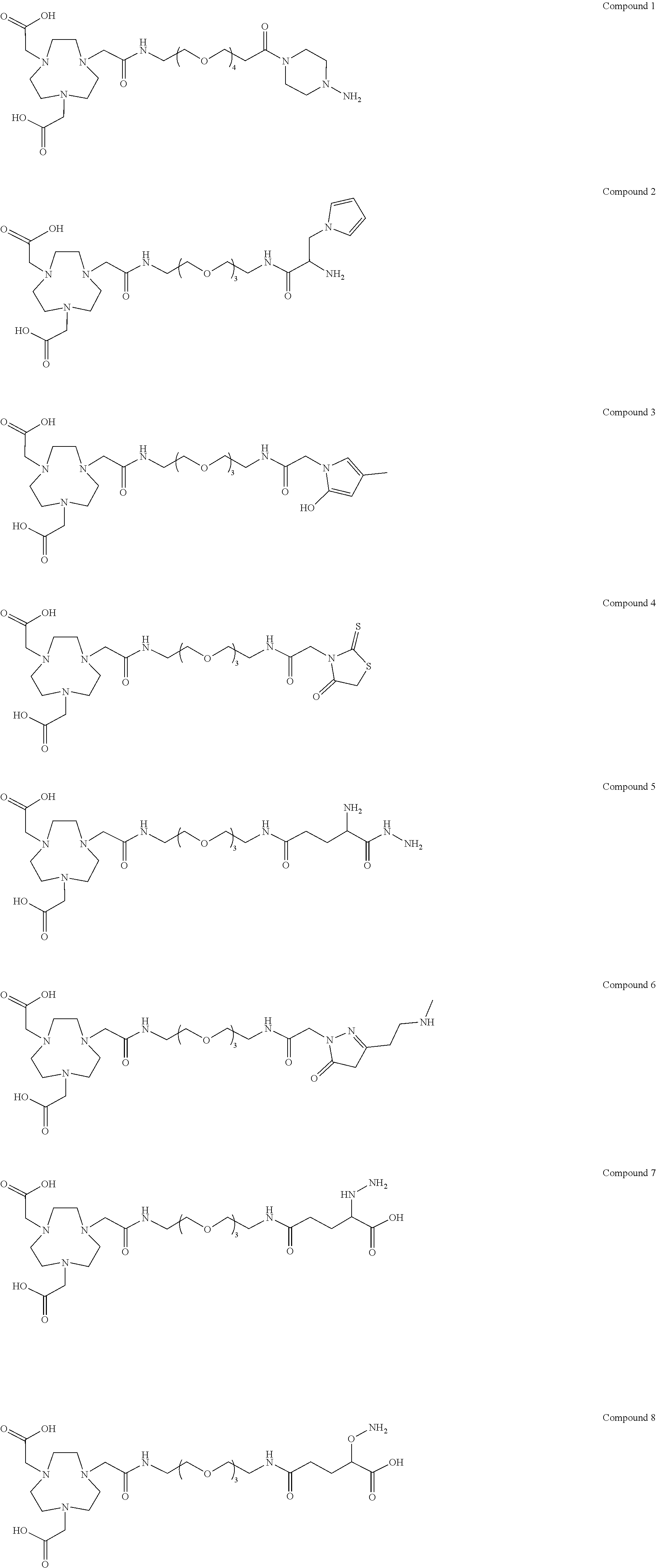

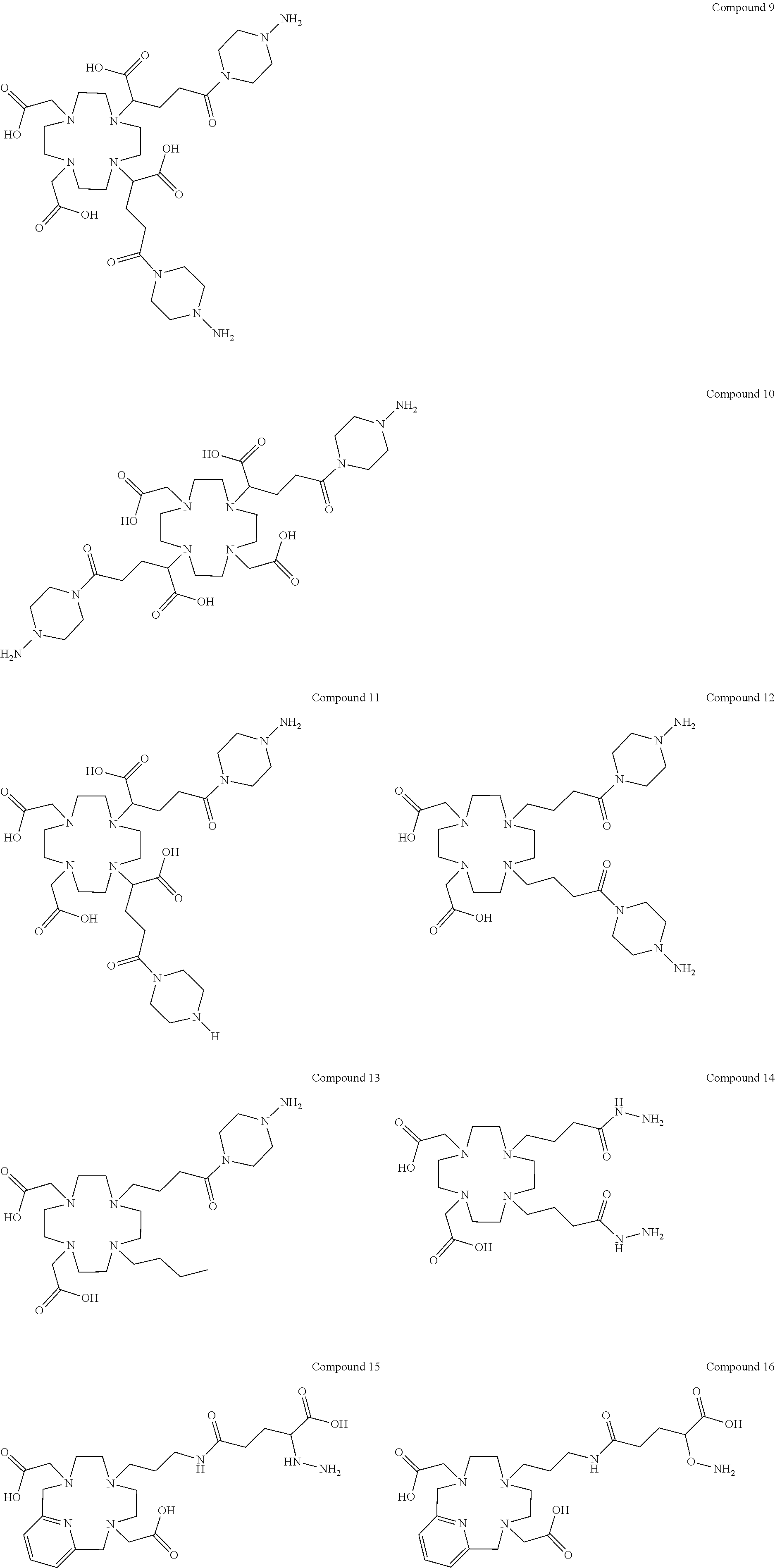

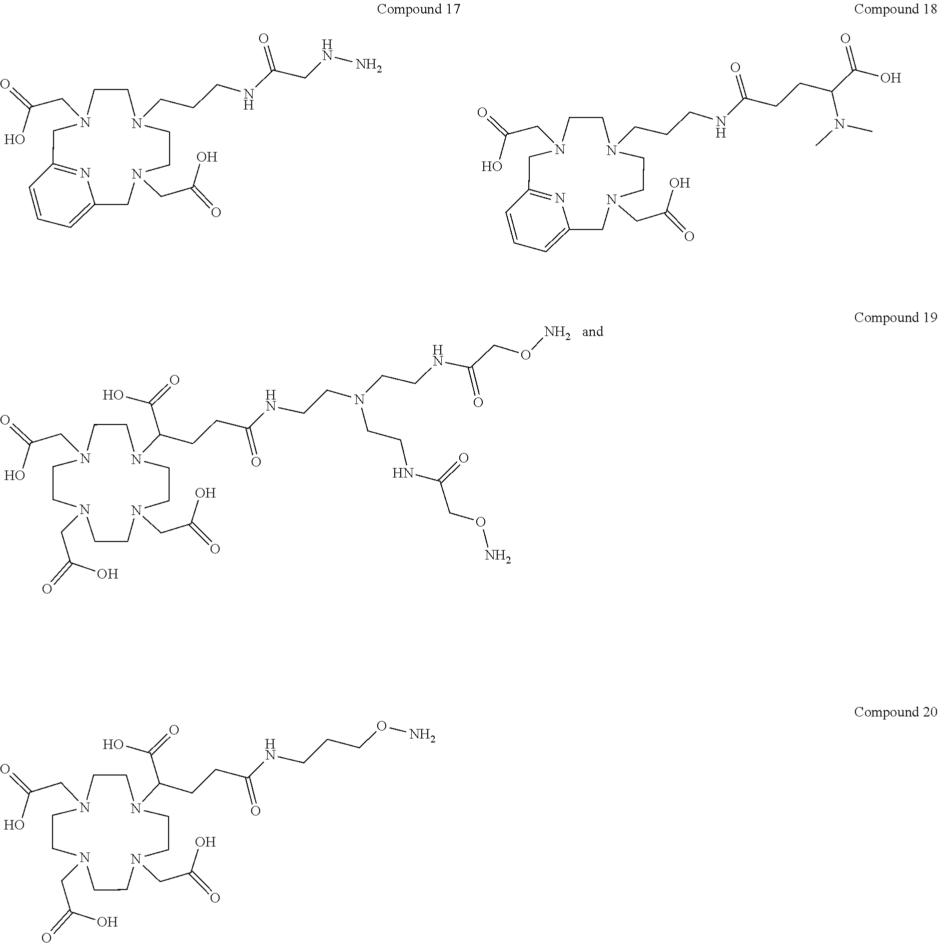 [SMILES: NNCC(=O)NCCCN1CCN(CC(=O)O)Cc2cccc(n2)CN(CC(=O)O)CC1 ; CN(C)C(CCC(=O)NCCCN1CCN(CC(=O)O)Cc2cccc(n2)CN(CC(=O)O)CC1)C(=O)O ; NOCC(=O)NCCN(CCNC(=O)CCC(C(=O)O)N1CCN(CC(=O)O)CCN(CC(=O)O)CCN(CC(=O)O)CC1)CCNC(=O)CON ; "and" ; NOCCCNC(=O)CCC(C(=O)O)N1CCN(CC(=O)O)CCN(CC(=O)O)CCN(CC(=O)O)CC1]

or a pharmaceutically acceptable salt thereof.

Embodiment 75: The compound of any one of embodiments 1-74, or a pharmaceutically acceptable salt thereof, wherein the compound further comprises a complexed metal cation.

Embodiment 76: The compound of embodiment 75, or a pharmaceutically acceptable salt thereof, wherein the metal cation is a Zn, Ga, Gd, Cu, Yb, Mn, Tc, or In cation.

Embodiment 77: The compound of embodiment 75 or 76, or a pharmaceutically acceptable salt thereof, wherein the metal cation is Zn²⁺, Ga³⁺, Gd³⁺, Cu²⁺, Yb³⁺, or Mn²⁺.

Embodiment 78: The compound of embodiment 75, wherein the compound of Formula (I) is

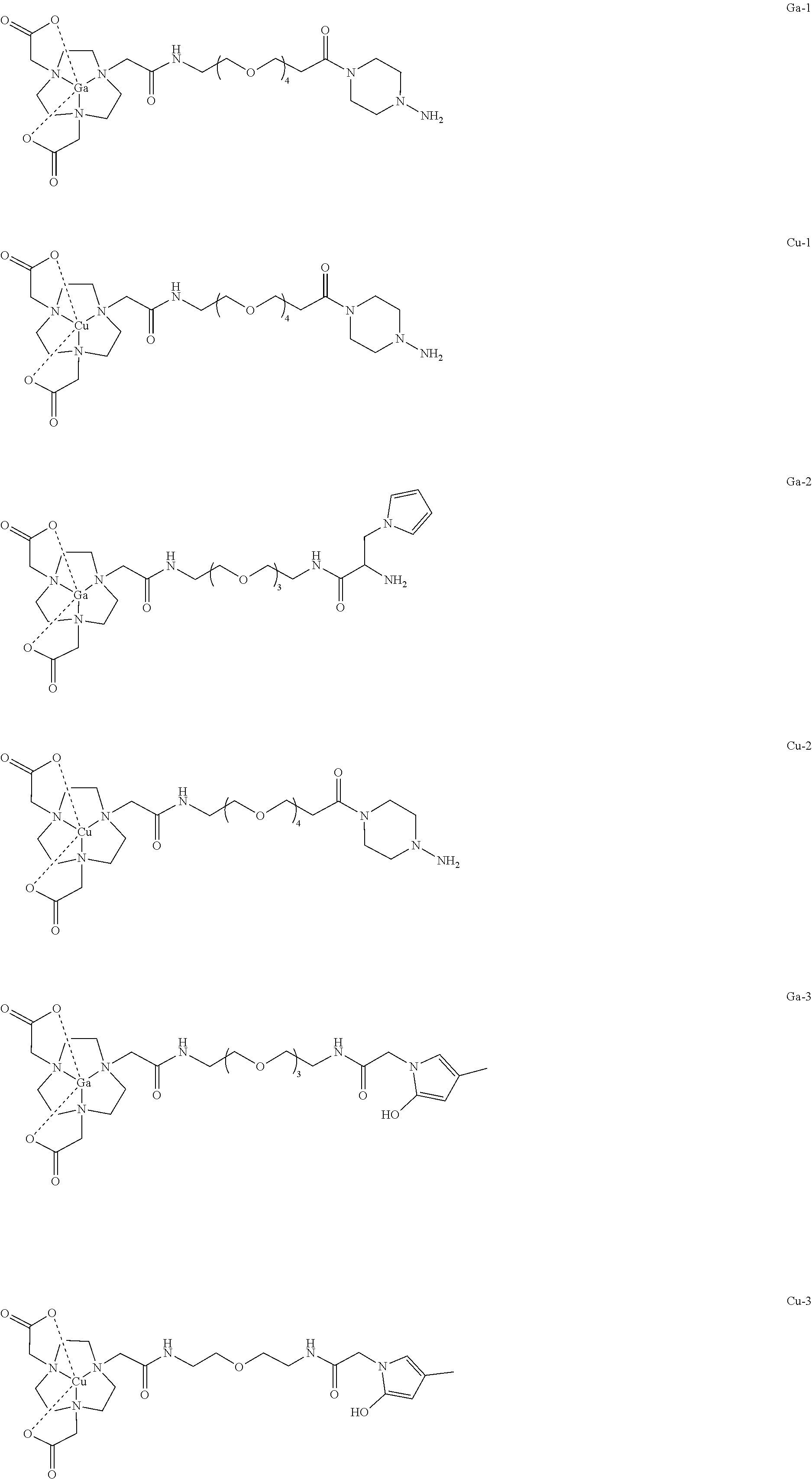

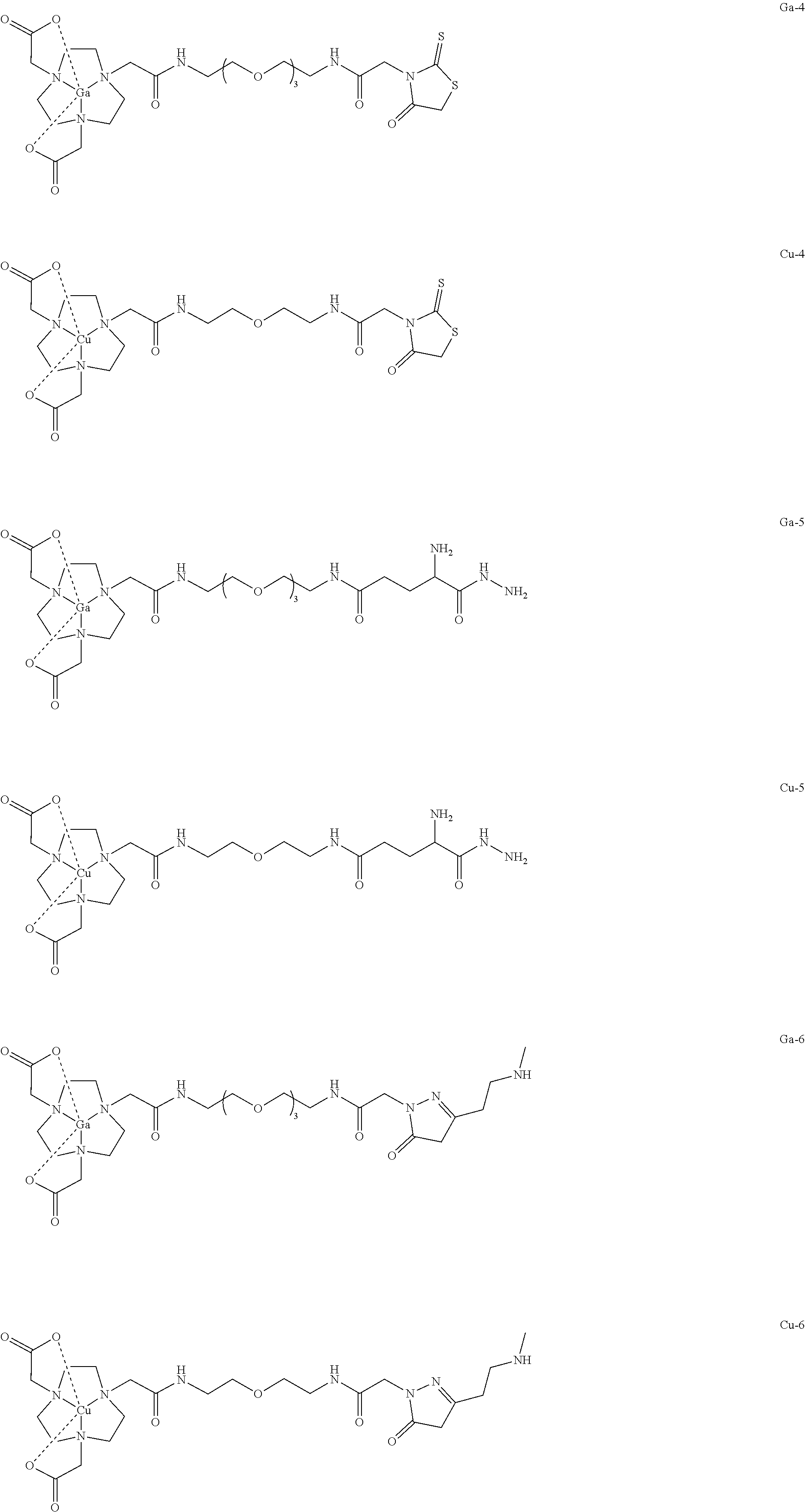

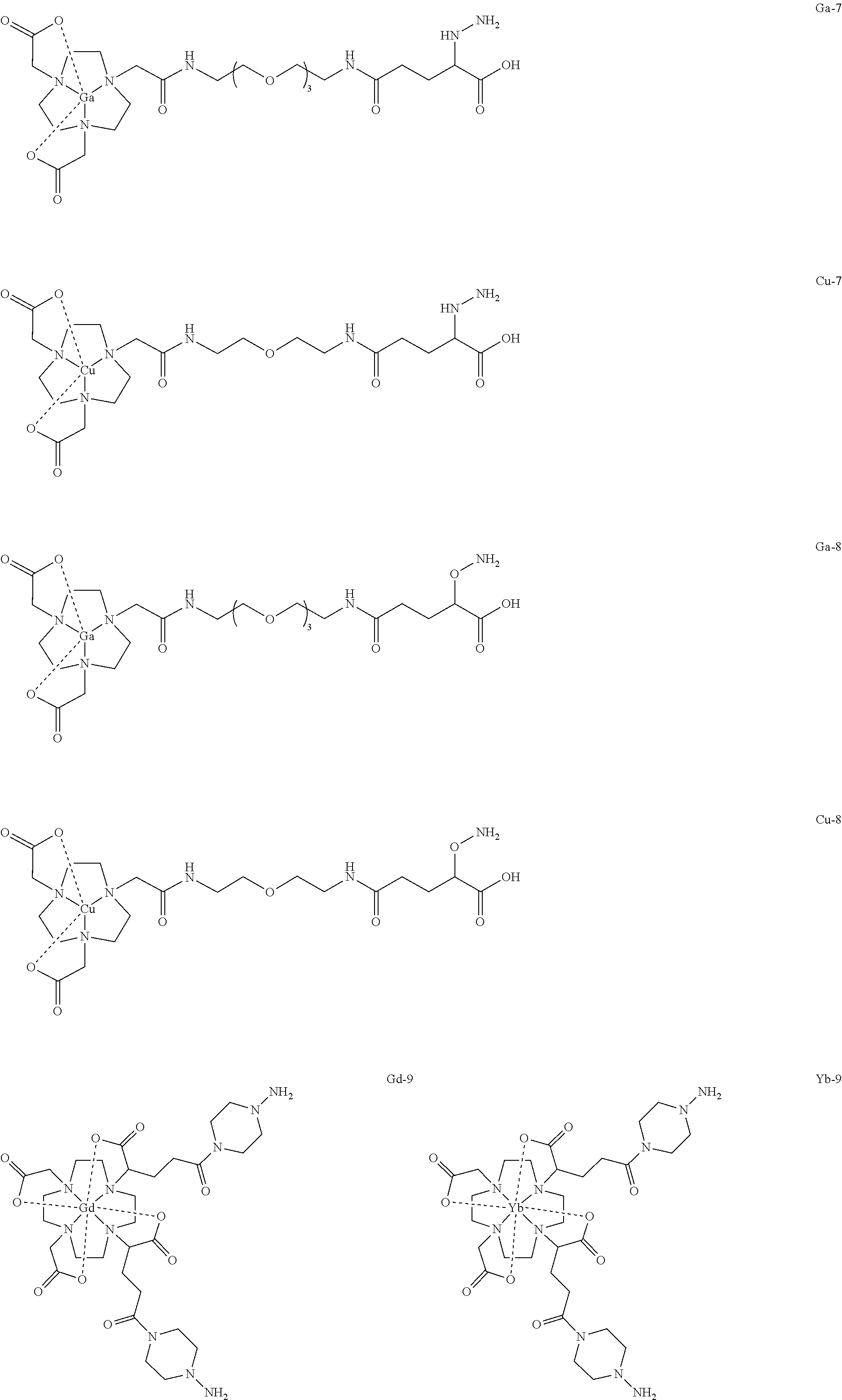

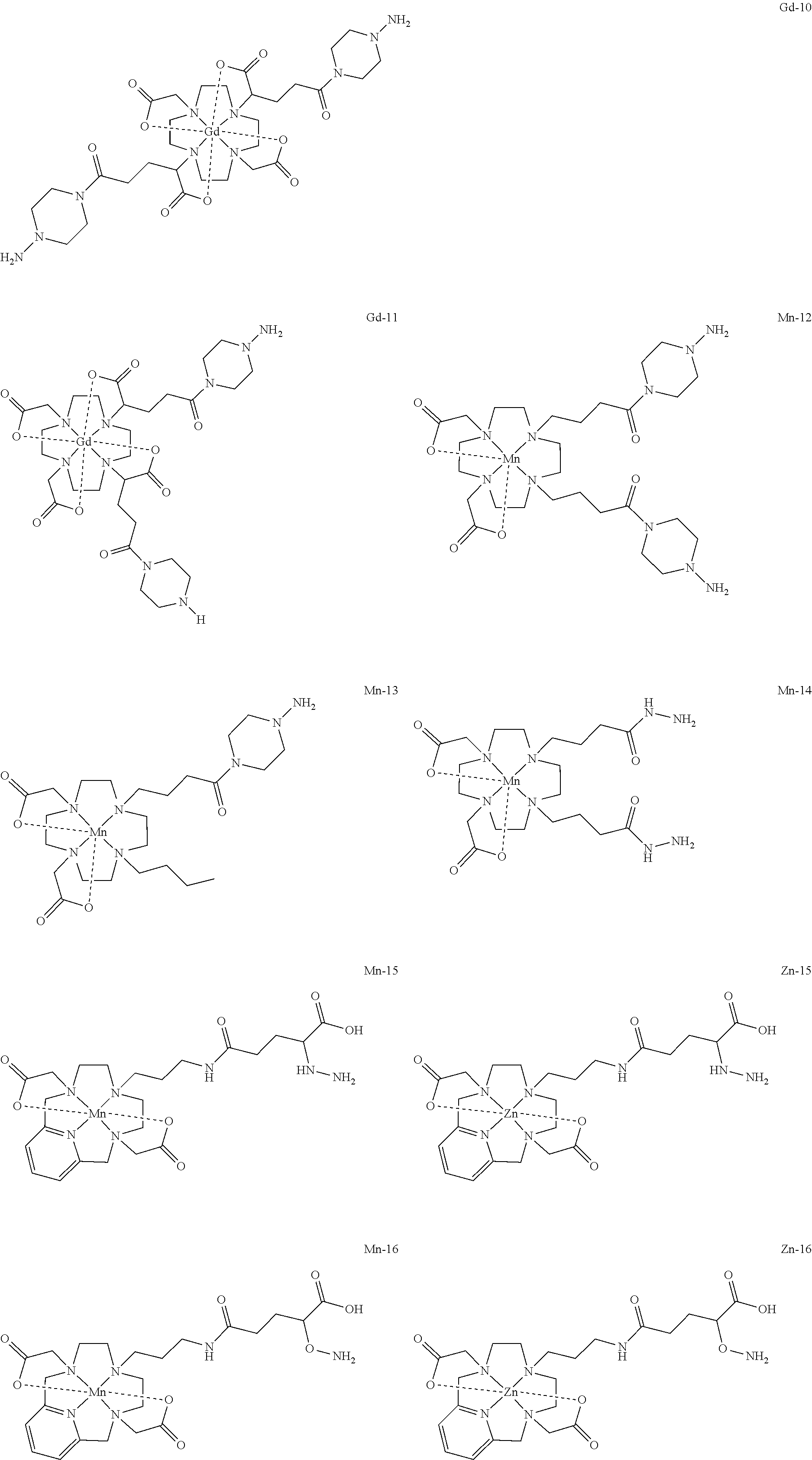

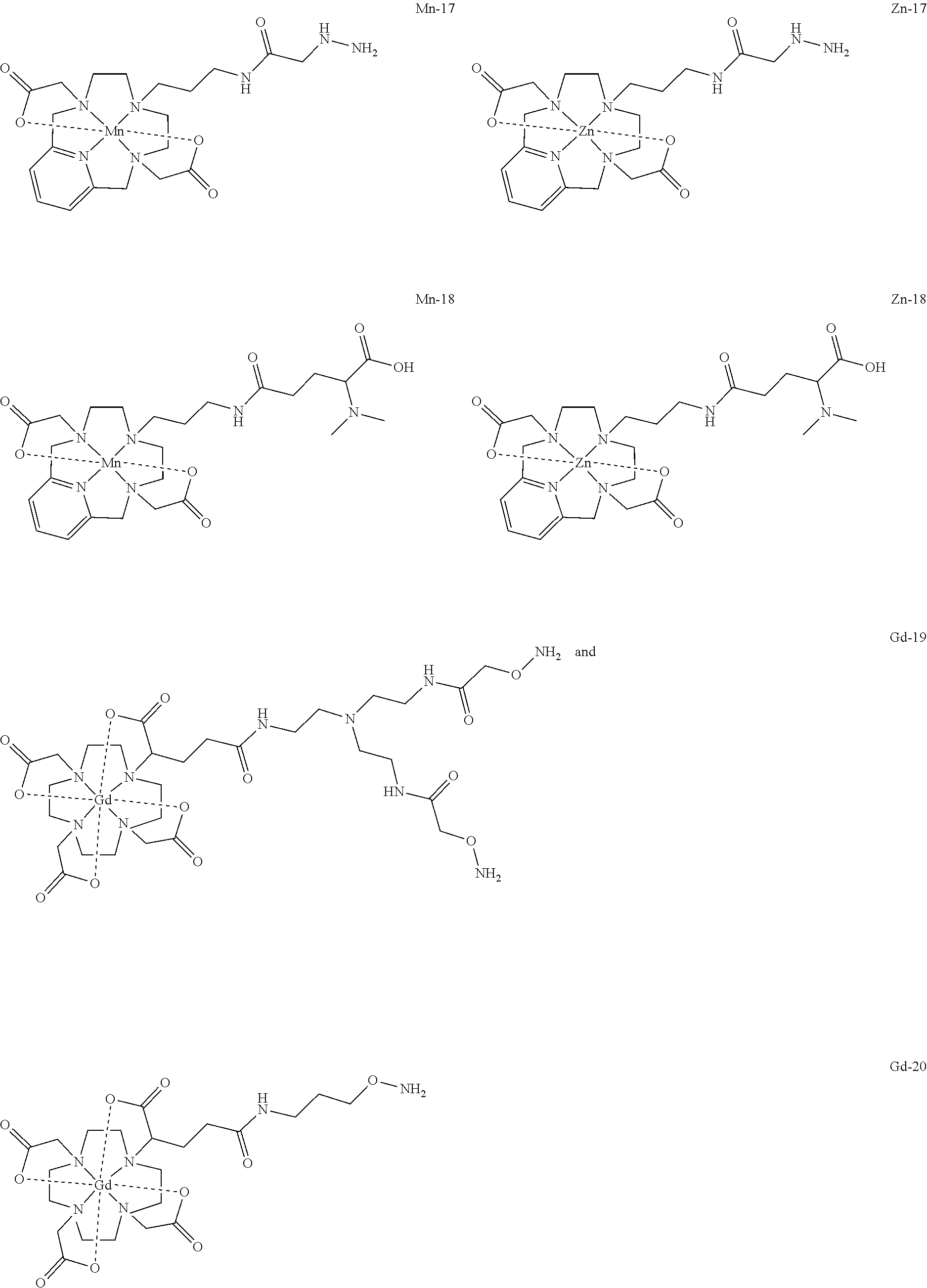

or a pharmaceutically acceptable salt thereof.

Embodiment 79: A composition comprising a compound of any one of embodiments 1-78, or a pharmaceutically acceptable salt thereof, and a pharmaceutically acceptable carrier.

Embodiment 80: The composition of embodiment 79, wherein the composition comprises a mixture of compounds of any one of embodiments 1-78, or a pharmaceutically acceptable salt thereof.

Embodiment 81: The composition of embodiment 79 or 80, wherein the composition is formulated for parenteral administration.

Embodiment 82: The composition of any one of embodiments 79-81, wherein the composition is a solid formulated for dissolution in a pharmaceutically acceptable liquid medium prior to administration.

Embodiment 83: A method of magnetic resonance (MR) imaging a subject comprising:
(a) administering to a subject a compound of any one of embodiments 1-78 or a composition of any one of embodiment 79-82; and
(b) obtaining a magnetic resonance image of the subject after a period of time.

Embodiment 84: A method of magnetic resonance (MR) imaging a subject comprising:
(a) obtaining a first magnetic resonance image of the subject;
(b) administering to a subject a compound of any one of embodiments 1-78 or a composition of any one of embodiments 79-82;
(c) obtaining a second magnetic resonance image of the subject after a period of time; and
(d) comparing the first magnetic resonance image of the subject and the second magnetic resonance image of the subject.

Embodiment 85: A method for imaging liver fibrogenesis in a subject comprising:
(a) administering to a subject a compound of any one of embodiments 1-78 or a composition of any one of embodiments 79-82; and
(b) obtaining a magnetic resonance image of the liver of the subject after a period of time.

Embodiment 86: A method of measuring liver fibrogenesis in a subject comprising:
(a) administering to the subject a compound of any one of embodiments 1-78 or a composition of any one of embodiments 79-82;
(b) obtaining a first magnetic resonance image of the subject after a period of time;
(c) administering to a subject a compound of any one of embodiments 1-78 or a composition of any one of embodiments 79-82 after a second period of time;
(d) obtaining a second magnetic resonance image of the subject after a period of time; and
(e) comparing the first magnetic resonance image of the subject and the second magnetic resonance image of the subject, thereby measuring liver fibrogenesis in the subject.

Embodiment 87: A method for detecting liver fibrogenesis in a subject comprising:
(a) administering to the subject a compound of any one of embodiments 1-78 or a composition of any one of embodiments 79-82; and
(b) obtaining a magnetic resonance image of the subject after a period of time, thereby detecting the presence or absence of liver fibrogenesis in the subject.

Embodiment 88: A method of detecting liver fibrogenesis in a subject comprising:
(a) administering to the subject a compound of any one of embodiments 1-78 or a composition of any one of embodiments 79-82;
(b) obtaining a first magnetic resonance image of the subject after a period of time;
(c) administering to a subject a compound of any one of embodiments 1-78 or a composition of any one of embodiments 79-82 after a second period of time;
(d) obtaining a second magnetic resonance image of the subject after a period of time; and
(e) comparing the first magnetic resonance image of the subject and the second magnetic resonance image of the subject, thereby detecting the presence or absence of liver fibrogenesis in the subject.

Embodiment 89: A method for detecting liver fibrogenesis in a subject comprising obtaining a magnetic resonance image of the subject within a period of time after the subject has been administered subject a compound of any one of embodiments 1-78 or a composition of any one of embodiments 79-82.

Embodiment 90: A method of positron emission tomography (PET) imaging a subject comprising:
(a) administering to the subject a compound of any one of embodiments 1-78 or a composition of any one of embodiments 79-82; and
(b) obtaining a positron emission tomography image of the subject after a period of time.

Embodiment 91: A method of positron emission tomography (PET) imaging a subject comprising:
(a) obtaining a first magnetic resonance image of the subject;
(b) administering to the subject a compound of any one of embodiments 1-78 or a composition of any one of embodiments 79-82;
(c) obtaining a second positron emission tomography image of the subject after a period of time; and
(d) comparing the first magnetic resonance image of the subject and the second positron emission tomography image of the subject.

Embodiment 92: A compound of Formula (II)

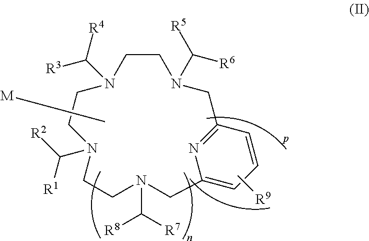

or a pharmaceutically acceptable salt thereof, wherein:

M is a metal cation;

each R¹, R³, R⁵, and R⁷ are independently hydrogen or —C(═O)OH;

each R², R⁴, R⁶, and R⁸ are independently hydrogen or C₃₋₂₅ alkyl, wherein the C₃₋₂₅ alkyl is optionally substituted with 1-2 substituents independently selected from the group consisting of —NR^{A}R^{B}, —OH, halogen, C₁₋₆ alkoxy, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of halogen, —NR^{A}R^{B}, —OH, C₁₋₆ alkyl, and —C₁₋₆ alkyl-(NR^{A}R^{B}); and one or more non-adjacent carbon atoms of the C₃₋₂₅ alkyl are optionally replaced by O, N, NH, or N(CH₃);

R⁹ is H, halogen, —NR^{A}R^{B}, —OH, C₁₋₆ alkyl, or —C₁₋₆ alkyl-(NR^{A}R^{B});

each R^{A} and R^{B} are independently hydrogen or C₁₋₆ alkyl;

n is 0 or 1; and

p is 0 or 1;

wherein if n is 0 at least two of R¹, R³, and R⁵ are —C(═O)OH and if n is 1 at least two of R¹, R³, R⁵, and R⁷ are —C(═O)OH.

### REFERENCES

A. Pellicoro, P. Ramachandran, J. P. Iredale, J. A. Fallowfield, Liver fibrosis and repair: immune regulation of wound healing in a solid organ. Nat Rev Immunol 14, 181-194 (2014).
S. K. Asrani, H. Devarbhavi, J. Eaton, P. S. Kamath, Burden of liver diseases in the world. J Hepatol 70, 151-171 (2019).
E. B. Tapper, A. S. Lok, Use of Liver Imaging and Biopsy in Clinical Practice. N Engl J Med 377, 756-768 (2017).
E. B. Tapper, R. Loomba, Noninvasive imaging biomarker assessment of liver fibrosis by elastography in NAFLD. Nat Rev Gastroenterol Hepatol 15, 274-282 (2018).
A. J. Sanyal et al., Non-Invasive Biomarkers of Nonalcoholic Steatohepatitis: the FNIH NIMBLE project. Nat Med, (2022).
A. C. Sheka et al., Nonalcoholic steatohepatitis: a review. Jama 323, 1175-1183 (2020).
V. Ratziu, Z. Goodman, A. Sanyal, Current efforts and trends in the treatment of NASH. J Hepatol 62, S65-75 (2015).
B. A. Davison et al., Suboptimal reliability of liver biopsy evaluation has implications for randomized clinical trials. J Hepatol 73, 1322-1332 (2020).
T. Kisseleva, D. Brenner, Molecular and cellular mechanisms of liver fibrosis and its regression. Nat Rev Gastroenterol Hepatol 18, 151-166 (2021).
N. Roehlen, E. Crouchet, T. F. Baumert, Liver Fibrosis: Mechanistic Concepts and Therapeutic Perspectives. Cells 9, 875 (2020).
C. Bonnans, J. Chou, Z. Werb, Remodelling the extracellular matrix in development and disease. Nat Rev Mol Cell Biol 15, 786-801 (2014).
M. E. Nimni, et, al, Collagen. M. Nimni, Ed., (CRC press, FL, USA, 2018).
C. T. Farrar et al., 3D molecular MR imaging of liver fibrosis and response to rapamycin therapy in a bile duct ligation rat model. J Hepatol 63, 689-696 (2015).
P. Désogère et al., Type I collagen-targeted PET probe for pulmonary fibrosis detection and staging in preclinical models. Sci. Transl. Med. 9, 384 (2017).
M. Salarian et al., Early detection and staging of chronic liver diseases with a protein MRI contrast agent. Nat Commun 10, 4777 (2019).
W. Chen et al., Lysyl Oxidase (LOX) Family Members: Rationale and Their Potential as Therapeutic Targets for Liver Fibrosis. Hepatology 72, 729-741 (2020).
K. Kawahara et al., Spatiotemporal regulation of PEDF signaling by type I collagen remodeling. Proc Natl Acad Sci USA 117, 11450-11458 (2020).
M. Yamauchi, M. Sricholpech, Lysine post-translational modifications of collagen. Essays Biochem 52, 113-133 (2012).
J. K. Mouw, G. Ou, V. M. Weaver, Extracellular matrix assembly: a multiscale deconstruction. Nat Rev Mol Cell Biol 15, 771-785 (2014).
P. Caravan, Strategies for increasing the sensitivity of gadolinium based MRI contrast agents. Chem Soc Rev 35, 512-523 (2006).
Akam, E. A., et al. Improving the reactivity of hydrazine-bearing MRI probes for in vivo imaging of lung fibrogenesis. Chemical Science 11, 224-231 (2020);
Foillard, S., Rasmussen, M. O., Razkin, J., Boturyn, D. & Dumy, P. 1-Ethoxyethylidene, a new group for the stepwise SPPS of aminooxyacetic acid containing peptides. J. Org. Chem. 73, 983-991 (2008);
Desogere, P., et al. Optimization of a Collagen-Targeted PET Probe for Molecular Imaging of Pulmonary Fibrosis. J Nuc Med 58, 1991-1996 (2017); and
González-Rosa, J. M. & Mercader, N. Cryoinjury as a myocardial infarction model for the study of cardiac regeneration in the zebrafish. Nat Protoc 7, 782-788 (2012).
Henderson, N. C., Rieder, F., & Wynn, T. A. Nature. 2020, 587, 555-566;
Chen, H. H., Waghorn, P. A., Wei, L., et al. JCI insight, 2017, 2, e91506;
Waghorn, P A., Jones, C. M. et al. Angew. Chem. Int. Ed. 2017, 56, 9825-9828;
Akam, E. A., Abston, E., Rotile, N. J., Slattery, H. R., Zhou, I. Y., Lanuti, M., & Caravan, P. Chem. Sci. 2020, 11, 224-231.
J. Wahsner, E. M. Gale, A. Rodriguez-Rodriguez, P. Caravan, Chemistry of MRI Contrast Agents: Current Challenges and New Frontiers. Chem Rev 119, 957-1057 (2019).
J. Ramalho et al., Gadolinium-Based Contrast Agent Accumulation and Toxicity: An Update. AJNR Am J Neuroradiol 37, 1192-1198 (2016).
B. X. Huang, H. Y. Kim, C. Dass, Probing three-dimensional structure of bovine serum albumin by chemical cross-linking and mass spectrometry. J Am Soc Mass Spectrom 15, 1237-1247 (2004).
Z. Zhang et al., Multilocus binding increases the relaxivity of protein-bound MRI contrast agents. Angew Chem Int Ed 44, 6766-6769 (2005).
J. P. Iredale, Models of liver fibrosis: exploring the dynamic nature of inflammation and repair in a solid organ. J Clin Invest 117, 539-548 (2007).
Y. Zhong et al., Lysyl oxidase regulation and protein aldehydes in the injured newborn lung. Am J Physiol Lung Cell Mol Physiol 322, L204-L223 (2022).
P. Rantakari et al., Stabilin-1 expression defines a subset of macrophages that mediate tissue homeostasis and prevent fibrosis in chronic liver injury. Proc Natl Acad Sci USA 113, 9298-9303 (2016).
C. Kuhn, J. Boldt, T. E. King, E. Crouch, T. Vartio and J. A. McDonald, Am. Rev. Respir. Dis., 1989, 140, 1693-1703;
Chen, H. H., Waghorn, P. A., Wei, L., et al. JCI insight, 2017, 2, e91506;
Waghorn, P. A., Jones, C. M. et al. Angew. Chem. Int. Ed. 2017, 56, 9825-9828;
Akam, E. A., Abston, E., Rotile, N. J., Slattery, H. R., Zhou, I. Y., Lanuti, M., & Caravan, P. Chem. Sci. 2020, 11, 224-231; and
Chapman, Harold A., et al. N. Engl. J. Med. 2020, 382, 1068-1070; 6. Desogere, P., et al. J Nuc Med 2017, 58, 1991-1996.
Bataller, R., & Brenner, D. A. J. Clin. Invest. 2005, 115, 209-218;
Chen, H. H., Waghorn, P. A., Wei, L., et al. JCI insight, 2017, 2, e91506;
Waghorn, P A., Jones, C. M. et al. Angew. Chem. Int. Ed. 2017, 56, 9825-9828;
Akam, E. A., Abston, E., Rotile, N. J., Slattery, H. R., Zhou, I. Y., Lanuti, M., & Caravan, P. Chem. Sci. 2020, 11, 224-231;
Grobner, T. Nephrol. Dial. Transplant. 2006, 21, 1104-1108; and
Garda, Z., Forgács, A., Do, Q. N., Kilman, F. K., et al. J. Inorg. Biochem. 2016, 163, 206-213.
M. Matsumoto et al., An improved mouse model that rapidly develops fibrosis in non-alcoholic steatohepatitis. Int J Exp Pathol 94, 93-103 (2013).
Z. J. Brown, B. Heinrich, T. F. Greten, Mouse models of hepatocellular carcinoma: an overview and highlights for immunotherapy research. Nat Rev Gastroenterol Hepatol 15, 536-554 (2018).
I. Y. Zhou et al., Advanced MRI of Liver Fibrosis and Treatment Response in a Rat Model of Nonalcoholic Steatohepatitis. Radiology 296, 67-75 (2020).
J. Wahsner, P. Desogere, E. Abston, K. A. Graham-O'Regan, J. Wang, N. J. Rotile, M. D. Schirmer, D. D. Santos Ferreira, J. Sui, B. C. Fuchs, M. Lanuti and P. Caravan, J Am Chem Soc 2019, 141, 5593-5596; and
H. H. Chen, P. A. Waghorn, L. Wei, L. F. Tapias, D. T. Schuhle, N. J. Rotile, C. M. Jones, R. J. Looby, G. Zhao, J. M. Elliott, C. K. Probst, M. Mino-Kenudson, G. Y Lauwers, A. M. Tager, K. K. Tanabe, M. Lanuti, B. C. Fuchs and P. Caravan, JCI Insight 2017, 2, e91506.
S. G. Levy et al., Development of a Multigram Asymmetric Synthesis of 2-(R)-2-(4,7,10-Tris tert-Butylcarboxymethyl-1,4,7,10-tetraazacyclododec-1-yl)-pentanedioic Acid, 1-tert-Butyl Ester, (R)-tert-Bu₄-DOTAGA. Org. Process Res. Dev. 13, 535-542 (2009).
C. Li, W. T. Wong, A simple, regioselective synthesis of 1,4-bis(tert-butoxycarbonylmethyl)-tetraazacyclododecane. J Org Chem 68, 2956-2959 (2003).
L. E. Hopper, M. J. Allen, Rapid synthesis of 1,7-bis(t-butoxycarbonylmethyl)-1,4,7,10-tetraazacyclododecane (DO2A-t-Bu ester). Tetrahedron Lett 55, 5560-5561 (2014).
E. A. Akam et al., Improving the reactivity of hydrazine-bearing MRI probes for in vivo imaging of lung fibrogenesis. Chem Sci 11, 224-231 (2020).
R. Janicki, A. Mondry, Structural and thermodynamic aspects of hydration of Gd(III) systems. Dalton Trans 48, 3380-3391 (2019).
J. P. Malone, A. George, A. Veis, Type I collagen N-telopeptides adopt an ordered structure when docked to their helix receptor during fibrillogenesis. Proteins 54, 206-215 (2004).
S. R. Brozell et al., AMBER 2018. (University of California, San Francisco, 2018).
J. A. Maier et al., ff14SB: Improving the Accuracy of Protein Side Chain and Backbone Parameters from ff99SB. J. Chem. Theory Comput. 11, 3696-3713 (2015).
J. Wang, R. M. Wolf, J. W. Caldwell, P. A. Kollman, D. A. Case, Development and testing of a general amber force field. J. Comput. Chem. 25, 1157-1174 (2004).
C. I. Bayly, P. Cieplak, W. D. Cornell, P. A. Kollman, A Well-Behaved Electrostatic Potential Based Method Using Charge Restraints for Deriving Atomic Charges—the Resp Model. J. Phys. Chem. 97, 10269-10280 (1993).
P. Li, K. M. Merz, Jr., MCPB.py: A Python Based Metal Center Parameter Builder. J Chem. Inf. Model 56, 599-604 (2016).
W. L. Jorgensen, J. Chandrasekhar, J. D. Madura, R. W. Impey, M. L. Klein, Comparison of Simple Potential Functions for Simulating Liquid Water. J. Chem. Phys. 79, 926-935 (1983).
J. P. Ryckaert, G. Ciccotti, H. J. C. Berendsen, Numerical integration of the cartesian equations of motion of a system with constraints: molecular dynamics of n-alkanes. J. Comput. Phys. 23, 327-341 (1977).
H. H. Chen et al., Molecular imaging of oxidized collagen quantifies pulmonary and hepatic fibrogenesis. JCI Insight 2, e91506 (2017).
A. Naba et al., The matrisome: in silico definition and in vivo characterization by proteomics of normal and tumor extracellular matrices. Mol Cell Proteomics 11, M111.014647 (2012).
H. Puchtler, F. S. Waldrop, S. N. Meloan, M. S. Terry, H. M. Conner, Methacarn (methanol-Carnoy) fixation. Practical and theoretical considerations. Histochemie 21, 97-116 (1970).
D. J. Erstad et al., Molecular magnetic resonance imaging accurately measures the antifibrotic effect of EDP-305, a novel farnesoid X receptor agonist. Hepatol Commun 2, 821-835 (2018).
P. R. Hutson, M. E. Crawford, R. L. Sorkness, Liquid chromatographic determination of hydroxyproline in tissue samples. J. Chromatogr. B 791, 427-430 (2003).
M. Sala, V. S. Selih, J. T. van Elteren, Gelatin gels as multi-element calibration standards in LA-ICP-MS bioimaging: fabrication of homogeneous standards and microhomogeneity testing. Analyst 142, 3356-3359 (2017).
B. Paul et al., CellSpace: A module for creating spatially registered laser ablation images within the Iolite freeware environment. J. Anal. At. Spectrom. 27, 700-706 (2012).
C. Paton, J. Hellstrom, B. Paul, J. Woodhead, J. Hergt, Iolite: Freeware for the visualisation and processing of mass spectrometric data. J. Anal. At. Spectrom. 26, 2508-2518 (2011).

### EXAMPLES

The following examples are illustrative and not intended to be limiting.

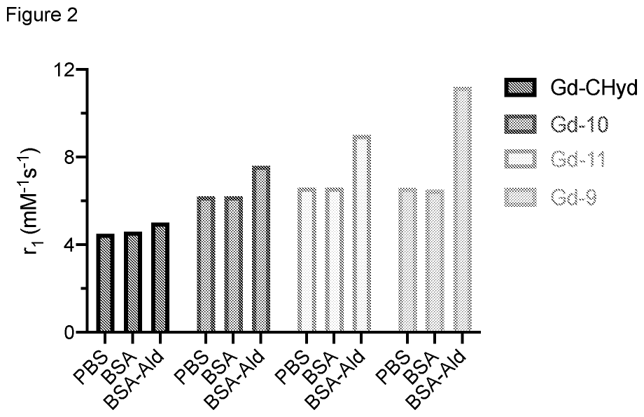

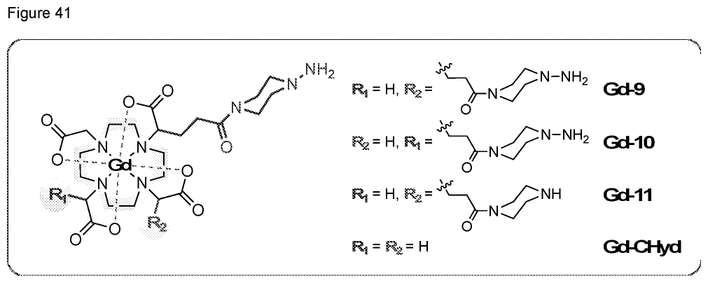

Precisely targeting Lys^{Ald} pairs requires the targeting groups to closely match the distance between the two Lys^{Ald}. Here, the Gd-DOTA core was used to design the probes (FIG. 41) since it is known to be extracellularly distributed and is one of the most thermodynamically stable and kinetically inert Gd³⁺ complex known (21, 22). Two piperazino-hydrazine moieties were introduced on the α-carbons of two of the Gd-DOTA carboxylate arms, to give either cis-1,4-Gd-(CHyd)₂ (Gd-9) or trans-1,7-Gd-(CHyd)₂ (Gd-10) that differed in the distance between the two hydrazine moieties (FIG. 2A). Two control compounds were prepared: Gd-11, which possess one piperazino-hydrazine and one piperazine arm, and Gd-CHyd, which only has one piperazino-hydrazine moiety (FIG. 41).

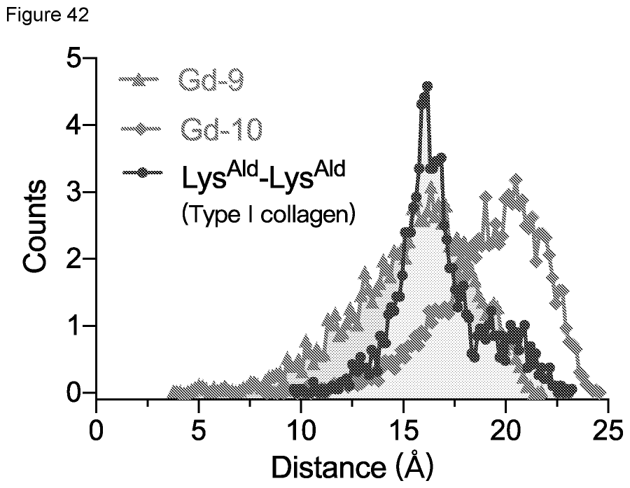

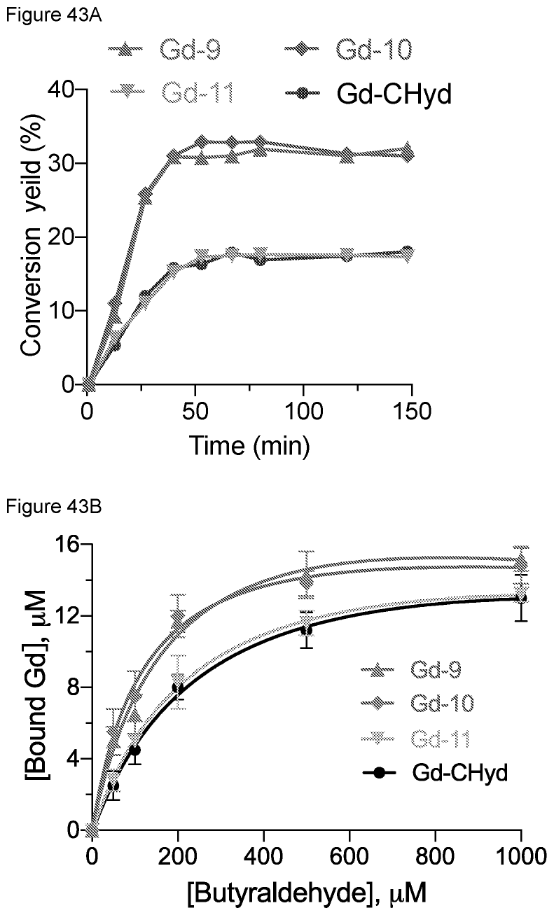

To estimate how the regioisomers might react with oxidized collagen, molecular dynamics simulations were performed. The O-O distance between two al-N⁹-Lys^{Ald} residues on oxidized type I collagen was found to be centered at 16.5 Å (FIG. 42). The N-N distance between the two piperazino-hydrazine groups in Gd-9 and Gd-10 was centered at 16.2 and 20.7 Å respectively. This suggested that Gd-9 may be preferred in targeting Lys^{Ald} pairs.

### Abbreviations

| | |
|---|---|
| HPLC-MS | high performance liquid chromatography-mass spectrometry |
| DCM | dichloromethane |
| DMF | N,N-dimethylformamide |
| DIPEA | di-isopropyl ethyleamine |
| ICP | inductively-coupled plasma |
| ACN | acetonitrile |
| BM | Bleomycin lung injury mouse model |
| HATU | 1-[bis(dimethylamino)methylene]-1H-1,2,3-triazolo[4,5-b]pyridinium 3-oxid |
| | hexafluorophosphate |
| THF | tetrahydrofuran |
| DCC | N,N′-Dicyclohexylcarbodiimide |
| Dpi | Days post injury |
| MWCO | Molecular weight cut-off |
| TAMRA | 5-Carboxytetramethylrhodamine |
| ROI | Region of interest |
| Mn-1,4-DO2A | 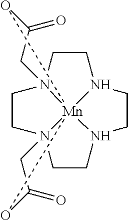 |
| 1,4-DO2A-t-Bu | 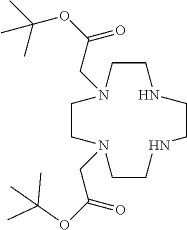 [SMILES: CC(C)(C)OC(=O)CN1CCNCCNCCN(CC(=O)OC(C)(C)C)CC1] |
| Fmoc-NH-PEG4- Acid | 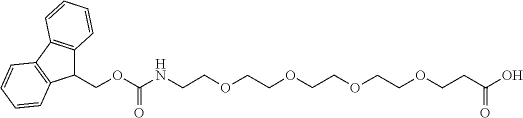 [SMILES: O=C(O)CCOCCOCCOCCOCCNC(=O)OCC1c2ccccc2-c2ccccc21] |
| NOTA-bis(t-Bu ester) | 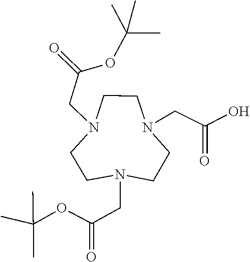 [SMILES: CC(C)(C)OC(=O)CN1CCN(CC(=O)O)CCN(CC(=O)OC(C)(C)C)CC1] |
| NOTA trihydro- chloride salt | 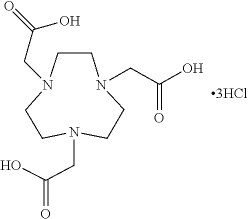 [SMILES: O=C(O)CN1CCN(CC(=O)O)CCN(CC(=O)O)CC1] |
| N-t-Boc-L-Glu- α-Bz ester | 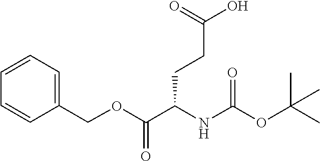 [SMILES: CC(C)(C)OC(=O)N[C@@H](CCC(=O)O)C(=O)OCc1ccccc1] |
| BSA-ALD | Oxidized bovine serum albumin containing allysine aldehyde groups |
| BSA | bovine serum albumin |
| GdDOTA | 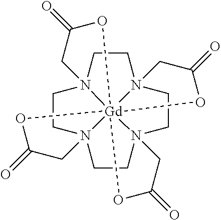 |
| Eu-DOTA | 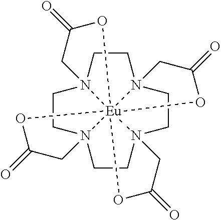 |
| Mn-PC2A | 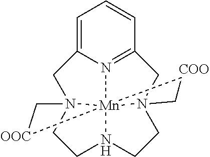 |
| EGCG | 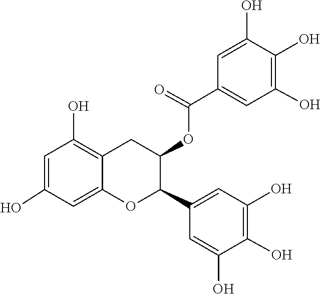 [SMILES: O=C(O[C@@H]1Cc2c(O)cc(O)cc2O[C@@H]1c1cc(O)c(O)c(O)c1)c1cc(O)c(O)c(O)c1] |
| ⁶⁸Ga-NODAGA- indole | 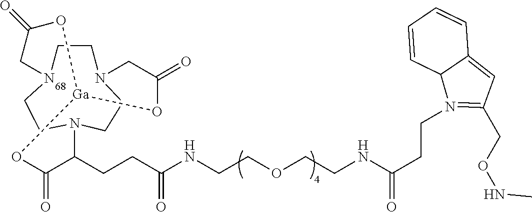 |
| GdCHyd | 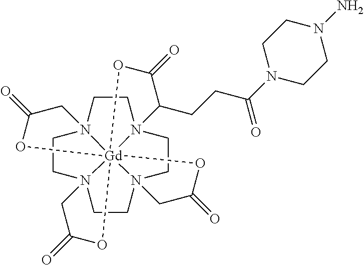 |
| Tf₂O | Trifluoromethanesulfonic anhydride |
| Mn-15-Ald | 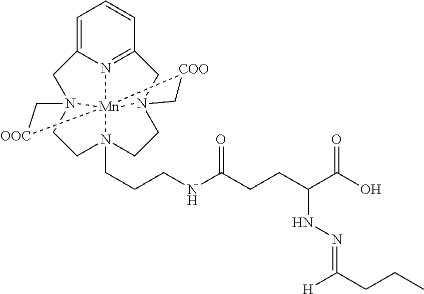 |
| Mn-16-Ald | 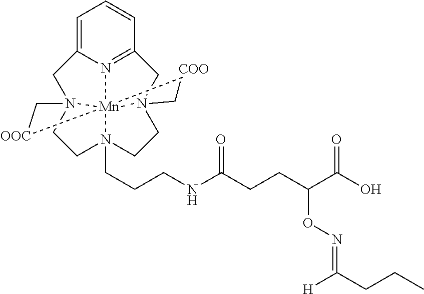 |
| Mn-17-Ald | 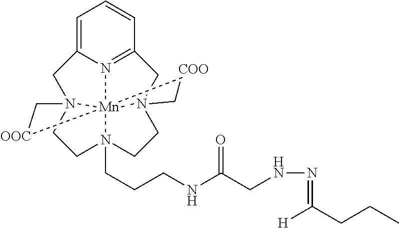 |

### Methods

NMR: NMR spectra were recorded on a JEOL FCZ 50OR 11.7 T NMR system equipped with a 5 mm broadband probe (¹H: 499.81 MHz, ¹³C: 125.68 MHz). Quantification of gadolinium was carried out using an Agilent 8800-QQQ ICP-MS system. Longitudinal (T₁) relaxation measurements were recorded using a Bruker mq60 Minispec at 1.41 T and 37′° C. High resolution electrospray ionization mass spectra (HR-ESI-MS) were acquired with Bruker Maxis Impact LC-q-TOF Mass Spectrometer. Animals were imaged on a 4.7 Tesla MRI scanner (Bruker, Biiierica MA) using a custom-built volume coil.

### Synthetic Protocols

(S)-5-Benzyl-1-tert-butyl 2-(methylsulfonyloxy)pentanedioate, 1,4-DO2A-t-Bu, 1,7-DO2A-t-Bu and Gd-CHyd were obtained as described previously. All other reactants and reagents were of commercial grade and used without further purification. Detail synthesis and characterizations can be found in the supplementary information.

### Molecular Dynamic Simulation

The systems with Gd³⁺ complexes were constructed on the basis of the crystal structure of Gd-DOTA (CCDC: 1882455) and modeling structure of type-I collagen. All the three chains of type-I collagen were considered in our study. The missing hydrogen atoms were added by the LEAP module in Amber 18. The Amber ff14SB force field was employed for the protein residues. The general AMBER force field (GAFF) was used for the ligand of Gd³⁺ complexes, while the partial atomic charges were quantified by the RESP method, using the HF/6-31G* level of theory. The force field for the Gd³⁺ complex was parametrized using MCPB. Then, Na⁺ ions were added to the surface of the protein to neutralize the total charge of the protein. Finally, the neutral system was solvated in a cube of TIP3P waters with a 15 Å water layer.

After setup, the system was minimized by a combined steepest descent and conjugate gradient method. Then the system was heated from 0 to 300 K under a canonical ensemble for 0.2 ns with a weak restraint of 15 kcal/(mol Å). To achieve a uniform density after heating dynamics, 1 ns of density equilibration was performed under the NPT ensemble at the target temperature of 300 K and target pressure of 1.0 atm. Afterward, the system was further equilibrated for 4 ns under the NPT ensemble to get an equilibrated pressure and temperature using Langevin thermostat and Berendsen barostat. Finally, a MD run was conducted for 50 ns. The covalent bonds containing hydrogen were constrained using SHAKE. 5000 snapshots were sampled from the MD trajectories every 10 ps to calculate the distance distribution between the interested groups. During all the minimization, equilibrium and NPT MD processes, both the N- and C-terminals of al, α1 and α2 chain of type-I collagen were fixed with a strong restraint of 500 kcal/(mol Å).

HPLC-MS: HPLC-MS analysis was carried out on Agilent 1260 system (UV detection at 220, 254 and 280 nm) coupled to an Agilent Technologies 6130 Quadrupole MS system. Mobile Phases: A: 0.1% formic acid in H₂O (v/v) B: 0.1% formic acid in CH₃CN (v/v) C: 10 mM NH₄OAc in H₂O, D: 90% CH₃CN+10% solvent C. UV detection at 220, 254 and 280 nm.

Method 1: Column: Phenomenex LUNA, C18(2), 5 μm,
100 × 2 mm, flow rate: 0.7 mL/min

| Time (min) | % A | % B |
|---|---|---|
| 0 | 95 | 5 |
| 3 | 5 | 95 |
| 4.5 | 5 | 95 |
| 5 | 95 | 5 |
| 7 | 95 | 5 |

Method 2: Column: Phenomenex LUNA, C18(2), 5 μm,
100 × 2 mm, flow rate: 0.7 mL/min

| Time (min) | % C | % D |
|---|---|---|
| 0 | 95 | 5 |
| 0.5 | 95 | 5 |
| 7.5 | 5 | 95 |
| 8.5 | 95 | 5 |
| 10 | 95 | 5 |

Method 3: Column: Restek, Ultra AQ C18, 5 μm,
250 × 4.6 mm column, flow rate: 0.7 mL/min.

| Time (min) | % A | % B |
|---|---|---|
| 0 | 95 | 5 |
| 3 | 5 | 95 |
| 4.5 | 5 | 95 |
| 5 | 95 | 5 |
| 7 | 95 | 5 |

Method 4: Column: Restek, Ultra AQ C18, 5 μm,
100 × 4.6 mm column, flow rate: 1.0 mL/min.

| Time (min) | % A | % B |
|---|---|---|
| 0 | 95 | 5 |
| 0.5 | 95 | 5 |
| 5 | 5 | 95 |
| 7 | 5 | 95 |
| 7.30 | 95 | 5 |
| 10 | 95 | 5 |

Method 5: Column: Restek, Ultra AQ C18, 5 μm
100 × 4.6 mm column, flow rate: 1.0 mL/min.

| min | % C | % D |
|---|---|---|
| 0 | 95 | 5 |
| 1 | 95 | 5 |
| 11 | 5 | 95 |
| 13 | 5 | 95 |
| 14 | 95 | 5 |
| 16 | 95 | 5 |

Method 6: Column: Restek, Ultra AQ C18, 5 μm
250 × 4.6 mm column, flow rate: 1.0 mL/min.

| min | % C | % D |
|---|---|---|
| 0 | 95 | 5 |
| 1 | 95 | 5 |
| 11 | 5 | 95 |
| 13 | 5 | 95 |
| 14 | 95 | 5 |
| 16 | 95 | 5 |

Method 7: Column: Restek, Ultra AQ C18, 5 μm,
250 × 10 mm column, flow rate: 0.7 mL/min.

| Time (min) | % C | % D |
|---|---|---|
| 0 | 95 | 5 |
| 0.5 | 95 | 5 |
| 7.5 | 5 | 95 |
| 8.5 | 95 | 5 |
| 10 | 95 | 5 |

Method 8: Column: Restek, UltraAqueous C18, 5 μm,
250 × 10 mm, flow rate: 0.7 mL/min

| Time (min) | % A | % B |
|---|---|---|
| 0 | 95 | 5 |
| 3 | 5 | 95 |
| 4.5 | 5 | 95 |
| 5 | 95 | 5 |
| 7 | 95 | 5 |

Flash chromatography: Large-scale reverse-phase purifications were carried out on a Teledyne ISCO CombiFlash system with UV-Vis detection at 220 and 254 nm. Mobile Phases: A: 0.1% formic acid in H₂O (v/v) B: 0.1% formic acid in CH₃CN (v/v). UV detection at 220 and 254 nm.

Method 9: Column: 150 g C18, flow rate: 70 mL/min.

| Time (min) | % A | % B |
|---|---|---|
| 0 | 95 | 5 |
| 5 | 95 | 5 |
| 30 | 30 | 70 |
| 35 | 5 | 95 |
| 40 | 5 | 95 |

Method 10: Column 50 g C18, flow rate: 40 mL/min

| Time (min) | % A | % B |
|---|---|---|
| 0 | 95 | 5 |
| 2 | 95 | 5 |
| 12 | 40 | 60 |
| 16 | 0 | 100 |
| 18 | 0 | 100 |
| 19 | 95 | 5 |
| 23 | 95 | 5 |

Method 11: Column 50 g C18-Aq, flow rate: 40 mL/min

| Time (min) | % A | % B |
|---|---|---|
| 0 | 100 | 0 |
| 2 | 100 | 0 |
| 12 | 60 | 40 |
| 16 | 0 | 100 |
| 18 | 0 | 100 |
| 19 | 100 | 0 |
| 23 | 100 | 0 |

Method 12: Column: 150 g C18gold, flow rate: 85 mL min⁻¹:

| Column | | |
|---|---|---|
| volume | % A | % B |
| 0 | 95 | 5 |
| 3 | 95 | 5 |
| 15 | 10 | 90 |
| 20 | 10 | 90 |
| 23 | 95 | 5 |
| 25 | 95 | 5 |

Method 13: Column: 150 g Ultra-aqueous
gold, flow rate: 70 mL/min

| Time (min) | % A | % B |
|---|---|---|
| 0 | 95 | 5 |
| 5 | 95 | 5 |
| 30 | 30 | 70 |
| 35 | 5 | 95 |
| 40 | 5 | 95 |

Preparative HPLC: Preparative reversed-phase HPLC with UV detection at 220, 254 and 280 nm was performed using Agilent 1260 system. Mobile Phases: A: 0.1% formic acid in H₂O (v/v) B: 0.1% formic acid in CH₃CN (v/v) C: 0.05% trifluoroacetic acid in H₂O (v/v) D: 0.05% trifluoroacetic acid in CH₃CN (v/v). UV detection at 220, 254 and 280 nm

Method 14: Column: Phenomenex LUNA C18(2) 10 μm,
250 × 21.2 mm, flow rate: 15 mL/min

| Time (min) | % A or C | % B or D |
|---|---|---|
| 0 | 95 | 5 |
| 20 | 55 | 45 |
| 40 | 5 | 95 |
| 48 | 5 | 95 |
| 50 | 95 | 5 |

Method 15: Column: Phenomenex LUNA C18(2) 10 μm,
250 × 21.2 mm, flow rate: 15 mL/min

| Time (min) | % A or C | % B or D |
|---|---|---|
| 0 | 95 | 5 |
| 5 | 95 | 5 |
| 35 | 5 | 95 |
| 40 | 5 | 95 |
| 41 | 95 | 5 |
| 46 | 95 | 5 |

Method 16: Column: Restek, UltraAqueous C18, 5 μm
250 × 21.2 mm, flow rate: 15 ml/min

| Time (min) | % A or C | % B or D |
|---|---|---|
| 0 | 95 | 5 |
| 20 | 70 | 30 |
| 27 | 5 | 95 |
| 28 | 95 | 5 |
| 30 | 95 | 5 |

Method 17: Column: Restek, UltraAqueous C18, 5 μm
250 × 21.2 mm, flow rate: 15 ml/min

| Time (min) | % A or C | % B or D |
|---|---|---|
| 0 | 100 | 0 |
| 5 | 100 | 0 |
| 35 | 5 | 95 |
| 40 | 5 | 95 |
| 41 | 100 | 0 |
| 46 | 100 | 0 |

Method 18: Column: Restek, UltraAqueous C18, 5 μm
250 × 21.2 mm, flow rate: 15 mL min⁻¹.

| min | % A | % B |
|---|---|---|
| 0 | 95 | 5 |
| 1 | 95 | 5 |
| 12 | 90 | 10 |
| 22 | 40 | 60 |
| 25 | 5 | 95 |
| 28 | 95 | 5 |
| 30 | 95 | 5 |

Method 19: Column: Phenomenex LUNA C18(2) 10 μm,
250 × 21.2 mm, flow rate: 15 mL/min

| Time (min) | % A | % B |
|---|---|---|
| 0 | 95 | 5 |
| 20 | 55 | 45 |
| 40 | 5 | 95 |
| 48 | 5 | 95 |
| 52 | 95 | 5 |

HPLC-ICP-MS: HPLC-ICP-MS was carried out on an Agilent 1260 HPLC system coupled to an Agilent 8800-QQQ ICP-MS system. Mobile Phases: A: 0.1% trifluoroacetic acid in H₂O (v/v), B: 0.1% trifluoroacetic acid in CH₃CN (v/v), C: 10 mM NH₄OAc in H₂O, D: 90 CH₃CN+10% solvent C.

Method 20: Column: Restek, UltraAqueous C18,
5 μm 250 × 10 mm column. Flow rate: 1 mL/min

| Time (min) | % A | % B |
|---|---|---|
| 0 | 100 | 0 |
| 2 | 70 | 30 |
| 5.5 | 5 | 95 |
| 8 | 5 | 95 |
| 8.1 | 100 | 0 |
| 10 | 100 | 0 |

Method 21: Column: Restek, UltraAqueous C18,
5 μm 250 × 10 mm column. Flow rate: 1 mL/min

| Time (min) | % A | % B |
|---|---|---|
| 0 | 95 | 5 |
| 2 | 95 | 5 |
| 8 | 5 | 95 |
| 9 | 5 | 95 |
| 12 | 95 | 5 |

Method 22: Column: XBridge, C18,
3.5 μm 150 × 4.6 mm column. Flow rate: 1 mL/min

| Time (min) | % C | % D |
|---|---|---|
| 0 | 95 | 5 |
| 2 | 95 | 5 |
| 7 | 5 | 95 |
| 8.5 | 5 | 95 |
| 9 | 95 | 5 |
| 10 | 95 | 5 |

Method 23: Column: Restek, UltraAqueous C18,
5 μm 250 × 10 mm column. Flow rate: 1 mL/min

| Time (min) | % A | % B |
|---|---|---|
| 95 | 100 | 5 |
| 2 | 95 | 5 |
| 7 | 5 | 95 |
| 8.5 | 5 | 95 |
| 10 | 95 | 5 |
| 10 | 100 | 0 |

Analytical HPLC: HPLC analysis was carried out on an Agilent 1260 system. Mobile phases: C: NH₄OAc 10 mM in H₂O, D: 90% ACN, 10% solvent C. UV detection at 220 nm, 254 nm and 280 nm.

Method 24: Column, Xbridge, 5 μm C18 3.5 mm,
150 × 4.6 mm, flow rate: 1.0 mL/min

| Time (min) | % C | % D |
|---|---|---|
| 0 | 95 | 5 |
| 1 | 95 | 5 |
| 11 | 5 | 95 |
| 13 | 5 | 95 |
| 14 | 95 | 5 |
| 16 | 95 | 5 |

Method 25: Column, Xbridge, 5μm C18 3.5 mm,
150 × 4.6 mm, flow rate: 1.0 mL/min

| Time (min) | % C | % D |
|---|---|---|
| 0 | 95 | 5 |
| 1 | 95 | 5 |
| 8 | 50 | 50 |
| 9 | 5 | 95 |
| 10 | 5 | 95 |
| 11 | 95 | 5 |
| 13 | 95 | 5 |

Method 26: Column, Xbridge, 5 μm C18 3.5 mm,
150 × 4.6 mm, flow rate: 1.0 mL/min

| Time (min) | % C | % D |
|---|---|---|
| 0 | 95 | 5 |
| 1 | 95 | 5 |
| 9.5 | 85 | 15 |
| 10 | 5 | 95 |
| 5 | 95 | 95 |
| 11.5 | 95 | 5 |
| 13 | 95 | 5 |

Method 27: Column, Xbridge, 5 μm C18 3.5 mm,
150 × 4.6 mm, flow rate: 1.0 mL/min

| Time (min) | % C | % D |
|---|---|---|
| 0 | 95 | 5 |
| 1 | 95 | 5 |
| 9.5 | 85 | 15 |
| 10 | 5 | 95 |
| 5 | 95 | 95 |
| 11.5 | 95 | 5 |
| 13 | 95 | 5 |

### Example 1. Compound 1

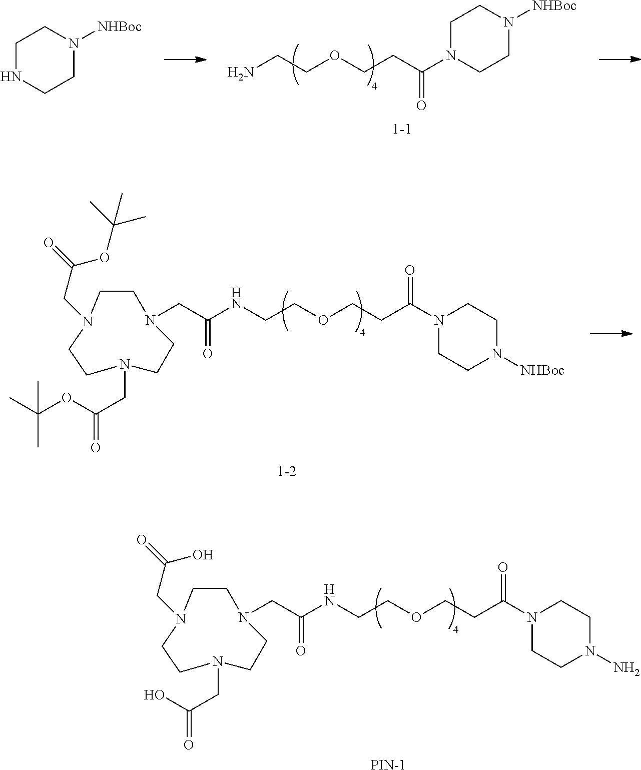

### Compound 1-1

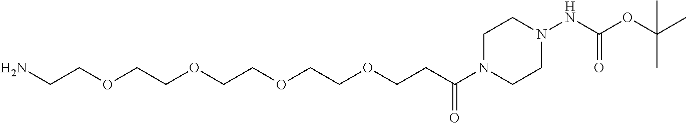 [SMILES: CC(C)(C)OC(=O)NN1CCN(C(=O)CCOCCOCCOCCOCCN)CC1]

Fmoc-NH-PEG₄-Acid (120 mg, 0.24 mmol) and HATU (137 mg, 0.36 mmol) were dissolved in anhydrous dimethylformamide (3 mL) and DIPEA (0.05 mL, 0.29 mmol) was added. The solution was stirred for 1 hour at room temperature, and tert-butyl piperazin-1-ylcarbamate (50 mg, 0.25 mmol) was added. The reaction mixture was stirred for 30 min at room temperature and the solution was subject to prep-HPLC purification (Method 6-1). The residue was dissolved in DMF/piperazine solution (4:1 v/v) and stirred for 30 min at room temperature. The solution was subject to prep-HPLC purification (Method 6-1) yielding Compound 1-1 as an oil (82 mg, 92% yield). ¹H NMR (500 MHz, CDCl₃) δ_{H} 3.70 (t, J=5.0 Hz, 2H), 3.67 (t, J=6.0 Hz, 2H), 3.60 (m, 4H), 3.55 (m, 10H), 3.50 (t, J=5.0 Hz, 2H), 3.08 (t, J=5.0 Hz, 2H), 2.75 (t, J=5.0 Hz, 2H), 2.71 (t, J=5.2 Hz, 2H), 2.55 (t, J=6.0 Hz, 2H), 1.36 (s, 9H); ¹³C NMR (126 MHz, CDCl₃) δ_{C} 169.8, 154.7, 80.4, 70.4-66.5, 55.3-55.0, 45.1, 41.3, 39.4, 33.2, 28.4. LC-MS (method 1): t_{R}=2.41 min, m/z [M+H]⁺ calcd for [C₂₀H₄₁N₄O₇]⁺, 449.3; found 449.4.

### Compound 1-2

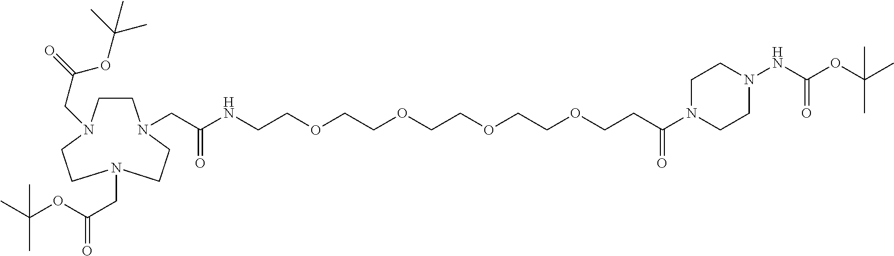 [SMILES: CC(C)(C)OC(=O)CN1CCN(CC(=O)NCCOCCOCCOCCOCCC(=O)N2CCN(NC(=O)OC(C)(C)C)CC2)CCN(CC(=O)OC(C)(C)C)CC1]

NOTA-bis(t-Bu ester) (76 mg, 0.18 mmol) and HATU (103 mg, 0.27 mmol) were dissolved in anhydrous dimethylformamide (4 mL) and DIPEA (0.05 mL, 0.29 mmol) was added. The reaction mixture was stirred for 30 min at room temperature, and Compound 1-1 (82 mg, 0.18 mmol) dissolved in anhydrous dimethylformamide (1 mL) was added. The reaction mixture was stirred for 30 min at room temperature and was subject to prep-HPLC purification (Method 6-1) yielding the Compound 1-2 (47 mg, 31% yield). ¹H NMR (500 MHz, CDCl₃) δ_{H} 3.74 (d, J=6.7 Hz, 2H), 3.68 (t, J=5.1 Hz, 2H), 3.57 (m, 14H), 3.51 (m, 4H), 3.41 (m, 2H), 3.33 (s, 4H), 2.89-2.81 (m, 12H), 2.78 (m, 2H), 2.73 (m, 2H), 1.41 (m, 27H); ¹³C NMR (126 MHz, CDCl₃) δ_{C} 175.7, 171.0, 169.7, 154.5, 81.3, 80.6, 70.6-70.0, 67.5, 60.1, 58.1, 55.5, 55.2-52.9, 45.2, 41.2, 39.3, 33.5, 28.4-28.2. LC-MS (method 2): t_{R}=5.72 min [M+H]⁺ calcd for [C₄₀H₇₆N₇O₁₂]⁺, 846.5; found 846.6.

### Compound 1

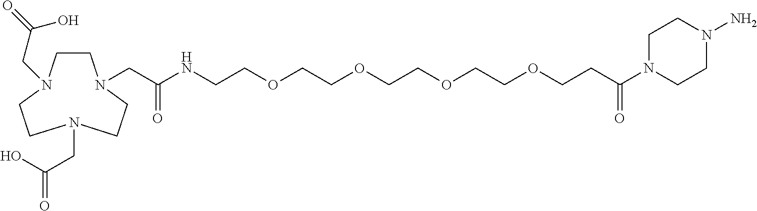 [SMILES: NN1CCN(C(=O)CCOCCOCCOCCOCCNC(=O)CN2CCN(CC(=O)O)CCN(CC(=O)O)CC2)CC1]

Compound 1-2 (47 mg, 0.06 mmol) was dissolved in dichloromethane (2 mL) and trifluoroacetic acid (2 mL) was added. The reaction mixture was stirred for 18 hours at room temperature, and the solvent was removed under reduced pressure. The residue was redissolved in water (4 mL) and was subject to prep-HPLC purification (Method 7-1) yielding the desired product Compound 1 as a white powder after lyophilization (20 mg, 55% yield). ¹H NMR (500 MHz, D₂O) δ_{H} 3.88 (s, 4H), 3.74 (s, 2H), 3.64 (br t, J=6.2 Hz, 4H), 3.53 (m, 8H), 3.52 (s, 4H), 3.50 (br m, 2H), 3.48 (t, J=5.4 Hz, 2H), 3.32 (m, 4H), 3.28 (m, 6H), 3.20 (m, 4H), 2.98 (m, 4H), 2.60 (t, J=6.1 Hz, 2H); ¹³C NMR (126 MHz, D₂O) δ_{C} 172.1, 171.5, 169.7, 69.6-68.6, 66.5, 58.1, 56.5, 53.7-53.5, 50.8, 50.5, 50.4, 43.8, 40.0, 39.1, 32.5. LC-MS (method 3-1): t_{R}=4.29 min, [M+H]⁺ calcd for [C₂₇H₅₂N₇O₁₀]⁺, 634.4; found 634.3.

### Example 2. Compound 2

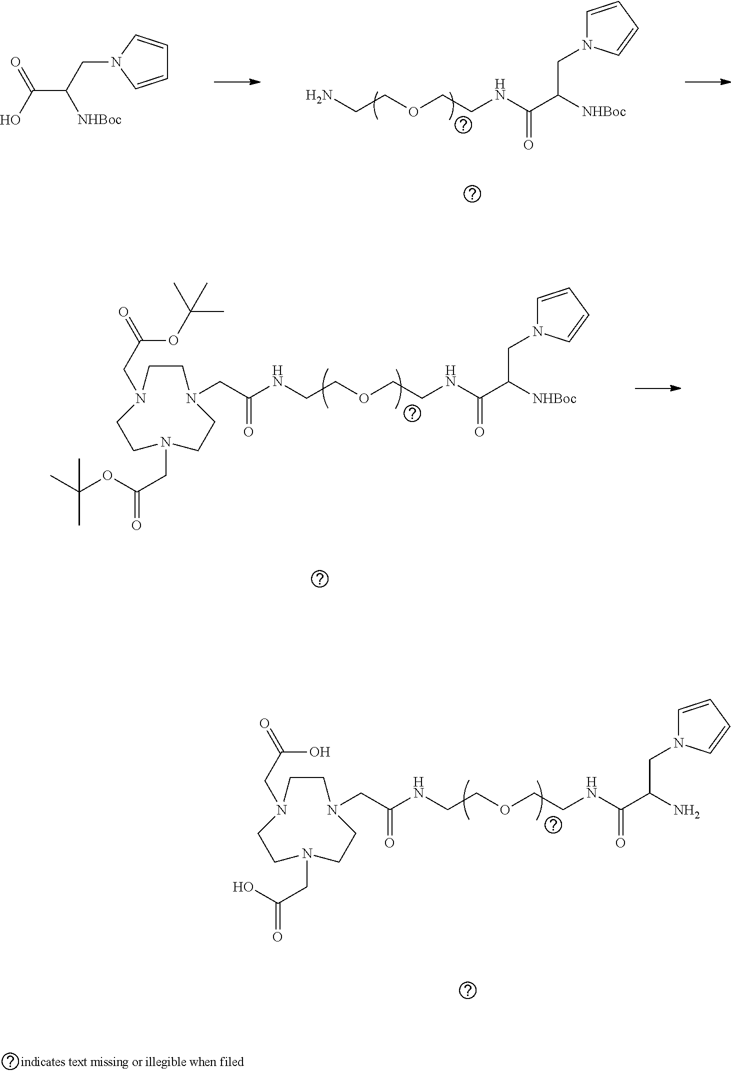

### Compound 2-1

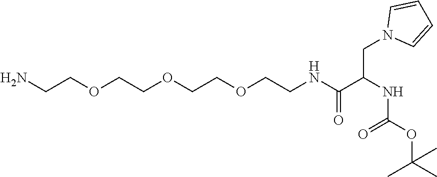 [SMILES: CC(C)(C)OC(=O)NC(Cn1cccc1)C(=O)NCCOCCOCCOCCN]

2-{(tert-butoxy)carbonyl]amino}-3-(1H-pyrrol-1-yl)propanoic acid (200 mg, 0.79 mmol), DCC (280 mg, 1.36 mmol) and N-hydroxysuccinimide (132 mg, 1.15 mmol) were dissolved in dichloromethane (12 mL) and stirred for 1 hour at room temperature. NH₂—PEG₃-NH₂ (200 mg, 1.04 mmol) dissolved in dichloromethane (1 mL) was quickly added to the reaction mixture under vigorous stirring. The solvent was evaporated under reduced pressure and an oily residue was resuspended in acetonitrile (6 mL) and filtered. The filtrate was purified on CombiFlash (Method 5-1) yielding the desired Compound 2-1 (300 mg, 89% yield). ¹H NMR (500 MHz, CD₃CN) δ_{H} 6.63 (t, J=2.1 Hz, 2H), 6.00 (t, J=2.0 Hz, 2H), 4.31 (m, 1H), 4.21 (dd, J=14.2, 5.0 Hz, 1H), 4.06 (m, 1H), 3.67 (t, J=4.9 Hz, 2H), 3.60 (m, 2H), 3.54 (m, 6H), 3.46 (m, 2H), 3.29 (m, 2H), 3.07 (t, J=5.1 Hz, 2H), 1.34 (s, 9H); ¹³C (146 MHz, CD₃CN) δ_{C} 170.3, 155.6, 121.4, 108.0, 79.4, 70.0-69.4, 67.0, 56.0, 50.3, 39.2, 27.6. LC-MS (method 1): t_{R}=2.59 min, m/z [M+H]⁺ calcd for [C₂₀H₃₇N₄O₆]⁺, 429.3; found 429.3.

### Compound 2-2

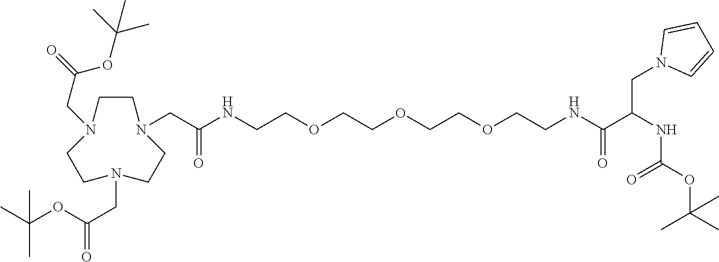 [SMILES: CC(C)(C)OC(=O)CN1CCN(CC(=O)NCCOCCOCCOCCNC(=O)C(Cn2cccc2)NC(=O)OC(C)(C)C)CCN(CC(=O)OC(C)(C)C)CC1]

NOTA-bis(t-Bu ester) (60 mg, 0.14 mmol) and HATU (100 mg, 0.26 mmol) were dissolved in anhydrous dimethylformamide (4 mL) and DIPEA (0.08 mL, 0.46 mmol) was added. The reaction mixture was stirred for 30 min at room temperature and Compound 2-1 (62 mg, 0.15 mmol) dissolved in anhydrous dimethylformamide (1 mL) was added. The reaction mixture was stirred for 30 min at room temperature and was subject to prep-HPLC purification (Method 6-1) yielding the Compound 1-2 (26 mg, 22% yield). ¹H NMR (500 MHz, CDCl₃) δ_{H} 6.62 (t, J=2.2 Hz, 2H), 6.08 (t, J=2.1 Hz, 2H), 5.43 (m, 1H), 4.40 (m, 1H), 4.30 (m, 1H), 4.11 (m, 1H), 3.58-3.35 (m, 20H), 3.35 (s, 4H), 2.88-2.78 (m, 12H), 1.43 (s, 18H), 1.39 (s, 9H); ¹³C (146 MHz, CDCl₃) δ_{C} 171.2, 169.7, 155.3, 121.4, 108.7, 81.3, 80.2, 70.6-69.5, 60.4, 58.3, 55.6-55.0, 51.2, 39.6-39.3, 28.4, 28.3. LC-MS (method 1): t_{R}=3.27 min, m/z [M+H]⁺ calcd for [C₄₀H₇₂N₇O₁₁]⁺, 825.6; found 825.8.

### Compound 2

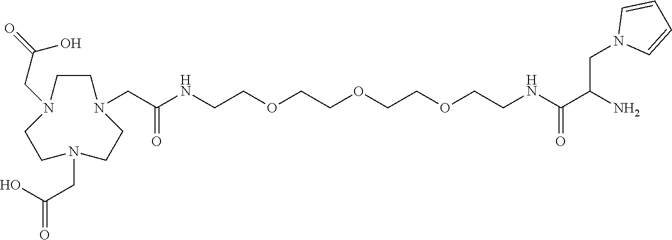 [SMILES: NC(Cn1cccc1)C(=O)NCCOCCOCCOCCNC(=O)CN1CCN(CC(=O)O)CCN(CC(=O)O)CC1]

Compound 2-2 (20 mg, 0.02 mmol) was dissolved in dichloromethane (2 mL) and trifluoroacetic acid (2 mL) was added. The reaction mixture was stirred for 18 hours at room temperature, and the solvent was removed under reduced pressure. The residue was redissolved in water (4 mL) and was subject to prep-HPLC purification (Method 7-1) yielding Compound 2 as a white powder after lyophilization (5 mg, 33% yield). ¹H NMR (500 MHz, D₂O) δ_{H} 6.68 (m, 2H), 6.12 (m, 2H), 4.33 (d, J=6.2 Hz, 2H), 4.23 (t, J=6.3 Hz, 1H), 3.61 (br s, 4H), 3.55 (br s, 8H), 3.49 (t, J=5.9 Hz, 4H), 3.38 (s, 2H), 3.30 (t, J=5.9 Hz, 4H), 3.17 (m, 4H), 3.03 (t, J=5.8 Hz, 4H), 2.78 (t, J=5.8 Hz, 4H); ¹³C NMR (126 MHz, D₂O) Sc 173.8, 173.3, 167.4, 121.9, 109.4, 69.7-68.5, 59.3, 57.3, 54.0, 51.0, 49.4, 48.9, 48.6, 39.3-38.7. LC-MS (method 3-1): t_{R}=4.72 min, m/z [M+H]⁺ calcd for [C₂₇H₄₈N₇O₉]⁺, 614.3; found 614.3.

### Example 3. Compound 3

### Compound 3-1

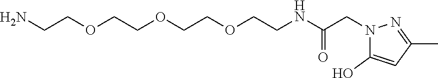 [SMILES: Cc1cc(O)n(CC(=O)NCCOCCOCCOCCN)n1]

(3-Methyl-5-oxo-4,5-dihydro-1H-pyrazol-1-yl)acetic acid hydrochloride (88 mg, 0.45 mmol), DCC (100 mg, 0.49 mmol), N-hydroxysuccinimide (60 mg, 0.52 mmol) and triethylamine (0.1 mL, 0.72 mmol) were dissolved in dichloromethane (5 mL). The reaction mixture was stirred at room temperature for 1 hour, and Boc-NH-PEG₃-NH₂ (133 mg, 0.46 mmol) and DIPEA (0.1 mL, 0.56 mmol) were added. The reaction was stirred at room temperature for another 18 hours, and the solution was filtered. The solvent was removed under reduced pressure, and the residue was purified on CombiFlash (Method 5-1) yielding the crude product (143 mg, 73% yield) that was used for the next step without further purification. The crude mixture (143 mg, 0.33 mmol) was dissolved in dichloromethane (3 mL), and trifluoroacetic acid (0.5 mL) was added. The reaction mixture was stirred at room temperature for 1 hour, and the solvent was removed under reduced pressure. The oily residue was redissolved in acetonitrile and purified on CombiFlash (Method 5-2) yielding the Compound 3-2 (40 mg, 36% yield). ¹H NMR (500 MHz, CD₃CN) δ_{H} 5.15 (s, 1H), 4.45 (s, 1.1H, rotamer 1), 4.19 (s, 1.1H, rotamer 2), 3.68 (t, J=5.1 Hz, 2H), 3.60 (m, 2H), 3.54 (m, 6H), 3.48 (t, J=5.2 Hz, 2H), 3.31 (q, J=5.4 Hz, 2H), 3.07 (t, J=5.1 Hz, 2H); ¹³C NMR (126 MHz, CD₃CN) δ_{C} 168.3, 160.6, 148.4, 89.6, 70.0-69.3, 66.4, 47.3-46.9, 39.4-49.1, 12.3. LC-MS (method 3-1): t_{R}=4.35 min, m/z [M+H]⁺ calcd for [C₁₄H₂₇N₄O₅]⁺, 331.2; found 331.2.

### Compound 3

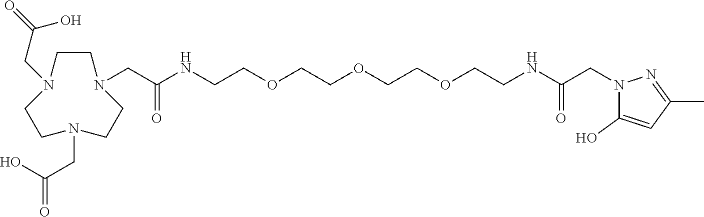 [SMILES: Cc1cc(O)n(CC(=O)NCCOCCOCCOCCNC(=O)CN2CCN(CC(=O)O)CCN(CC(=O)O)CC2)n1]

NOTA trihydrochloride salt (100 mg, 0.24 mmol) was suspended in acetonitrile (5 mL) followed by addition of pentafluorophenol (61 mg, 0.33 mmol), DCC (70 mg, 0.34 mmol) and triethylamine (0.10 mL, 0.72 mmol). The reaction mixture was stirred for 3 hours, and Compound 3-1 (40 mg, 0.12 mmol) dissolved in acetonitrile (1 mL) was quickly added under vigorous stirring. The reaction mixture was stirred for another 1 h and the solution was filtered. The filtrate was concentrated and subject to prep-HPLC purification (Method 7-1) yielding the desired product Compound 3 as a white powder after lyophilization (20 mg, 27% yield). ¹H NMR (500 MHz, D₂O) δ_{H} 4.46 (s, 1.6H, rotamer 1), 4.27 (s, 0.4H, rotamer 2), 3.69 (s, 4H), 3.55 (br s, 8H), 3.50 (q, J=5.2 Hz, 4H), 3.45 (br s, 2H), 3.30 (q, J=5.6 Hz, 4H), 3.21-2.86 (m, 12H), 2.97 (m, 1.6H, rotamer 1), 2.78 (m, 04H, rotamer 2), 2.07 (s, 2.4H, rotamer 1), 1.99 (s, 0.6H, rotamer 2); ¹³C NMR (126 MHz, D₂O) δ_{C} 173.0, 172.5, 168.8, 164.1, 149.2, 69.7-68.6, 58.8 57.1, 51.0-48.9, 49.3 (rotamer 2), 48.3 (rotamer 1), 46.5 (rotamer 2), 46.0 (rotamer 1), 39.1-38.9, 15.8, 11.3. LC-MS (method 3): t_{R}=2.12 min, m/z [M+H]⁺ calcd for [C₂₆H₄₆N₇O₁₀]⁺, 616.3; found 616.3.

### Example 4. Compound 4

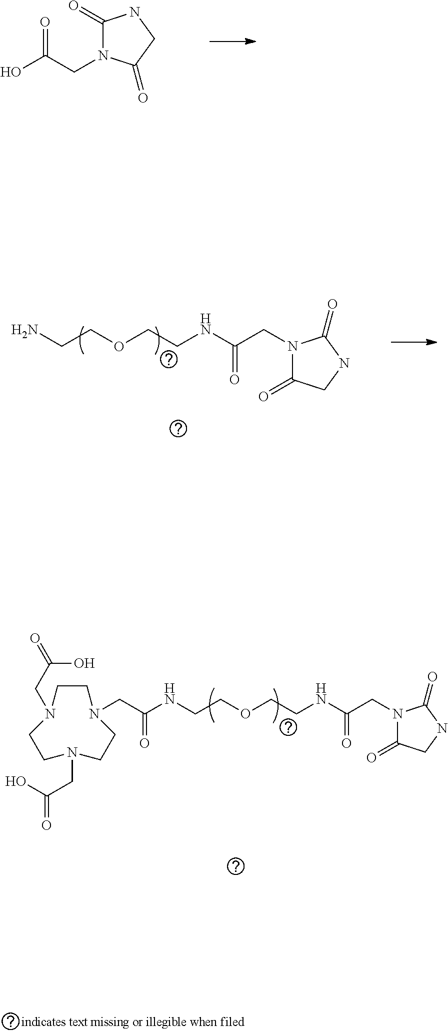

### Compound 4-1

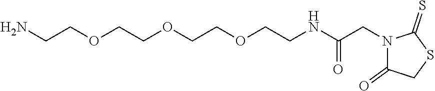 [SMILES: NCCOCCOCCOCCNC(=O)CN1C(=O)CSC1=S]

Rhodanine-3-acetic acid (100 mg, 0.52 mmol), DCC (160 mg, 0.78 mmol) and N-hydroxysuccinimide (80 mg, 0.70 mmol) were dissolved in dichloromethane (5 mL) and stirred for 1 hour at room temperature. NH₂—PEG₃-NH₂ (200 mg, 1.04 mmol) dissolved in dichloromethane (1 mL) was quickly added to the reaction mixture under vigorous stirring. The solvent was evaporated under reduced pressure, and an oily residue was resuspended in acetonitrile (6 mL) and filtered. The filtrate was purified on CombiFlash (Method 5-1) yielding the Compound 4-1 (18 mg, 5% yield). ¹H NMR (500 MHz, CDCl₃) δ_{H} 4.69 (s, 2H), 4.06 (s, 2H), 3.78 (m, 2H), 3.71 (m, 2H), 3.60 (m, 8H), 3.41 (m, 2H), 3.14 (m, 2H); ¹³C NMR (126 MHz, CDCl₃) δ_{C} 201.6, 174.2, 165.6, 70.2-69.6, 67.2, 46.5, 39.5-39.4, 35.9. LC-MS (method 1): t_{R}=2.19 min, m/z [M+H]⁺ calcd for [C₁₃H₂₄N₃O₅S₂]⁺, 366.1; found 366.3.

### Compound 4

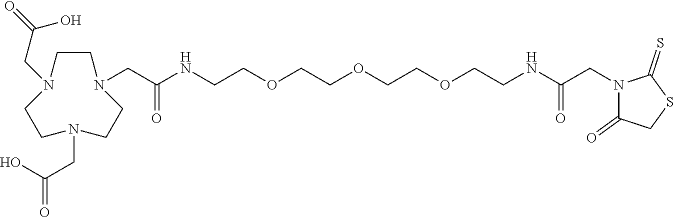 [SMILES: O=C(O)CN1CCN(CC(=O)O)CCN(CC(=O)NCCOCCOCCOCCNC(=O)CN2C(=O)CSC2=S)CC1]

NOTA trihydrochloride salt (110 mg, 0.26 mmol) was suspended in acetonitrile (5 mL) followed by addition of pentafluorophenol (90 mg, 0.49 mmol), DCC (80 mg, 0.39 mmol), and triethylamine (0.20 mL, 1.44 mmol). The reaction mixture was stirred for 3 hours, and Compound 4-1 (70 mg, 0.19 mmol) dissolved in acetonitrile (1 mL) was quickly added under vigorous stirring. The reaction mixture was stirred for another 1 hour, and the solution was filtered. The filtrate was concentrated and subject to prep-HPLC purification (Method 7-1) yielding the desired product Compound 4 as a white powder after lyophilization (8 mg, 6% yield). ¹H NMR (500 MHz, D₂O) δ_{H} 4.57 (s, 2H), 3.69 (s, 4H), 3.55 (m, 8H), 3.49 (m, 4H), 3.46 (s, 2H), 3.30 (m, 4H), 3.21 (s, 4H), 3.08 (t, J=5.8 Hz, 4H), 2.86 (t, J=5.9 Hz, 4H); ¹³C NMR (126 MHz, D₂O) δ_{C} 204.5, 176.2, 173.0, 172.5, 168.2, 69.7-68.6, 58.8, 57.1, 50.1, 49.5, 48.9, 46.1, 39.2-38.9. LC-MS (method 3-1): t_{R}=5.31 min, [M+H]⁺ calcd for [C₂₅H₄₃N₆O₁₀S₂]⁺, 651.2; found 651.2.

### Example 5. Compound 5

### Compound 5-1

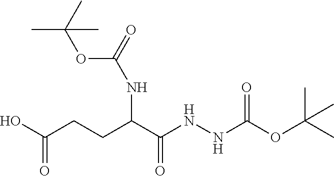 [SMILES: CC(C)(C)OC(=O)NNC(=O)C(CCC(=O)O)NC(=O)OC(C)(C)C]

N-t-Boc-L-Glu-α-Bz ester (2.0 g, 5.9 mmol), N-hydroxysuccinimide (0.68 g, 5.9 mmol), and DCC (1.5 g, 7.3 mmol) were dissolved in dichloromethane (40 mL) and stirred for 1 hour at room temperature. Tert-butyl carbazate (0.78 g, 5.9 mmol) and DIPEA (2 mL, 11.5 mmol) was added to the reaction mixture, and the solution was stirred for 30 min at room temperature. The solvent was evaporated under reduced pressure, and an oily residue was resuspended in acetonitrile (10 mL) and filtered. The filtrate was purified on CombiFlash (Method 5), giving a partially purified product (2 g, 4.4 mmol) after solvent evaporation. The solid residue was dissolved in methanol (15 mL) and 2M KOH was added (5 mL). The solution was stirred at room temperature for 2 hours and was subsequently neutralized. The solution was concentrated under reduced pressure and purified on CombiFlash (Method 5), giving the Compound 5-1 as a white solid (0.4 g, 19% yield over two steps). ¹H NMR (500 MHz, CD₃CN) δ_{H} 4.05 (m, 1H), 2.36 (t, J=7.4 Hz, 1H), 2.22 (t, J=7.4 Hz, 1H), 2.03 (m, 1H), 1.82 (m, 1H), 1.39 (m, 18H); ¹³C NMR (126 MHz, CD₃CN) δ_{C} 173.7-172.2, 155.9-155.6, 80.4-79.2, 53.0-52.4, 29.6-29.4, 27.6-27.4, 27.3-26.9. LC-MS (method 1): t_{R}=3.16 min, m/z [M−H]⁻ calcd for [C₁₅H₂₆N₃O₇]⁻, 360.2; found 360.3.

### Compound 5-2

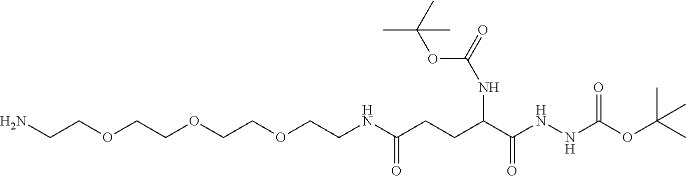 [SMILES: CC(C)(C)OC(=O)NNC(=O)C(CCC(=O)NCCOCCOCCOCCN)NC(=O)OC(C)(C)C]

Compound 5-1 (150 mg, 0.42 mmol), DCC (128 mg, 0.62 mmol), and N-hydroxysuccinimide (55 mg, 0.48 mmol) were dissolved in dichloromethane (8 mL) and stirred for 1 hour at room temperature. NH₂—PEG₃-NH₂ (220 mg, 1.15 mmol) dissolved in dichloromethane (1 mL) was quickly added to the reaction mixture under vigorous stirring. The solvent was evaporated under reduced pressure and an oily residue was resuspended in acetonitrile (6 mL) and filtered. The filtrate was purified on CombiFlash (Method 5-1) yielding Compound 5-2 (100 mg, 45% yield). ¹H NMR (500 MHz, CDCl₃) δ_{H} 4.14 (m, 1H), 3.74 (m, 2H), 3.60 (m, 1OH), 3.43 (m, 2H), 3.13 (m, 2H), 2.28 (m, 2H), 1.97 (m, 2H), 1.40 (two s, 18H); ¹³C NMR (126 MHz, CDCl₃) δC 173.2, 172.1, 156.3-156.2, 81.6-80.0, 70.5-69.6, 67.2, 53.3, 39.3, 32.3-30.5, 30.1-29.2, 28.4-28.3. LC-MS (method 1): t_{R}=2.58 min, m/z [M+H]⁺ calcd for [C₂₃H₄₆N₅O₉]⁺, 536.3; found 536.4.

### Compound 5-3

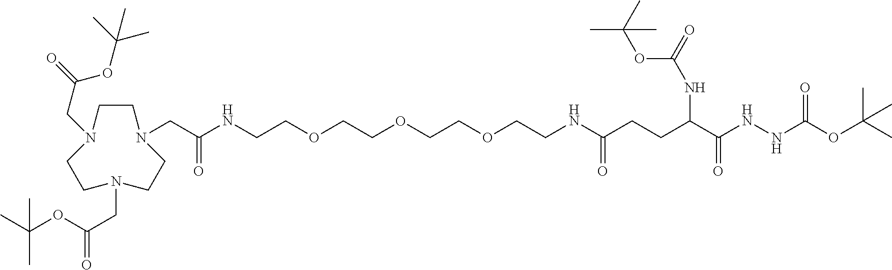 [SMILES: CC(C)(C)OC(=O)CN1CCN(CC(=O)NCCOCCOCCOCCNC(=O)CCC(NC(=O)OC(C)(C)C)C(=O)NNC(=O)OC(C)(C)C)CCN(CC(=O)OC(C)(C)C)CC1]

NOTA-bis(t-Bu ester) (30 mg, 0.07 mmol) and HATU (40 mg, 0.11 mmol) were dissolved in anhydrous dimethylformamide (3 mL), and DIPEA (0.15 mL, 0.74 mmol) was added. The reaction mixture was stirred for 30 min at room temperature, and Compound 5-2 (34 mg, 0.06 mmol) dissolved in anhydrous dimethylformamide (1 mL) was added. The reaction mixture was stirred for 30 min at room temperature and was subject to prep-HPLC purification (Method 6-1) yielding the Compound 5-3 (20 mg, 33% yield). H NMR (500 MHz, CDCl₃) δ_{H} 3.58 (m, 8H), 3.51 (t, J=5.7 Hz, 4H), 3.43 (m, 5H), 3.33 (m, 4H), 3.27 (m, 2H), 2.88-2.81 (m, 12H), 2.23 (m, 4H), 1.41 (m, 36H); ¹³C NMR (126 MHz, CDCl₃) δC 173.1-170.8, 156.5-155.8, 81.8-79.6, 81.3, 70.6-69.5, 60.1, 58.2, 55.5-53.2, 39.4-39.1, 32.5-29.1, 28.4-28.2. LC-MS (method 1): t_{R}=3.22 min, m/z [M+H]⁺ calcd for [C₄₃H₈₁N₈O₁₄]⁺, 933.6; found 933.6.

### Compound 5

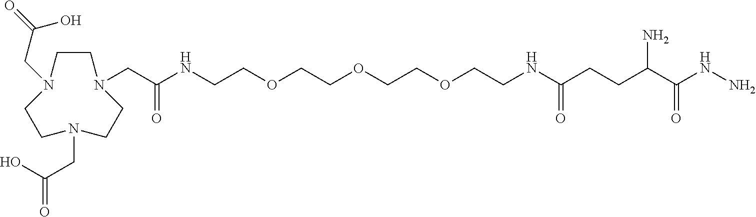 [SMILES: NNC(=O)C(N)CCC(=O)NCCOCCOCCOCCNC(=O)CN1CCN(CC(=O)O)CCN(CC(=O)O)CC1]

Compound 5-3 (40 mg, 0.04 mmol) was dissolved in dichloromethane (2 mL) and trifluoroacetic acid (2 mL) was added. The reaction mixture was stirred for 18 hours at room temperature, and the solvent was removed under reduced pressure. The residue was redissolved in water (4 mL) and was subject to prep-HPLC purification (Method 7-1) yielding the desired product Compound 5 as a white powder after lyophilization (25 mg, quantitative yield). ¹H NMR (500 MHz, D₂O) δ_{H} 3.84 (dt, J=33.6, 6.8 Hz, 1H), 3.61 (s, 4H), 3.53 (m, 8H), 3.48 (m, 4H), 3.45 (s, 2H), 3.29 (m, J=5.4 Hz, 4H), 3.16 (m, 4H), 3.03 (t, J=5.8 Hz, 4H), 2.77 (t, J=5.8 Hz, 4H), 2.25 (m, 2H), 2.02 (m, 2H); ¹³C NMR (126 MHz, D₂O) δC 174.0, 173.8, 173.2, 169.1, 168.2, 69.6-68.6, 59.3, 57.3, 52.6-51.5, 51.0, 49.4, 48.6, 39.1-38.7, 30.8-28.7, 26.5-26.3. LC-MS (method 3-1): t_{R}=7.54 min, m/z [M+H]⁺ calcd for [C₂₅H₄₉N₈O₁₀]⁺, 621.3; found 621.3.

### Example 6. Compound 6

### Compound 6-1

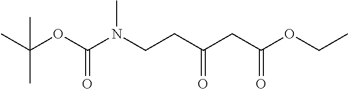 [SMILES: CCOC(=O)CC(=O)CCN(C)C(=O)OC(C)(C)C]

3-((tert-Butoxycarbonyl)(methyl)amino)propanoic acid (2.0 g, 9.9 mmol) was dissolved in dichloromethane (40 mL). Meldrum's acid (1.4 g, 9.9 mmol), DCC (2.0 g, 9.9 mmol) and DMAP (1.2 g, 9.8 mmol) were added at 0° C. The reaction mixture was allowed to reach room temperature and was stirred for 5 hours. The solvent was removed under reduced pressure, and the residue was redissolved in ethanol (20 mL) and refluxed for 18 hours. The solvent was removed under reduced pressure. The residue was redissolved in dichloromethane and purified on CombiFlash (80 g SiO₂ column), yielding Compound 6-1 (1.9 g, 71% yield). ¹H NMR (500 MHz, CDCl₃) δ_{H} 4.16 (q, J=7.0 Hz, 2H), 3.44 (m, 4H), 2.82 (s, 3H), 2.77 (m, 2H), 1.41 (s, 9H), 1.24 (H¹¹, t, J=7.1 Hz, 3H); ¹³C NMR (126 MHz, CDCl₃) δ_{C} 201.9-201.5, 167.1, 155.7-155.6, 79.7, 61.5, 49.7-49.4, 43.9, 41.6, 35.2-34.7, 28.5, 14.2. LC-MS (method 1): t_{R}=3.73 min, m/z [M+Na]⁺ calcd for [C₁₃H₂₃NO₅Na]⁺, 296.1; found 296.1.

### Compound 6-2

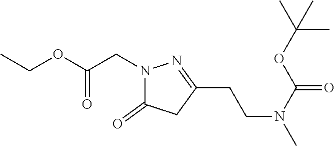 [SMILES: CCOC(=O)CN1N=C(CCN(C)C(=O)OC(C)(C)C)CC1=O]

Ethyl 2-hydrazinylacetate hydrochloride (1.1 g, 7.0 mmol) was suspended in ethanol (20 mL) and triethylamine (2 mL, 14.4 mmol) was added. The solution was stirred for 1 hour and Compound 6-1 (1.9 g, 7.0) was added. The reaction mixture was stirred at 50° C. for 2 hours, was cooled down and purified via CombiFlash (Method 5-3) giving Compound 6-2 (1.2 g, 51% yield) as a yellow oil. ¹H NMR (500 MHz, CDCl₃) δ_{H} 4.38 (s, 2H), 4.19 (q, J=7.1 Hz, 2H), 3.47 (m, 2H), 3.31 (m, 2H), 2.84 (s, 3H), 2.60 (t, J=6.9 Hz, 2H), 1.41 (s, 9H), 1.25 (t, J=7.1 Hz, 3H); 13C NMR (126 MHz, CDCl₃) δ_{C} 173.0-172.6, 157.6-155.9, 79.9, 61.7, 46.2-45.6, 45.5, 40.0, 34.3, 29.6, 28.4, 14.2. LC-MS (method 1): t_{R}=3.21 min, m/z [M+H]⁺ calcd for [C₁₅H₂₆N₃O₅]⁺, 328.2; found 328.3.

### Compound 6-3

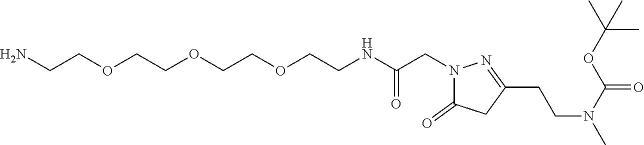 [SMILES: CN(CCC1=NN(CC(=O)NCCOCCOCCOCCN)C(=O)C1)C(=O)OC(C)(C)C]

Compound 6-2 (1.2 g, 3.57 mmol) was dissolved in methanol (15 mL) and 2M KOH (10 mL) was added. The reaction mixture was stirred at room temperature for 4 hours, neutralized, and concentrated under reduced pressure. The residue was redissolved in methanol (6 mL) and purified on CombiFlash (Method 5-1) yielding a crude mixture (0.7 g) that was used for the next step without further purification. This crude product (200 mg, 0.67 mmol), DCC (180 mg, 0.87 mmol), and N-hydroxysuccinimide (90 mg, 0.79 mmol) were dissolved in dichloromethane (8 mL) and stirred for 1 hour at room temperature. NH₂-PEG₃-NH₂ (230 mg, 1.20 mmol) dissolved in dichloromethane (1 mL) was quickly added to the reaction mixture under vigorous stirring. The solvent was evaporated under reduced pressure and an oily residue was resuspended in acetonitrile (6 mL) and filtered. The filtrate was purified on CombiFlash (Method 5-1) yielding the Compound 6-4 (123 mg, 25% yield over two steps). ¹H NMR (500 MHz, CDCl₃) δ_{H} 7.89 (NH, s, 1H), 5.20 (s, 1H), 4.51 (s, 2H), 3.69 (m, 2H), 3.61 (m, 2H), 3.56 (m, 6H), 3.50 (m, 2H), 3.37 (m, 4H), 3.10 (m, 2H), 2.80 (s, 3H), 2.62 (t, J=7.2 Hz, 2H), 1.38 (s, 9H); ¹³C NMR (126 MHz, CDCl₃) δ_{C} 167.8, 155.9, 149.6, 89.2, 79.8, 70.3-69.5, 66.9, 48.0, 47.6, 39.5-39.3, 34.5, 28.4, 26.2. LC-MS (method 1): t_{R}=2.47 min, m/z [M+H]⁺ calcd for [C₂₁H₄₀N₅O₇]⁺, 474.3; found 474.3.

### Compound 6

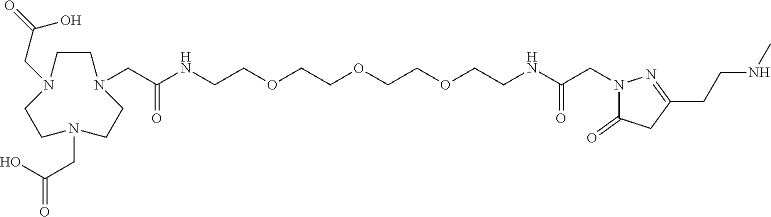 [SMILES: CNCCC1=NN(CC(=O)NCCOCCOCCOCCNC(=O)CN2CCN(CC(=O)O)CCN(CC(=O)O)CC2)C(=O)C1]

NOTA-bis(t-Bu ester) (15 mg, 0.04 mmol), DCC (20 mg, 0.10 mmol), and N-hydroxysuccinimide (14 mg, 0.12 mmol) were dissolved in dichloromethane (4 mL) and stirred for 1 hour at room temperature. Compound 6-4 (230 mg, 1.20 mmol) dissolved in dichloromethane (1 mL) was quickly added to the reaction mixture under vigorous stirring. The solvent was evaporated under reduced pressure and an oily residue was resuspended in acetonitrile (5 mL) and filtered. The filtrate was subject to prep-HPLC purification (Method 6-1) yielding the crude mixture containing some unreacted NOTA-bis(t-Bu ester). The solvent was removed under reduced pressure, and the residue was redissolved in dichloromethane (2 mL) followed by addition of trifluoroacetic acid (2 mL). The reaction mixture was stirred at room temperature for 18 hours, and the solvent was removed under reduced pressure. The residue was redissolved in water (4 mL) and was subject to prep-HPLC purification (Method 7-1) yielding the desired product Compound 6 as a white powder after lyophilization (12 mg, 45% yield). ¹H NMR (500 MHz, D₂O) δ_{H} 4.51 (s, 1.68H, rotamer 1), 4.32 (s, 0.32H, rotamer 2), 3.65 (s, 4H), 3.55 (br s, 8H), 3.50 (t, J=5.8 Hz, 4H), 3.40 (s, 2H), 3.30 (m, 6H), 3.20 (m, 6H), 3.05 (t, J=5.8 Hz, 4H), 2.83 (t, J=7.1 Hz, 2H), 2.80 (m, 4H), 2.61 (0.55H, rotamer 1), 2.60 (2.45H, rotamer 2); ¹³C NMR (126 MHz, D₂O) δ_{C} 173.6, 172.8, 169.5 (rotamer 2), 169.0 (rotamer 1), 161.1, 147.1, 69.6-68.6, 59.1 57.3, 51.1, 50.8, 49.4, 48.7, 47.4, 46.6 (rotamer 1), 45.0 (rotamer 2), 39.1-38.8, 32.9, 23.7. LC-MS (method 3-1): t_{R}=4.37 min, m/z [M+H]⁺ calcd for [C₂₈H₅₁N₈O₁₀]⁺, 659.4; found 659.3.

### Example 7. Compound 7

### Compound 7-1

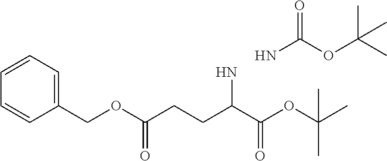

To a solution of 5-benzyl 1-(tert-butyl) 2-hydroxypentanedioate (2.94 g, 10 mmol) in anhydrous dichloromethane (50 mL), 2,6-lutidine (3.21 g, 30 mmol) and Tf₂O (3.38 g, 12 mmol) were successively added at 0° C., and the reaction mixture was stirred for 1 hour. The reaction mixture was added dropwise to a solution of tert-butyl carbazate (6.6 g, 50 mmol) in anhydrous dichloromethane (20 mL) at 0° C. After stirring for 1 hour, the solution was washed with aqueous solution of citric acid (10%) and brine. The organic layer was dried over Na₂SO₄ and concentrated under reduced pressure. The crude product was purified on CombiFlash method 5-3 with A and B as solvents to give Compound 7-1 as a yellow oil (1.76 g, 43%). ¹H NMR (500 MHz, CDCl₃) δ 7.40-7.29 (m, 5H), 6.15 (s, 1H), 5.11 (s, 2H), 4.21 (s, 1H), 3.53 (s, 1H), 2.52 (t, J=7.4 Hz, 2H), 2.15-2.04 (m, 1H), 1.92 (dd, J=14.3, 7.2 Hz, 1H), 1.44 (d, J=11.4 Hz, 18H). ¹³C NMR (126 MHz, CDCl₃) δ 173.3, 171.9, 156.3, 136.0, 128.6-128.2, 82.0, 80.4, 66.4, 62.45, 30.3, 28.4-28.1, 25.2. LC-MS (method 1): t_{R}=4.50 min, m/z [M+H]⁺ calcd for [C₂₁H₃₃N₂O₆]⁺, 409.2; found 409.3.

### Compound 7-2

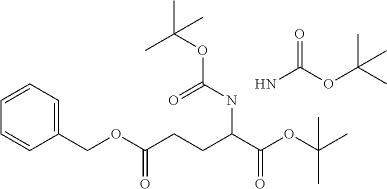

Compound 7-1 (0.82 g, 2 mmol) and di-tert-butyl decarbonate (0.88 g, 4 mmol) were dissolved in dichloromethane (30 mL), and a solution of 4-dimethylaminopyridine (24 mg, 0.2 mmol) in dichloromethane (4 mL) was added dropwise. The reaction mixture was stirred for 18 hours at room temperature and then extracted with aqueous solution of citric acid (10%) and brine. The organic layer was dried over Na₂SO₄ and concentrated under reduced pressure. The crude product was purified on CombiFlash method 5-3 with A and B as solvents to give Compound 7-2 as a yellow oil (0.88 g, 87%). ¹H NMR (500 MHz, CDCl₃) δ 7.39-7.28 (5H), 5.10 (d, J=3.1 Hz, 2H), 3.53 (t, J=5.5 Hz, 1H), 2.74 (m, 1H), 2.36 (m, 1H), 2.01 (m, 2H), 1.47 (s, 18H), 1.44 (s, 9H). ¹³C NMR (126 MHz, CDCl₃) δ 173.11, 170.67, 152.16, 136.05, 128.61, 128.29, 128.26, 83.79, 82.33, 66.31, 62.54, 29.81, 28.06, 25.83. LC-MS (method 1): t_{R}=4.90 min, m/z [M+H]⁺ calcd for [C₂₆H₄₁N₂O₈]⁺, 509.3; found 509.3.

### Compound 7-3

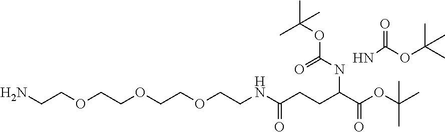

Compound 7-2 (0.51 g, 1.0 mmol) was added to a slurry of palladium on carbon (50% water, 50 mg) in methanol (20 mL). The solution was purged twice with hydrogen and stirred for 12 hours under hydrogen at room temperature. Celite was added to the reaction mixture, and the slurry was filtered through a Celite bed preliminary wetted with methanol. The filtrate was concentrated under reduced pressure to give a colorless oil (0.25 g, 0.5 mmol), which was used for the next reaction without further purification. This crude product (0.20 g, 0.4 mmol), DCC (150 mg, 0.73 mmol), and N-hydroxysuccinimide (70 mg, 0.61 mmol) were dissolved in dichloromethane (8 mL) for 1 hour at room temperature. NH₂—PEG₃-NH₂ (300 mg, 1.56 mmol) dissolved in dichloromethane (1 mL) was quickly added to the reaction mixture under vigorous stirring. The solvent was evaporated under reduced pressure, and an oily residue was resuspended in acetonitrile (6 mL) and filtered. The filtrate was purified on CombiFlash (Method 5-1) yielding Compound 7-3 (80 mg, 17% yield over two steps). ¹H NMR (500 MHz, CDCl₃) δ_{H} 3.74 (m, 2H), 3.65 (m, 2H), 3.58 (m, 6H), 3.52 (t, J=5.0 Hz, 2H), 3.48 (t, J=5.6 Hz, 1H), 3.37 (m, 2H), 3.14 (m, 2H), 2.41 (dt, J=15.5, 7.6 Hz, 1H), 2.25 (dt, J=15.0, 7.4 Hz, 1H), 1.96 (m, 2H), 1.44 (m, 27H); ¹³C NMR (126 MHz, CDCl₃) δ_{C} 173.1, 171.5, 152.8, 83.9, 82.1, 70.5-69.9, 67.2, 63.0, 39.8, 39.3, 31.8, 28.3-28.0, 26.8. LC-MS (method 1): t_{R}=3.05 min, m/z [M+H]⁺ calcd for [C₂₇H₅₃N₄O₁₀]⁺, 593.4; found 593.5.

### Compound 7-4

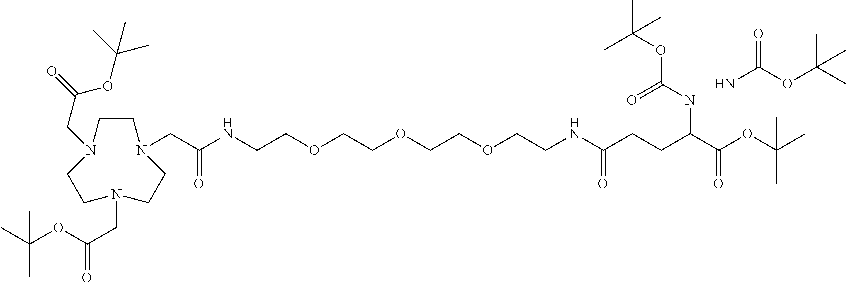

NOTA-bis(t-Bu ester) (30 mg, 0.07 mmol) and HATU (30 mg, 0.09 mmol) were dissolved in anhydrous dimethylformamide (3 mL), and DIPEA (0.10 mL, 0.49 mmol) was added. The reaction mixture was stirred for 30 min at room temperature, and Compound 7-3 (40 mg, 0.07 mmol) dissolved in anhydrous dimethylformamide (1 mL) was added. The reaction mixture was stirred for 30 min at room temperature and was subject to prep-HPLC purification (Method 6-1) yielding the Compound 7-4 (30 mg, 43% yield). ¹H NMR (500 MHz, CDCl₃) δ_{H} 3.60 (m, 8H), 3.53 (dt, J=10.5, 5.5 Hz, 6H), 3.43 (m, 4H), 3.37 (m, 5H), 2.87 (m, 1H), 2.56 (ddd, J=14.8, 9.7, 5.7 Hz, 1H), 2.18 (ddd, J=14.4, 9.9, 5.8 Hz, 1H), 1.96 (m, 2H), 1.46 (m, 45H); ¹³C NMR (126 MHz, CDCl₃) δ_{C} 173.0, 171.2, 171.0, 152.7, 83.6, 82.0-81.5, 70.6-69.9, 62.7, 60.0, 58.0, 55.1-52.7, 39.3-39.2, 31.9, 28.3-28.1, 27.2. LC-MS (method 1): t_{R}=3.53 min, m/z [M+H]⁺ calcd for [C₄₇H₈₈N₇O₁₅]⁺, 990.6; found 990.7.

### Compound 7

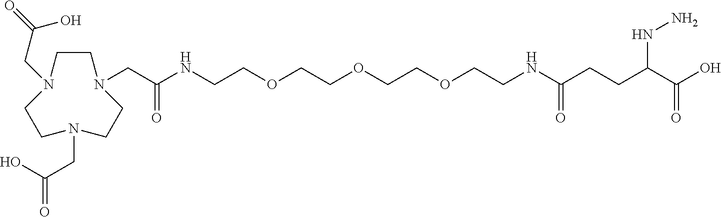 [SMILES: NNC(CCC(=O)NCCOCCOCCOCCNC(=O)CN1CCN(CC(=O)O)CCN(CC(=O)O)CC1)C(=O)O]

Compound 7-4 (60 mg, 0.06 mmol) was dissolved in concentrated phosphoric acid (0.4 mL). The reaction mixture was stirred for 1 hour at room temperature, and the reaction mixture was diluted with water (4 mL). The solution was subject to prep-HPLC purification (Method 7-1) yielding Compound 7 as a white powder after lyophilization (10 mg, 52% yield). ¹H NMR (500 MHz, D₂O) δ_{H} 3.66 (s, 4H), 3.51 (m, 9H), 3.46 (t, J=5.1 Hz, 4H), 3.41 (s, 2H), 3.26 (t, J=5.7 Hz, 2H), 3.22 (t, J=5.4 Hz, 2H), 3.19 (m, 4H), 3.05 (t, J=5.9 Hz, 4H), 2.82 (t, J=6.0 Hz, 4H), 2.24 (m, 2H), 1.91 (m, 2H); ¹³C NMR (126 MHz, D₂O) δ_{C} 175.1, 174.7, 173.2, 172.5, 69.6-68.6, 62.5, 58.8, 57.2, 51.0, 49.5, 48.8, 39.0-38.8, 31.6, 25.1. LC-MS (method 3-1): t_{R}=4.53 min, m/z [M+H]⁺ calcd for [C₂₅H₄₈N₇O₁₁]⁺, 622.3; found 622.3.

### Example 8. Compound 8

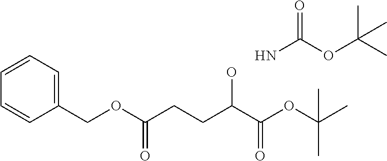

### Compound 8-1

To a stirring solution of N-Boc hydroxylamine (1.99 g, 15 mmol) in dimethylformamide (20 mL) was added DBU (3.04 g, 20 mmol) in dimethylformamide (1 mL). Then 5-benzyl 1-(tert-butyl) 2-((methylsulfonyl)oxy)pentanedioate (3.72 g, 10 mmol) was added dropwise. The reaction was allowed to stir for 20 hours at 50° C. The reaction solution was then concentrated, and the residue was purified via CombiFlash method 5-3 with A and B as solvents to give Compound 8-1 as a yellow oil (2.17 g, 53%). ¹H NMR (500 MHz, CDCl₃) δ 7.40-7.29 (m, 5H), 5.11 (s, 2H), 4.24 (dd, J=9.2, 3.8 Hz, 1H), 2.63 (m, 2H), 2.22-2.12 (m, 1H), 2.07-1.89 (m, 1H), 1.46 (two s, 18H). ¹³C NMR (126 MHz, CDCl₃) δ 172.83, 170.48, 156.37, 136.00, 128.63, 128.27, 82.76-82.00, 66.42, 29.99, 28.27-28.17, 26.00. LC-MS (method 1): t_{R}=4.45 min, m/z=432.1 [M+Na]⁺; calcd: 432.2.

### Compound 8-2

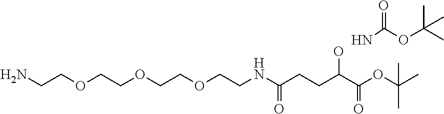

Compound 8-1 (0.41 g, 1.0 mmol) was added to a slurry of palladium on carbon (50% water, 50 mg) in methanol (10 mL). The mixture was purged twice with hydrogen and then stirred at room temperature under argon for 12 hours. Celite was added to the reaction mixture, and the slurry was filtered through a Celite bed preliminary wetted with methanol. The filtrate was concentrated under reduced pressure to give a colorless oil (0.25 g, 0.5 mmol), which was used for the next reaction without further purification. This crude product (180 mg, 0.57 mmol), DCC (180 mg, 0.87 mmol), and N-hydroxysuccinimide (80 mg, 0.70 mmol) were dissolved in dichloromethane (8 mL) for 1 hour at room temperature. NH₂—PEG₃-NH₂ (200 mg, 1.04 mmol) dissolved in dichloromethane (1 mL) was quickly added to the reaction mixture under vigorous stirring. The solvent was evaporated under reduced pressure, and an oily residue was resuspended in acetonitrile (6 mL) and filtered. The filtrate was purified on CombiFlash (Method 5-1) yielding Compound 8-2 (210 mg, 75% yield). ¹H NMR (500 MHz, CDCl₃) δ_{H} 4.28 (dd, J=8.3, 3.9 Hz, 1H), 3.76 (t, J=4.9 Hz, 2H), 3.67 (m, 2H), 3.57 (m, 8H), 3.39 (m, 2H), 3.13 (m, 2H), 2.38 (m, 2H), 2.22 (m, 1H), 1.98 (m, 1H), 1.44 (two s, 18H); ¹³C NMR (126 MHz, CDCl₃) δ_{C} 173.0-172.9, 171.0, 157.2, 82.8-82.0, 70.3-69.7, 67.3, 39.5-39.1, 31.4, 28.3-28.1, 26.4. LC-MS (method 1): t_{R}=2.77 min, m/z [M+H]⁺ calcd for [C₂₂H₄₄N₃₀₉]⁺, 494.3; found 494.3.

### Compound 8-3

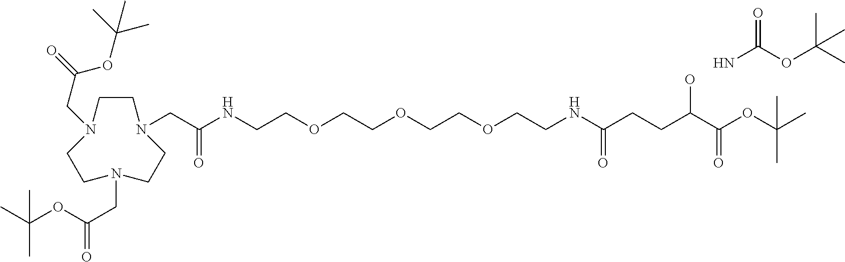

NOTA-bis(t-Bu ester) (50 mg, 0.12 mmol) and HATU (60 mg, 0.17 mmol) were dissolved in anhydrous dimethylformamide (3 mL) and DIPEA (0.15 mL, 0.74 mmol) was added. The reaction mixture was stirred for 30 min at room temperature, and Compound 8-2 (58 mg, 0.12 mmol) dissolved in anhydrous dimethylformamide (1 mL) was added. The reaction mixture was stirred for 30 min at room temperature and was subject to prep-HPLC purification (Method 6-1) yielding the Compound 8-3 (48 mg, 45% yield). ¹H NMR (500 MHz, CDCl₃) δ_{H} 4.18 (dd, J=10.7, 3.2 Hz, 1H), 3.58 (m, 8H), 3.51 (m, 4H), 3.40 (m, 4H), 3.30 (s, 4H), 3.26 (s, 2H), 2.84 (m, 8H), 2.65 (m, 4H), 2.48 (ddd, J=13.9, 9.4, 6.9 Hz, 1H), 2.34 (ddd, J=14.0, 7.2, 5.1 Hz, 1H), 2.18 (dddd, J=14.5, 10.0, 7.2, 3.2 Hz, 1H), 1.88 (dddd, J=14.5, 9.4, 7.2, 3.8 Hz, 1H), 1.43 (two s, 36H); ¹³C NMR (126 MHz, CDCl₃) Sc 172.4, 171.4, 170.7, 156.8, 82.6-82.1, 81.1, 70.5-69.8, 61.1, 58.7, 56.4-56.0, 54.7, 39.2, 32.3, 28.3-28.1, 27.2. LC-MS (method 1): t_{R}=3.32 min, m/z [M+H]⁺ calcd for [C₄₂H₇₉N₆O₁₄]⁺, 891.6; found 891.6.

### Compound 8

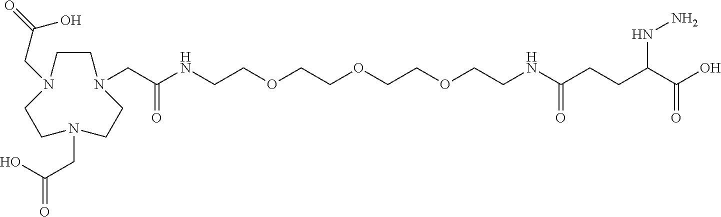 [SMILES: NNC(CCC(=O)NCCOCCOCCOCCNC(=O)CN1CCN(CC(=O)O)CCN(CC(=O)O)CC1)C(=O)O]

Compound 8-3 (22 mg, 0.02 mmol) was dissolved in concentrated phosphoric acid (0.4 mL). The reaction mixture was stirred for 1 hour at room temperature, and the reaction mixture was diluted with water (4 mL). The solution was subject to prep-HPLC purification (Method 7-1) yielding Compound 8 as a white powder after lyophilization (8 mg, 52% yield). ¹H NMR (500 MHz, D₂O) δ_{H} 4.29 (dd, J=7.9, 4.0 Hz, 1H), 3.69 (s, 4H), 3.54 (m, 8H), 3.49 (t, J=5.5 Hz, 4H), 3.45 (s, 2H), 3.30 (t, J=5.7 Hz, 2H), 3.26 (t, J=5.4 Hz, 2H), 3.22 (m, 4H), 3.08 (t, J=5.9 Hz, 4H), 2.86 (t, J=6.0 Hz, 4H), 2.24 (m, 2H), 2.00 (m, 2H); ¹³C NMR (126 MHz, D₂O) δ_{C} 175.5, 175.4, 173.2, 172.6, 82.2, 69.6-68.6, 58.8, 57.2, 51.0, 49.5, 48.6, 39.0-38.8, 31.4, 26.9. LC-MS (method 3-1): t_{R}=4.54 min, m/z [M+H]⁺ calcd for [C₂₅H₄₇N₆O_{12]}⁺, 623.3; found 623.3.

### Example 9. Compound 9

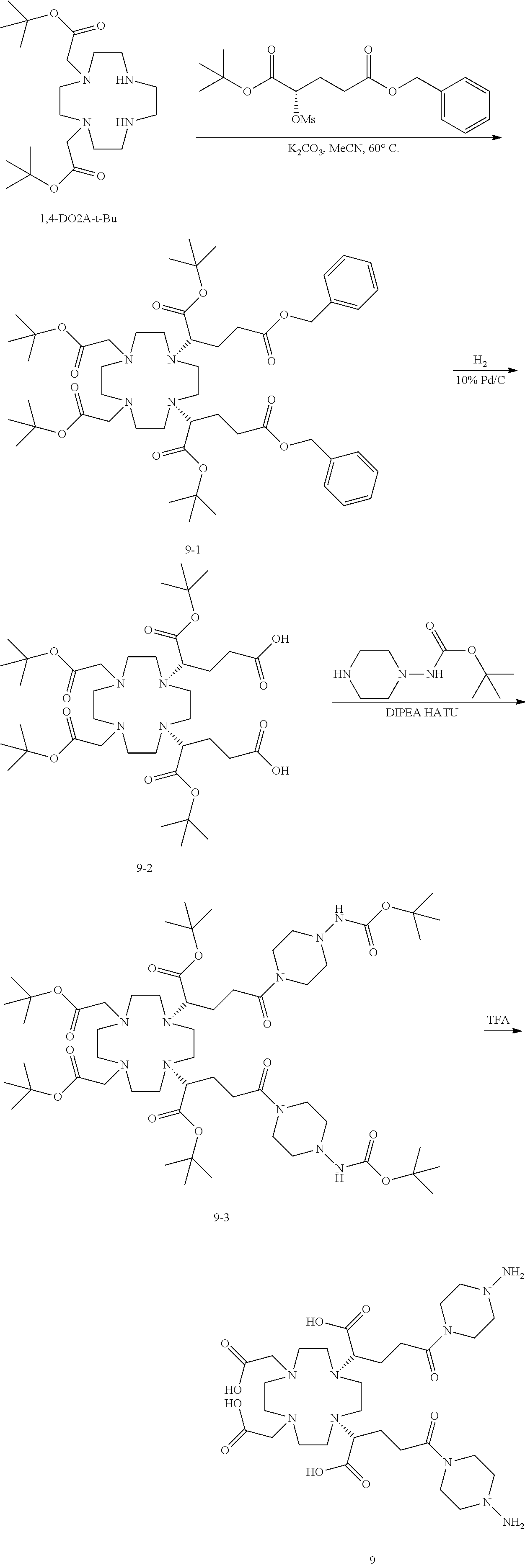

### Compound 9-1

(S)-5-Benzyl-1-tert-butyl 2-(methylsulfonyloxy)pentanedioate (928 mg, 2.5 mmol), as a solution in CH₃CN (2 mL), was added to a stirred mixture of 1,4-DO2A-t-Bu (400 mg, 1.0 mmol) and potassium carbonate (345 mg, 2.5 mmol) in CH₃CN (20 mL) preheated to 60° C. Then the reaction was heated to 80° C. After 12 hours, the reaction mixture was cooled to room temperature and filtered. The filtrate was concentrated and then purified using method 9 to give Compound 9-1 as a yellow oil (180 mg, 19% yield). ¹H NMR (500 MHz, chloroform-d) δ (ppm): 7.36-7.28 (m, 10H), 5.07 (s, 4H), 3.35-3.14 (m, 6H), 2.95-2.73 (m, 12H), 2.60 (d, J=9.3 Hz, 4H), 2.53-2.33 (m, 4H), 1.98 (dq, J=14.5, 7.5, 7.0 Hz, 2H), 1.82 (dq, J=14.3, 7.8 Hz, 2H), 1.42 (d, J=2.5 Hz, 36H). ¹³C NMR (126 MHz, Chloroform-d) δ (ppm): 173.21, 171.98, 170.48, 136.12, 128.60, 128.29, 128.24, 81.23, 81.16, 66.21, 63.53, 55.67, 52.70, 51.96, 50.35, 48.89, 30.99, 28.37, 28.27, 24.92. LC-MS (method 1, ESI^{†}): t_{R}=3.51 min, m/z [M+H]⁺ calcd for [C₅₂H₈₁N₄O₁₂]⁺, 953.6; found 953.5.

### Compound 9-2

Compound 9-1 (150 mg, 0.16 mmol) was dissolved in anhydrous ethanol (10 mL) and activated palladium on carbon (10%, 15 mg) was added to the solution. The suspension was placed under vacuum then connected to a balloon containing hydrogen gas. The reaction mixture was stirred for 2 hours. Then the reaction mixture was passed through a Celite pad and the filtrate was subjected to rotary evaporation, giving the Compound 9-2 as a slight yellow solid (119 mg, 98% yield). ¹H NMR (500 MHz, Acetonitrile-d₃) δ (ppm): 3.65 (d, J=17.2 Hz, 411), 3.52 (dd, J=9.8, 3.5 Hz, 2H), 3.15-2.89 (m, 12H), 2.80-2.67 (m, 4H), 2.35 (hept, J=8.4, 7.4 Hz, 4H), 2.00 (tt, J=12.9, 6.3 Hz, 2H), 1.87 (dq, J=12.4, 7.7 Hz, 2H), 1.46 (d, J=5.8 Hz, 36H). ¹³C NMR (126 MHz, Acetonitrile-d₃) δ (ppm): 175.25, 170.68, 168.86, 82.05, 82.02, 63.03, 54.51, 51.20, 49.98, 48.47, 48.02, 31.87, 27.53, 27.46, 25.11. LC-MS (method 2, ESI^{†}): t_{R}=5.67 min, m/z [M+H]⁺ calcd for [C₃₈H₆₉N₄O₁₂]⁺, 773.5; found 773.5.

### Compound 9-3

Compound 9-2 (100 mg, 0.13 mmol) and N,N-diisopropylethylamine (50 mg, 0.39 mmol) were dissolved in dry CH₃CN (5 mL). After 15 minutes, O-(7-azabenzotriazol-1-yl)-N,N,N′,N′-tetramethyluronium hexafluorophosphate (HATU, 98.8 mg, 0.26 mmol) was added. After stirring for another 1 hour, tert-butyl piperazin-1-ylcarbamate (52.3 mg, 0.26 mmol) was added, and the stirring was continued for 4 hours. Then the solvent was evaporated, and the residue was purified using Method 14 to yield 140 mg (95% yield) of Compound 9-3 as a white solid product. ¹H NMR (500 MHz, Methanol-d₄) δ (ppm): 3.78-3.69 (m, 2H), 3.68-3.43 (m, 10H), 3.11 (dt, J=18.6, 14.1 Hz, 3H), 2.98 (dt, J=17.4, 12.9 Hz, 2H), 2.85-2.59 (m, 16H), 2.52 (d, J=14.2 Hz, 2H), 2.29-2.14 (m, 3H), 2.11-1.96 (m, 3H), 1.77 (tt, J=14.6, 7.9 Hz, 1H), 1.63-1.55 (m, 4H), 1.55-1.39 (m, 54H). ¹³C NMR (126 MHz, Methanol-d₄) δ (ppm): 173.23, 171.29, 165.44, 156.02, 84.60, 81.86, 79.73, 57.89, 55.23, 54.90, 54.58, 53.29, 50.69, 44.32, 42.27, 40.95, 29.47, 27.34, 27.26, 19.27. LC-MS (method 1, ESI^{†}): t_{R}=3.18 min, m/z [M+H]⁺ calcd for [C₅₆H₁₀₃N₁₀O₁₄]⁺, 1139.8; found. 1139.7.

### Compound 9

Compound 9-3 (100 mg, 88 μmol) was dissolved in a mixture of trifluoroacetic acid (TFA, 2 mL), triisopropyl silane (150 μL), 1-dodecathiol (150 μL), and water (10 μL). The mixture was stirred at room temperature overnight. The volatiles were removed by vacuum. To the residue was added diethyl acetate to precipitate out the product as a white solid. The solid was isolated and washed with diethyl acetate three times. The residue was purified using HPLC Method 16 to give Compound 9 as a white solid (56.5 mg, 90% yield). ¹H NMR (500 MHz, D₂O) δ (ppm): 4.33-2.37 (m, 50H), 2.06 (d, J=42.5 Hz, 2H). ¹³C NMR (126 MHz, D₂O) δ (ppm): 175.85, 173.33, 169.57, 58.10, 55.50, 53.51, 51.83, 50.91, 45.11, 43.59, 40.04, 29.52, 19.71. LC-MS (method 3 or method 7, ESI^{†}): t_{R}=2.28 min, m/z [M+H]⁺ calcd for [C₃₀H₅₄N₁₀O₁₀]⁺, 715.4; found 715.3.

### Example 10. Compound 10

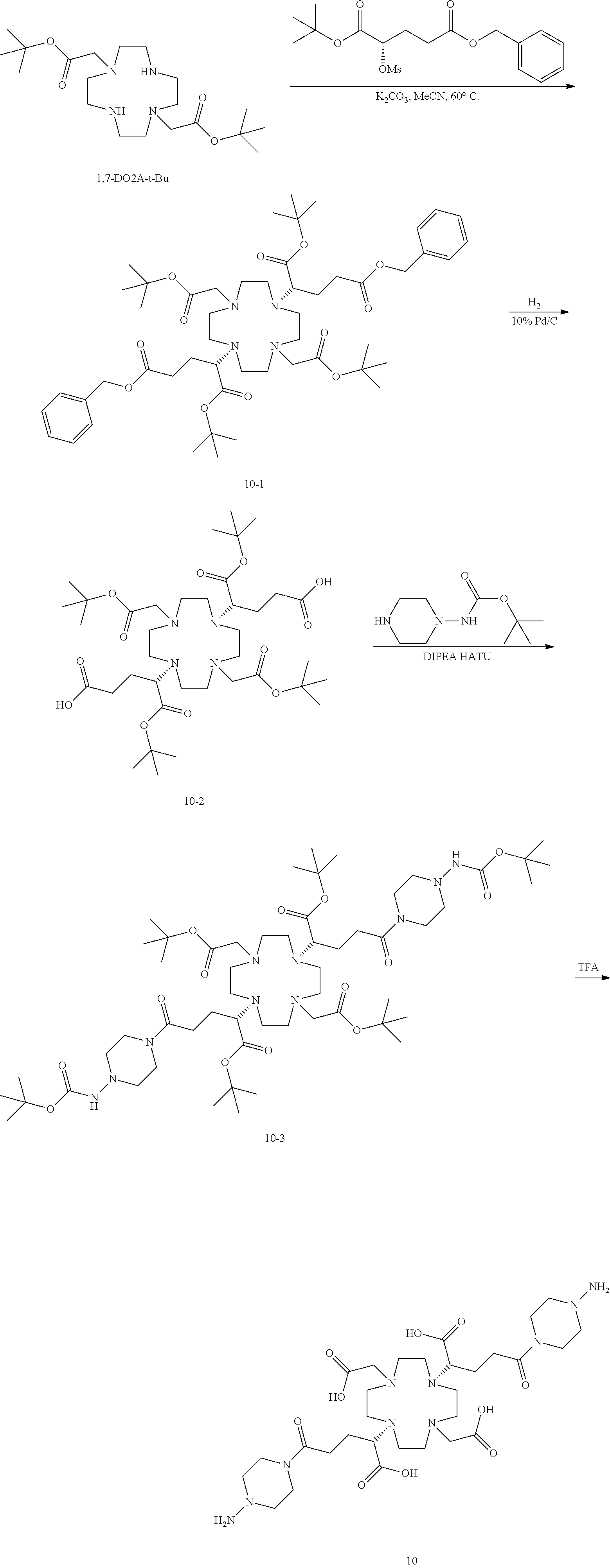

### Compound 10-1

(S)-5-Benzyl-1-tert-butyl 2-(methylsulfonyloxy)pentanedioate (928 mg, 2.5 mmol), as a solution in CH₃CN (2 mL), was added to a stirred mixture of 1,7-DO2A-t-Bu (400 mg, 1.0 mmol) and potassium carbonate (345 mg, 2.5 mmol) in CH₃CN (20 mL) preheated to 60° C. Then the reaction was heated to 80° C. After 12 hours, the reaction mixture was cooled to room temperature and filtered. The filtrate was concentrated, and then purified using method 9 to give Compound 9-1 as a yellow oil (420 mg, 44% yield). ¹H NMR (500 MHz, Chloroform-d) δ (ppm): 7.36-7.24 (m, 10H), 5.10-5.01 (m, 4H), 3.30 (d, J=16.2 Hz, 2H), 3.25-3.14 (m, 4H), 2.94-2.69 (m, 12H), 2.62 (d, J=14.0 Hz, 4H), 2.46 (h, J=9.6, 8.9 Hz, 4H), 1.95 (dq, J=14.2, 7.1 Hz, 2H), 1.80 (dq, J=14.7, 7.9 Hz, 2H), 1.41 (d, J=2.2 Hz, 36H). ³C NMR (126 MHz, Chloroform-d) δ (ppm): 173.61, 172.25, 170.45, 136.39, 128.89, 128.63, 81.55, 66.51, 63.38, 55.84, 53.50, 49.00, 31.05, 28.64, 28.55, 25.03. LC-MS (method 1, ESI^{†}): t_{R}=3.53 min, m/z [M+H]⁺ calcd for [C₅₂H₈₁N₄O₁₂]⁺, 953.6; found 953.5.

### Compound 10-2

Compound 10-1 (150 mg, 0.16 mmol) was dissolved in anhydrous ethanol (10 mL), and activated palladium on carbon (10%, 15 mg) was added to the solution. The suspension was placed under vacuum then connected to a balloon containing hydrogen gas. The reaction mixture was stirred for 2 hours. Then the reaction mixture was passed through a Celite pad, and the filtrate was subjected to rotary evaporation, giving Compound 10-2 as a slight yellow solid (119 mg, 99% yield). ¹H NMR (500 MHz, Acetonitrile-d₃) δ (ppm): 3.81-3.61 (m, 4H), 3.38 (dd, J=9.4, 4.5 Hz, 2H), 3.10 (s, 8H), 2.88 (q, J=14.8 Hz, 8H), 2.37 (d, J=5.8 Hz, 4H), 2.01 (dq, J=18.2, 9.7, 8.6 Hz, 2H), 1.83 (dq, J=12.3, 7.1 Hz, 2H), 1.47 (s, 19H), 1.46 (s, 18H). ¹³C NMR (126 MHz, Acetonitrile-d₃) δ (ppm): 173.83, 171.16, 168.05, 82.94, 81.93, 64.02, 54.42, 53.95, 46.43, 31.06, 27.50, 27.41, 24.14. LC-MS (method 2, ESI^{†}): t_{R}=5.40 min, m/z [M+H]⁺ calcd for [C₃₈H₆₉N₄O₁₂]⁺, 773.5; found 773.5.

### Compound 10-3

Compound 10-2 (100 mg, 0.13 mmol) and N,N-diisopropylethylamine (50 mg, 0.39 mmol) were dissolved in dry CH₃CN (5 mL). After 15 minutes, O-(7-azabenzotriazol-1-yl)-N,N,N′,N′-tetramethyluronium hexafluorophosphate (HATU, 98.8 mg, 0.26 mmol) was added. After stirring for another 1 h, tert-butyl piperazin-1-ylcarbamate (52.3 mg, 0.26 mmol) was added, and the stirring was continued for 4 hours. Then the solvent was evaporated, and the residue was purified using Method 14 to yield 143 mg (97% yield) of Compound 10-3 as a white solid product. ¹H NMR (500 MHz, Methanol-d₄) δ (ppm): 4.46 (d, J=16.8 Hz, 1H), 4.00 (dd, J=13.6, 7.5 Hz, 3H), 3.84 (dd, J=14.4, 3.6 Hz, 2H), 3.76 (q, J=14.1, 13.3 Hz, 4H), 3.64 (dtt, J=18.7, 10.0, 4.1 Hz, 3H), 3.53 (ddt, J=12.8, 7.7, 3.9 Hz, 2H), 3.49-3.41 (m, 2H), 3.28 (d, J=11.4 Hz, 4H), 3.20 (d, J=14.0 Hz, 4H), 2.94 (ddd, J=16.2, 10.9, 4.4 Hz, 3H), 2.79 (ddd, J=9.8, 6.0, 3.2 Hz, 3H), 2.72 (tq, J=7.5, 3.8 Hz, 4H), 2.68-2.57 (m, 5H), 2.07 (t, J=9.8 Hz, 2H), 1.75 (ddt, J=14.0, 8.8, 4.1 Hz, 1H), 1.62-1.41 (m, 58H). ¹³C NMR (126 MHz, Methanol-d₄) δ 176.46, 174.47, 173.16, 157.28, 83.44, 82.74, 81.02, 56.73, 55.89, 53.86, 49.17, 45.92, 45.44, 42.21, 32.35, 28.64, 28.37, 20.43. LC-MS (method 1, ESI^{†}): t_{R}=3.21 min, m/z [M+H]⁺ calcd for [C₅₆H₁₀₃N₁₀O₁₄]⁺, 1139.8; found 1139.7.

### Compound 10

Compound 10-3 (100 mg, 88 μmol) was dissolved in a mixture of trifluoroacetic acid (TFA, 2 mL), triisopropyl silane (150 μL), 1-dodecathiol (150 μL), and water (10 μL). The mixture was stirred at room temperature overnight. The volatiles were removed by vacuum. To the residue was added diethyl acetate to precipitate out the product as a white solid. The solid was isolated and washed with diethyl acetate three times. The residue was purified using HPLC Method 16 to give Compound 10 as a white solid (57 mg, 90% yield). ¹H NMR (500 MHz, D2O) δ (ppm): 4.30-2.38 (m, 50H), 1.96 (m, 4H). ¹³C NMR (126 MHz, D₂O) δ (ppm): 176.73, 173.35, 170.62, 63.21, 56.02, 50.45, 46.61, 44.82, 41.33, 30.21, 25.45. LC-MS (method 3 or method 7, ESI^{†}): t_{R}=2.29 min, m/z [M+H]⁺ calcd for [C₃₀H₅₄N₁₀O₁₀]⁺, 715.4; found 715.3.

### Example 11. Compound 11

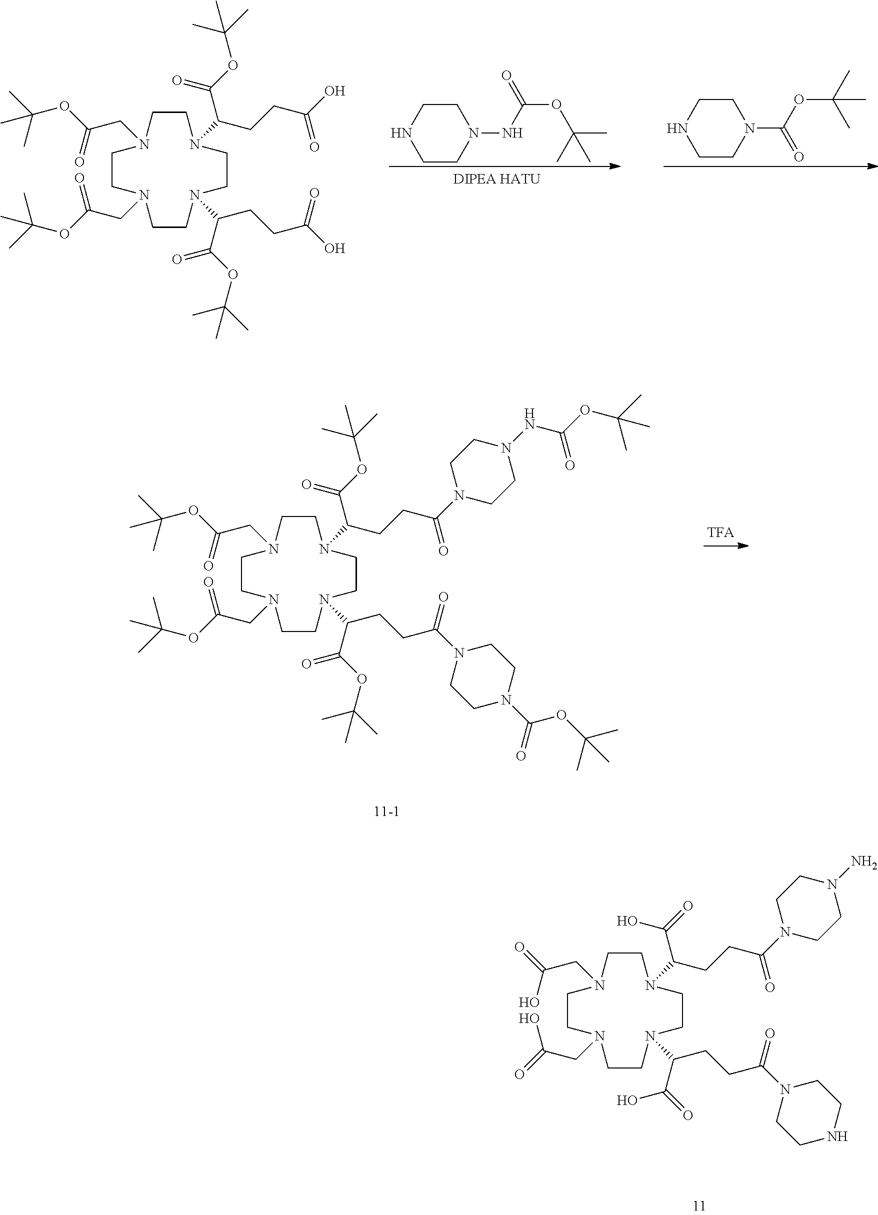

### Compound 11-1

Compound 9-2 (100 mg, 0.13 mmol) and N,N-diisopropylethylamine (50 mg, 0.39 mmol) were dissolved in dry CH₃CN (20 mL). After 15 minutes, O-(7-azabenzotriazol-1-yl)-N,N,N′,N′-tetramethyluronium hexafluorophosphate (HATU, 98.8 mg, 0.26 mmol) was added. After stirring for another 1 hour, the solution was incubated in ice. Then tert-butyl piperazin-1-ylcarbamate (26 mg, 0.13 mmol) dissolved in 10 mL CH₃CN was slowly added in 30 minutes. The reaction was warmed to room temperature. After 1 hour, 1-boc-piperazine (37 mg, 0.2 mmol) was added, and the stirring was continued for another 4 hours. Then the solvent was evaporated, the residue was purified using Method 14 to yield 37 mg (25% yield) of a white solid product. ¹H NMR (500 MHz, Chloroform-d) δ 3.66-3.24 (m, 16H), 3.07-2.46 (m, 18H), 2.38-1.63 (m, 12H), 1.43 (dd, J=8.8, 3.7 Hz, 54H). ¹³C NMR (126 MHz, Methanol-d₄) δ 176.61, 174.66, 173.35, 173.01, 157.46, 156.55, 83.56, 82.85, 81.72, 81.21, 81.06, 61.45, 56.83, 56.34, 55.96, 53.96, 49.60, 49.60, 49.43, 46.24, 45.95, 45.47, 44.72, 42.57, 42.26, 32.25, 28.65, 28.42, 20.52. LC-MS (method 1, ESI): t_{R}=3.25 min, m/z [M+H]⁺ calcd for [C₅₆H₁₀₃N₉O₁₄]⁺, 1124.8; found 1124.7.

### Compound 11

Compound 11-1 (30 mg, 26 μmol) was dissolved in a mixture of trifluoroacetic acid (TFA, 1.5 ml), triisopropyl silane (100 μl), 1-dodecathiol (100 μl) and water (10 l). The mixture was stirred at room temperature overnight. The volatiles were removed by vacuum. To the residue was added diethyl acetate to precipitate out the product as a white solid. The solid was isolated and washed with diethyl acetate for three times. The residue was purified using HPLC Method 1 to give Compound 11-1 as a white solid (18 mg, 95% yield). ¹H NMR (500 MHz, D₂O) δ (ppm): 3.93-3.49 (m, 101H), 3.48-3.02 (m, 28H), 2.56 (d, J=63.5 Hz, 5H), 2.40-2.08 (m, 1H), 1.96 (s, 2H). ¹³C NMR (126 MHz, Methanol-d₄) δ (ppm): 178.99, 176.75, 176.55, 174.53, 173.26, 172.67, 64.70, 62.03, 61.50, 56.82, 55.96, 53.99, 52.56, 46.36, 45.44, 44.33, 42.61, 35.58, 34.46, 32.77, 32.17, 30.76, 26.28, 22.31, 21.14, 20.49. LC-MS (method 3 or method 7, ESI^{†}): t_{R}=2.31 min, m/z [M+H]⁺ calcd for [C₃₀H₅₄N₉O₁₀]⁺, 700.4; found 700.3.

### Example 12. Compound 12

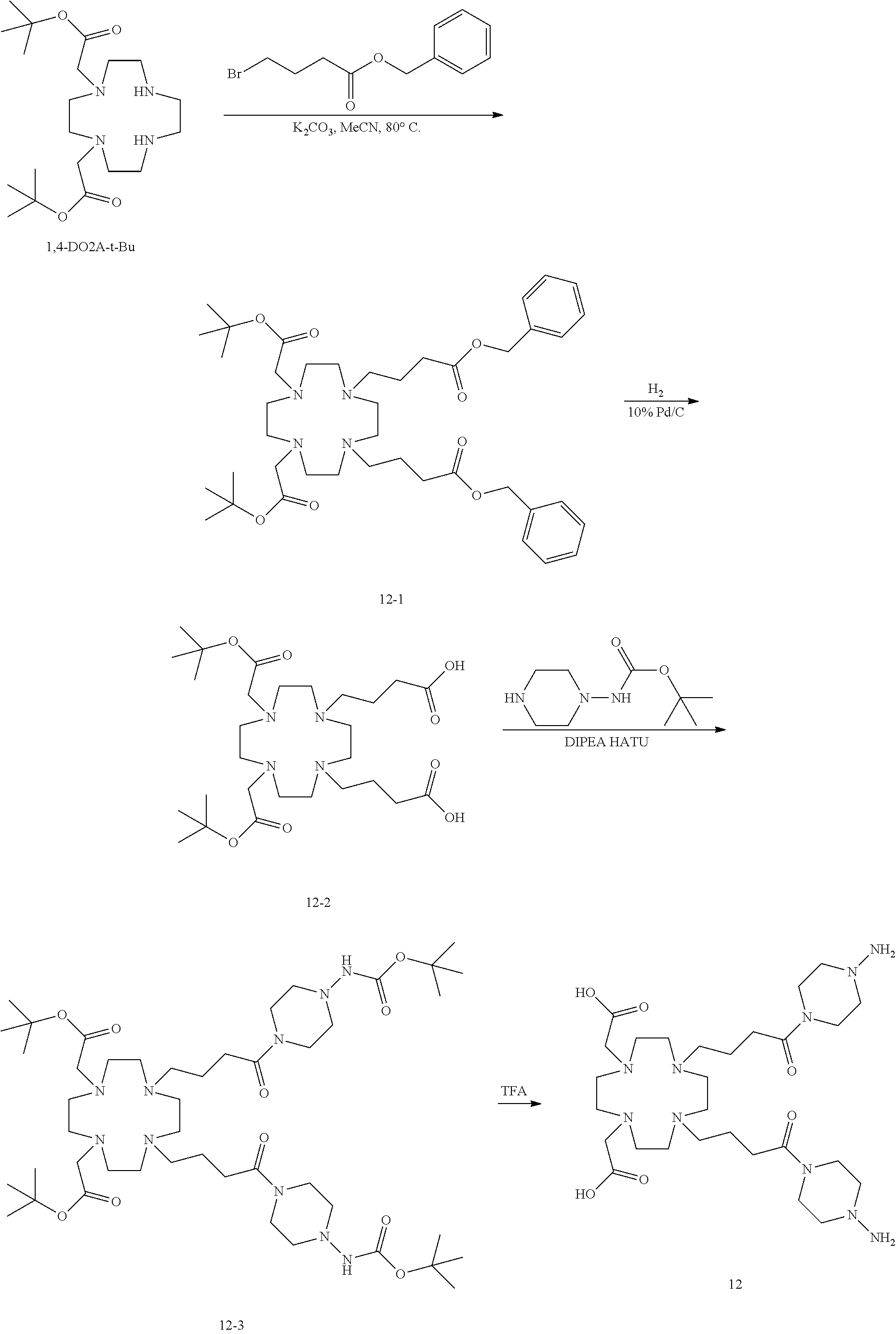

### Compound 12-1

Benzyl 4-bromobutanoate (565 mg, 2.2 mmol) as a solution in CH₃CN (2 mL) was added to a stirred mixture of 1,4-DO2A-t-Bu (400 mg, 1.0 mmol) and potassium carbonate (345 mg, 2.5 mmol) in CH₃CN (20 mL) preheated to 80° C. After 12 hours, the reaction mixture was cooled to room temperature and filtered. The filtrate was concentrated and then purified using method 9 to give Compound 12-1 as a yellow oil (692 mg, 92% yield). ¹H NMR (500 MHz, Chloroform-d) δ (ppm): 7.34 (hd, J=7.4, 6.5, 1.7 Hz, 10H), 5.10 (s, 4H), 3.29 (s, 4H), 3.14 (s, 4H), 3.06-2.96 (m, 4H), 2.95-2.81 (m, 8H), 2.70 (s, 4H), 2.49-2.38 (m, 4H), 1.89 (p, J=6.9 Hz, 4H), 1.44 (s, 18H). ¹³C NMR (126 MHz, Chloroform-d) δ (ppm): 172.65, 170.39, 135.83, 128.88, 128.57, 128.35, 56.11, 53.51, 52.19, 50.94, 50.62, 50.30, 31.10, 28.18, 19.73. LC-MS (method 1, ESI^{†}): t_{R}=3.29 min, m/z [M+H]⁺ calcd for [C₄₂H₆₅N₄O₈]⁺, 753.5; found 753.4.

### Compound 12-2

Compound 12-1 (200 mg, 0.26 mmol) was dissolved in anhydrous ethanol (10 mL), and activated palladium on carbon (10%, 20 mg) was added to the solution. The suspension was placed under vacuum then connected to a balloon containing hydrogen gas. The reaction mixture was stirred for 2 hours. Then the reaction mixture was passed through a Celite pad, and the filtrate was subjected to rotary evaporation, giving Compound 12-2 as a slight yellow solid (146 mg, 96% yield). ¹H NMR (500 MHz, Methanol-d₄) δ (ppm): 3.42 (s, 4H), 3.14 (s, 4H), 3.01 (s, 4H), 2.98-2.93 (m, 4H), 2.91 (s, 4H), 2.74 (s, 4H), 2.36 (t, J=6.7 Hz, 4H), 1.89 (p, J=6.8 Hz, 4H), 1.48 (s, 18H). ¹³C NMR (126 MHz, Methanol-d₄) δ (ppm): 176.10, 170.83, 81.32, 55.36, 53.42, 51.46, 50.64, 50.36, 49.99, 31.28, 27.08, 19.62. LC-MS (method 2, ESI^{†}): t_{R}=4.37 min, m/z [M+H]⁺ calcd for [C₂₈H₅₃N₄O₈]⁺, 573.4; found 573.5.

### Compound 12-3

Compound 12-2 (100 mg, 0.17 mmol) and N,N-diisopropylethylamine (55 mg, 0.42 mmol) were dissolved in dry CH₃CN (10 mL). After 15 minutes, O-(7-azabenzotriazol-1-yl)-N,N,N′,N′-tetramethyluronium hexafluorophosphate (HATU, 129.2 mg, 0.34 mmol) was added. After stirring for another 1 hour, tert-butyl piperazin-1-ylcarbamate (68.4 mg, 0.34 mmol) was added, and the stirring was continued for 4 hours. Then the solvent was evaporated, and the residue was purified using Method 14 to yield 146 mg (92% yield) Compound 12-3 of a white solid product. ¹H NMR (500 MHz, Chloroform-d) δ (ppm): 3.69 (s, 2H), 3.55 (dt, J=10.5, 4.8 Hz, 2H), 3.49 (s, 3H), 3.30 (d, J=26.1 Hz, 2H), 3.17 (s, 11H), 3.08-2.74 (m, 10H), 2.69 (d, J=20.1 Hz, 211), 2.42 (t, J=6.5 Hz, 1H), 1.90 (dt, J=13.7, 6.1 Hz, 1H), 1.45 (s, 18H). ¹³C NMR (126 MHz, Methanol-d₄) δ (ppm): 172.40, 157.13, 85.37, 81.25, 56.22, 55.93, 55.68, 54.48, 51.73, 51.19, 50.21, 45.74, 42.23, 30.76, 28.70, 28.43, 20.78. LC-MS (method 1, ESI^{†}): t_{R}=2.90 min, m/z [M+H]⁺ calcd for [C₄₆H₈₇N₁₀O₁₀]⁺, 939.7; found 939.5.

### Compound 12

Compound 12-3 (95 mg, 0.10 mmol) was dissolved in a mixture of trifluoroacetic acid (TFA, 2 ml), triisopropyl silane (150 μl), 1-dodecathiol (150 μl), and water (10 μl). The mixture was stirred at room temperature overnight. The volatiles were removed by vacuum. To the residue was added diethyl acetate to precipitate out the product as a white solid. The solid was isolated and washed with diethyl acetate for three times. The residue was purified using HPLC Method 16 to give the product Compound 12 as a white solid (56 mg, 90% yield). ¹H NMR (500 MHz, D₂O) δ (ppm): 3.68 (d, J=26.5 Hz, 10H), 3.34-2.76 (m, 30H), 2.50 (t, J=6.1 Hz, 4H), 1.86 (s, 4H). ¹³C NMR (126 MHz, Methanol-d₄) δ (ppm): 175.11, 172.59, 57.96, 54.41, 52.14, 51.77, 50.97, 49.74, 45.58, 42.03, 30.50, 20.68. LC-MS (method 3, ESI^{†}): t_{R}=2.33 min, m/z [M+H]⁺ calcd for [C₂₈H₅₅N₁₀O₆]⁺, 627.4; found 627.5.

### Example 13. Compound 13

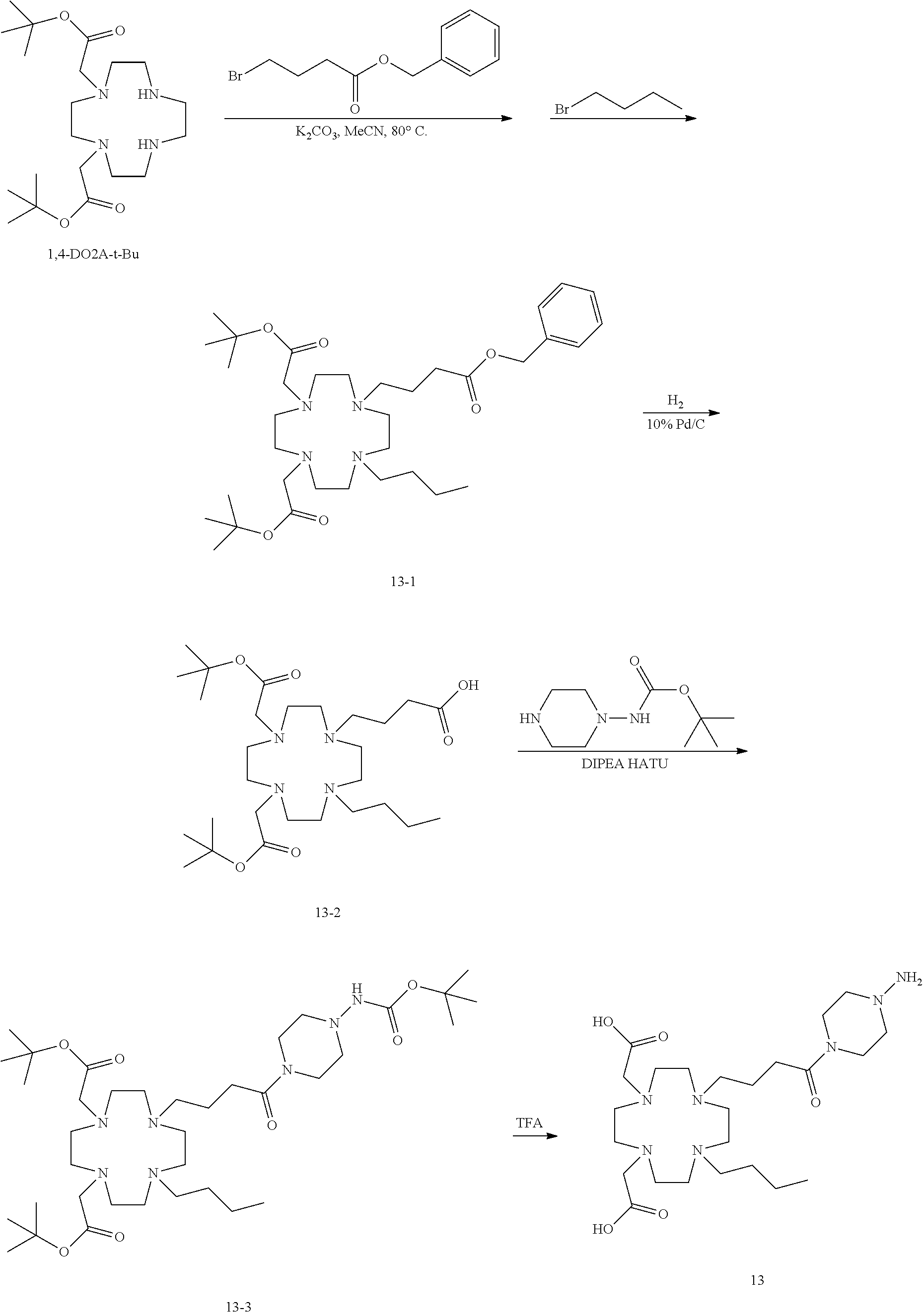

### Compound 13-1

Benzyl 4-bromobutanoate (257 mg, 1.0 mmol) as a solution in CH₃CN (6 mL), was added dropwise to a stirred mixture of 1,4-DO2A-t-Bu (400 mg, 1.0 mmol) and potassium carbonate (345 mg, 2.5 mmol) in CH₃CN (20 mL) preheated to 80° C. After 6 hours, 1-bromobutane (137 mg, 1.0 mmol) was added, and the reaction continued for another 2 hours. Then the reaction mixture was cooled to room temperature and filtered. The filtrate was concentrated and then purified using method 9 to give Compound 13-1 as a yellow oil (348 mg, 55% yield). ¹H NMR (500 MHz, Chloroform-d) δ (ppm): 7.39-7.28 (m, 5H), 5.10 (s, 2H), 3.28 (s, 4H), 2.97-2.01 (m, 22H), 1.79 (p, J=8.2, 7.5 Hz, 2H), 1.44 (s, 22H), 1.29 (dp, J=14.1, 8.4, 7.4 Hz, 2H), 0.90 (t, J=7.3 Hz, 3H). ¹³C NMR (126 MHz, Chloroform-d) δ (ppm): 173.48, 172.07, 170.97, 136.12, 128.68, 128.35, 81.93, 81.11, 66.33, 64.48, 56.59, 56.25, 54.83, 52.39, 52.02, 31.99, 30.75, 28.31, 28.15, 21.13, 20.71, 14.12, 13.82. LC-MS (method 1, ESI^{†}): t_{R}=3.29 min, m/z [M+H]⁺ calcd for [C₃₅H₆₁N₄O₆]⁺, 633.5; found 633.5.

### Compound 13-2

Compound 13-1 (200 mg, 0.32 mmol) was dissolved in anhydrous ethanol (10 mL), and activated palladium on carbon (10%, 20 mg) was added to the solution. The suspension was placed under vacuum then connected to a balloon containing hydrogen gas. The reaction mixture was stirred for 2 hours. Then the reaction mixture was passed through a Celite pad, and the filtrate was subjected to rotary evaporation, giving Compound 13-2 as a slight yellow solid (160 mg, 92% yield). ¹H NMR (500 MHz, Methanol-d₄) δ (ppm): 3.41 (d, J=12.4 Hz, 2H), 3.19-2.81 (m, 6H), 2.73 (s, 1H), 2.25 (t, J=6.7 Hz, 1H), 1.86 (dq, J=13.2, 7.1 Hz, 1H), 1.64 (td, J=10.7, 10.2, 5.7 Hz, 1H), 1.48 (s, 8H), 1.41 (h, J=7.3 Hz, 1H), 0.99 (t, J=7.3 Hz, 1H). ¹³C NMR (126 MHz, Methanol-d₄) δ (ppm): 179.60, 178.35, 172.09, 82.59, 56.87, 55.37, 55.13, 52.80, 52.69, 52.30, 51.96, 51.81, 51.67, 51.17, 50.83, 49.34, 34.73, 28.55, 28.37, 27.42, 23.01, 21.98, 21.35, 14.33, 14.21. LC-MS (method 2, ESI^{†}): t_{R}=6.03 min, m/z [M+H]⁺ calcd for [C₂₈H₅₅N₄O₆]⁺, 543.4; found 543.5.

### Compound 13-3

Compound 13-2 (100 mg, 0.18 mmol) and N,N-diisopropylethylamine (28 mg, 0.22 mmol) were dissolved in dry CH₃CN (10 mL). After 15 minutes, O-(7-azabenzotriazol-1-yl)-N,N,N′,N′-tetramethyluronium hexafluorophosphate (HATU, 68.4 mg, 0.18 mmol) was added. After stirring for another 1 hour, tert-butyl piperazin-1-ylcarbamate (36.2 mg, 0.18 mmol) was added, and the stirring was continued for 4 hours. Then the solvent was evaporated, and the residue was purified using Method 14 to yield 123 mg (93% yield) of Compound 13-3 a white solid product. ¹H NMR (500 MHz, Methanol-d₄) δ (ppm): 3.82-3.56 (m, 4H), 3.11-2.02 (m, 30H), 1.83 (m, 2H), 1.58 (m, 2H), 1.52 (s, 9H), 1.45 (s, 18H), 1.31 (dq, J=13.0, 6.7, 6.2 Hz, 2H), 0.96 (t, J=6.7 Hz, 3H). ¹³C NMR (126 MHz, Methanol-d₄) δ 174.55, 174.27, 172.92, 157.38, 83.28, 83.08, 81.07, 57.16, 56.36, 56.07, 55.61, 54.41, 53.30, 50.97, 50.10, 45.76, 42.29, 31.57, 31.13, 28.68, 28.31, 27.41, 22.06, 21.68, 14.49. LC-MS (method 1, ESI^{†}): t_{R}=2.86 min, [M+H]⁺ calcd for [C₃₈H₇₄N₇O₇]⁺, 726.5; found 726.5.

### Compound 13

Compound 13-3 (100 mg, 0.13 mmol) was dissolved in a mixture of trifluoroacetic acid (TFA, 2 ml), triisopropyl silane (150 μl), 1-dodecathiol (150 μl) and water (10 μl). The mixture was stirred at room temperature overnight. The volatiles were removed by vacuum. To the residue was added diethyl acetate to precipitate out the product as a white solid. The solid was isolated and washed with diethyl acetate for three times. The residue was purified using HPLC Method 16 to give the product Compound 13 as a white solid (67 mg, 97% yield). ¹H NMR (500 MHz, Methanol-d₄) δ (ppm): 3.61 (s, 6H), 3.37 (s, 4H), 3.11 (m, 16H), 2.89-2.66 (m, 6H), 2.45 (t, J=6.6 Hz, 2H), 1.86 (dq, J=14.2, 7.6 Hz, 2H), 1.70 (q, J=8.1, 7.0 Hz, 2H), 1.39 (h, J=7.3 Hz, 2H), 1.00 (t, J=7.4 Hz, 3H). ³C NMR (126 MHz, D₂O) δ (ppm): 177.23, 173.41, 171.32, 56.39, 53.99, 52.01, 50.58, 50.04, 49.57, 49.22, 48.32, 47.45, 44.77, 41.07, 29.76, 24.92, 13.24, 12.81. LC-MS (method 3, ESI^{†}): t_{R}=3.40 min, m/z [M+H]⁺ calcd for [C₂₄H₄₈N₇O₅]⁺, 514.4; found 514.3.

### Example 14. Compound 14

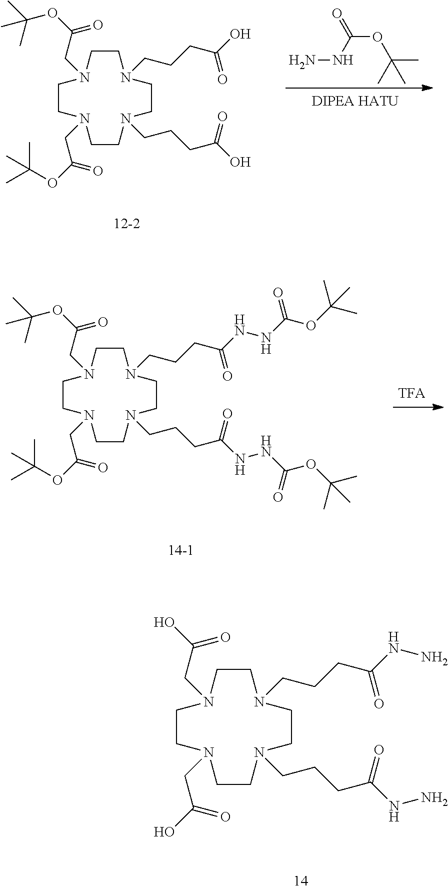

### Compound 14-1

Compound 12-2 (100 mg, 0.17 mmol) and N,N-diisopropylethylamine (55 mg, 0.42 mmol) were dissolved in dry CH₃CN (10 mL). After 15 minutes, O-(7-azabenzotriazol-1-yl)-N,N,N′,N′-tetramethyluronium hexafluorophosphate (HATU, 129.2 mg, 0.34 mmol) was added. After stirring for another 1 hour, tert-Butyl carbazate (45 mg, 0.34 mmol) was added, and the stirring was continued for 4 hours. Then the solvent was evaporated, and the residue was purified using Method 14 to yield 129 mg (95% yield) of Compound 14-1 as a white solid product. ¹H NMR (500 MHz, Methanol-d₄) δ (ppm): 3.54 (s, 4H), 3.09 (m, 20H), 2.30 (s, 4H), 1.95 (s, 4H), 1.48 (d, J=6.1 Hz, 36H). ¹³C NMR (126 MHz, Methanol-d₄) δ (ppm): 174.39, 171.60, 157.52, 83.48, 81.71, 56.27, 53.99, 52.05, 51.39, 50.53, 31.39, 28.57, 28.38, 21.12, 0.86. LC-MS (method 1, ESI^{†}): t_{R}=2.82 min, m/z [M+H]⁺ calcd for [C₃₈H₇₃N₈O₁₀]⁺, 801.5; found 801.6.

### Compound 14

Compound 14-1 (100 mg, 0.12 mmol) was dissolved in a mixture of trifluoroacetic acid (TFA, 2 ml), triisopropyl silane (150 μl), 1-dodecathiol (150 μl), and water (10 μl). The mixture was stirred at room temperature overnight. The volatiles were removed by vacuum. To the residue was added diethyl acetate to precipitate out the product as a white solid. The solid was isolated and washed with diethyl acetate three times. The residue was purified using HPLC Method 16 to give Compound 14 as a white solid (55 mg, 90% yield). ¹H NMR (500 MHz, D₂O) δ (ppm): 3.27 (td, J=134.6, 127.1, 71.2 Hz, 26H), 2.28 (d, J=6.1 Hz, 4H), 1.91 (s, 4H). ¹³C NMR (126 MHz, Methanol-d₄) δ (ppm): 173.67, 173.10, 56.49, 53.01, 50.84, 50.45, 50.00, 48.53, 47.39, 30.49, 20.38. LC-MS (method 1, ESI): t_{R}=2.50 min, m/z [M+H]⁺ calcd for [C₂₀H₄₁N₈O₆]⁺, 489.3; found 489.3.

### Example 15. Compound 15

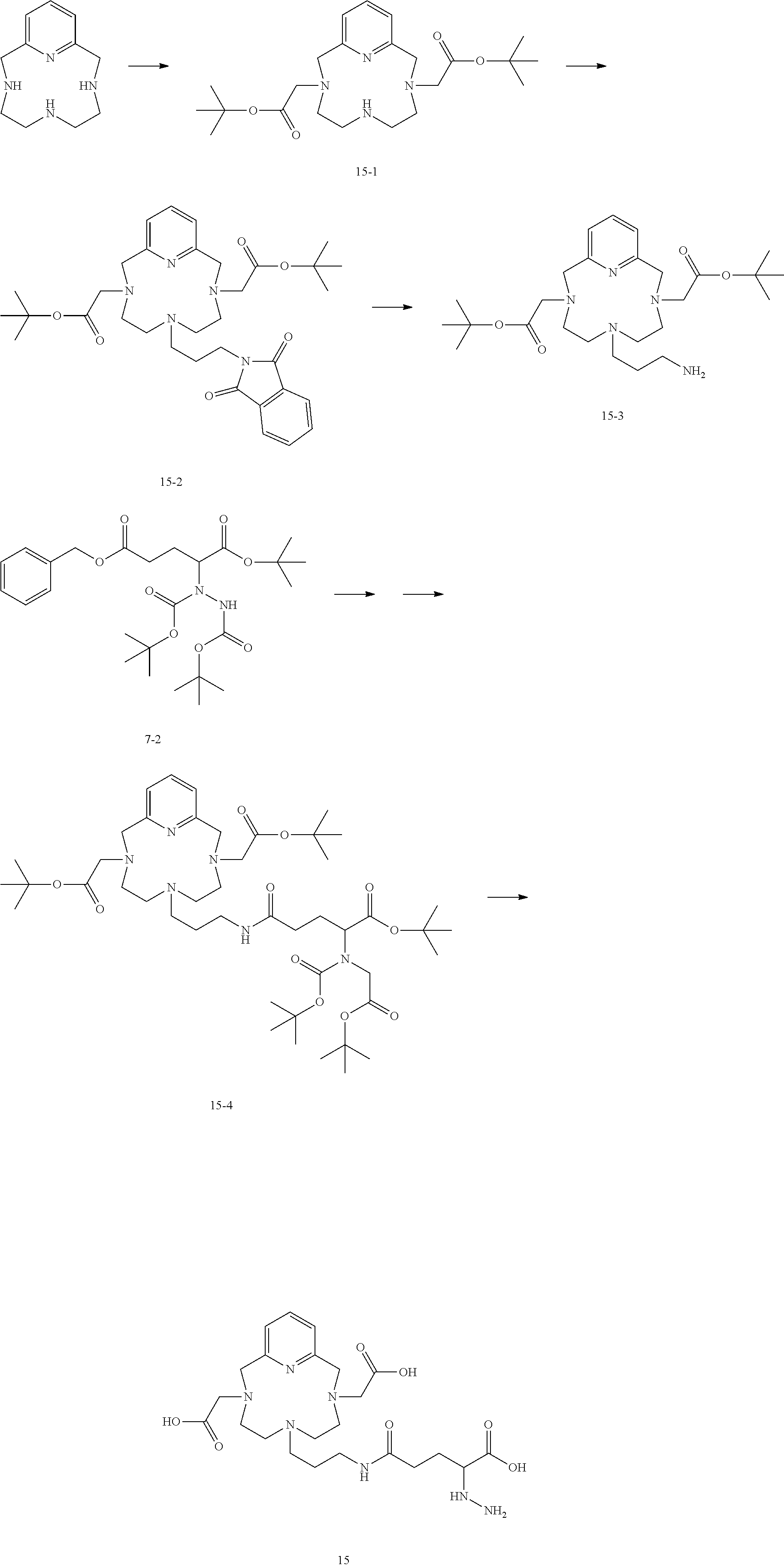

### Compound 15-1

The starting material 3,6,9-triaza-1(2,6)-pyridinacyclodecaphane (1.03 g, 5.0 mmol) was dissolved in a mixed solvent contained 50 mL dionized water and 25 mL 1,4-dioxine, and the pH was adjusted to 8.5 with concentrated HCl. tert-Butyl bromoacetate (1.37 g, 7.0 mmol) dissolved in 1,4-dioxine (25 mL) was added dropwise. The addition of more tert-butyl bromoacetate was repeated twice (2×0.23 g, 0.48 mmol) after 12 hours and 24 hours, and the pH was adjusted to 8.5 with IN NaOH. Reaction completion was monitored by LC-MS (method 1). The reaction mixture was extracted with CHCl₃ (3×50 mL), and combined organic layers were concentrated under reduced pressure. The obtained residue was purified on CombiFlash (method 9) to give Compound 15-1 as a pale brown oil (1.37 g, 63%). ¹H NMR (500 MHz, CDCl₃) δ 7.63-7.49 (m, 1H), 7.01 (dd, J=7.7, 1.7 Hz, 2H), 3.96 (s, 4H), 3.50 (s, 4H), 3.36 (t, J=5.5 Hz, 4H), 2.98 (t, J=6.1 Hz, 4H), 1.44 (s, 18H). ¹³C NMR (126 MHz, CDCl₃) δ 171.20, 160.12, 137.70, 120.42, 81.64, 57.72, 57.45, 51.83, 46.24, 28.29. LC-MS (method 1): t_{R}=3.35 min, m/z=435.3 [M+H]⁺; calcd: 435.3.

### Compound 15-2

Compound 15-1 (0.86 g, 2.0 mmol) and K₂CO₃ (0.54 g, 4.0 mmol) were suspended in dry ACN (40 mL), and N-(3-Bromopropyl)phthalimide (0.8 g, 3.0 mmol) in dry ACN (20 mL) was added dropwise. The suspension was brought to reflux under argon for 12 hours. Following removal of the precipitate by filtration, the reaction mixture was concentrated under reduced pressure and then purified on CombiFlash (method 9) to give Compound 15-2 as yellow oil (1.18 g, 95%). ¹H NMR (500 MHz, CDCl₃) δ 7.82 (dt, J=5.6, 2.9 Hz, 2H), 7.71 (dt, J=5.7, 2.8 Hz, 2H), 7.59 (t, J=7.7 Hz, 1H), 7.02 (d, J=7.6 Hz, 2H), 3.97 (s, 4H), 3.79 (q, J=6.3 Hz, 2H), 3.69-3.49 (m, 4H), 3.39 (s, 6H), 3.16 (s, 4H), 2.16 (dt, J=15.3, 7.0 Hz, 2H), 1.40 (s, 18H). ¹³C NMR (126 MHz, CDCl₃) δ 169.95, 168.31, 159.29, 138.18, 134.40, 131.86, 123.58, 120.91, 82.10, 58.54, 58.00, 52.39, 50.20, 45.79, 35.59, 28.20, 21.84. LC-MS (method 1): t_{R}=3.32 min, m/z=622.3 [M+H]⁺; calcd: 622.4.

### Compound 15-3

Compound 15-2 (0.62 g, 1 mmol) and hydrazine hydrate (2.5 g, 50 mmol) were dissolved in ethanol (20 mL), and the solution was stirred for 1 hour at 45° C. After dilution with 20 mL ACN, the precipitate was removed by filtration, and the solvent was removed by reduced pressure. The obtained crude product was purified on CombiFlash (method 9) to give Compound 15-3 as a yellow oil (0.41 g, 85%). ¹H NMR (500 MHz, CDCl₃) δ 7.60 (t, J=7.7 Hz, 1H), 7.03 (d, J=7.7 Hz, 2H), 3.93 (s, 4H), 3.60-3.39 (m, 6H), 3.38-3.26 (m, 4H), 3.23-3.12 (m, 4H), 3.12-3.00 (m, 2H), 2.22 (dd, J=10.2, 6.5 Hz, 2H), 1.41 (s, 18H). ¹³C NMR (126 MHz, CDCl₃) δ 170.51, 159.56, 138.25, 120.87, 81.99, 58.24, 57.97, 51.92, 50.18, 43.69, 36.89, 28.08, 19.56. LC-MS (method 1): t_{R}=2.742 min, m/z=492.5 [M+H]⁺; calcd: 492.4.

### Compound 15-4

Compound 7-2 (0.51 g, 1 mmol) was added to a slurry of palladium on carbon (50% water, 50 mg) in methanol (20 mL). The mixture was purged twice with hydrogen and then stirred at room temperature under argon for 12 hours. Celite was added into the reaction mixture and the slurry was filtered through a celite bed preliminary wetted with methanol. The filtrate was concentrated under reduced pressure to give a colorless oil, which was used for the next step without further purification. The obtained colorless oil, Compound 15-3 (0.25 g, 0.5 mmol), and DIPEA (0.13 g, 1.0 mmol) were dissolved in dry ACN (20 mL), and HATU (0.29 g, 0.75 mmol) in dry ACN (10 mL) was added. The reaction mixture was stirred at room temperature for 45 min. After the solvent was removed under reduced pressure, the obtained oil was redissolved in dichloromethane and extracted with citric acid (10% in H₂O) and brine. The organic layer was dried over Na₂SO₄ and concentrated under reduced pressure. The crude product was purified on CombiFlash (method 9) to give Compound 15-4 as a yellow oil (0.88 g, 87%). ¹H NMR (500 MHz, CDCl₃) δ 7.56 (t, J=7.7 Hz, 1H), 7.00 (d, J=7.7 Hz, 2H), 5.09 (s, 1H), 3.91 (s, 4H), 3.45-3.03 (m, 18H), 2.51-2.38 (m, 1H), 2.34-2.15 (m, 1H), 2.07-1.78 (m, 4H), 1.43 (s, 18H), 1.39 (s, 27H). ¹³C NMR (126 MHz, CDCl₃) δ 152.47, 139.12, 121.82, 83.54, 82.51, 62.97, 58.05, 52.18, 50.43, 36.72, 31.84, 29.71, 28.18, 28.08, 27.02, 22.46. LC-MS (method 1): t_{R}=3.67 min, m/z=892.5 [M+H]⁺; calcd: 892.6.

### Compound 15

To Compound 9 (178.3 mg, 0.2 mmol) in DCM (5 mL), cooled to 0° C., was added anisole (0.4 mL, 2 mL/mmol) followed by the slow addition of trifluoroacetic acid (5 mL), and the mixture was stirred at 0° C. for 1 hour followed by room temperature for 4 hours. The solvent and trifluoroacetic acid were carefully evaporated under reduced pressure, the resulting oily residue was dissolved in water (5 mL), the organic byproducts were pipetted away with diethyl ether (3×5 mL), the aqueous layer was freeze-drying, and the obtained crude product was purified by preparative HPLC (method 7-2 with A and B as solvents) to give Compound 15 as a white solid (88.9 mg, 85%). ¹H NMR (500 MHz, D₂O) δ 8.16 (m, 1H), 7.56 (d, J=7.6 Hz, 2H), 4.39 (s, 4H), 3.75 (s, 4H), 3.62 (t, J=6.5 Hz, 1H), 3.26-2.92 (m, 12H), 2.26 (m, 2H), 1.95 (m, 1H), 1.88 (m, 1H), 1.84-1.74 (m, 2H). ¹³C NMR (126 MHz, D₂O) δ 175.02, 174.71, 171.25, 150.65, 141.00, 122.98, 62.50, 59.13, 57.44, 52.62, 51.90, 50.15, 37.09, 31.74, 25.17, 24.08. LC-MS (method 18): t_{R}=5.58 min, m/z=524.2 [M+H]⁺; calcd: 524.3.

### Example 16. Compound 16

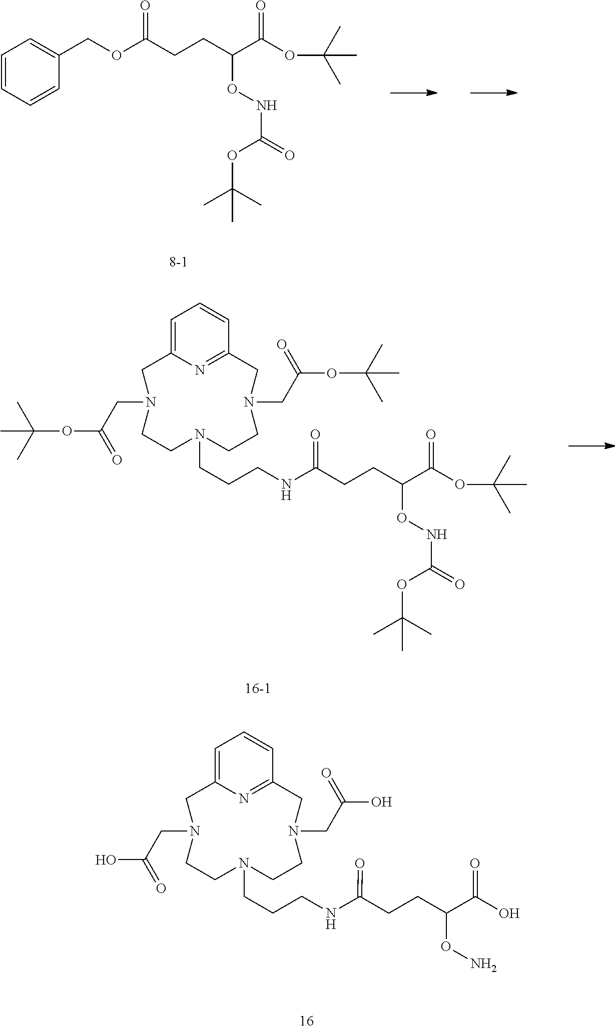

### Compound 16-1

Compound 8-1 (0.41 g, 1.0 mmol) was added to a slurry of palladium on carbon (50% water, 50 mg) in methanol (10 mL). The mixture was purged twice with hydrogen and then stirred at room temperature under argon for 12 hours. Celite was added into the reaction mixture and the slurry was filtered through a celite bed preliminary wetted with methanol. The filtrate was concentrated under reduced pressure to give a colorless oil of product which was used for the next step without further purification. The obtained colorless oil, Compound 15-3 (0.25 g, 0.5 mmol), and DIPEA (0.13 g, 1.0 mmol) were dissolved in dry ACN (20 mL), and HATU (0.29 g, 0.75 mmol) in dry ACN (10 mL) was added. The reaction mixture was stirred at room temperature for 45 min. After the solvent was removed under reduced pressure, the obtained oil was redissolved in dichloromethane and extract with 10% citric acid aqueous solution and brine. The organic layer was dried over Na₂SO₄ and concentrated under reduced pressure. The crude product was purified on CombiFlash (method 9) to give Compound 16-1 as a yellow oil (0.29 g, 73%). ¹H NMR (500 MHz, CDCl₃) δ 7.60 (t, J=7.6 Hz, 1H), 7.03 (d, J=7.7 Hz, 2H), 4.19 (dd, J=9.5, 3.7 Hz, 1H), 3.97 (s, 4H), 3.58-3.09 (m, 16H), 2.57-2.46 (m, 1H), 2.44-2.34 (m, 1H), 2.20-2.08 (m, 1H), 2.06-1.91 (m, 3H), 1.44 (m, 36H). ¹³C NMR (126 MHz, CDCl₃) δ 173.55, 170.28, 167.01, 159.54, 138.16, 120.86, 82.95, 82.13, 81.78, 58.39, 58.01, 52.35, 50.41, 45.41, 36.54, 32.00, 28.32, 28.24, 28.14, 26.96, 22.06. LC-MS (method 1): t_{R}=3.49 min, m/z=793.7 [M+H]⁺; calcd: 793.5.

### Compound 16

To Compound 16-1 (155.5 mg, 0.2 mmol) in DCM (5 mL), cooled to 0° C., was added anisole (0.4 mL, 2 mL/mmol) followed by the slow addition of trifluoroacetic acid (5 mL). The mixture was stirred for 1 hour at 0° C. and then at room temperature for 4 hours. The solvent and trifluoroacetic acid were carefully evaporated under reduced pressure, the resulting oily residue was redissolved in water (5 mL), and the organic byproducts were decanted with diethyl ether (3×5 mL). The aqueous layer was lyophilized, and the obtained crude product was purified by preparative HPLC (method 18) to give Compound 16 as a white solid (84.9 mg, 81%). ¹H NMR (500 MHz, D₂O) δ 7.61 (t, J=7.7 Hz, 1H), 7.07 (d, J=7.8 Hz, 2H), 3.88 (s, 4H), 3.82 (dd, J=8.1, 4.3 Hz, 1H), 3.27 (s, 4H), 3.21-2.84 (m, 13H), 2.25-2.10 (m, 2H), 1.90-1.77 (m, 3H), 1.74-1.70 (m, 1H). ¹³C NMR (126 MHz, D₂O) δ 179.23, 179.07, 176.12, 159.03, 138.73, 121.20, 84.38, 60.51, 58.37, 51.45, 49.95, 45.04, 36.56, 32.05, 27.58, 20.98. LC-MS (method 6): t_{R}=5.58 min, m/z=525.3 [M+H]⁺; calcd: 525.3.

### Example 17. Compound 17

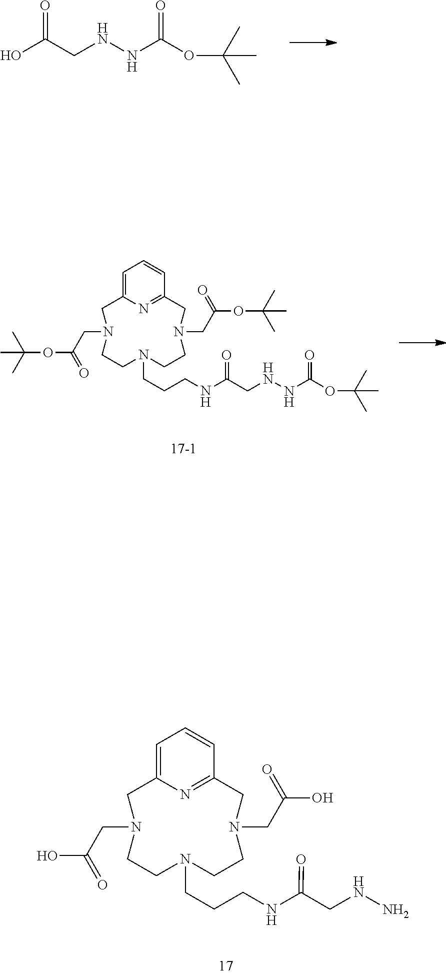

### Compound 17-1

Compound 15-3 (0.25 g, 0.5 mmol), ((tert-butoxycarbonyl)amino)glycine (0.14 g, 0.75 mmol), and DIPEA (0.13 g, 1.0 mmol) were dissolved in dry ACN (20 mL), and HATU (0.29 g, 0.75 mmol) in dry ACN (10 mL) was added. The reaction mixture was stirred at room temperature for 45 min. After the solvent was removed under reduced pressure, the obtained oil was redissolved in DCM and extracted with 10% citric acid aqueous solution and brine. The organic layer was dried over Na₂SO₄ and concentrated under reduced pressure. The crude product was purified on CombiFlash (method 9) to give Compound 17-1 as a yellow oil (0.26 g, 79%). ¹H NMR (500 MHz, CDCl₃) δ 7.61 (t, J=7.7 Hz, 1H), 7.04 (d, J=7.7 Hz, 2H), 4.08-3.86 (m, 4H), 3.54-3.30 (m, 12H), 3.28-2.96 (m, 6H), 2.05 (m, 2H), 1.42 (m, 27H). ¹³C NMR (126 MHz, CDCl₃) δ 170.34, 159.65, 138.24, 120.87, 82.19, 58.46, 57.99, 54.99, 51.95, 50.48, 44.47, 36.04, 28.45, 28.23, 28.17, 21.68. LC-MS (method 1): t_{R}=3.27 min, m/z=664.4 [M+H]⁺; calcd: 664.6.

### Compound 17

To Compound 17-1 (132.7 mg, 0.2 mmol) in DCM (5 mL), cooled to 0° C., was added anisole (0.4 mL, 2 mL/mmol) followed by the slow addition of trifluoroacetic acid (5 mL), and the mixture was stirred for 1 hour at 0° C. and at room temperature for 4 hours. The solvent and trifluoroacetic acid were carefully evaporated under reduced pressure, the resulting oily residue was redissolved in water (5 mL), the organic byproducts were pipetted away with diethyl ether (3×5 mL), the aqueous layer was lyophilized, and the obtained crude product was purified by preparative HPLC (method 18) to give Compound 17 as a white solid (69.5 mg, 77%). ¹H NMR (500 MHz, D₂O) δ 8.25 (t, J=7.9 Hz, 1H), 7.63 (d, J=7.9 Hz, 2H), 4.39 (s, 4H), 3.77 (s, 4H), 3.62 (s, 2H), 3.33-2.86 (m, 12H), 2.00-1.76 (m, 2H). ¹³C NMR (126 MHz, D₂O) δ 175.13, 169.90, 152.05, 146.46, 124.00, 57.76, 56.67, 52.85, 51.74, 51.69, 50.96, 50.90, 50.59, 36.34, 22.66. LC-MS (method 6): t_{R}=2.39 min, m/z=452.2 [M+H]⁺; calcd: 452.3.

### Example 18. Compound 18

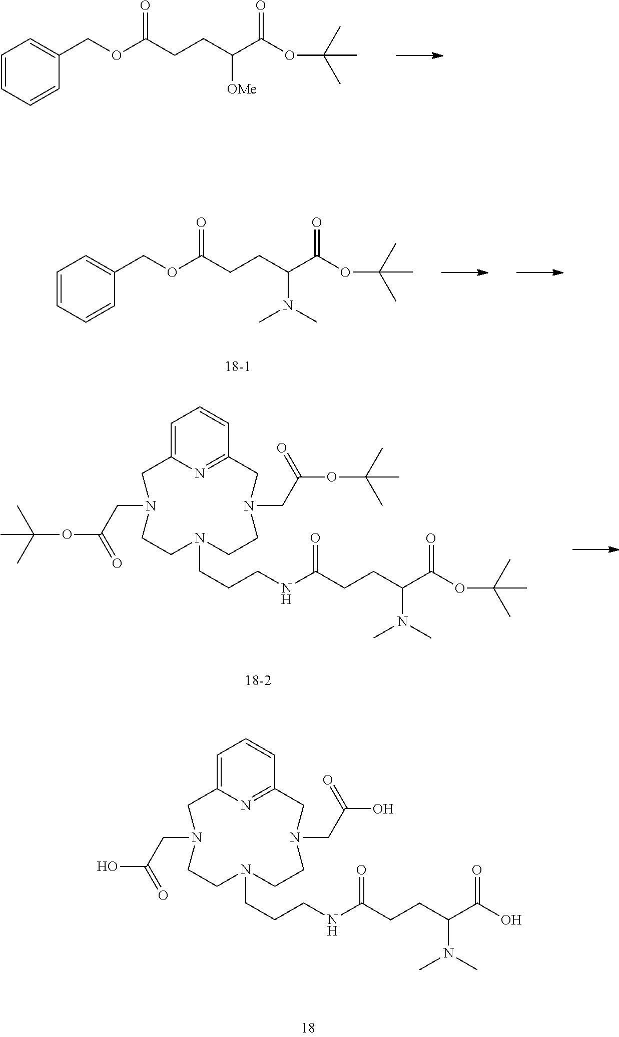

### Compound 18-1

5-benzyl 1-(tert-butyl) 2-((methylsulfonyl)oxy)pentanedioate (3.72 g, 10.0 mmol) and diethylamine (2 M in THF, 7.5 mL, 15.0 mmol) were mixed together in 40 mL dry ACN, followed by addition of K₂CO₃ (2.76 g, 20.0 mmol) and NaI (0.15 g, 1.0 mmol). The reaction mixture was stirred under reflux for 12 hours. The solid was removed by filtration and washed with DCM. The crude product was purified on CombiFlash (method 9) to give Compound 18-1 as a yellow oil (1.70 g, 53%). ¹H NMR (500 MHz, CDCl₃) δ 7.39-7.25 (m, 5H), 5.09 (s, 2H), 3.03 (t, J=7.6 Hz, 1H), 2.40 (t, J=7.5 Hz, 2H), 2.31 (s, 6H), 1.95 (q, J=7.5 Hz, 2H), 1.44 (s, 9H). ¹³C NMR (126 MHz, CDCl₃) δ 173.14, 171.09, 136.12, 128.62, 128.24, 81.26, 67.05, 66.26, 41.48, 30.90, 28.37, 24.74. LC-MS (method 1): t_{R}=4.15 min, m/z=322.4 [M+H]⁺; calcd: 322.2.

### Compound 18-2

Compound 18-1 (0.32 g, 1 mmol) was added to a slurry of palladium on carbon (50% water, 50 mg) in methanol (20 mL). The mixture was purged twice with hydrogen and then stirred at room temperature under argon for 12 hours. Celite was added into the reaction mixture, and the slurry was filtered through a celite bed preliminary wetted with methanol. The filtrate was concentrated under reduced pressure to give a colorless oil of product, which was used for the next step without further purification. The obtained colorless oil, Compound 4 (0.25 g, 0.5 mmol), and DIPEA (0.13 g, 1.0 mmol) were dissolved in dry ACN (20 mL), and HATU (0.29 g, 0.75 mmol) in dry ACN (10 mL) was added. The reaction mixture was stirred at room temperature for 45 min. After the solvent was removed under reduced pressure, the obtained oil was redissolved in DCM and extract with citric acid (10% in H₂O) and brine. The organic layer was dried over Na₂SO₄ and concentrated under reduced pressure. The crude product was purified on CombiFlash (method 9) to give Compound 18-2 as a yellow oil (0.27 g, 81%). ¹H NMR (500 MHz, CDCl₃) δ 7.58 (t, J=7.7 Hz, 1H), 7.01 (d, J=7.7 Hz, 2H), 5.25 (s, 1H), 3.91 (s, 4H), 3.51-2.98 (m, 16H), 2.34-2.15 (m, 8H), 1.92 (ddt, J=22.6, 8.4, 6.4 Hz, 4H), 1.40 (d, J=1.9 Hz, 27H). ¹³C NMR (126 MHz, CDCl₃) δ 173.88, 170.25, 167.01, 138.23, 120.88, 82.12, 81.12, 67.22, 58.36, 57.95, 52.05, 50.38, 44.89, 41.32, 36.43, 32.62, 28.35, 28.21, 25.46, 21.73. LC-MS (method 1): t_{R}=2.84 min, m/z=705.5 [M+H]⁺; calcd: 705.5.

### Compound 18

Compound 18-2 (132.7 mg, 0.2 mmol) in DCM (5 mL), cooled to 0° C., was added anisole (0.4 mL, 2 mL/mmol) followed by the slow addition of trifluoroacetic acid (5 mL), and the mixture was stirred at 0° C. for 1 hour and at room temperature for 4 hours. The solvent and trifluoroacetic acid were carefully evaporated under reduced pressure, the resulting oily residue was dissolved in water (5 mL), the organic byproducts were pipetted away with diethyl ether (3×5 mL), the aqueous layer was lyophilized, and the obtained crude product was purified by preparative HPLC (method 18) to give Compound 18 as a white solid (89.0 mg, 83%). ¹H NMR (500 MHz, D₂O) δ 8.24 (t, J=7.9 Hz, 1H), 7.63 (d, J=7.9 Hz, 2H), 4.37 (s, 4H), 3.84 (dd, J=9.6, 3.8 Hz, 1H), 3.75 (s, 4H), 3.28-2.88 (m, 12H), 2.78 (d, J=15.1 Hz, 6H), 2.43-2.24 (m, 2H), 2.15 (ddt, J=15.9, 8.0, 3.9 Hz, 1H), 2.01 (qt, J=8.0, 5.8 Hz, 1H), 1.90-1.77 (m, 2H). ¹³C NMR (126 MHz, D₂O) δ 175.10, 173.93, 169.94, 151.97, 146.63, 124.08, 66.60, 57.70, 56.60, 52.86, 51.82, 50.83, 42.61, 40.07, 36.40, 31.06, 22.62, 22.00. LC-MS (method 6): t_{R}=6.64 min, m/z=537.2 [M+H]⁺; calcd: 537.3.

### Example 19. Gd-19

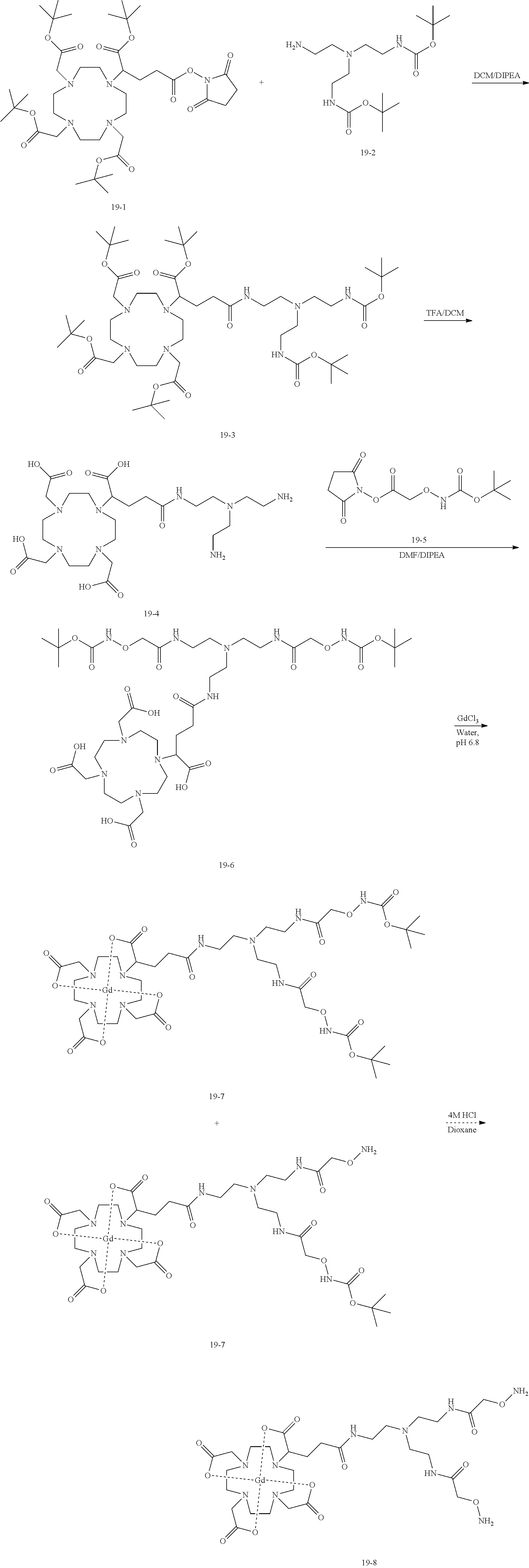

### Compounds 19-1, 19-2 and 19-5

Compounds 19-1, 19-2 and 19-5 were synthesized as previously reported in Akam, E. A., et al. Improving the reactivity of hydrazine-bearing MRI probes for in vivo imaging of lung fibrogenesis. Chemical Science 11, 224-231 (2020) and Foillard, S., Rasmussen, M. O., Razkin, J., Boturyn, D. & Dumy, P. 1-Ethoxyethylidene, a new group for the stepwise SPPS of aminooxyacetic acid containing peptides. J. Org. Chem. 73, 983-991 (2008).

### Compound 19-3

To a solution of Compound 19-2 (70 mg, 0.20 mmol) in anhydrous dichloromethane (5 mL) was added di-isopropyl ethylamine (DIPEA, 180 μL, 1.0 mmol) followed by Compound 19-1 (NHS-DOTAGA, 153 mg, 0.20 mmol). The mixture was stirred for one hour then evaporated to dryness, redissolved in acetonitrile, and purified by Combiflash using Method 10 to result in Compound 19-3 as a white solid (159 mg, 77%). ¹H NMR (500 MHz, Chloroform-d) δ 3.63-3.38 (m, 8H), 3.38-3.29 (m, 2H), 3.20 (h, J=6.6, 6.1 Hz, 2H), 3.13-2.76 (m, 21H), 2.57 (ddd, J=14.8, 7.3, 2.5 Hz, 1H), 2.50 (dt, J=14.8, 5.2 Hz, 2H), 2.45-2.30 (m, 2H), 1.99 (ddt, J=14.3, 7.3, 3.7 Hz, 1H), 1.93-1.81 (m, 1H), 1.43 (dt, J=3.7, 1.7 Hz, 55H). ¹³C NMR (126 MHz, Chloroform-d) δ 173.15, 171.38, 170.05, 169.81, 168.15, 83.00, 82.66, 82.58, 82.27, 63.43, 55.54, 54.93, 52.76, 52.68, 49.71, 37.66, 36.02, 32.51, 32.37, 28.22, 28.16. LC-MS (Method 1): t_{R}=2.9 min, purity >90% (220 nm), m/z=1029.65 [M+H]; calcd: 1029.71

### Compound 19-6

Compound 19-3 (208 mg, 0.20 mmol) was dissolved in a 1:1 mixture of dichloromethane and trifluoroacetic acid (5 mL). The mixture was stirred at room temperature for 3 hours, with frequent monitoring by LC-MS for reaction completion. The solution was concentrated by rotary evaporation, and the product was precipitated using diethyl ether. The resulting white residue was washed with diethyl ether (3×20 mL), isolated by centrifugation, and dried under vacuum, resulting in Compound 19-4 an off-white powder. This powder (113 mg, 0.12 mmol) was suspended in dimethylformamide (1 mL) and DIPEA (80 μL, 0.45 mmol) was added followed by Compound 19-5. The mixture was stirred at room temperature for 1 hour, then purified with CombiFash using Method 10 resulting in Compound 19-6 as a white powder 113 mg, 59.4% yield over two steps. ¹H NMR (500 MHz, Methanol-d₄) δ 4.41 (d, J=4.9 Hz, 1H), 4.33-4.30 (m, 4H), 4.07 (s, 4H), 3.80-3.57 (m, 9H), 3.45 (bm, 16H), 3.10 (m, 5H), 2.79-2.39 (m, 3H), 2.04-1.88 (m, 1H), 1.45 (m, 18H). LC-MS (Method 4): t_{R}=5.4 min, purity >95% (220 nm), m/z=951.45 [M+H]; calcd: 951.49

### Compound 19-7

To a solution of Compound 19-6 (113 mg, 0.12 mmol) in water (1 mL) was added GdCl₃ (50 mg, 0.13 mmol), and the pH was adjusted 6.8 with a solution of 1M NaOH. The mixture was stirred for one hour at room temperature, then purified by CombiFlash using Method 11. Compound 19-7 was isolated as a white solid after freeze-drying, mass=105 mg, 78% yield. This product was isolated as a mixture of the di- and mono-BOC protected product. LC-MS (Method 4): t_{R}=6.4 min, mono-BOC t_{R}=5.2 min. m/z di-BOC=1106.25 [M+H]; calcd: 1106.38. m/z mono-BOC=1006.25 [M+H]; calcd: 1006.38. LC-ICP (Method 18): Di-BOC t_{R}=7.4 min, mono-BOC t_{R}=5.7 min.

### Gd-19

Compound 19-7 (105 mg, 0.09 mmol) was dissolved in 4M HCl in Dioxane and stirred for 30 minutes. The solution was evaporated to dryness, re-dissolved in water, and freeze-dried. The product was then passed through a chelex column to remove any free gadolinium. Compound 19-8 (Gd-19) was isolated in quantitative yield as a white solid after freeze-drying and used without further purification. LC-MS (Method 4): t_{R}=4.5 min, m/z=905.25 [M+2H]; calcd: 905.28. LC-ICP (Method 11): t_{R}=4.9 min, purity >95%

### Example 20. Gd-20

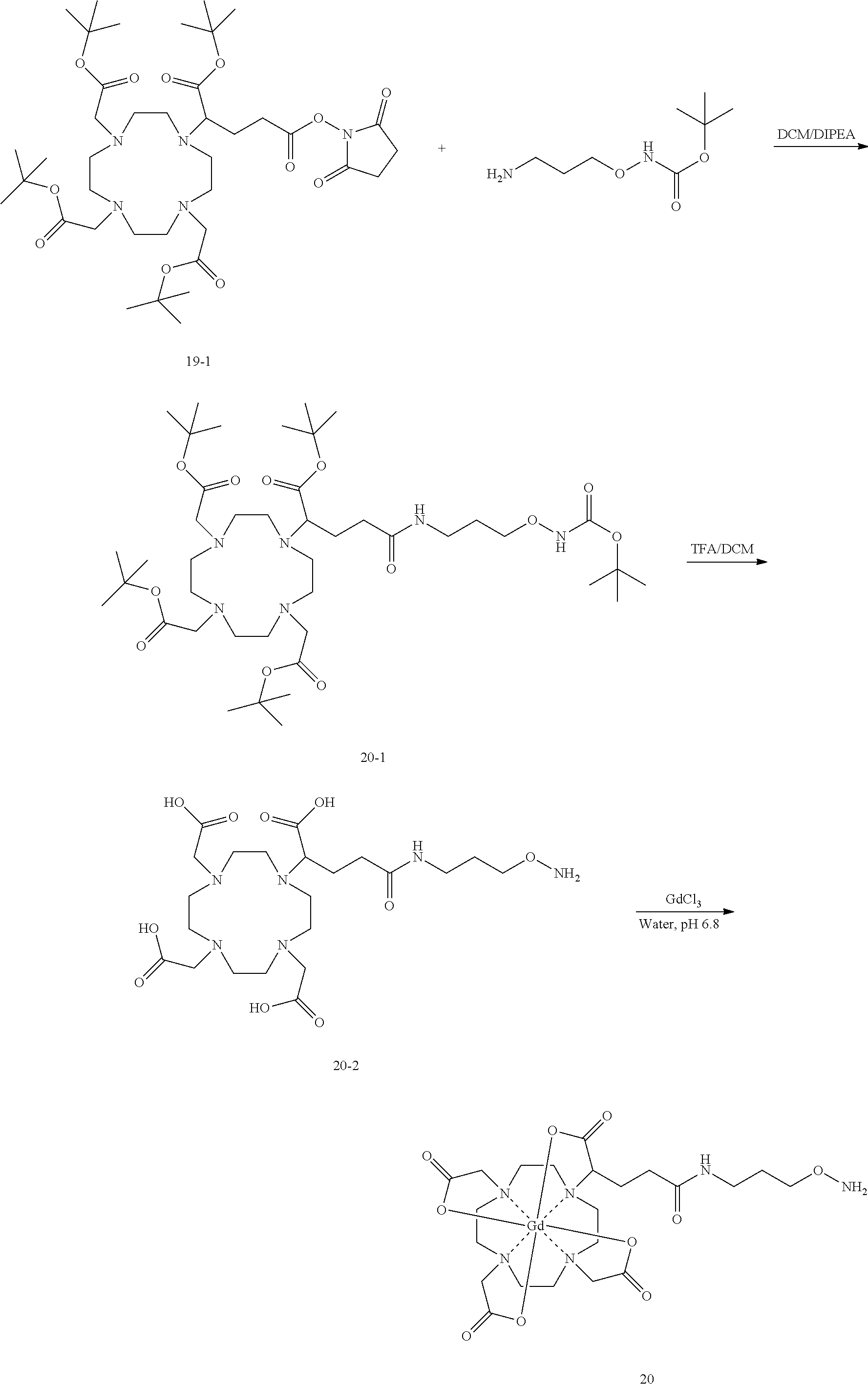

### Compound 20-1

To a solution of Compound 19-1 (209 mg, 0.26 mmol) in anhydrous dichloromethane (5 mL) was added a solution of tert-butyl N-(3-aminopropoxy)carbamate (50 mg, 0.26 mmol) and diisopropyl ethylamine (DIPEA, 180 μL, 1.0 mmol) in 1 mL dichloromethane. The mixture was stirred for one hour then evaporated to dryness, redissolved in acetonitrile, and purified by CombiFlash using Method 10 to result in Compound 20-1 as a white powder, mass=206 mg, 910% yield. ¹H NM/R (500 MHz, Chloroform-d) δ 3.90 (t, J=5.6 Hz, 2H), 3.71 (s, 4H), 3.55-3.41 (m, 3H), 3.35 (hept, J=6.9 Hz, 2H), 3.14 (dt, J=14.2, 8.1 Hz, 8H), 2.94 (t, J=5.3 Hz, 8H), 2.33 (dp, J=29.7, 7.6 Hz, 2H), 2.06 (ddd, J=12.7, 9.1, 6.5 Hz, 1H), 1.97-1.84 (m, 1H), 1.76 (p, J=6.0 Hz, 2H), 1.44 (d, J=6.4 Hz, 45H). ¹³C NMR (126 MHz, CHLOROFORM-D) δ 172.52, 171.36, 169.58, 168.57, 157.50, 83.09, 83.04, 82.53, 82.39, 81.64, 77.38, 77.38, 77.13, 76.87, 75.25, 63.47, 56.35, 55.44, 55.43, 53.19, 52.57, 49.77, 47.24, 37.17, 32.89, 28.33, 28.26, 28.23, 28.15, 27.26, 24.37. LC-MS (Method 1): t_{R}=3.36 min, purity >90% (220 nm), m/z=873.45 [M+H]; calcd: 873.58

### Compound 20-2

Compound 20-1 (256 mg, 0.21 mmol) was dissolved in a 1:1 mixture of dichloromethane and trifluoroacetic acid (5 mL). The mixture was stirred at room temperature for 4 hours, with frequent monitoring my LC-MS for reaction completion. The solution was concentrated by rotary evaporation, and the product was precipitated using diethyl ether. The resulting white residue was washed with diethyl ether (3×30 mL) and isolated by centrifugation, dried under vacuum, resulting in Compound 20-2 as a white powder (104 mg, 90% yield) that was used without further purification. ¹H NMR (500 MHz, Deuterium Oxide) δ 3.96 (m, 2H), 3.88-3.57 (m, 5H), 3.54-2.74 (m, 18H), 2.65-2.20 (m, 2H), 1.99 (d, J=106.3 Hz, 2H), 1.79-1.57 (m, 3H), 1.40-0.96 (m, 1H). LC-MS (Method 3): t_{R}=3.23 min, m/z=873.45 [M+H]; calcd: 873.58

### Compound 20

Compound 20-2 164 mg, 0.29 mmol) was dissolved in water. To this solution was added GdCl₃ (123 mg, 0.38 mmol), and the pH was adjusted to 6.8 with a solution of 1M NaOH. The solution was stirred for one hour at room temperature, then purified by CombiFlash using method 10. Compound 20 was isolated as a white solid after freeze-drying. Mass=187 mg, 89%. LC-MS (Method 3): t_{R}=4.5 min, m/z=704.15 [M+2H]; calcd: 704.18. LC-ICP (Method 11): t_{R}=5.03 min, purity >95%

### Example 26. Complexation of Compounds 9, 10, and 11 with Gd³⁺

Compound 9, 10, or 11 (70 or 71 μmol) was dissolved in water (5 mL) and the pH of the solution was adjusted to 5.5 with 0.1 M NaOH. Then GdCl₃·6H₂O or YbCl₃·6H₂O (1.1 equiv., 77 or 79 μmol) dissolved in water (2 mL) was added dropwise. The pH of the solution was kept at 5.5, and the solution was stirred for 2 hours. Then the solution was passed through a chelex column to remove any free metal ion, and lyophilized to yield the title compound. Xylenol orange test were negative, demonstrating the absence of non-chelated Gd/Yb(III). Corresponding Gd/Yb(III) complex was characterized by LC-MS and HR-ESI-MS. The purity was further characterized by LC-ICP:

Gd-9: 60 mg, 98% yield; LC-MS Method 3 or method 7, t_{R}=3.77 min, m/z [M]⁻ calcd for [C₃₀H₅₀GdN₁₀O₁₀]⁻, 868.3; found 868.1. HR-ESI-MS m/z (%) [M]⁻ calcd for [C₃₀H₅₀GdN₁₀O₁₀]⁻, 868.2958; found 868.2983. LC-ICP: method 20 or method 23, detection of Gd at m/z=157, t_{R}=5.09 min, purity=98%.

Gd-10: 58 mg, 97% yield; LC-MS Method 3 or method 7, t_{R}=3.79 min, m/z [M]⁻ calcd for [C₃₀H₅₀GdN₁₀O₁₀]⁻, 868.3; found 868.2. HR-ESI-MS m/z (%) [M]⁻ calcd for [C₃₀H₅₀GdN₁₀O₁₀]⁺, 868.2958; found 868.2965. The purity was further characterized by LC-ICP: method 20 or method 23, detection of Gd at m/z=157, t_{R}=5.10 min, purity=99%.

Gd-11: 60 mg, 97% yield; LC-MS Method 3 or method 7, t_{R}=3.78 min, m/z (%) [M]⁻ calcd for [C₃₀H₄₉GdN₉O₁₀]⁻, 853.3; found 853.3. HR-ESI-MS m/z (%) [M]⁻ calcd for [C₃₀H₄₉GdN₉O₁₀], 853.2849; found 853.2884. The purity was further characterized by LC-ICP: method 20 or method 23, detection of Gd at m/z=157, t_{R}=5.05 min, purity=98%.

Yb-9: 95% yield; LC-MS Method 3 or method 7, t_{R}=3.77 min, m/z [M]⁻ calcd for [C₃₀H₅₀YbN₁₀O₁₀]⁻, 884.3; found 884.1. HR-ESI-MS m/z (%) [M]⁻ calcd for [C₃₀H₅₀YbN₁₀O₁₀], 884.3105; found 884.3115. LC-ICP: method 20 or method 23, detection of Yb at m/z=173, t_{R}=5.08 min, purity=99%.

### Example 27. Complexation of Compounds 12, 13, 14 with Mn²⁺

Compound 12, 13, or 14 (0.2 mmol) and MnCl₂ (0.2 mmol) were dissolved in 4 mL of water. The solution was adjusted to pH 5.5 using 0.1 M NaOH and transferred to a Schelenk tube. After degassing by five N₂-pump cycles, the reaction was heated to 60° C. and stirred for 1 hour. Then the solution was passed through a chelex column to remove any free metal ion. The concentration of corresponding Mn complexes was determined by ICP-MS. The purity was further characterized by LC-ICP.

Mn-12: Yield 88%. LC-MS (method 2, ESI^{†}): t_{R}=2.99 min, m/z [M+H]⁺ calcd for [C₂₈H₅₃MnN₁₀O₆]⁺, 680.4; found 680.3. LC-ICP: method 22, detection of Mn at m/z=55, t_{R}=5.42 min, purity=96%.

Mn-13: Yield 85%. LC-MS (method 2, ESI^{†}): t_{R}=3.57 min, m/z [M+H]⁺ calcd for [C₂₄H₄₆MnN₇O_{5]}⁺, 567.3; found 567.2. LC-ICP: method 22, detection of Mn at m/z=55, t_{R}=5.97 min, purity=98%.

Mn-14: Yield 90%. LC-MS (method 7, ESI^{†}): t_{R}=4.89 min, m/z [M+H]⁺ calcd for [C₂₀H₃₉MnN₈O₆]⁺, 542.2; found 542.2. LC-ICP: method 22, detection of Mn at m/z=55, t_{R}=3.86 min, purity=95%.

### Example 28. Complexation of Compounds 15, 16, 17, and 18 with Mn²⁺

Compound 15, 16, 17, or 18 (0.02 mmol) was dissolved in water (2.0 mL), and the pH adjusted to 6.5 with 0.1 M NaOH. Then MnCl₂.4H₂O (0.02 mmol) was added under stirring. The pH decreased to ˜3.5, and the completion of the complexation was confirmed by LC-MS. The excess free Mn²⁺ were removed by Chelex 100 resin (pH 6.5). The complexes were then lyophilized to a powdery solid and redissolved in water as necessary.

Mn-15: Yield 87%. LC-MS (method 6): t_{R}=7.49 min, m/z [M+H]⁺ calcd for [C₂₃H₃₆MnN₇O₇]⁺, 577.2; found 577.2, purity=96% (250 nm UV detector).

Mn-16: Yield 91%. LC-MS (method 6): t_{R}=6.58 min, m/z [M+H]⁺ calcd for [C₂₃H₃₅MnN₆O₈]⁺, 578.2; found 577.1, purity=97% (250 nm UV detector).

Mn-17: Yield 85%. LC-MS (method 6): t_{R}=9.53 min, m/z [M+H]⁺ calcd for [C₂₀H₃₂MnN₇O₅]⁺, 505.1; found 505.0, purity=92% (250 nm UV detector).

Mn-18: Yield 90%. LC-MS (method 6): t_{R}=8.01 min, m/z [M+H]⁺ calcd for [C₂₅H₃₉MnN₆O₇]⁺, 590.2; found 590.1, purity=97% (250 nm UV detector).

### Example 29. Complexation of Compounds 15, 16, 17, and 18 with Zn²⁺

Compound 15, 16, 17, or 18 (0.02 mmol) was dissolved in water (2.0 mL), and the pH adjusted to 6.5 with 0.1 M NaOH. Then ZnCl₂ (0.02 mmol) was added under stirring. The pH decreased to ˜3.5, and the completion of the complexation was confirmed by LC-MS. The excess free Zn²⁺ were removed by Chelex 100 resin (pH 6.5). The complexes were then lyophilized to a powdery solid and redissolved in water as necessary.

Zn-15: Yield 90%. ¹H NMR (500 MHz, D₂O) δ 7.88 (t, J=7.9 Hz, 1H), 7.30 (d, J=7.7 Hz, 2H), 4.20-4.01 (m, 4H), 3.64-3.39 (m, 4H), 3.23 (t, J=6.2 Hz, 1H), 3.08 (s, 2H), 2.90 (t, J=12.2 Hz, 2H), 2.82-2.68 (m, 4H), 2.61 (t, J=13.6 Hz, 2H), 2.36-2.21 (m, 2H), 2.01 (s, 2H), 1.90 (q, J=6.1 Hz, 2H), 1.57 (s, 2H). LC-MS (method 6): t_{R}=7.19 min, m/z [M+H]⁺ calcd for [C₂₃H₃₆N₇O₇Zn]⁺, 586.2; found 586.0, purity=95% (250 nm UV detector).

Zn-16: Yield 92%. ¹H NMR (500 MHz, D₂O) δ 7.96-7.83 (m, 1H), 7.30 (d, J=7.8 Hz, 2H), 4.25-4.03 (m, 4H), 3.86 (dd, J=7.9, 4.3 Hz, 1H), 3.66-3.38 (m, 4H), 3.04 (t, J=6.7 Hz, 2H), 2.97-2.86 (m, 2H), 2.85-2.68 (m, 4H), 2.61 (dt, J=14.8, 4.3 Hz, 2H), 2.29-2.11 (m, 2H), 2.09-1.96 (m, 2H), 1.93-1.83 (m, 1H), 1.76 (m, 1H), 1.57 (t, J=8.2 Hz, 2H). LC-MS (method 6): t_{R}=6.27 min, m/z [M+H]⁺ calcd for [C₂₃H₃₅N₆O₈Zn]⁺, 587.2; found 587.1, purity=96% (250 nm UV detector).

Zn-17: Yield 90%. ¹H NMR (500 MHz, D₂O) δ 7.89 (t, J=7.7 Hz, 1H), 7.31 (d, J=7.7 Hz, 2H), 4.19-4.06 (m, 4H), 3.62-3.53 (m, 4H), 3.45 (d, J=17.0 Hz, 2H), 3.17 (t, J=6.0 Hz, 2H), 2.91 (m, 2H), 2.82-2.70 (m, 4H), 2.60 (m, 2H), 2.03 (m, 2H), 1.61 (m, 6.5 Hz, 2H). LC-MS (method 6): t_{R}=9.75 min, m/z [M+H]⁺ calcd for [C₂₀H₃₂N₇O₅Zn]⁺, 514.2; found 514.0, purity=94% (250 nm UV detector).

Zn-18: Yield 91%. ¹H NMR (500 MHz, D₂O) δ 7.92 (t, J=7.7 Hz, 1H), 7.34 (d, J=7.8 Hz, 2H), 4.26-4.06 (m, 4H), 3.72-3.43 (m, 5H), 3.09 (td, J=6.8, 2.7 Hz, 2H), 2.94 (m, 2H), 2.88-2.72 (m, 11H), 2.65 (dt, J=14.2, 4.1 Hz, 2H), 2.37-2.21 (m, 2H), 2.13 (m, 1H), 2.07-1.84 (m, 3H), 1.68-1.52 (m, 2H). LC-MS (method 3-3): t_{R}=7.79 min, m/z [M+H]⁺ calcd for [C₂₅H₃₉N₆O₇Zn]⁺, 599.2; found 599.1, purity=96% (250 nm UV detector).

### Example 30. Radiolabeling of Compounds 1-8 with ⁶⁸Ga

Bond Elut SCX cartridge (100 mg, Agilent) was slowly washed with 5.5M HCl (1 mL) and H₂O (10 mL). ⁶⁸GaCl₃ (6 mCi) was eluted from the Ga-68 generator (Eckert&Ziegler) with 0.1M HCl and was loaded onto the preactivated cartridge. The cartridge was purged with air and ⁶⁸GaCl₃ was eluted with 3M NaCl (0.3 mL in 140 mM HCl). Solution of Compounds 1-8 (50 μL, 0.5 mg in 1 mL of 10 mM HCl) was diluted with 1.5 M NaOAc (pH=4.2) and 150 μL of ⁶⁸GaCl₃ was added. The reaction mixture was heated at 60° C. (Compounds 5, 7, 8) or 90° C. (Compounds 1, 2, 3, 4, 6) for 10 min. The reaction mixture was allowed to cool down for 1 min (Compounds 5, 7, 8) or 3 min (Compounds 1, 2, 3, 4, 6) and was diluted with 0.65 mL of sterile H₂O.

### Example 31. Radiolabeling of Compounds 1-8 with ⁶⁴Cu

⁶⁴CuCl₂ (28 mCi) was received from the cyclotron facility at the University of Wisconsin Madison and diluted to 1 mL with H₂O. Solution of Compounds 1-8 (50 μL, 0.5 mg in 1 mL of 10 mM HCl) was diluted with 1.5 M NaOAc (pH=4.2) and 50 μL of ⁶⁴CuCl₂ was added. The reaction mixture was heated at 60° C. (Compounds 5, 7, 8) or 90° C. (Compounds 1, 2, 3, 4, 6) for 10 min. The reaction mixture was allowed to cool down for 1 min (Compounds 5, 7, 8) or 3 min (Compounds 1, 2, 3, 4, 6) and was diluted with 0.65 mL of sterile H₂O.

### Example 32. Radiolabeling of Compounds 15-18 with ⁵²Mn and Preparation of ^{52/nat}Mn Complex

⁵²MnCl₂ (5 mCi) was received from the cyclotron facility at the Alabama at Birmingham and diluted to 500 μL with 0.1 M HCl. Solution of Compounds 1-8 (50 μL, 1.0 mg in 1 mL of 1.5 M NaOAc buffer, pH 4.5) was mixed with 50 μL of ⁵²MnCl₂. The reaction mixture was kept at room temperature for 15 min and diluted with 200 μL of sterile PBS. The injected dose of ^{52/nat}Mn complex was obtained by mixing solution of ⁵²Mn labeled Compound 15-18 with stock solution of ^{nat}Mn labeled Compound 15-18 (^{nat}Mn final concentration: 30 mM).

### Example A. In Vitro and Animal Model Data

### In Vitro Reactivity of Compounds

Quantitative Measurement of Reaction Rates with Aldehydes:
Method A: Reaction Rates of Compounds with 2-Formylpyridine Measured by HPLC-ICP-MS

Reactivity of aldehyde-binding probes were assessed in pH 7.40 phosphate-buffered saline under pseudo-first order conditions with 2-formylpyridine concentration of 1000 μM and probes at 25 μM. Concentrations of unreacted starting material and condensation products were determined at 12 min time intervals based on their relative integrations. The values were fit to a standard first order rate constant linear equation and the rate constant was extracted from the slope.

Method B: Reaction Rates of Compounds with Butyraldehyde Measured by HPLC-ICP-MS

The reactions were conducted in pH 7.40 phosphate-buffered with butyraldehyde concentration of 100 μM and hydrazine probes at 25 μM. Concentrations of unreacted starting material and condensation products were determined at 10 min time intervals over 2 hours based on their relative integrations. The values were fit to a standard first order rate constant linear equation and the rate constant was extracted from the slope.

Method C: Reaction Rates of Fluorescent Compounds with Butyraldehyde Measured by HPLC with UV-Vis and Fluorescence Detection.

Condensations reactions were conducted in pH 7.40 phosphate-buffered saline with butyraldehyde concentration of 100 μM and fluorescent probes at 10 μM. Concentrations of unreacted starting material and condensation products were determined at 10 min time intervals over 2 hours based on their relative integrations. The values were fit to a standard first order rate constant linear equation and the rate constant was extracted from the slope.

Method D: Reaction Rates with UV-Visible Measurements.

The molar extinction coefficient at 220 nm (ε₂₂₀) of Mn-15, Mn-16 and Mn-17 was calculated using Beer-Lambert Law: A₂₂₀=ε₂₂₀Cl; where A₂₂₀ is the absorbance at wavelength 220 nm, C is the concentration (M), and l is the cell path length (cm).

The extinction coefficients of the reaction products of Mn-15, Mn-16 and Mn-17 with butyraldehyde (Mn-15-Ald, Mn-16-Ald, and Mn-17-Ald) were calculated by the following method:

The HPLC trace of each of 1 mM Mn—N(where Mn—N refers to Mn-15, Mn-16, or Mn-17) were first obtained by using Method 24. Then, another batch of Mn—N(final concentration: 1 mM) with 200 equivalents of butyraldehyde were added to drive the reaction to completion. The HPLC traces of the reaction product of Mn—N with butyraldehyde, denoted as Mn—N-Ald (where Mn-Ald refers to the reaction product with either Mn-15, Mn-16, or Mn-17) were then obtained by method 24. The ε₂₂₀ values of Mn—N-Ald were calculated using the following equation:

$${ε}_{Mn - N - Ald} = {ε}_{Mn - N} \times \frac{{AUC}_{Mn - N - Ald}}{{AUC}_{Mn - N}} \qquad ( 1 )$$

where AUC is area under curve of the desired peak on HPLC trace, ε_{Mn-N-Ald} is the extinction coefficient of the aldehyde reaction product (for compound N) and ε_{Mn-N} is the extinction coefficient of the Mn-15, Mn-16 and Mn-17 starting material. ε values are listed in Table A1:

TABLE A1
Molar extinction coefficient of Mn-15, Mn-16, Mn-17 and corresponding reaction
product with butyraldehyde (Mn-15-Ald, Mn-17-Ald, Mn-18-Ald) at 220 nm.

| | Mn-15 | Mn-16 | Mn-17 | Mn-15-Ald | Mn-16-Ald | Mn-17-Ald |
|---|---|---|---|---|---|---|
| ε₂₂₀ | 4800 M⁻¹cm⁻¹ | 10,700 M⁻¹cm⁻¹ | 5200 M⁻¹cm⁻¹ | 5200 M⁻¹cm⁻¹ | 1800 M⁻¹cm⁻¹ | 20,400 M⁻¹cm⁻¹ |

The kinetics for reactions of Mn-15, Mn-16 and Mn-17 with butyraldehyde were monitored spectrophotometrically by measuring the change in absorbance at 220 nm. All reactions were carried out at 25° C. in PBS. In each experiment [butyraldehyde]=1 mM and the concentrations of Mn-complex, 0.04 mM, 0.06 mM, 0.08 mM, and 0.10 mM, were varied to estimate the rate law.

At the beginning of the reaction (t=0), the concentration of the starting Mn complex is a₀ and the concentration of the aldehyde condensation product is zero. After time t the concentration of the product is x, and the concentration of remaining Mn complex starting material is a₀-x. For a first-order reaction

$$\frac{d [ x ]}{dt} = {k}_{obs} ( {a}_{0} - x ) \qquad ( 2 )$$

Separation of the variables gives

$$\frac{dx}{{a}_{0} - x} = {k}_{obs} dt \qquad ( 3 )$$

and integration gives

$$ln ( {a}_{0} - x ) = - {k}_{obs} t - I \qquad ( 4 )$$

where I is the constant of integration and k_{obs} is the observed rate constant. The rate law and a corresponding second order rate constant k were estimated by plotting reaction velocity as a function of Mn—N(where Mn—N refers to Mn-15, Mn-16, or Mn-17) concentration.

The concentration of Mn—N-Ald (where Mn-Ald refers to the reaction product with either Mn-15, Mn-16, or Mn-17) can be estimated by the following equations, where A₀ is the absorbance of Mn—N and A is the absorbance of reaction mixture after time t,

$${A}_{0} = {ε}_{MnL} {a}_{0} l \qquad ( 5 )$$

The absorbance of the reaction mixture is

$$A = {ε}_{Mn - N} ( {a}_{0} - x ) l + {ε}_{Mn - N - Ald} xl \qquad ( 6 )$$

After subtraction

$$Δ A = A - {A}_{0} = {ε}_{M n N} ( {a}_{0} - x ) l + {ε}_{M n - N - Ald} x l - {ε}_{M n - N} {a}_{0} l \qquad ( 7 )$$

$$Δ A = ( {ε}_{M n - N} - {ε}_{M ? ι - N} ) x l \qquad ( 8 )$$

TABLE A2
Examples of second order rate constants of selected
compounds and corresponding methods used

| Compound | k (M⁻¹s⁻¹) | Method |
|---|---|---|
| 19 | 0.55 | a |
| 20 | 1.27 | a |
| GdCHyd* | 1.7 | b |
| Gd-9 | 3.6 | b |
| Gd-10 | 3.5 | b |
| Mn-12 | 4.5 | b |
| Mn-13 | 1.9 | b |
| Mn-14 | 1.4 | b |
| Mn-15 | 12.1 | d |
| Mn-16 | 2.5 | d |
| Mn-17 | 4.6 | d |
| Ga-1 | 0.06 | a |
| Ga-2 | 0.11 | a |
| Ga-4 | 0.34 | a |
| 21 | 0.68 | c |
| 22 | 1.52 | c |
| 23 | 0 (no binding) | c |
| 25 | 3.03 | c |

*From Akam, E. A., et al. Improving the reactivity of hydrazine-becring MRI probes for in vivo imaging of lung fibrogenesis. Chemical Science 11, 224-231 (2020).

### Quantitative Measurements of Kinetics of Hydrolysis:

One mM solutions of Mn-15-Ald, Mn-16-Ald and Mn-17-Ald (where Mn-Ald refers to the reaction product with either Mn-15, Mn-16, or Mn-17) were prepared by the following procedure: 1 mM (1 mL) of Mn-15, Mn-16 or Mn-17 were prepared in PBS (pH 7.4) followed by addition of butyraldehyde (14.4 mg, 200 mmol). The mixture was rocked gently under room temperature for 10 min and then freeze-dried for 12 hours. Then, the obtained white solid was redissolved in 1 mL water.

The HPLC trace of each butyraldehyde reaction product was measured by Method 25 (Mn-15-Ald) and Method 26 (Mn-16-Ald, Mn-17-Ald). Then, a 37% stock solution of formaldehyde was added to give a final formaldehyde concentration of 200 mM.

$$\% hydrolysis = 100 \frac{{A}_{0}^{′} - {A}^{′}}{{A}_{0}^{′}} \qquad ( 9 )$$

where A₀′ is the AUC of the butyraldehyde reaction product before addition of formaldehyde, A′ is the AUC of the butyraldehyde reaction product after addition of formaldehyde at time t.

The hydrolysis of the hydrazone/oxime is reversible. An excess of formaldehyde was added to trap the liberated nitrogen base and thereby push the hydrolysis reaction to completion, allowing the hydrolysis reaction to be monitored without interference from the reverse reaction. So, the kinetics for hydrolysis of Mn-15-Ald, Mn-16-Ald and Mn-17-Ald follows the rate law.

$$\frac{d [ y ]}{dt} = {k}_{obs , H} ( {b}_{0} - y ) \qquad ( 10 )$$

where the concentration of Mn—N-Ald at t=0 is b₀ and the concentration of hydrolysis product is zero. After time t the concentration of hydrolysis product is y, and the concentration of remaining Mn—N-Ald is b₀-y.

Separation of the variables gives

$$\frac{d y}{{b}_{0} - y} = {k}_{obs , H} d t \qquad ( 11 )$$

and integration gives

$$ln ( {b}_{0} - y ) = - {k}_{obs , H} t - {I}_{H} \qquad ( 12 )$$

the constant-H can be evaluated using the boundary condition that y=0 when t=0; hence

$$- ln ( {b}_{0} ) = {I}_{H} \qquad ( 13 )$$

and insertion of equation 13 to 12 lead to

$$ln \frac{{b}_{0}}{{b}_{0} - y} = {k}_{obs , H} t \qquad ( 14 )$$

this equation can also be written as

$${b}_{0} - y = {b}_{0} {e}^{- {k}_{o b s , H} t} \qquad ( 15 )$$

$$or y = {b}_{0} ( 1 - {e}^{- {k}_{obs , H} t} ) \qquad ( 16 )$$

when the hydrolysis reaction reaches plateau, value of yₘₐₓ at equilibrium

$${y}_{max} = {b}_{0} \qquad ( 17 )$$

and insertion of equation 17 into equation 16 leads to

$$y = {y}_{max} ( 1 - {e}^{- {k}_{obs , H} t} ) \qquad ( 18 )$$

divide by the initial concentration of Mn—N-Ald, b₀, leads to

$$Y = {Y}_{max} ( 1 - {e}^{- {k}_{obs , H} t} ) \qquad ( 19 )$$

where Y is the % hydrolysis, t is time, k_{obs,H} is the first-order rate constant, and Yₘₐₓ is the % hydrolysis at t=∞. Kinetic traces were obtained in triplicate, and half-lives were calculated with equation 20:

$${t}_{1 / 2} = \frac{0.693}{{k}_{o b s , H}} \qquad ( 20 )$$

TABLE A3
Hydrolysis rates of selected compounds

| | Mn-15 | Mn-16 | Mn-17 |
|---|---|---|---|
| Hydrolysis rate constant | 1.03 ± 0.15 | 0.79 ± 0.03 | 0.41 ± 0.01 |
| (s⁻¹) | (×10⁻³) | (×10⁻⁶) | (×10⁻³) |

### Preparation of Allysine Containing Bovine Serum Albumin, BSA-ALD:

To a solution of bovine serum albumin (BSA) (100 mg) dissolved in phosphate buffered saline (4 mL, pH 7.4, 0.25 mM), sodium ascorbate (20 mg), ferric chloride (120 μL, 10 mM) and 20 μL H₂O₂ (30% w) were added. The reaction was stirred at 37° C. for 24 hours, and sodium ascorbate (20 mg) was added repeatedly every 8 hours. After the reaction, protein was purified using PD-10 Sephadex G25 desalting columns (GE Healthcare), eluted with PBS. Protein concentration was assessed using the ‘BCA Protein Assay Kit’ (Thermo Scientific). Protein carbonyl concentration was determined by ‘Protein Carbonyl Content Assay Kit’ (Sigmal-Aldrich). BSA-ALD had an aldehyde concentration of 4 aldehyde/protein. BSA had an aldehyde concentration on ˜0.3 aldehyde/protein.

Alternatively, preparation of BSA^{Ald} and binding of Gd³⁺ probe to protein was carried out according to modified procedures. To a solution of bovine serum albumin (BSA) (100 mg) dissolved in phosphate buffered saline (4 mL, pH 7.4, 0.25 mM) was added sodium ascorbate (20 mg), ferric chloride (120 μL, 10 mM) and 20 μL H₂O₂ (30% w). The reaction was stirred at 37° C. for 24 h, and sodium ascorbate (20 mg) was added repeatedly every 8 h. After 24 h, the protein was purified using PD-10 Sephadex G25 desalting columns (GE Healthcare), eluted with PBS. Protein concentration was assessed using the ‘Micro BCA Protein Assay Kit’ (Thermo Scientific, 23235). Protein carbonyl concentration was determined by ‘Protein Carbonyl Content Assay Kit’ (Sigma-Aldrich, MAK094-1KT). BSA^{Ald} had an aldehyde concentration of 4 aldehyde/protein. The protein solutions were kept at a concentration of 20 mg/mL for further use.

### Relaxivity Measurements:

BSA-ALD (10 mg/mL) or BSA (10 mg/mL) were treated for 24 hours at 37° C. with the corresponding Gd or Mn complex at a range of concentrations (0.01-0.2 mM), with a total volume of 300 μL maintained for all samples. After 24 hours, an aliquot of the solution (50 μL) was used for longitudinal (T₁) relaxation measurements, recorded using a Bruker mq60 Minispec at 1.41 T and 37° C. Longitudinal (T₁) relaxation times were measured via an inversion recovery experiment using 10 inversions of duration ranging between 0.05×T₁ and 10×T₁. Solutions (concentration range: 0.1 mM-1.0 mM) in PBS were run in parallel. To measure the relaxivity of BSA-bound species, sodium cyanoborohydride (10 mg) was added to the solution to irreversibly bind the probe to the protein. After a further 2h incubation at 37° C., protein-bound and protein-free solutions were separated by ultrafiltration. Then 200 μL PBS was added to the residue to dissolve the protein. Longitudinal (T₁) relaxation times for the bounded species were measured using a Bruker mq60 Minispec at 1.41 T and 37° C. After the measurement, concentration of corresponding metal ion (Gd/Mn) in the residue and filter were both determined using an Agilent 8800 ICP-MS system. Relaxivity (r₁) was determined from the slope of a plot of 1/T₁ vs metal concentration for 5 concentrations.

On Off Rate with Oxidized BSA-ALD:

BSA-ALD (10 mg/mL) was incubated with 200 μM corresponding Gd or Mn complex at 37° C. in pH 7.4 PBS), with a total volume of 300 μL. The dynamic longitudinal (T₁) relaxation time were measured using a Bruker mq60 Minispec at 1.41 T and 37° C. After 24 hours, protein-bound and protein-free solutions were separated by ultrafiltration. Then 200 μL PBS was added to the residue to dissolve the protein bound species. The change of longitudinal (T₁) relaxation time was then measured in Bruker mq60 Minispec at 1.41 T and 37° C.

### Binding of Fluorescent Compounds to BSA-ALD.

BSA-ALD (25 μM) was incubated with 10 μM fluorescent probe for 3 hours at 37° C. in pH 7.4 PBS with a total volume of 300 μL. UV-vis spectra were collected of each solution. Free dye and BSA-Ald-bound dye were then separated by ultrafiltration using 10 kDal MWCO. UV-vis spectra were then collected for the resulting solutions to determine the concentration of unbound/unreacted dye based on the extinction coefficient of TAMRA of 89,000 M⁻¹ cm⁻¹ at 555 nm. For blocking experiments, BSA-ALD was incubated with 100 mole equivalents of hydrazine and o-methylhydroxylamine at 37 degrees for 24 hours.

TABLE A4
Binding of aldehyde-reactive fluorescent probes
to BSA-Ald with and without blocking

| | % Bound | |
|---|---|---|
| Probe | BSA-Ald | Blocked BSA-Ald |
| 21 | 40 | 0 |
| 22 | 27 | 3 |
| 23 | 0.9 | 0.6 |
| 25 | 74 | 11 |

### Stability of Compounds in Human Plasma:

A 16 μL aliquot of the Gd or Mn complex (1.0 mM) was added to 500 μL of human plasma (Lampire Biological laboratories). The solution was then incubated for 2 or 3 hours at 37° C. Aliquots (50 μL) were removed at 1, 2 or 3 hours. Proteins were precipitated by the addition of 150 μL of acetonitrile and removed following centrifugation. The supernatant was then analyzed using HPLC-ICP-MS. No unchelated gadolinium was observed nor the appearance of any new Gd/Mn-containing species. FIGS. 49A and 49B show stability characterization of Gd-9 and Gd-10, showing a lack of de-chelation or formation of Gd-containing metabolites after 3 hours.

TABLE A5
Relaxivities measured in different media at 1.4 T, 37° C.

| | r₁ (mM⁻¹s⁻¹) | | | |
|---|---|---|---|---|
| | | | | Bound to |
| Probe | PBS | +BSA | +BSA-Ald | BSA-Ald |
| Gd-9 | 6.6 | 6.5 | 11.2 | 17.7 |
| Gd-10 | 6.6 | 6.6 | 9.0 | 18.7 |
| Gd-11 | 6.2 | 6.2 | 7.6 | 13.8 |
| Mn-12 | 1.6 | 2.2 | 4.5 | 7.7 |
| Mn-13 | 1.6 | 1.9 | 2.3 | 6.2 |
| Mn-14 | 1.5 | 1.8 | 2.5 | 7.3 |
| Mn-15 | 3.1 | 3.6 | 5.9 | 12.7 |
| Mn-16 | 3.1 | 3.4 | 7.7 | 12.7 |
| Mn-17 | 3.0 | 3.5 | 5.6 | 11.4 |
| Mn-18 | 3.1 | 3.5 | 3.6 | — |

### Animal Studies

### Animal Models:

All experiments were performed in accordance with the NIH Guide for the Care and Use of Laboratory Animals and in compliance with the ARRIVE guidelines and approved by the MGH Institutional Animal Care and Use Committee. All animals were sacrificed after the imaging for biochemical analysis and histological evaluations.

### CCl₄ Liver Fibrosis Model:

Male C57BL/6 mice (Charles River Laboratories, Wilmington, MA) were treated with carbon tetrachloride for 12 weeks (0.1 ml of 20% CCl₄ in olive oil the first week, 30% the second week, and 40% from weeks 3-12) by oral gavage, 2-3 times per week. Control mice were fed vehicle (olive oil) only.

A total of 53 C57BL/6 mice were included to study CCl₄ induced hepatic fibrosis:
A. CCl₄ for 12 weeks (n=20). Each mouse was imaged with probe 1 and then imaged again 24 hours later with probe 2. Probe 1 and Probe 2 were randomly chosen from GdDOTA, GdCHyd, Mn-12, Gd-9 and Gd-10.
B. Vehicle treated control (n=20). Each mouse was imaged with probe 1 and then imaged again 24 hours later with probe 2. Probe 1 and Probe 2 were randomly chosen from GdDOTA, Mn-12, GdCHyd, Gd-9, Gd-10.
C. CCl₄ for 12 weeks (n=3, another 3 pair-wise comparison have been done in group A). Each mouse was imaged with probe 1 and then imaged again 24 hours later with probe 2. Probe 1 and Probe 2 were randomly choosing from Gd-9 and Gd-10.
D. Vehicle treated control (n=2, another 2 pair-wise comparison have been done in group B). Each mouse was imaged with probe 1 and then imaged again 24 hours later with probe 2. Probe 1 and Probe 2 were randomly choosing from Gd-9 and Gd-10.
E. CCl₄ for 12 weeks (n=3). Each mouse was imaged with Gd-9. After 24 hours, the same mouse was first injected with 1000 μmol/kg of Yb-9 and then injected with 100 μmol/kg of Gd-9 15 min after the Yb-9 injection.

### Non-Alcoholic Steatohepatitis Liver Fibrosis Model:

A total of 40 C57BL/6 mice were used and randomized to each study group. To induce NASH, 6-week old, male C57BL/6 mice (Charles River Labs, Wilmington, MA) were fed a choline deficient, high fat diet (CDAHFD) consisting of 60 kcal % fat and 0.1% methionine (A06071302; Research Diets, New Brunswick, NJ) by weight. No animals were excluded from the study.

Three groups of mice were studied:

Group A, mice were fed CDAHFD for 2 (n=6), 6 (n=6), 10 (n=6) weeks and imaged with probe Gd-9.

Group B, mice were fed CDAHFD for 10 weeks followed by normal chow for 1 (n=6) or 4 weeks (n=6) and imaged with probe Gd-9.

Group C, mice were fed normal chow for 2, 6, 10, 11 or 14 weeks (n=10) and imaged with probe Gd-9.

### Rat Liver Fibrosis Model:

Liver fibrosis was induced in male Sprague Dawley rats (n=7) by ligation of the common bile duct (CD®, Charles River Labs, Wilmington, MA). Control animals (n=4) underwent a sham procedure. The BDL and sham rats were imaged 10 days following ligation with a dose of 100 mol/kg of Gd-9 based on body surface area.

### Lung Fibrosis Model:

C57Bl/6 adult male mice at 8 weeks of age (Jackson Laboratories, Barr Harbor, ME) received a single intratracheal dose of bleomycin, 1 units/kg body weight (50 μL total volume) (Fresenius Kabi, Lake Zurich, Il) as previously described in Desogere, P., et al. Optimization of a Collagen-Targeted PET Probe for Molecular Imaging of Pulmonary Fibrosis. J Nuc Med 58, 1991-1996 (2017).

### Combined Heart and Lung Fibrosis Model:

Left ventricular dysfunction with pulmonary hypertension was induced by left thoracotomy transverse aortic constriction surgery (TAC) in 6-month-old senescence-accelerated-prone/resistant mice (SAMP8/SAMR1), which is a well-established model of pressure overload-induced cardiac hypertrophy that can induce heart and lung fibrosis. The transverse aorta was encircled with 7-0 nylon suture and tied tightly around a pre-sterilized, blunt-end of a 27-gauge needle. After securing the knot, the needle was removed, and aortic flow resumed. Another two groups of SAMP8 and SAMR1 mice received sham surgery as control. 3-weeks post-surgery, mice were anesthetized with isoflurane (1.5%) and imaged at a 4.7 Tesla Bruker MRI scanner.

### Kidney Ischemia-Reperfusion (IRI) Model:

Male C57BL/6 mice (10-12 weeks old; Charles River Laboratories) were anesthetized with ketamine/xylazine (100/10 mg/kg; IP). Animals were then placed prone while maintaining rectal temperature strictly at 37° C. with a feedback-regulated heating pad. The skin incision was performed on the left lower flank while adhering to strict aseptic procedures. After accessing the retroperitoneal space, the left kidney, and the left renal artery-vein was identified. Ischemia was induced by applying a micro serrefine clip onto the renal artery and vein. Successful ischemia was visually confirmed by a gradual uniform darkening of the kidney. The clamp was removed 26 minutes later, and a rapid change verified successful reperfusion in kidney color from a dark maroon to a healthy dark pink. The skin was closed using surgical staples, and animals were returned to their home cages. Analgesia was provided by buprenorphine (0.1 mg/kg, SC, bid for three days starting 1 hour before IRI surgery).

Animals were imaged 14 days after IRI and were euthanized after imaging. The right and left kidneys were removed, cortex and medulla regions of each kidney were separated for further analysis.

### Tumor Xenograft Model:

Male NU(NCr)-Foxn1nu nude mice at 6-8 weeks of age were provided by Charles River Labs, Wilmington, MA. A total of 1×10⁷ patient derived metastatic pancreatic cancer cells (PDAC6) were injected into the subcutaneous space of the right lower back of the mice (n=4 per group). Mice were imaged with Mn-17 (100 μmol/kg) 4 weeks after implanting the tumor for pretreatment imaging, and another group of mice were imaged 3-days post treatment with FOLFIRINOX (Folinate 50 mg/kg, Oxaliplaton 2.5 mg/kg, Irinotecan 25 mg/kg, Fluorouracil 25 mg/kg).

### Imaging and Biodistribution Study for Selectivity of Allysine-Reactive Probes In Vivo:

Bleomycin-injured mice at 14 days after injury were sedated and then injected with Compound 19, Compound 20, Gd-CHyd (Akam, E. A., et al. Improving the reactivity of hydrazine-bearing MRI probes for in vivo imaging of lung fibrogenesis. Chemical Science 11, 224-231 (2020)) formulated to contain an equal concentration of Eu-DOTA as a non-binding control probe. Animals were then euthanized at 30 minutes after injection. The lungs were harvested separately, along with blood, tail and liver. Each sample was analyzed by ICP-MS for gadolinium and europium content, and the lungs were also assessed for hydroxyproline content. Selectivity is reported as the ratio of Gd to Eu. All animals were dosed via tail vein injection at 100 nmol/g body weight from 30 mM solutions of gadolinium probes with 30 mM Eu-DOTA as determined by ICP-MS. For PET probes, Compounds 1-8 were radiolabeled with either ⁶⁸Ga or ⁶⁴Cu and the dose was delivered as a bolus injection through tail vein. Animals were euthanized 90 min post injection and their organs and tissues were harvested and counted using a gamma counter. For an imaging study with a PET tracer, animals were injected inside PET/MRI scanner and scanned dynamically for 60 min under anesthesia. The animals were euthanized 90 min post injection and their organs and tissues were counted using a gamma counter. The biodistribution of Mn-15, Mn-16, Mn-17, and Mn-18 was determined by injecting a mixture of ^{52/nat} complex into naïve mice and activity in each tissue were measured 24 hours post injection.

### Pair-Wise Comparison Between Gd-CHyd and Gd-9 in Lung Fibrosis Model:

A series of baseline images were first acquired. Then a bolus of Gd-CHyd was administered i.v. and imaging performed for a period of 40 min p.i.. Then anesthesia was removed, and the mouse was allowed to awake and return to cage. After 4 hours, the same mouse was anesthetized again. A series of baseline images were acquired, then a bolus of Gd-9 was administered i.v. and imaging performed for a period of 40 min p.i.

Pair-wise comparison between Mn-15 and Mn-17 in lung fibrosis model: The bleomycin-injured mice were first randomly imaged with Mn-15 or Mn-17 and then 24 hours later same imaging protocol was performed in the same mice with the other probe.

TABLE A6
The uptake of Compounds 19, 20 and Gd-CHyd in fibrotic
lung tissue of bleomycin-injured mice (n = 5 each) as determined
by ICP-MS. Gd content is reported as fold uptake over that
of Eu-DOTA, a non-binding control, also determined by ICP.
The Gd:Eu ratio was greater than 1 for all the probes
tested indicating specifici binding to the injured lung.

| | Gd:Eu ratio | |
|---|---|---|
| Compound | Right lung | Left lung |
| Gd-CHyd | 1.5 ± 0.4 | 1.9 ± 0.5 |
| 19 | 1.3 ± 0.2 | 1.4 ± 0.1 |
| 20 | 1.8 ± 0.5 | 1.9 ± 0.5 |

TABLE A7
PET probe lung uptake in naïve and bleomycin-injured mice
(‘bleo’, 14 days post bleomycin injury) expressed as (% ID/g)
measured 90 min post injection of probe. Probe uptake was
higher in the lungs of injured mice compared to the normal
lungs of naïve mice for all probes tested.

| | Lung uptake (% ID/g) | |
|---|---|---|
| Probe ID | Bleo | Naïve |
| ⁶⁴Cu-2 | 0.42 | 0.26 |
| ⁶⁴Cu-4 | 0.66 | 0.38 |
| ⁶⁸Ga-1 | 0.99 | 0.27 |
| ⁶⁸Ga-2 | 0.96 | 0.45 |
| ⁶⁸Ga-3 | 2.13 | 0.82 |
| ⁶⁸Ga-4 | 0.61 | 0.24 |
| ⁶⁸Ga-5 | 0.88 | 0.30 |
| ⁶⁸Ga-6 | 1.32 | 0.84 |

TABLE A8
⁶⁸Ga-7 probe uptake in lungs of fibrotic (n = 5) and naïve
(n = 4) animals as determined from PET scans (% ID/cc,
55 min p.i.) and organ counts (% ID/g, 90 min p.i.).
Probe uptake was 4 to 5-fold higher in the injured lung
compared to normal lung indicating specific targeting.

| | 55 min p.i. | 90 min p.i. |
|---|---|---|
| | (% ID/cc) | (% ID/g) |
| Bleomycin injured | 0.51 | 2.65 |
| mice | | |
| Naïve mice | 0.23 | 0.886 |

TABLE A9
Biodistribution of ^{nat/52}Mn-based probe in naïve mice at
24 h post injection (% ID/g). Low values indicate the
4 probes are all efficiently eliminated from the animal
after administration and do not accumulate in organs.

| | Mn-15 | Mn-16 | Mn-17 | Mn-18 |
|---|---|---|---|---|
| Brain | 0.0156 | 0.0275 | 0.0170 | 0.0145 |
| Blood | 0.0018 | 0.0051 | 0.0020 | 0.0013 |
| Heart | 0.0532 | 0.1579 | 0.0675 | 0.0364 |
| Lung | 0.1312 | 0.4469 | 0.1954 | 0.0599 |
| Liver | 0.2186 | 0.3950 | 0.2125 | 0.1833 |
| Kidney | 0.7021 | 0.9664 | 0.5387 | 0.5290 |
| Stomach | 0.0759 | 0.1638 | 0.0681 | 0.0477 |
| Spleen | 0.0843 | 0.1978 | 0.0965 | 0.0616 |
| Intestine | 0.0665 | 0.1330 | 0.0556 | 0.0355 |
| Muscle | 0.0154 | 0.0669 | 0.0196 | 0.0132 |
| Bone | 0.0385 | 0.1378 | 0.0414 | 0.0227 |
| Skin | 0.0719 | 0.9457 | 0.0925 | 0.0243 |

### MR Imaging and Analysis:

Animals were anesthetized with isoflurane (1-2%) and placed in a specially designed cradle with body temperature maintained at 37° C. The inhaled isoflurane level was adjusted to maintain a respiration rate of 60±5 breaths per minute. The tail vein was cannulated for intravenous (i.v.) delivery of the contrast agent while the animal was positioned in the scanner. Imaging was performed at 4.7 T using a small bore animal scanner with a custom-built volume coil. Mice were imaged with a dose of 100 μmol/kg from 30 mM stock solution of Gd or Mn complex.

For mice liver imaging, a series of baseline images (3D T1 weighted fast low angle shot magnetic resonance imaging (FLASH)) were first acquired (repetition time/echo time=15/2 ms; flip angle, 30°; field of view, 48×30 mm²; matrix size, 136×136 mm²; slice thickness, 0.25 mm; acquisition time, 3 minutes 20 seconds), then a bolus of Gd or Mn complex was administered i.v. and imaging performed for a period of 45 min p.i. with the repetition acquisition of 3D T1 weighted FLASH sequences. Following the imaging session, animals were sacrificed (75 min p.i.) and liver tissue was subjected to histopathologic analysis.

For rat liver imaging, a series of baseline images (T1-map and 3D T1 weighted FLASH) were first acquired, then a bolus of molecular probe was administered i.v. and imaging performed for a period of 30 min p.i. with the repetition acquisition of T1-mapping and 3D T1 weighted FLASH sequences. T1-mapping was performed with a flow sensitive alternating inversion recovery (FAIR) sequence: repetition time/echo time=11000/33.8 ms; flip angle, 90°; inversion times: 100, 200, 400, 500, 600, 1000, 1500, 2000 ms; field of view, 60×60 mm²; matrix size, 140×140; slice thickness, 2 mm; acquisition time, 3 minutes 34 seconds. T1 weighted imaging with 3D FLASH: repetition time/echo time=20/2.3 ms; flip angle, 30°; field of view, 60×60 mm²; matrix size: 127×127; slice thickness, 0.5 mm; acquisition time, 3 minutes 10 seconds. Following the imaging session, animals were sacrificed (30 min p.i.) and liver tissue was subjected to histopathologic analysis.

For lung imaging, images were acquired with following sequences and parameters: 3D Ultrashort TE (3D-UTE, TR/TE/FA=4 ms/11.75 μs/16°, 0.6 mm isotropic spatial resolution, field of view (FOV) 75 mm×75 mm, one average, acquisition time=6.1 min) images were acquired prior to and dynamically post injection of contrast agent; Rapid Acquisition with Relaxation Enhancement (RARE, TR/TE/FA=1.5 s/8 ms/180°, 0.3 mm isotropic spatial resolution, field of view (FOV) 60 mm×50 mm, four average, acquisition time=6.1 min) and T1-weighted 3D Fast Low Angle Shot (FLASH, TR/TE/FA=10 ms/2.5 ms/30°, 0.6 mm isotropic spatial resolution, field of view (FOV) 60 mm×50 mm, one average, acquisition time=6.1 min) images were also acquired.

For combined heart and lung imaging, a series of baseline images (2D T1 weighted FLASH for heart and 3D-UTE for lung) were first acquired. 2D T1 weighted FLASH: repetition time/echo time=18.24/3.54 ms; flip angle, 50°; field of view, 36×36 mm²; matrix size, 138×138 mm²; slice thickness, 1 mm; acquisition time, 3 minutes 50 seconds. 3D-UTE: repetition time/echo time=4/0.01225 ms; flip angle, 16°; field of view, 32×32 mm²; matrix size, 128×128 mm²; slice thickness, 0.25 mm; acquisition time, 3 minutes 36 seconds. Then a bolus of Gd-9 complex was administered i.v. and imaging performed for a period of 40 min p.i. with the repetition acquisition of 2D T1 weighted FLASH and 3D-UTE sequences. Following the imaging session, animals were sacrificed and heart and lung tissue was subjected to histopathologic analysis.

For kidney imaging, a series of three-dimensional inversion-recovery FLASH images were acquired at baseline and 4 hours after Mn-16 administration with inversion recovery times of 7, 307, 557, 707, 857, 1507, 3007, 5007, and 7007 msec. (TR/TE/FA=5 see/72 msec, field of view (FOV) 30 mm×3 mm, dimension 128×128, slice thickness 0.7 mm).

For PDAC6 xenograft tumor imaging, images were acquired with following sequences and parameters: 3D T1 weighted FLASH (TR/TE/FA=14 ms/2 μs/30°, 0.4 mm isotropic spatial resolution, field of view (FOV) 35 mm×35 mm, two average, acquisition time=4.3 min) images were acquired prior to and dynamically post injection of contrast agent; 3D RARE (RARE, TR/TE/FA=1400 s/57 ms/180°, 0.3 mm isotropic spatial resolution, field of view (FOV) 35 mm×60 mm, one average, acquisition time=8.1 min) image was also acquired.

For liver imaging analysis, a region of interest (ROI) was manually traced encompassing the liver parenchyma while avoiding major blood vessels. A second ROI was placed on the dorsal muscle visible in the same image slice to quantify the signal intensity in the muscle for comparison. Seven ROIs were placed in the field of view without any tissue (air) to measure the variation in background signal. More than 20 axial slices per mouse across the entire liver were analyzed in this fashion. The same analysis was performed on the pre- and 45-minute post injection images acquired with the FLASH sequence. Contrast to noise ratio (CNR) was calculated by measuring the difference in signal intensity (SI) between liver and muscle and normalizing to the standard deviation of the signal in the air, eq 21. ΔCNR was calculated by subtracting CNR measured prior to probe injection (CNR_{Pre}) from CNR measured after injection (CNR_{Post}), eq 22.

$$C N R = ( {SI}_{liver} - {SI}_{m u s c l e} ) / {SD}_{a i r} \qquad ( 21 )$$

$$Δ C N R = C N {R}_{post} - C N {R}_{p r e} \qquad ( 22 )$$

For lung imaging analysis, RARE image, FLASH image pre-contrast and immediately post-contrast were used to define regions of interest (ROIs) in the lung that excluded vessels and airways. A total of 6 lung ROIs were defined on axial UTE images to obtain signal intensity (SI); ROIs in the dorsal muscle in each slice were also defined as reference; ROIs in the field of view without any tissue (air) were used to measure the variation in background signal. Then the lung-to muscle ratio (LMR) or contrast to noise ratio (CNR) was averaged from the 6 slices to calculate changes in LMR (ΔLMR) or CNR (ΔCNR) at each time point.

For heart imaging analysis, FLASH image pre-contrast and immediately post-contrast were used to define regions of interest (ROIs) in the myocardium that excluded blood vessels. The percentage of signal intensity change (% SI) was calculated by comparing the SI measured after probe injection to SI measured prior to probe injection.

For kidney imaging analysis, longitudinal relaxation time (T1) was obtained by fitting inversion recovery data to a T1 relaxation equation (M0×(1−2 exp(−TI/T1))) and change in T1 (ΔT1) was calculated by subtracting T1 measured prior to probe injection (T1ₚᵣₑ) from T1 measured after injection (T1ₚₒₛₜ), eq 23.

$$Δ T 1 = T {1}_{post} - T {1}_{p r e} \qquad ( 23 )$$

For tumor imaging analysis, RARE image and first FLASH image post-contrast were used to define volumetric region of interest (ROIs) in the tumor that excluded necrosis region. ΔCNR was calculated by eq 22.

TABLE A10
ΔCNR of living in CCl₄ injured mice at 45 min post injection (n =
6 in each group). The probes described herein had much higher signal in
fibrotic liver tissue compared to negative control GdDOTA and prior art GdCHyd.

| Probe | GdDOTA | GdCHyd | Gd-10 | Gd-9 | Mn-12 | Mn-15 |
|---|---|---|---|---|---|---|
| ΔCNR | 0.1 ± 0.2 | 0.2 ± 0.4 | 1.5 ± 0.9 | 2.2 ± 0.9 | 1.5 ± 0.8 | 1.2 ± 0.2 |

TABLE A11
Pair-wise comparison of ΔCNR of liver in CCl₄ injured mice
imaged with Gd-9 or Gd-10 at 45 min post injection (n = 6).
Gd-9 is superior to Gd-10 for imaging liver fibrogenesis.

| Mice ID | Gd-10 | Gd-9 |
|---|---|---|
| 1 | 0.9 | 1.3 |
| 2 | 1.4 | 2.1 |
| 3 | 1.0 | 1.5 |
| 4 | 1.3 | 1.9 |
| 5 | 2.3 | 3.2 |
| 6 | 1.2 | 1.9 |

TABLE A12
Pair-wise comparison of ΔCNR of liver in CCl₄ injured
mice imaged with Gd-9 at 45 min post injection with or
without blocking dose of Yb-9 (n = 3). Blocking with Yb-9
indicates that the uptake seen with Gd-9 is specific.

| | Without | With |
|---|---|---|
| Mice ID | blocking | blocking |
| 1 | 3.0 | 0.1 |
| 2 | 3.6 | 0.1 |
| 3 | 2.7 | 0.3 |

TABLE A13
Change in lung to muscle ratio (ΔLMR) in lungs of fibrotic
(n = 13 for Mn-15, n = 8 for Mn-16, n = 12 for Mn-17, n = 5 for
Mn-18) and naïve (n = 6 for Mn-15, n-6 for Mn-16, n = 6 for
Mn-17, n = 4 for Mn-18) mice as determined from MR imaging.
Targeted probes Mn-15, Mn-16, and Mn-17 specifically
enhance injured lung but not normal lung. The untargeted probe
Mn-18 does not significantly enhance injure lung.

| | Mn-15 | Mn-16 | Mn-17 | Mn-18 |
|---|---|---|---|---|
| Bleomycin injured | 0.13 | 0.18 | 0.15 | 0.04 |
| mice | | | | |
| Naïve mice | 0.00 | 0.00 | 0.00 | 0.01 |

TABLE A14
Washout T_{1/2} (min) of Mn-15, Mn-16, Mn-17 and Mn-18 in
lungs of bleomycin-injured and naïve mice. The residency time
of the probes is similar in normal lung but dramatically increased
in injured lungs with fibrogenesis for the targeted probes Mn-15,
Mn-16, and Mn-17, but not for the untargeted probe Mn-18.

| | Mn-15 | Mn-16 | Mn-17 | Mn-18 |
|---|---|---|---|---|
| Belomycin injured | 33.6 ± 5.1 | >4 h | 41.5 ± 6.3 | 18.9 ± 2.0 |
| mice | | | | |
| Naïve mice | 13.7 ± 1.4 | 17.9 ± 3.2 | 15.1 ± 1.9 | 15.7 ± 1.8 |

TABLE A15
Pair-wise comparison of ΔLMR in lungs of
fibrotic and naïve mice at 60 min post injection
with Mn-15 or Mn-17 (n = 6 in each group)

| Mice ID | Mn-15 | Mn-17 |
|---|---|---|
| 1 | 0.18 | 0.18 |
| 2 | 0.08 | 0.15 |
| 3 | 0.15 | 0.15 |
| 4 | 0.06 | 0.09 |
| 5 | 0.11 | 0.18 |
| 6 | 0.18 | 0.27 |

TABLE A16
ΔCNR of liver in CDAHFD mice at 20 min post injection imaged with Gd-9
(n = 6 in each group) and corresponding ex vivo analysis of the percentage
of lysine aldehyde and lysyl oxidase positive tissues. Gd-9 MRI signal
enhancement increases in liver tissue and tracks with levels of fibrogenesis
markers like lysine aldehyde staining and lysyl oxidase staining.

| | | 2 wk | 6 wk | 10 wk | 1 wk | 4 wk |
|---|---|---|---|---|---|---|
| Group | Control | CDAHFD | CDAHFD | CDAHFD | Withdrawal | Withdrawal |
| ACNR | 0.1 ± 0.4 | 1.3 ± 0.3 | 2.1 ± 0.4 | 2.5 ± 0.5 | 0.9 ± 0.3 | 0.3 ± 0.2 |
| Lysine | 8.3 ± 1.8 | 15.4 ± 2.4 | 17.8 ± 2.0 | 17.3 ± 2.2 | 12.0 ± 1.0 | 12.0 ± 1.8 |
| aldehyde | | | | | | |
| (%) | | | | | | |
| Lysyl | 2.3 ± 0.6 | 8.3 ± 2.0 | 13.0 ± 1.3 | 17.7 ± 2.1 | 6.7 ± 1.5 | 3.7 ± 1.2 |
| oxidase | | | | | | |
| (%) | | | | | | |

TABLE A17
ΔCNR of liver in sham (n = 4 or bile duct ligated (BDL,
n = 7) rats at 30 min post injection imaged with Gd-9.
Gd-9 detects liver fibrogenesis in this rat model.

| Group | Sham rats | BDL rats |
|---|---|---|
| ΔCNR | 0.6 ± 0.2 | 3.4 ± 1.6 |

TABLE A18
Pair-wise comparison of ΔCNR of lung in bleomycin injured
mice imaged with Gd-CHyd or Gd-9 at 25 min post injection (n = 4).
Gd-9 is superior to Gd-CHyd for imaging lung fibrogenesis.

| Mice ID | GdCHyd | Gd-9 |
|---|---|---|
| 1 | 2.2 | 4.3 |
| 2 | 1.2 | 3.0 |
| 3 | 2.0 | 4.0 |
| 4 | 2.9 | 3.7 |

TABLE A19
% SI change of myocardium in SAMP8 or SAMR1 mice with or without transverse
aortic constriction (TAC) imaged with Gd-9 at 20 min post injection (n = 4 for each
group), and corresponding ex vivo analysis of the allysine and hydroxyproline levels.
TAC animals have cardiac fibrosis and fibrogenesis as indicated by higher hydroxyproline
and allysine levels compared to animals undergoing a sham procedure. MRI signal
enhancement with Gd-9 is 3-fold higher in TAC animals with cardiac fibrogenesis.

| Group | SAMP8-TAC | SAMP8-Sham | SAMP1-TAC | SAMP1-Sham |
|---|---|---|---|---|
| % SI | 22 ± 7.9 | 8.2 ± 5.3 | 21.5 ± 8.4 | 7.8 ± 4.7 |
| Allysine | 22.5 ± 4.7 | 13.1 ± 6.3 | 8.3 ± 5.0 | 5.2 ± 3.1 |
| (nmol/g) | | | | |
| Hydroxyproline | 545 ± 71 | 402 ± 23 | 610 ± 78 | 408 ± 89 |
| (μg/g) | | | | |

TABLE A20
ΔCNR of lung in SAMP8 or SAMR1 mice with or without transverse aortic constriction
(TAC) imaged with Gd-9 at 20 min post injection (n = 4 for each group), and
corresponding ex vivo analysis of the allysine and hydroxyproline levels. TAC animals
also have pulmonary fibrosis and fibrogenesis as indicated by higher
hydroxyproline and allysine levels compared to animals undergoing a sham procedure.
MRI signal enhancement in lung with Gd-9 is 3-fold higher in TAC animals.

| Group | SAMP8-TAC | SAMP8-Sham | SAMP1-TAC | SAMP1-Sham |
|---|---|---|---|---|
| ΔCNR | 3.1 ± 0.9 | 0.8 ± 0.5 | 1.2 ± 0.3 | 0.4 ± 0.2 |
| Allysine | 5.5 ± 4.1 | 5.1 ± 2.3 | 4.2 ± 2.0 | 1.6 ± 1.5 |
| (nmol/g) | | | | |
| Hydroxyproline | 189 ± 76 | 148 ± 16 | 217 ± 35 | 144 ± 19 |
| (μg/g) | | | | |

TABLE A20
Change in longitudinal relaxation time (ΔT1) in cortex of
IRI and contralateral kidney at 4 h post injection of Mn-16
(n = 11). Mn-16 significantly shortens the T1 value of
the injured, fibrogenic kidney in preference to the uninjured
contralateral kidney and Mn levels are
significantly higher in the injured kidney cortex.

| | IRI kidney | contralateral | t-test |
|---|---|---|---|
| | cortex | cortex | p-value |
| ΔT1 | −395 ms | −230 ms | <0.01 |
| Hydroxyproline | 1060 | 380 | <0.0001 |
| (μg/g) | | | |
| Mn concentration | 90 | 60 | <0.05 |
| (nmol/g) | | | |

TABLE A21
ΔCNR of PDAC6 xenograft tumor pre- and 3-days post-treatment
injected with targeted probe Mn-17 and non-targeted control probe
Mn-18 at 60 min post injection (n = 4 for each group). Targeted
Mn-17 shows much higher tumor enhancement than untargeted
Mn-18. Tumor enhancement is further increased with Mn-17 after
Folfirinox treatment with creates additional tumor fibrogenesis.

| | Mn-17 | Mn-18 |
|---|---|---|
| Pre-treatment | 4.6 ± 1.0 | 0.4 ± 0.3 |
| 3-days post treatment | 6.4 ± 1.2 | 0.4 ± 0.3 |

### Conclusion

Gd-9 and Gd-10 are Gd-DOTA derivatives with two hydrazine arms but different orientation. Gd-CHyd contains only one hydrazine arm. The dual binding probe Gd-9 has faster on-rate (600%), slower off-rate (50%), higher protein-bound relaxivity (50%) than a monobinder Gd-CHyd and markedly superior performance (10-fold higher ΔCNR) in vivo for measuring liver fibrogenesis. Both Gd-9 and Gd-10 enhanced MRI show significantly higher liver-to-muscle contrast to noise ratio (ΔCNR) and slower liver washout rate in CCl₄ injured mice than Gd-CHyd and Gd-DOTA. At 45 min p.i., ΔCNR for Gd-9 and Gd-10 was 2.2±0.8 and 1.3±0.4 respectively in CCl₄ injured mice, while there was no significant liver enhancement in the Gd-CHyd or Gd-DOTA groups, nor in any of the vehicle-treated mice. Pair-wise comparison between Gd-9 and Gd-10 in CCl₄ injured mice showed that ΔCNR was consistently and significantly higher in the mice imaged with Gd-9 compared to Gd-10. The blocking study using Yb-9 showed that the 10-fold dose of Yb-9 completely eliminated liver MRI enhancement with Gd-9, demonstrating the specific binding of Gd-9 to the livers of fibrotic mice. In addition, Gd-9 could also specifically detect liver fibrogenesis in dietary-induced mouse models, a cholestasis rat model of liver fibrogenesis and bleomycin injured pulmonary fibrogenesis.

In a mouse model of non-alcoholic steatohepatitis, Gd-9 molecular MRI could detect the early onset of liver fibrosis (prior to significant increases in liver hydroxyproline) and was very sensitive to a reduction in fibrogenesis following a therapeutic intervention. Gd-9 ΔCNR tracked well with measures of fibrogenesis like expression of lysyl oxidase and lysine aldehyde. The change in molecular MRI signal preceded the presence or resolution of fibrosis assessed biochemically and histologically which depend on the change of collagen concentration, highlighting this probe for early disease detection and as an early readout of response to effective therapy.

Mn-12 is an analogue of Gd-9. It showed similar in vitro reactivity to Gd-9 in reaction with lysine aldehyde. Mn-12 is stable and kinetically inert, has low signal enhancement in normal liver, but has high affinity and marked turn-on relaxivity (4-folds) upon lysine aldehyde binding. The probe shows significantly enhanced liver signal in CCl₄ induced liver fibrosis mice than vehicle-treated mice.

Mn-15, Mn-16, and Mn-17 are reversible allysine-targeting Mn-PC2A derivatives differed in their association and dissociation kinetics and Mn-18 was synthesized as allysine non-reactive probe sharing same Mn-PC2A core.

The α-carboxylate moiety in Mn-15 results in a 3-fold higher condensation rate constant (12.1 vs 2.5 M⁻¹s⁻¹) as well as 2-times higher hydrolysis rate constant (1.03×10⁻³ vs 0.41×10⁻³ s⁻¹) compared to Mn-17. Mn-16 exhibited slowest condensation rate constant (2.5 M⁻¹ s⁻¹) but 103 greater hydrolytic stability (hydrolysis rate constant=0.79×10⁻⁶ s⁻¹). Mn-15, Mn-16, and Mn-17 exhibited specific binding to allysine, and 4-fold turn on in relaxivity upon binding. The hydrazine/oxyamine bearing probe exhibited specific uptake in a disease model of pulmonary fibrosis resulting in significantly enhanced MRI signal and prolonged MRI signal enhancement in fibrotic tissue. Compared with Mn-15, Mn-17 with greater hydrolytic stability (but slower on-rate) exhibited longer washout T_{1/2} in fibrotic lung (For Mn-15, 33.5±5.1 min in fibrotic lung and 13.7±1.4 min in normal lung. For Mn-16, >4 h in fibrotic lung and 17.9±3.2 min in normal lung. For Mn-17, 41.5±6.3 min in fibrotic lung and 15.1±1.9 min in normal lung). and was significantly superior in detecting fibrogenesis, highlighting the significance of modulating bond dissociation kinetics in achieving higher probe sensitivity.

### Example B. Optimization of Gd(III) Probes for Quantitative Imaging of Liver Fibrogenesis: Impact of Dual Targeting Groups on Relaxivity, On-Rate, and Off-Rate

Reaction with Butyraldehyde Monitored by HPLC-ICP-MS

Reactions were conducted in pH 7.4 phosphate-buffered saline with butyraldehyde concentration of 100 μM and hydrazine probes at 25 μM. Concentrations of unreacted starting material and condensation products were determined based on their relative integrations at 12 min time intervals over 2 hours.

### Sodium Dodecyl Sulfate Polyacrylamide Gel Electrophoresis Analysis

BSA^{Ald} or BSA (10 mg/mL, 300 μL) was incubated with 200 μM Gd-9 at 37° C. in pH 7.4 PBS for 24 h. Then sodium cyanoborohydride (10 mg) was added to the solution and reacted for an additional further 2 h to irreversibly bind the probe to the protein. After the protein species were collected by ultrafiltration, 200 μL PBS was added to redissolve the protein. An aliquot of the solution was diluted in Tris-Glycine SDS sample buffer (1×) to give a final protein concentration of ˜4 μg/μL, with a total volume of 40 μL. Then 5 μL of the samples and a molecular weight standard (PageRuler Prestained Protein Ladder No. 26619) were resolved electrophoretically using a 4-20% Tris-Glycine Gel (Thermo Fisher Scientific, XP04200BOX). Subsequently the proteins were then identified in the gel using silver staining (Thermo Fisher Scientific, 24600).

### ECM Extraction

Liver fibrosis was induced in male Sprague Dawley rats by ligation of the common bile duct (CD®, Charles River Labs, Wilmington, MA). Ten days after common bile duct ligation, the fibrotic liver was harvested and sequential extractions were performed using the CNMCS (Cytosol/Nucleus/Membrane/Cytoskeleton) Compartmental Protein Extraction kit (Cytomol, Union City, CA) according to manufacturer's instructions. In brief, frozen tissues (150-200 mg) were homogenized and with solvents provided in the kit to sequentially to remove: cytosolic proteins, nuclear proteins, membrane proteins, and cytoskeletal proteins, leaving a final insoluble fraction enriched for ECM proteins.

### Relaxivity Measurements in ECM

The enriched ECM proteins from 800 mg of fibrotic rat liver were suspended in 1 ml PBS. The ECM protein concentration was about 10% (5 mg in a 50 μL aliquot determined by weight after lyophilization). An aliquot of the solution (50 μL) was treated for 2 h at 37° C. with the corresponding molecular probe at a range of concentrations (0.01-0.1 mM) and then longitudinal (T₁) relaxation measurements was recorded using a Bruker mq60 Minispec at 1.41 T and 37° C. For the blocking experiment with N₂H₄, a 100 fold-excess of N₂H₄ referred to the concentration of molecular probe was added to the solution before the incubation. Longitudinal (T₁) relaxation times were measured via an inversion recovery experiment using 10 inversions of duration ranging between 0.05×T₁ and 10×T₁. Relaxivity (r₁) was determined from the slope of a plot of 1/T₁ vs metal concentration for 4 concentrations.

### Tissue Analysis for Mice and Rats

General method. After imaging, the animals were sacrificed under anesthesia, and the liver tissues were harvested. A piece of left lobe of the liver was fixed in 4% paraformaldehyde in PBS, dehydrated, embedded in paraffin, and then sectioned into 5-μm-thick slices for later staining with Sirius red and hematoxylin and eosin (H&E). Another piece of left lobe was fixed in methacarn (methanol-Carnoy), dehydrated, embedded in paraffin, and sectioned into 7-μm-thick slices for detection of aldehyde. A remaining piece of left lobe was quickly frozen in liquid nitrogen for later hydroxyproline analysis.

The collagen proportional area (CPA), defined as the percentage of the area stained positive by Sirius red, was measured with ImageJ (Fiji, version 1.0) as previously described. For NASH components, H&E sections were evaluated and morphometric quantitation of hepatic steatosis, expressed as percentage of lipid vacuolization, was performed using ImageJ (Fiji, version 1.0).

LOX protein detection. LOX protein expression in the liver tissue was detected using immunohistochemistry assays with antibody against LOX (NB100-2527, 1:100, Novus Biologicals, Littleton, CO, USA). After deparaffinization and hydration of the paraffin-embedded liver sections, the endogenous peroxidase activity was inhibited using 0.3% hydrogen peroxide and antigens were retrieved using 10 mM sodium citrate buffer (pH 6.0) at 110° C. for 30 min. After the tissue was blocked using 2% triton, they were reacted with the anti-LOX antibodies at room temperature overnight. Subsequently, the slices were incubated with anti-goat IgG conjugated with peroxidase for 1 h at room temperature and then treated with DAB and counterstained with hematoxylin before dehydration and mounting.

Hydroxyproline assay. Hydroxyproline in liver was quantified by HPLC analysis using a reported method, and was expressed as amounts per wet weight of tissue.

DNPH reactivity assay for determining Lys^{Ald} levels. The staining was carried out as previously described. After deparaffinization and hydration were performed on the 7 μm-thick methacarn-fixed liver sections, the tissue was reacted with 1 mg/mL dinitrophenylhydrazine (DNPH; TCI D0846, VWR) in 2 M HCl for 30 min. After the sections were neutralized with PBS, they were blocked with horse serum and then sequentially exposed to anti-DNP antibody (D9656, 1:2,000, Sigma-Aldrich, St. Louis MO), biotinylated secondary antibody, enzyme linked avidin-biotin complex, and Vector Red substrate (SK-5100, Vector Laboratories, Burlingame CA) diluted in levamisole (SP-5000, Vector Laboratories) and 100 mM Tris-HCl, pH 8.8 for 4 min before being counterstained with methyl green, dehydrated, and having a coverslip applied. The slides were scanned using NanoZoomer Slide Scanner (Hamamatsu) at 40× original magnification. Subsequently, four nonoverlapping, randomly oriented, 1000×600, 250×150, 1000×600 μm image fields were obtained for the CCl₄, NASH and BDL study, respectively, were picked for each tissue, avoiding large blood vessels and the tissue edge. For CCl₄ and BDL study, the percentage of DNPH-reactive tissue in the image fields was then measured with ImageJ (Fiji, version 1.0), similar to the calculation of CPA. For NASH study, the images were segmented using a uniform method to exclude cell nucleus and lipids, and the mean integrated intensities were determined using ImageJ (Fiji, version 1.0). During the image sampling, segmentation, and staining intensity determination, the involved investigator that was masked with respect to the animal treatment group.

### Human Sample Collection

Liver tissues were obtained from a total of 9 anesthetized patients who underwent surgical resection at Massachusetts General Hospital, and analyzed in accordance with a protocol approved by the Massachusetts General Hospital's Institutional Review Board. The clinical characteristics of the subjects are summarized in Table C.

### Tissue Analysis for Human Tissues

The dissected tissues were immediately snap frozen in OCT using liquid nitrogen, sectioned into 10-μm-thick slices and stored at −80° C. Subsequently, the tissue sections were first allowed to warm to room temperature for 10 min and then fixed in 60% ethanol for 30 min, and washed with PBS. The following steps were the same as described above for the Sirius red staining and DNPH-reactivity assay.

Protein expression of LOX in the human tissue sections was detected using indirect immunofluorescence assays using with antibodies against LOX (ab174316, 1:50, Abcam), fluoroprobe-conjugated secondary antibody (A32733, 1:300, Invitrogen). The nuclei were identified using DNA-staining with DAPI. Finally, LOX-positive cells were detected using a fluorescence microscope (EVOS FL).

### LA-ICP-MS Gadolinium Imaging Analysis

Sample preparation. Harvested rat livers after MR imaging were immediately snapfrozen in OCT using liquid nitrogen, sectioned into 10-μm-thick slices, stored in −80° C. One slice was assessed by LA-ICP-MS directly. One adjacent slide was incubated with 0.1 mM Gd-9 at 37° C. for 2 h, followed with 0.2 mM NaBH₃CN for 2 h and washed with PBS (3×10 min). Another adjacent slide was incubated with 0.1 mM Gd-9 and 10 mM hydrazine at 37° C. for 2 h, followed with 0.2 mM NaBH₃CN for 2 hand washed with PBS (3×10 min). Similar procedures were carried out for human tissues. Slides were incubated with 0.1 mM Gd-9 at 37° C. for 2h, followed with 0.2 mM NaBH₃CN for 2 h and washed with PBS (3×10 min).

Gadolinium mapping. Elemental images were collected from sections of liver tissue via laser ablation inductively coupled plasma mass spectrometry at the Biomedical National Elemental Imaging Resource (BNEIR). The instrumentation used was a New Wave Research 213 nm laser ablation system with a 10 cm² sample chamber, interfaced with an Agilent 7900 ICP-MS system. Samples were ablated in no-gas mode, with helium as a carrier gas at a flow rate of 600 L min⁻¹. The laser power was 65% and the frequency was 20 Hz. Ablation patterns consisted of continuous lines, with either a 12 μm or 20 m square beam and a scan speed of 120 μm sec⁻¹ or 200 μm sec⁻¹ respectively, as noted. The acquisitions time for gadolinium (mass 157) was 0.015 s. Elemental imaging data was quantified using metal-doped gelatin standards prepared at BNEIR and National Institute of Standards and Technology (NIST) certified references material 1515 (apple leaves). Data reduction was performed in the Iolite software application, using carbon (mass 13) as an internal standard to normalize the data.

### Statistics

Data are displayed as box plots with the dark band inside the box representing the mean, the bottom and top of the box representing the first and third quartiles, and the whiskers the minimum and maximum values. Data are reported as the mean±standard deviation. Differences between two groups for un-paired study were tested with two-tailed unpaired t-Test. Differences between two groups for paired study were tested with two-tailed paired t-Test. Differences among more than two groups were tested with one-way analysis of variance (ANOVA) followed by Tukey's post-hoc test with P<0.05 considered as significant.

### Results

Fibrosis is an outcome of tissue repair response following tissue injury, which can result in organ dysfunction and failure. It is accompanied by upregulation of lysyl oxidase and Lox-like enzymes which catalyze oxidation of lysine e-amino groups on extracellular matrix proteins (chiefly collagens) to form the aldehyde containing amino acid allysine which then undergoes cross-linking reactions with other proteins to stabilize the matrix. Gadolinium probes functionalized with a hydrazine moiety can bind to allysine in vivo to robustly stage and quantify fibrogenesis To boost the sensitivity of these probes, two hydrazine groups were introduced in one molecule to increase the reaction on-rate with allysine, increase the relaxivity of the protein-bound product, and lower its off rate. Interestingly, systematic study shows both the number and orientation of the hydrazine groups strongly impacts these properties which is manifested in a mouse model of liver fibrosis.

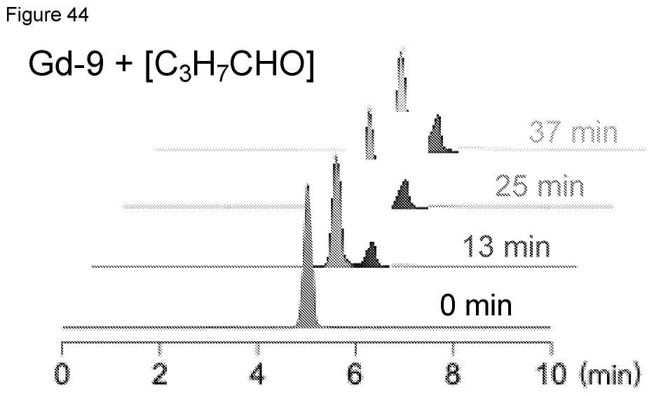

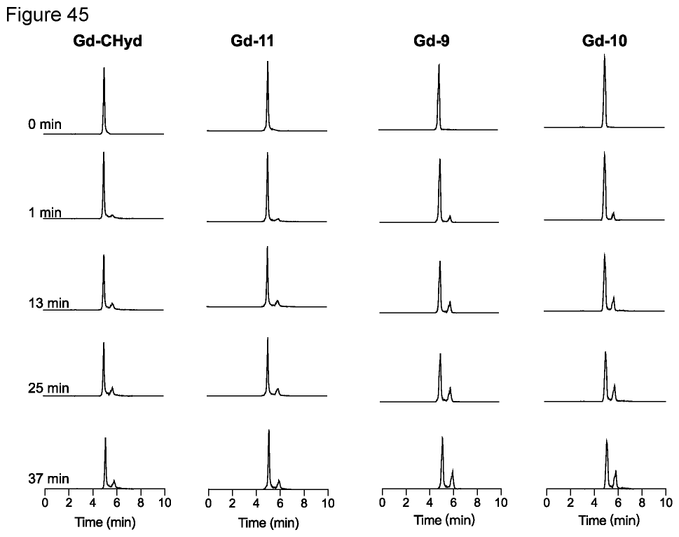

Using butyraldehyde as a small-molecule model of Lys^{Ald}, hydrazone formation was measured dynamically (FIG. 44 and FIG. 45). Introducing a second hydrazine moiety increased the on-rate and conversion yield by a factor of two and improved affinity 2-fold for Gd-9 and Gd-10 over Gd-11 and Gd-CHyd (FIGS. 44A and 44B).

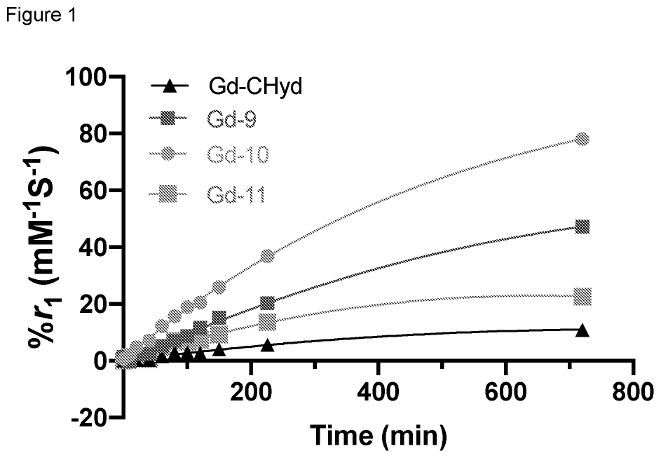

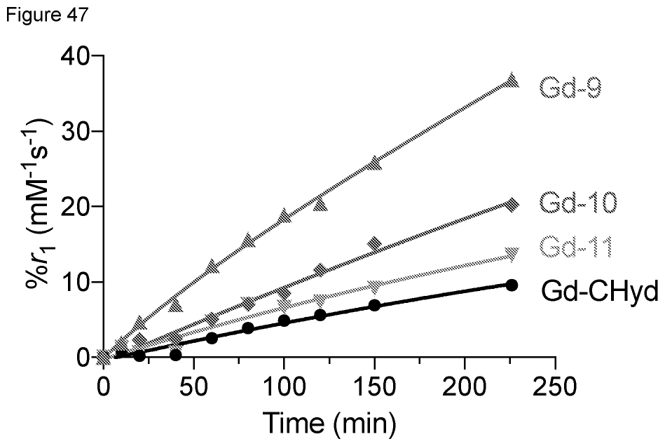

Gd-9 and Gd-10 are Gd-DOTA derivatives with two hydrazine arms but different orientation. Gd-11 is a control compound with one hydrazine and one piperazine. Compared to Gd-CHyd which only has one hydrazine moiety, Gd-9 and Gd-10 showed 6- and 3-fold higher initial on rate in the binding with allysine-modified BSA (BSA-Ald, FIG. 1 and FIG. 47).

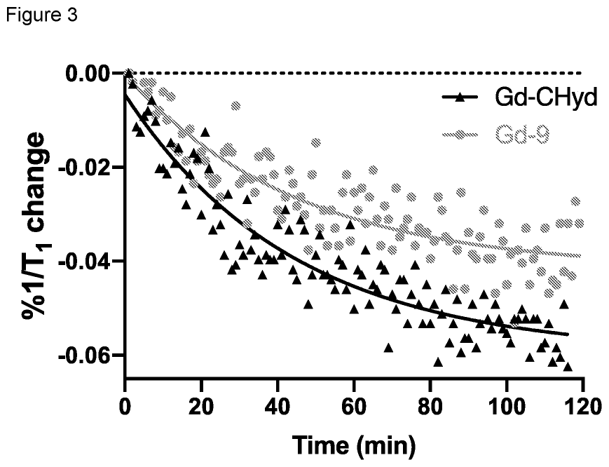

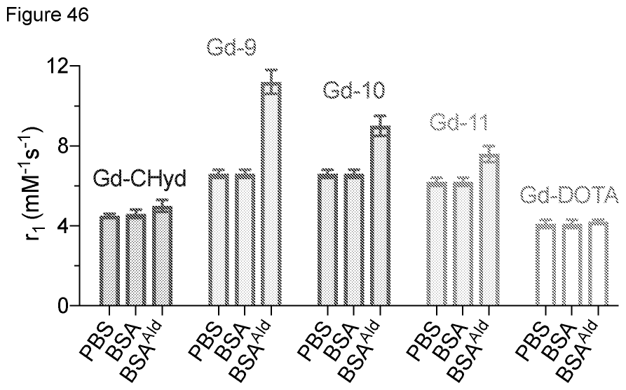

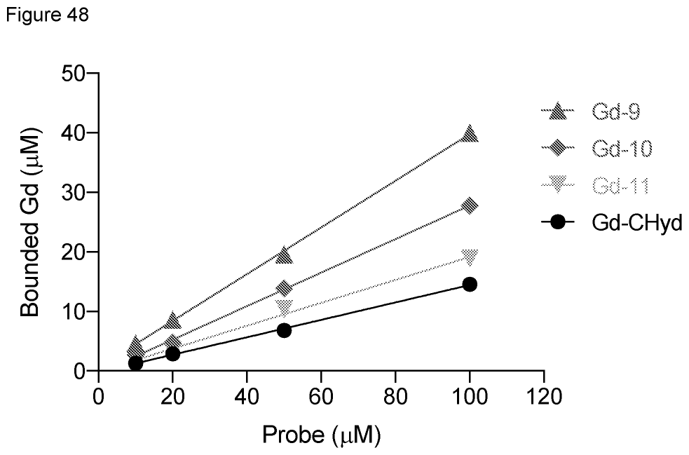

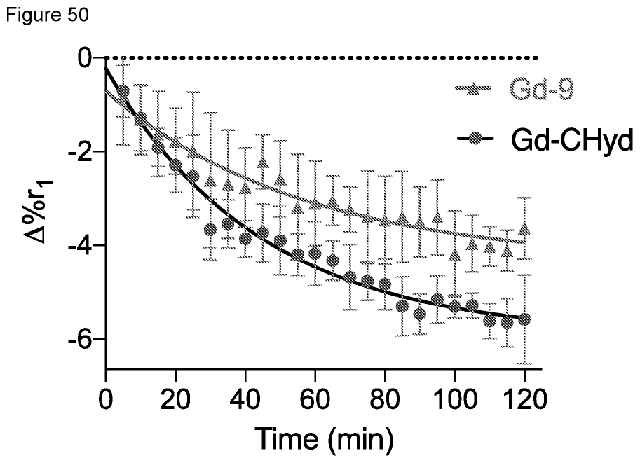

Under these conditions, equilibrium binding to BSA^{Ald} was 14.6, 19.2, 39.2, and 28.1% for Gd-CHyd, Gd-11, Gd-9 and Gd-10, respectively (FIG. 48). The relaxivity in the presence of BSA-Ald increased from 5.0 mM⁻¹s⁻¹ for GdCHyd to 11.7 mM-Is-1 for Gd-9 and 9.0 mM-1s-1 for Gd-10 (FIG. 2). The longitudinal relaxivities (r₁, 1.41 T, 37° C.) of the probes were measured in PBS alone, with excess unmodified BSA, or with excess of BSA^{Ald}. An increase in r₁ compared to the PBS value is indicative of protein binding. In unmodified BSA, r₁ values of Gd-CHyd, Gd-11, Gd-9 and Gd-10 were 4.5, 6.2, 6.6, 6.6 mM⁻¹s⁻¹ respectively, and unchanged from those in PBS (4.5, 6.2, 6.6, 6.6 mM⁻¹s⁻¹), indicating no appreciable nonspecific protein binding of the complexes. However, relaxivities (FIG. 46) increased in BSA^{Ald}, in the order of Gd-CHyd (5.0 mM⁻¹s⁻¹, 11% increase compared to PBS value)<Gd-11 (7.6 mM⁻¹s⁻¹, 23% increase)<Gd-10 (9.0 mM⁻¹s⁻¹, 36% increase)<Gd-9 (11.7 mM⁻¹s⁻¹, 77% increase). Gd-DOTA which lacks a hydrazine moiety showed no change in relaxivity (4.0 mM⁻¹s⁻¹) between PBS, BSA and BSA^{Ald} Dual binding complexes also show slower off-rate with BSA-Ald (FIG. 3 and FIG. 50).

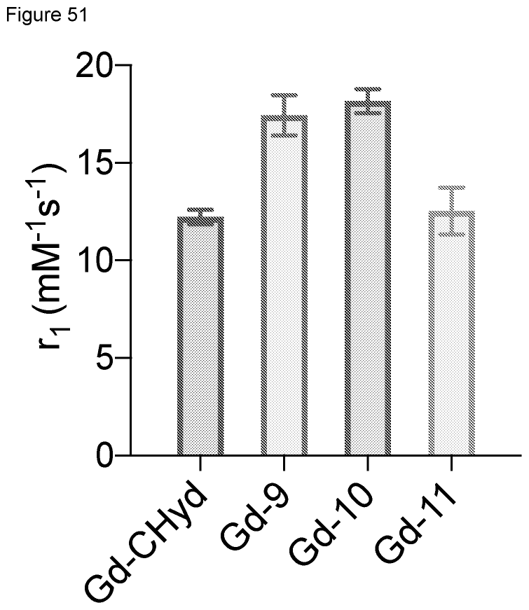

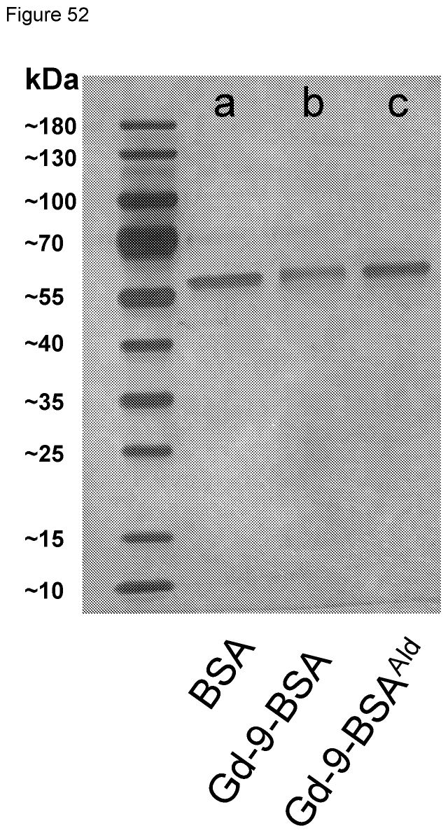

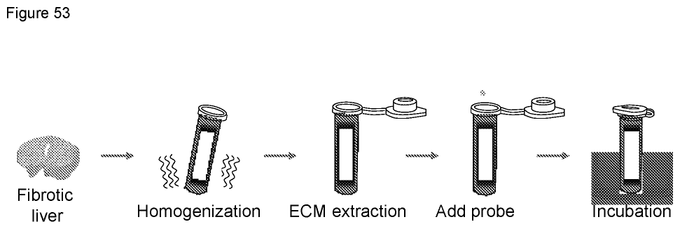

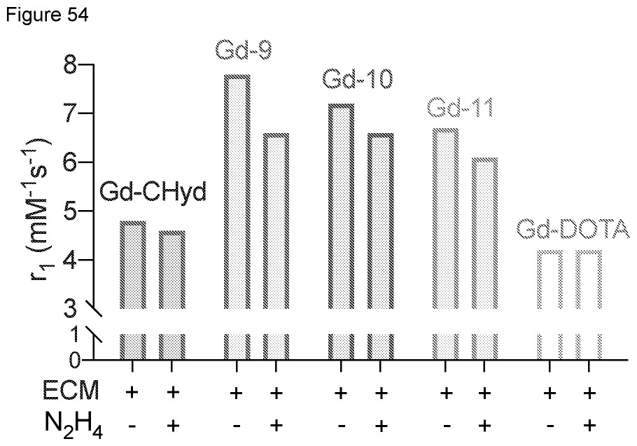

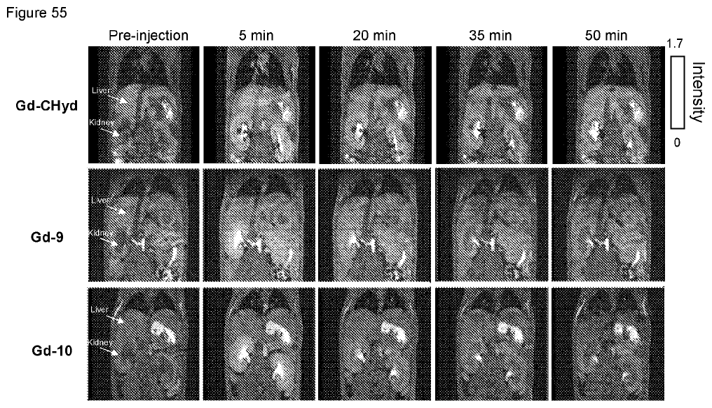

Interestingly, the cis-isomer Gd-9 shows a significantly higher on rate and relaxivity than the trans-isomer Gd-10 in the presence BSAAld, revealing the importance of tuning the orientation of targeting groups in designing dual binding probes. The difference reactivity between these two isomers was attributed to the different binding rate of the second hydrazine arm. To support this hypothesis, Gd-11 which has only one hydrazine was synthesized. Gd-11 shows similar protein-bound relaxivity and on-rate to the monohydrazine Gd-CHyd in BSA-Ald solution, indicating that the improved properties of Gd-9 stem from both hydrazine moieties binding allysine residues. This is further supported by the higher protein bound relaxivity for Gd-9 (17.7 mM-1s-1) and Gd-10 (18.7 mM-1s-1) compared to GdCHyd (11.8 mM-1s-1) and Gd-11 (13.7 mM-1s-1), which results from restricted internal rotation of the dual binders which promotes high relaxivity (see FIG. 51). Sodium dodecyl sulfate polyacrylamide gel electrophoresis analysis of the protein-bound Gd-9 showed that Gd-9 was only bound to one BSA^{Ald} and did not crosslink two proteins (FIG. 52). ECM suspension extracted from fibrotic rat liver was incubated and its relaxivity measured (FIG. 53). Gd-CHyd (4.8), Gd-11 (6.7), Gd-9 (7.8) and Gd-10 (7.2 mM⁻¹s⁻¹) all showed enhanced relaxivity in ECM (FIG. 54) compared to PBS, and relaxivity was significantly decreased by blocking ECM aldehydes with 100-fold excess of hydrazine (lowered to 4.6, 6.1, 6.6, 6.7 mM⁻¹s-1 respectively). Gd-DOTA, as negative control, showed similar relaxivity in ECM as in PBS.

Following intravenous (i.v.) administration (100 μmol/kg) of Gd-CHyd, Gd-9 or Gd-10 to normal mice, dynamic whole-body MRI showed that all probes were eliminated exclusively via the kidneys into the bladder (FIG. 55). The probes all showed an extracellular distribution with only transient liver enhancement consistent with blood pool. At 24 h post-injection (p.i.), >99% of the injected dose of gadolinium was eliminated from the body (Table B).

TABLE B
Retention of gadolinium in mice after 24 h injection. The gadolinium content in various
tissues and organs in normal mice 24 h after injection of corresponding probe (100 μmol/kg).
The mean ± standard deviation for each organ is tabulated (n = 4). ID = Injected Dose.
These probes are almost completely (>99%) eliminated from the body within 24 h.

| Gd-9 | | | Gd-10 | | |
|---|---|---|---|---|---|
| Organ | % ID per Gram | % ID per Organ | Organ | % ID per Gram | % ID per Organ |
| Serum | 0.00138 ± 0.000861 | 0.00376 ± 0.00113 | Serum | 0.00169 ± 0.000259 | 0.00398 ± 0.000175 |
| Brain | 0.00336 ± 0.00277 | 0.00130 ± 0.00125 | Brain | 0.00206 ± 0.000288 | 0.000776 ± 0.000169 |
| Lung | 0.166 ± 0.100 | 0.0289 ± 0.00394 | Lung | 0.157 ± 0.00422 | 0.0197 ± 0.00107 |
| Liver | 0.0686 ± 0.0102 | 0.0341 ± 0.0250 | Liver | 0.105 ± 0.00715 | 0.0572 ± 0.0426 |
| Muscle | 0.0132 ± 0.00294 | 0.105 ± 0.0225 | Muscle | 0.00886 ± 0.000914 | 0.0709 ± 0.00730 |
| Heart | 0.0788 ± 0.0151 | 0.00708 ± 0.000802 | Heart | 0.0219 ± 0.00212 | 0.00203 ± 0.000132 |
| Spleen | 0.140 ± 0.0558 | 0.00618 ± 0.000767 | Spleen | 0.165 ± 0.0817 | 0.0086 ± 0.00085 |
| Stomach | 0.114 ± 0.0288 | 0.0157 ± 0.00274 | Stomach | 0.0414 ± 0.02775 | 0.00869 ± 0.00207 |
| Bone | 0.0144 ± 0.0125 | 0.0455 ± 0.0174 | Bone | 0.0180 ± 0.00486 | 0.0469 ± 0.0126 |
| Kidneys | 1.33 ± 0.152 | 0.377 ± 0.0103 | Kidneys | 1.45 ± 0.262 | 0.365 ± 0.0315 |
| Sum | | 0.625 | | | 0.584 |

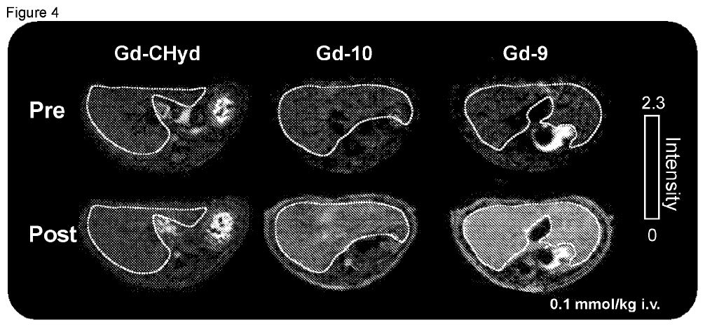

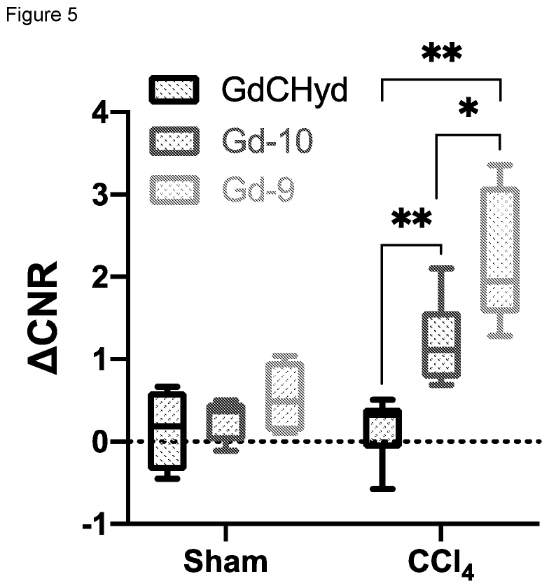

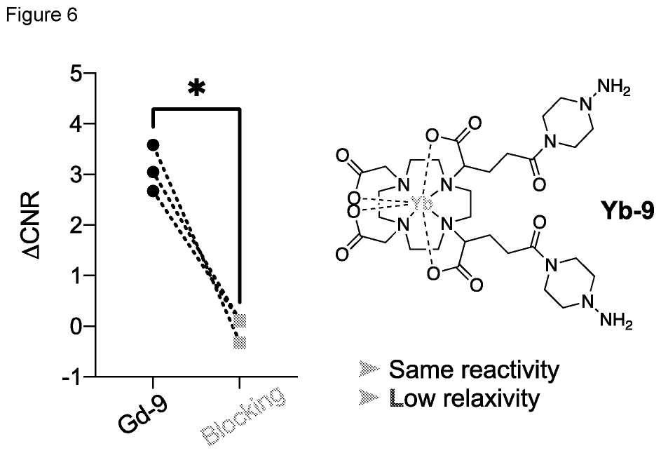

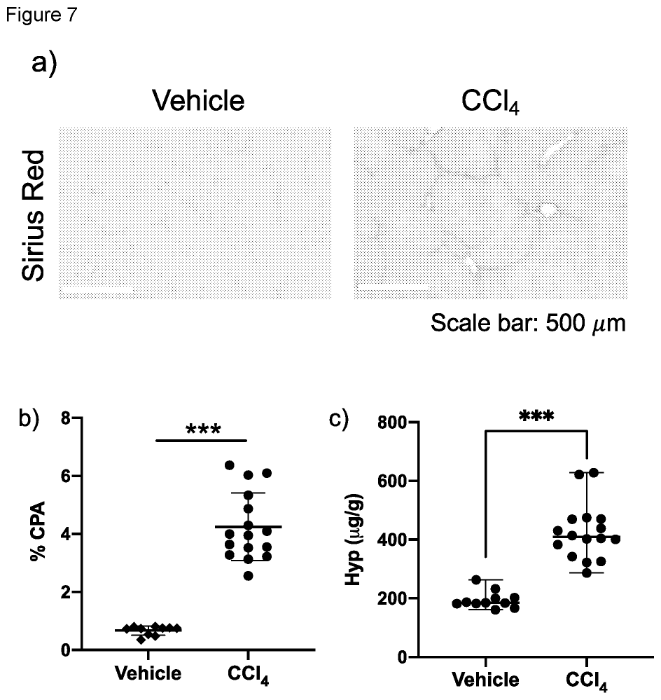

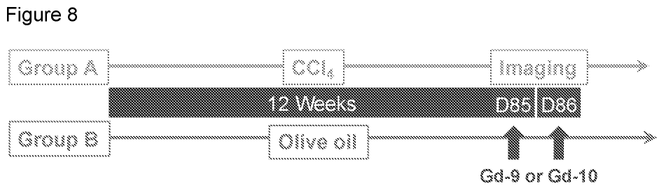

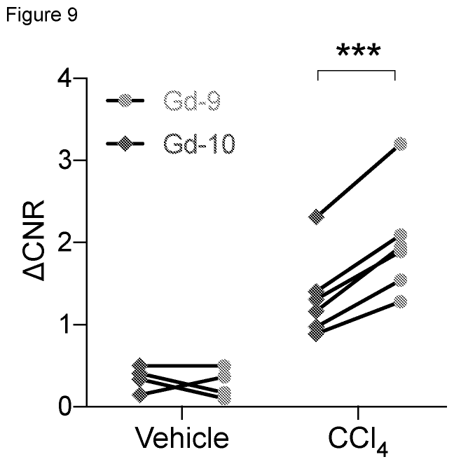

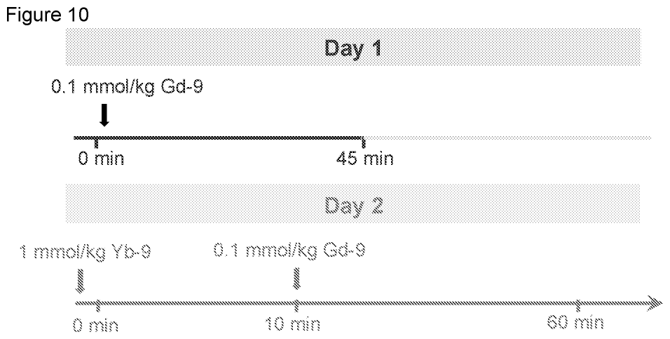

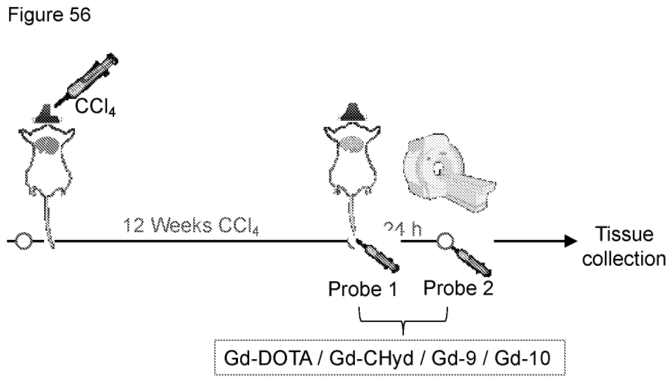

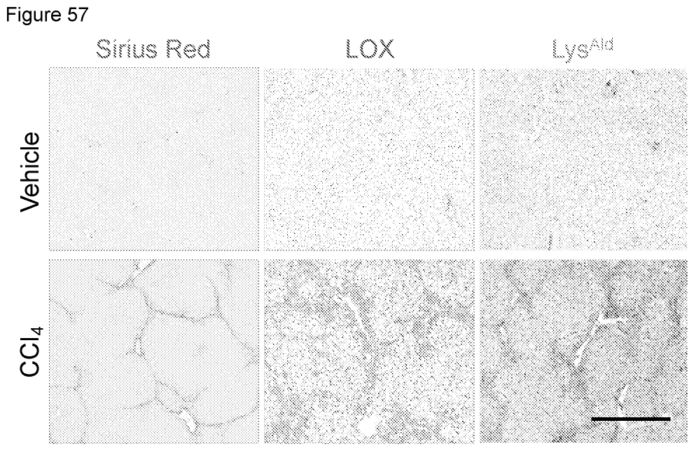

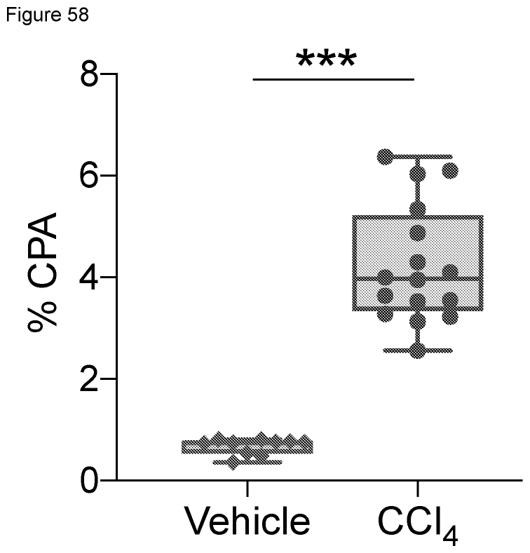

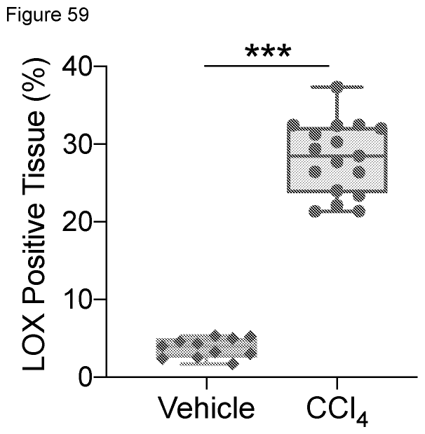

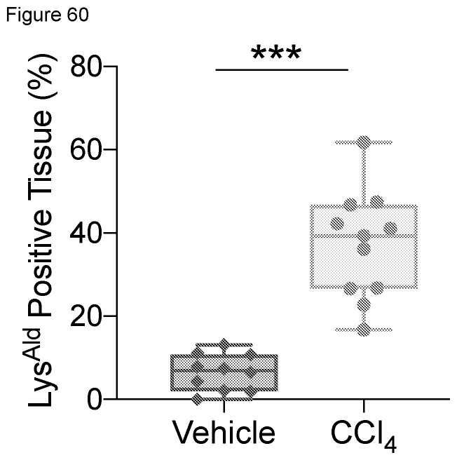

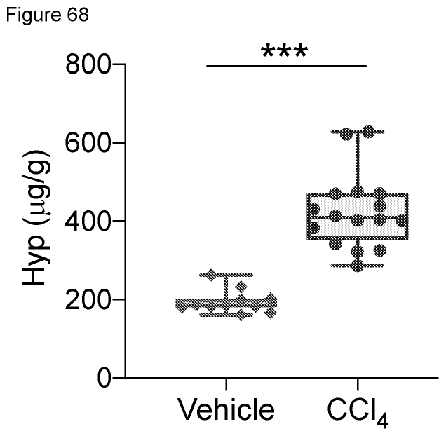

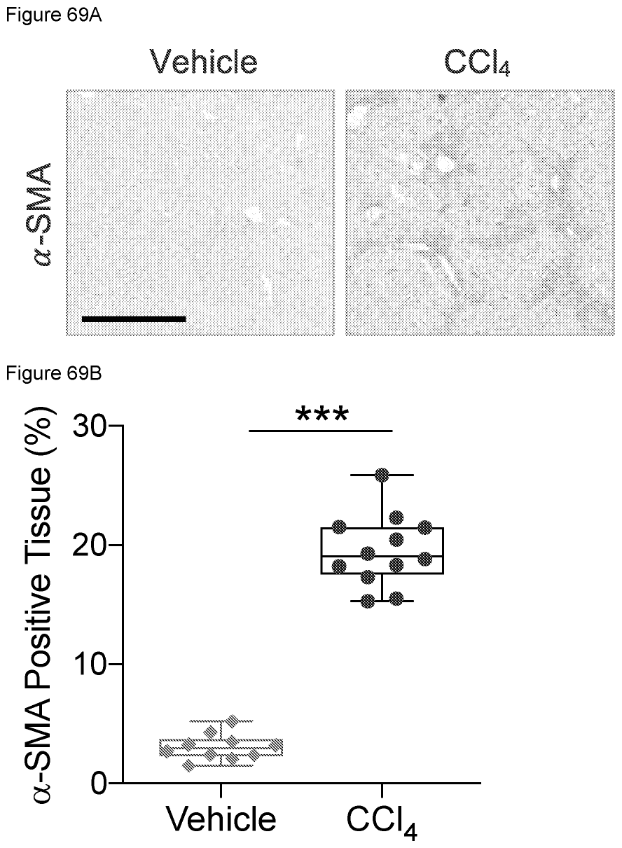

Next, Gd-DOTA, Gd-CHyd, Gd-9, and Gd-10 were tested in mice treated with CCl₄ or olive oil vehicle for 12 weeks to induce liver fibrosis. Ex vivo analyses of liver hydroxyproline (marker of collagen) and Sirius Red staining indicated consistent fibrosis in the CCl₄ group (FIG. 7). Both Gd-9 and Gd-10 enhanced MRI show significantly higher liver-to-muscle contrast to noise ratio (DCNR) in CCl4 injured mice than Gd-CHyd, and Gd-9 shows significantly higher DCNR than Gd-10 (FIG. 4 and FIG. 5). Gd-9 and Gd-10 were also compared in the same mice, imaged 24 hours apart, and this pairwise study showed Gd-9 giving consistently higher DCNR than Gd-10 (FIG. 8 and FIG. 9). To further demonstrate the specificity of Gd-9 for liver fibrogenesis, a blocking study was performed using Yb-9 where the Gd³⁺ ion is replaced by Yb³⁺ (FIG. 10). FIG. 6 shows DCNR for CCl₄ injured mice imaged first with Gd-9 and then the next day imaged 15 minutes after a 10-fold higher blocking dose of Yb-9 showing a complete block with Yb-9 and demonstrating the specificity of Gd-9 for fibrotic liver. In additional studies, all mice were imaged twice with different probes given randomly one day apart (FIG. 56). Collagen staining of liver tissue with Sirius Red (SR, FIG. 57) indicated consistent fibrosis in the CCl₄ group, with collagen proportional area (CPA) elevated to 4.2±1.2% vs. 0.7±0.2% in vehicle group (FIG. 58). Immunohistochemistry (IHC) of LOX on the liver sections revealed that the proportion of LOX positive tissue, hardly detectable in vehicle group (3.7±1.2%), was strongly increased in the CCl₄ group (28.2±4.6%, FIG. 59). The proportion of Lys^{Ald} positive tissue evaluated by dinitrophenylhydrazine (DNPH) reactivity assay also significantly increased in CCl₄ injured mice (37.1±13.0%) compared to vehicle controls (6.5±4.4%, FIG. 60). LOX immunoreactivity and Lys^{Ald} were both primarily observed along the fibrotic septa (FIG. 57) in a pattern similar to the distribution of collagen in SR and α-smooth muscle actin (α-SMA, FIG. 69). Hydroxyproline, a marker of total tissue collagen, was elevated in mice that received CCl₄ (425±95 μg/g vs. vehicle 195±29 μg/g liver, FIG. 68).

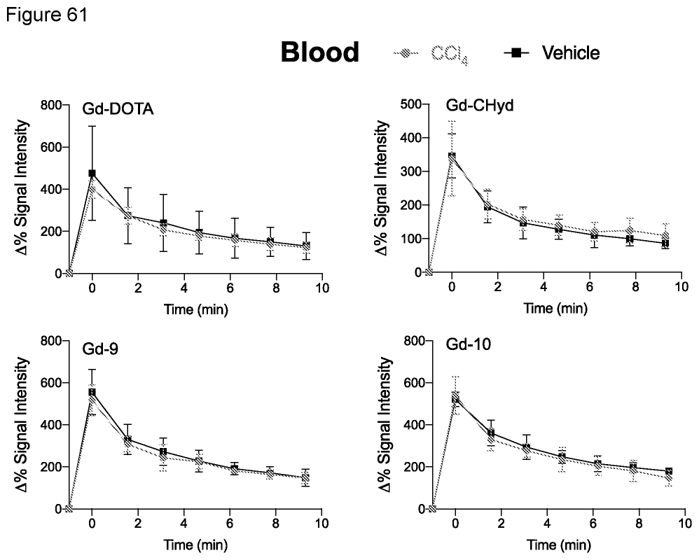

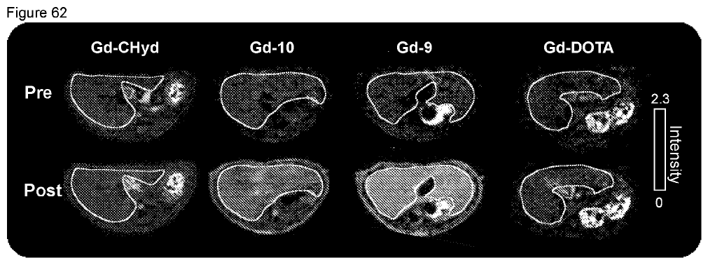

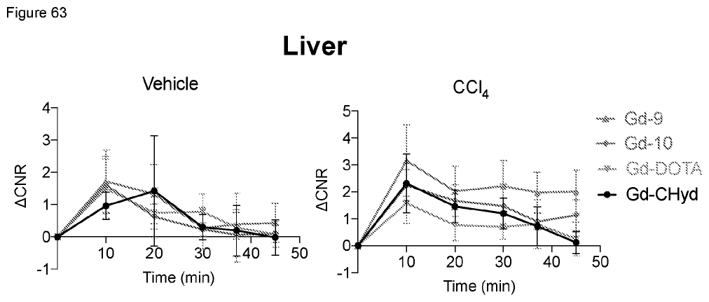

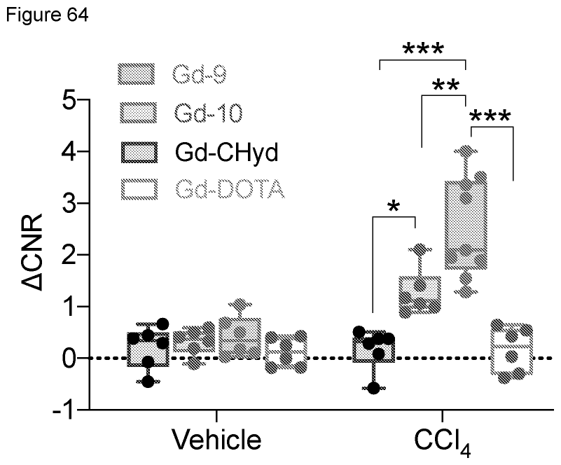

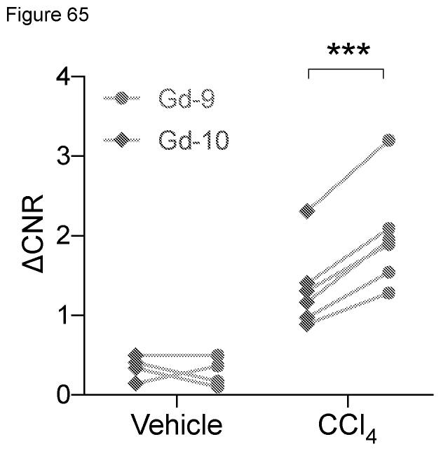

Blood elimination of the probes in CCl₄ injured mice was similar to that in the vehicle group (FIG. 61), but marked differences were observed in the liver. Both Gd-9 and Gd-10 enhanced MRI show significantly higher liver-to-muscle contrast to noise ratio (ΔCNR, FIG. 62) and slower liver washout rate in CCl₄ injured mice than Gd-CHyd and Gd-DOTA (FIG. 63). At 45 min p.i., ΔCNR for Gd-9 and Gd-10 was 2.2+0.8 and 1.3+0.4 respectively in CCl₄ injured mice, while there was no significant liver enhancement in the Gd-CHyd or Gd-DOTA groups, nor in any of the vehicle-treated mice (FIG. 64). Representative images at pre- and 45 min post-injection of each Gd³⁺ probe are shown in FIG. 62. Compared to Gd-CHyd, Gd-9 shows 10-fold higher ΔCNR. Importantly, Gd-9 showed significantly higher ΔCNR than Gd-10 (FIG. 64). To confirm this difference between isomers, mice were imaged with either Gd-9 or Gd-10 and then again the next day with the other probe. In CCl₄ injured mice, but not vehicle-treated animals, ΔCNR was consistently and significantly higher in the mice imaged with Gd-9 compared to Gd-10 (P<0.001, FIG. 65).

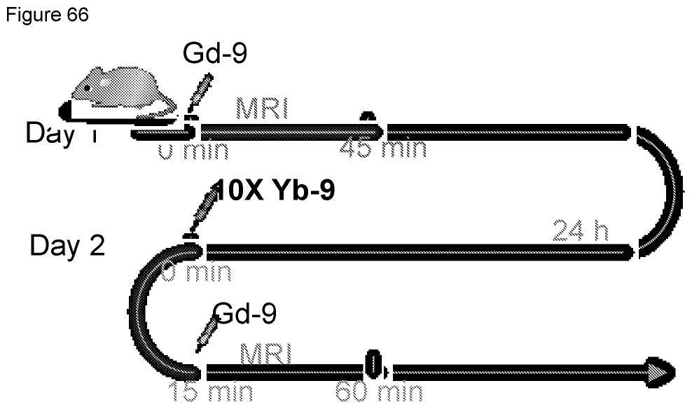

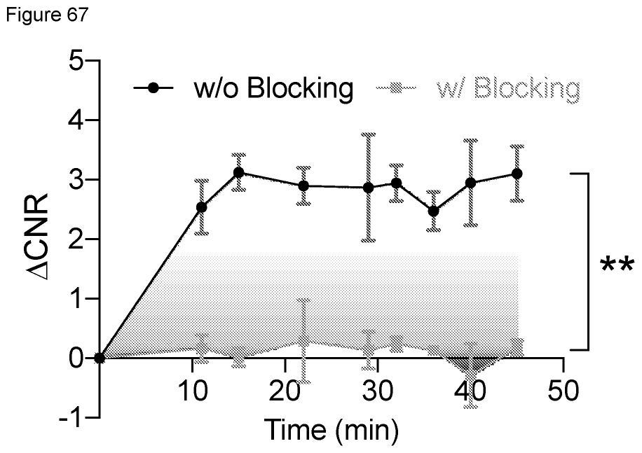

Using Yb-9, blocking study was performed in fibrotic mice. Yb-9 has the same structure as Gd-9 but Gd³⁺ is replaced by the MRI-inactive Yb³⁺ ion. CCl₄ injured mice were imaged with 100 μmol/kg Gd-9 and then the next day gave a blocking dose of 1000 μmol/kg Yb-9, followed 15 minutes later by 100 μmol/kg of Gd-9 (FIG. 66). The 10-fold dose of Yb-9 completely eliminated liver MRI enhancement with Gd-9 (P=0.0045), demonstrating the specific binding of Gd-9 to the livers of fibrotic mice (FIG. 67).

Rationally introducing dual binding hydrazine groups in aldehyde-reactive gadolinium probes boosts on-rate, allysine protein bound relaxivity, and lowers off-rate. Structural optimization on the orientation of targeting moieties gives Gd-9 as the highest sensitivity probe. Gd-9 shows 6-fold higher reactivity, 2.3-fold higher relaxivity and 1.5-fold slower off-rate than previously published Gd-CHyd and this translates to 10-fold higher DCNR in vivo. This study reveals the efficiency of dual binding and the importance of orientation modulation in designing targeted MRI probes in vivo.

### Example C. Quantitative and Noninvasive Detection of Treatment Response in Pulmonary Fibrogenesis by Molecular MRI

Pulmonary fibrosis results in thickening of the lung interstitium, abolition of alveolar spaces and eventual respiratory failure. It is accompanied by upregulation of lysyl oxidase and Lox-like enzymes which catalyze oxidation of lysine e-amino groups on extracellular matrix proteins (chiefly collagens) to form the aldehyde containing amino acid allysine which then undergoes cross-linking reactions with other proteins to stabilize the matrix. Gadolinium probes functionalized with a hydrazine moiety can bind to allysine in vivo to robustly stage and quantify fibrogenesis. Here, a new probe, Gd-9, was designed with two hydrazine moieties where dual binding to allysine may enhance on-rate and slow the off-rate relative to the previous reported probe GdCHyd and assessed whether Gd-9 can be used to monitor treatment response in a model of pulmonary fibrosis with a promising natural product EGCG being developed clinically.

Gd-9 is a Gd-DOTA derivative with two hydrazine arms synthesized by amide coupling of two tBu-piperazin-1-ylcarbamate groups to 1,4,7,10-tetraazacyclododecane-1,4-bis(tBu-acetate)-4,10-bis(glutaric acid 1-tBu ester), followed by acid deprotection and gadolinium complexation. C57Bl/6 adult male mice at 8 weeks of age received a single intratracheal dose of bleomycin, 1 U/kg body weight (50 μL total volume) as previously described. Pair-wise imaging study of GdCHyd and Gd-9 was carried out in mice at day 14 post bleomycin injury on a 4.7 T MRI with a dose of 100 μmol/kg of each probe. For the treatment study, mice were imaged first at day 10 post bleomycin injury, then treated daily with oral gavage of either epigallocatechin gallate (EGCG, 100 mg/kg) or PBS (vehicle) for 11 days. At day 21 post bleomycin injury, the animals were imaged again. MRI consisted of T1-weighted UTE (TR/TE/flip angle=4 ms/0.011 ms/15°) images acquired prior to and up to 30 minutes following intravenous administration of Gd-9 (100 mol/kg). Following the second imaging session, animals were euthanized (60 minutes post injection), and lung tissues were removed for further analysis.

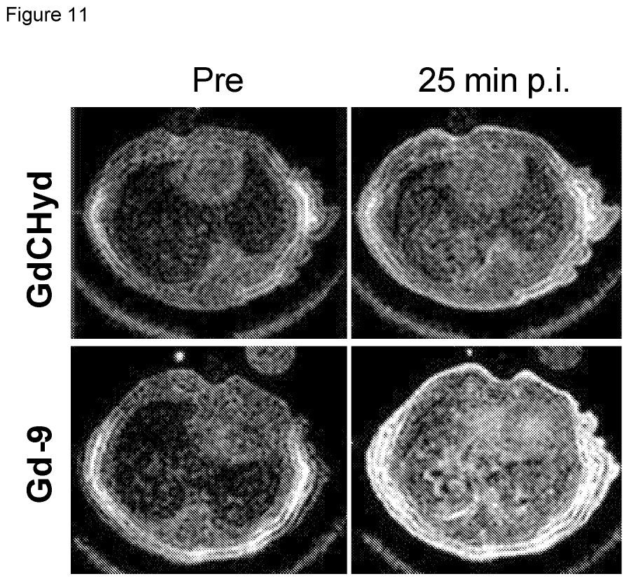

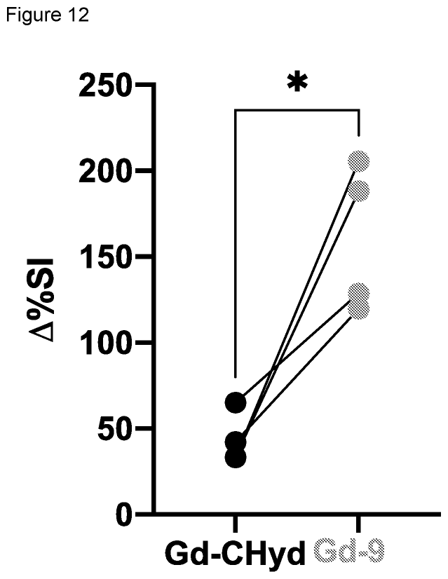

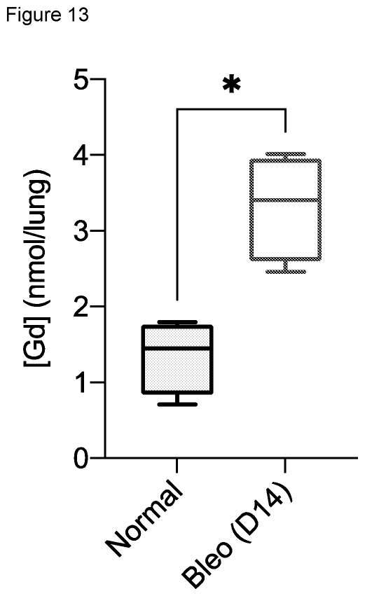

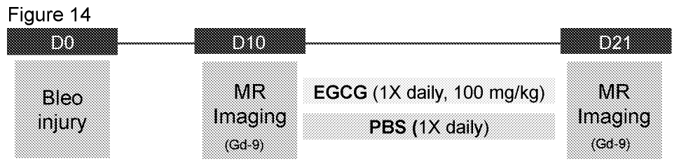

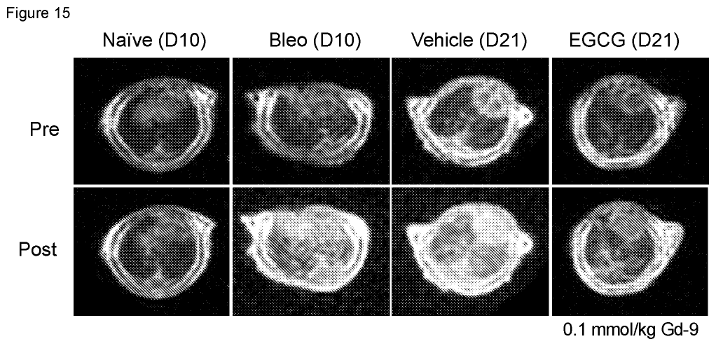

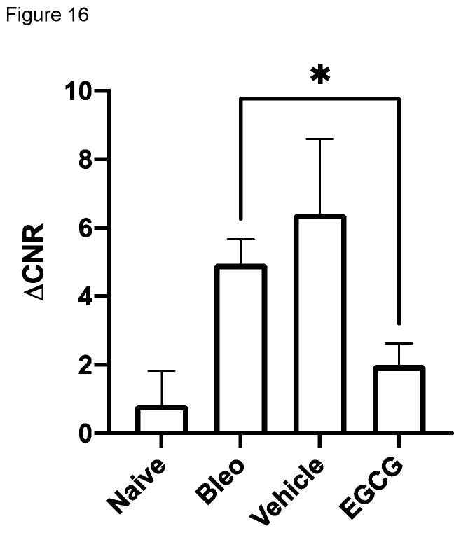

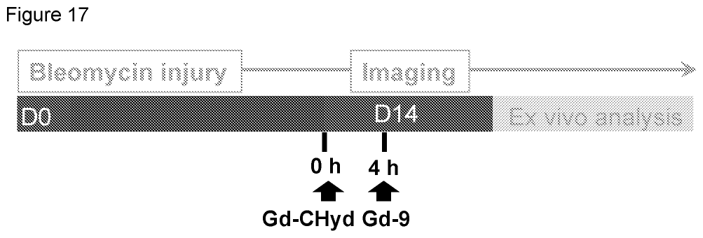

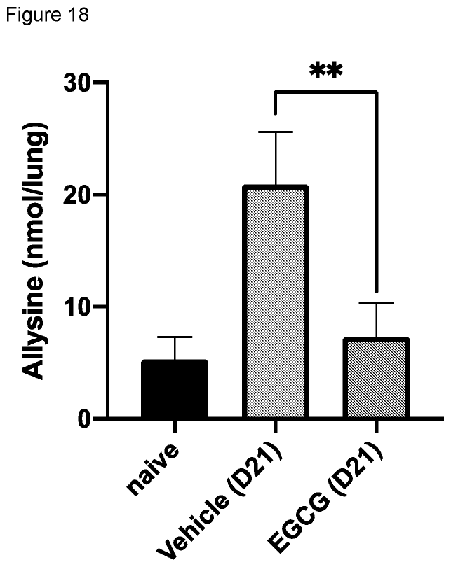

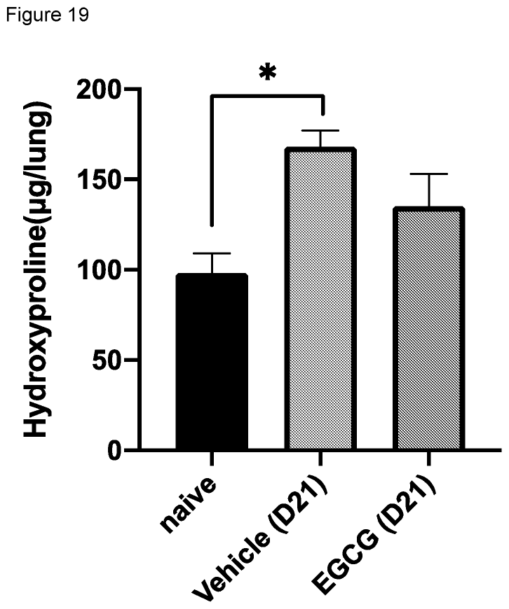

The dual hydrazine probe Gd-9 was first compared to GdCHyd in a head-to head comparison in vivo. Mice were imaged 14 days after bleomycin injury first with Gd-CHyd and then 4 h later with Gd-9. This pairwise study showed that the signal generated by Gd-9 in fibrotic lung is 3-fold higher (P<0.05) than that generated by GdCHyd at the same time point post injection of probe (FIG. 11 and FIG. 12). Gadolinium concentration determined by ICP also showed higher uptake in fibrotic lung than normal lung with the administration of Gd-9 (FIG. 13), demonstrating the high affinity of Gd-9 towards pulmonary fibrogenesis. Gd-9 was nexted used to monitor response to EGCG treatment in bleomycin-injured mice. Mice were imaged at 10 days after bleomycin injury and then the mice were treated with EGCG (100 mg/kg) or PBS (vehicle) for 11 days, and then the mice were imaged again (FIG. 14). Compared to mice imaged at day 10, the change in lung to muscle contrast to noise ratio (ΔCNR) after Gd-9 injection was significantly decreased in mice at day 21 after EGCG treatment (FIG. 15 and FIG. 16), while the vehicle treated group showed no reduction. Lung hydroxyproline (total collagen) and allysine were also reduced in the EGCG treated group compared to the vehicle group. These results indicate that Gd-9 is able to track disease status in drug treatment.

### Example D. Hydrazine Equipped Turn-on Manganese-Based MIRI Probes for Imaging Liver Fibrogenesis

Liver fibrosis can occur in most chronic liver diseases, and can lead to cirrhosis, liver failure, primary liver cancer, and/or organ failure. Fibrogenesis is accompanied by upregulation of lysyl oxidase enzymes which catalyze oxidation of lysine amino groups on extracellular matrix proteins to form the aldehyde containing amino acid allysine which then undergoes cross-linking. MRI probes functionalized with a hydrazine moiety can bind to allysine in vivo to detect fibrogenesis, but these are all gadoliniumbased and there is growing concern about the long term safety of Gd-based probes. Here, the design and synthesis of novel manganese-based MRI probes with high signal amplification for imaging liver fibrogenesis are described.

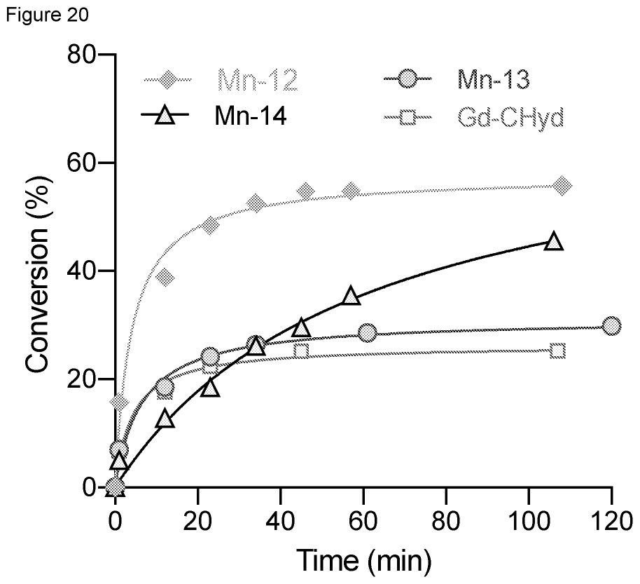

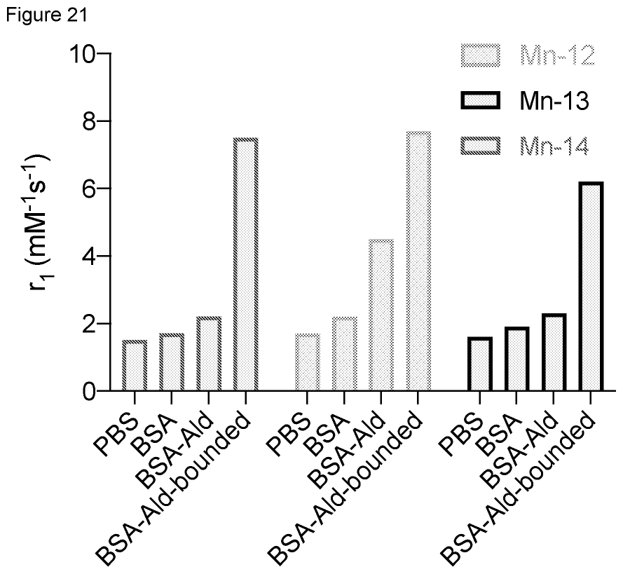

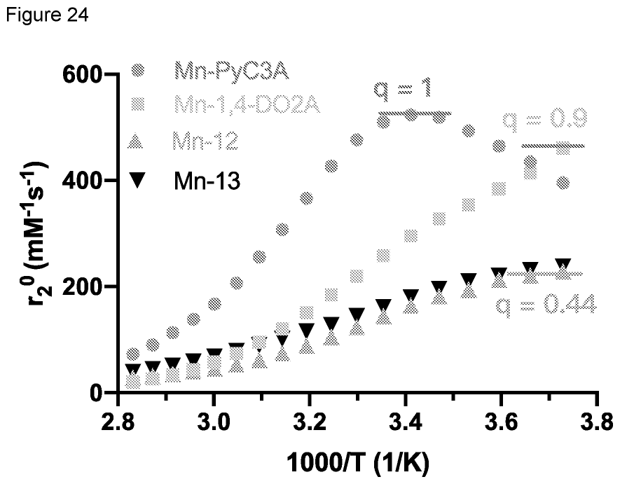

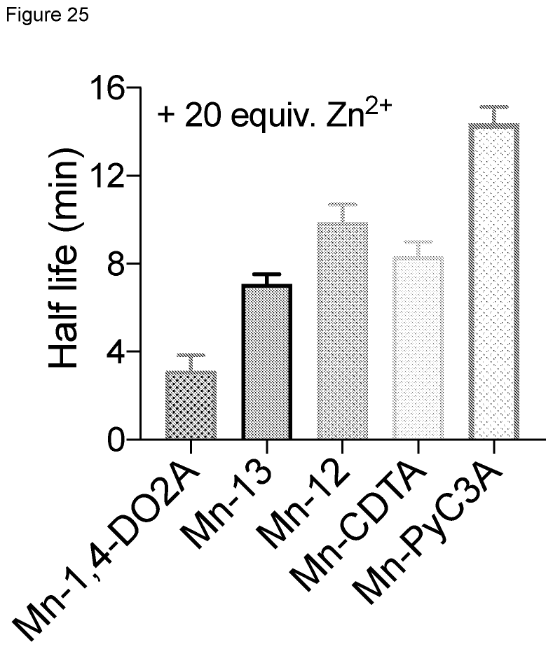

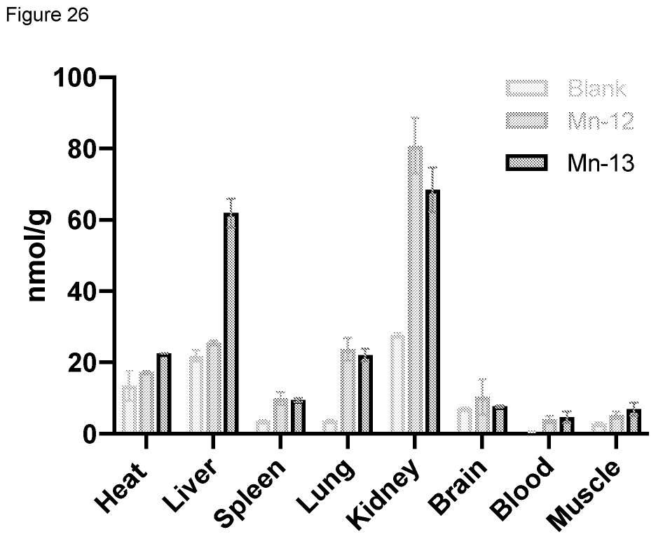

The design of hydrazine equipped manganese probes for imaging liver fibrogenesis must meet several requirements: 1) chemically stable Mn²⁺ complex that does not release free Mn²⁺ or undergo redox chemistry; 2) limited hepatobiliary elimination to minimize liver background signal; 3) contain coordinated water co-ligand to promote high relaxivity; 4) achieve higher relaxivity when bound to fibrotic tissue to increase signal at site of fibrosis. To meet these requirements, the probes Mn-12, Mn-13 and Mn-14 were designed based on the stable Mn-1,4-DO2A chelate. In model reactions with butyraldehyde, Mn-12 showed the highest on-rate and conversion yield (FIG. 20). Temperature dependence of the H₂17O tranverse relaxivity showed that the inner-sphere number of water molecule decreases from 0.90 in Mn-1,4-DO2A to 0.44 in Mn-12 (FIG. 24), resulting in a lower relaxivity of Mn-12 in PBS (1.6 mM⁻¹s⁻¹). However, Mn-12 exhibits an almost 5-fold turn-on relaxivity when bound to allysine modified BSA protein (7.7 mM⁻¹s⁻¹, FIG. 21), indicating high affinity towards allysine residues and increased relaxivity upon binding. Mn-12 is more inert to Mn²⁺ release than Mn-1,4-DO2A (FIG. 25). Unlike Mn-13, which showed high Mn liver levels in healthy mice at 60 minutes post-injection, Mn-12 showed little accumulation in the healthy liver (FIG. 26). This combination of stability, turn-on relaxivity, and low uptake in healthy liver indicate that Mn-12 is quite promising as a novel probe for liver fibrogenesis detection.

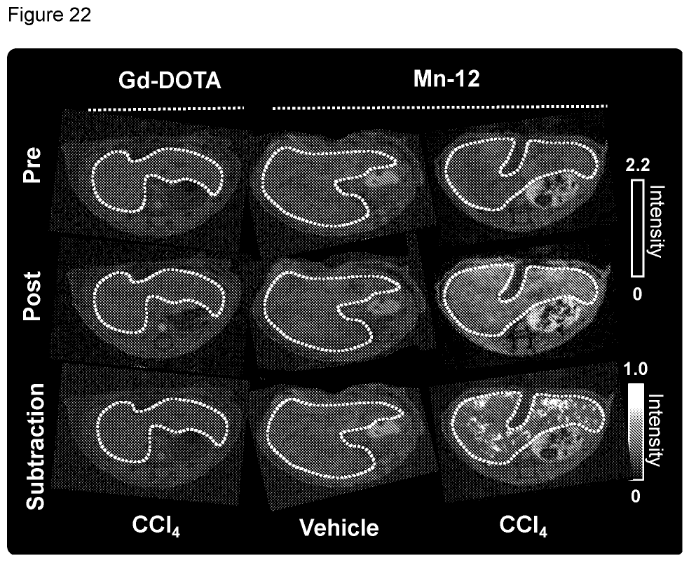

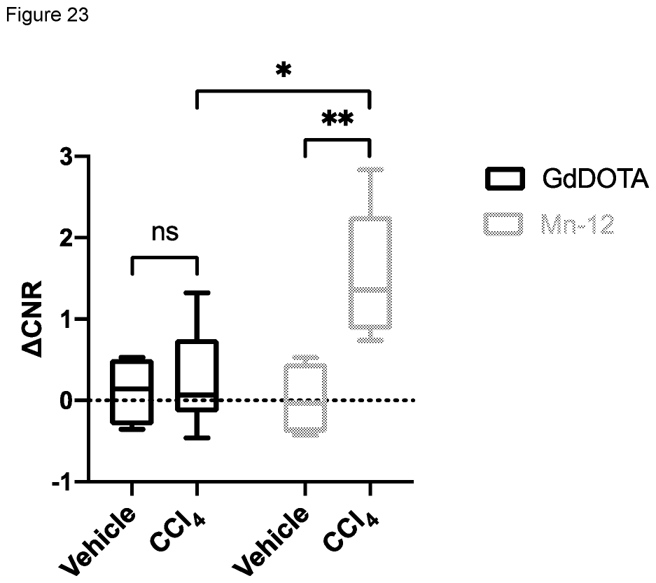

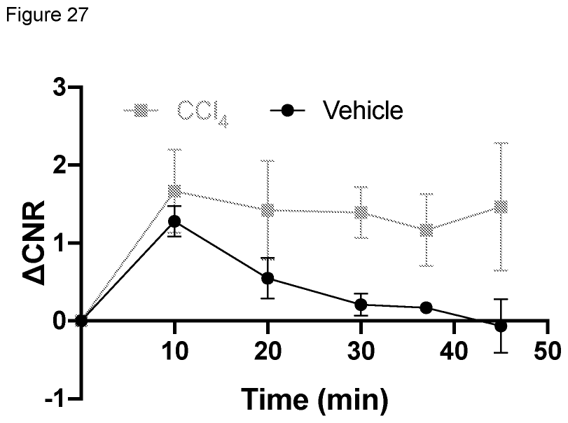

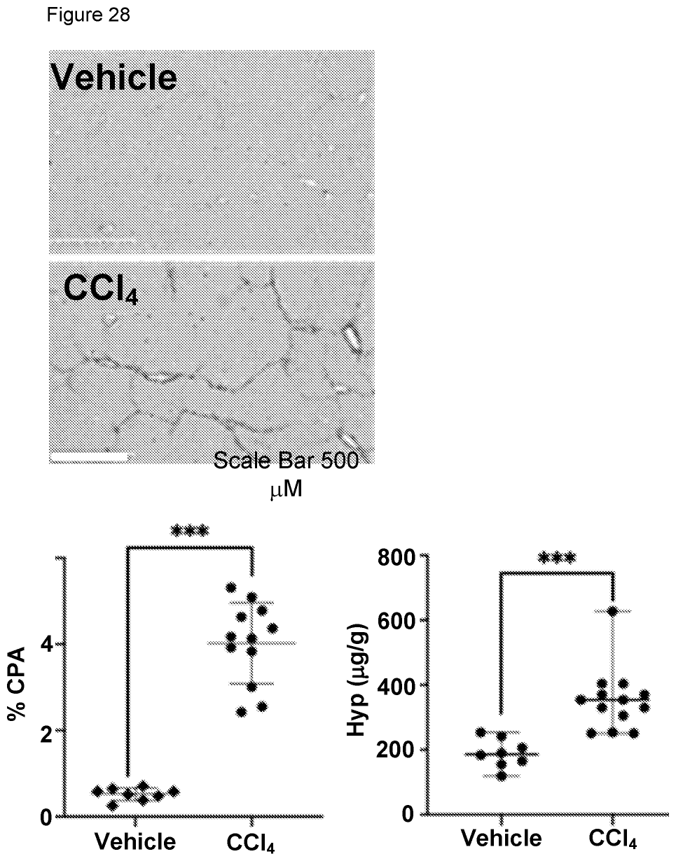

Mn-12 was tested in mice treated with CCl₄ or olive oil vehicle for 12 weeks to induce liver fibrosis. Ex vivo analyses of liver hydroxyproline and Sirius red staining indicated consistent fibrosis in the CCl₄ group (FIG. 28). Gd-DOTA was used as a comparison to demonstrate the specificity of Mn-12 towards fibrosis. At 45 minutes post-injection, there is no liver enhancement with Gd-DOTA in CCl₄ mice, but strong signal enhancement and high liver-to-muscle contrast to noise ratio (DCNR) in mice injected with Mn-12 (FIG. 22 and FIG. 23). The vehicle group imaged with Mn-12 showed significantly lower liver enhancement (FIG. 27). These data indicated that Mn-12 shows high sensitivity and is specific for in vivo detection of liver fibrogenesis.

### Example E. Mn(II)-Based MR Probes for Imaging Hepatic Fibrogenesis

Hepatic fibrogenesis, the active process of liver fibrosis, is a hallmark of many chronic liver diseases, which if left untreated results in cirrhosis, primary liver cancer, and/or organ failure. Molecular MR imaging of fibrogenesis has the potential for early diagnosis of liver fibrosis and to monitor disease progression and treatment response. Oxidized collagen with aldehyde containing allysine residues is a marker of fibrogenesis. Here novel macrocyclic Mn(II) chelates containing hydrazine moieties for targeting aldehydes are described. Rational design results in MR probes that are stable in vivo, have low signal enhancement in normal liver, but are highly reactive toward aldehydes resulting in a marked turn-on in relaxivity upon binding and enhancement of fibrotic liver.

The novel Mn-PC2A derivatives (FIG. 1A) were synthesized in 6 steps from the pyclen macrocycle. Hydrazone formation kinetics with butyraldehyde as a model compound were measured by UV spectroscopy at 220 nm, 25° C., pH 7.4, PBS. Hydrazone hydrolysis kinetics were measured by HPLC with UV detection at 220 nm using excess formaldehyde to trap the liberated hydrazine and prevent the reverse reaction. Allysine-modified BSA (BSA-Ald, 17.4 mg/mL, 260 M aldehyde) as a soluble model protein or BSA (17.3 mg/mL, 16.8 μM aldehyde) and Mn-15 or Mn-17 (50-200 μM) were incubated at 37° C. for 3 hours, and 1/T1 was measured at 1.4 T, 37° C., and plotted vs concentration to obtain relaxivity. The protein bound probe was isolated by ultrafiltration and the relaxivity of the protein-bound fraction measured. All Mn concentrations were measured by ICP-MS. Liver fibrosis was induced in male C57BL/6 mice by oral gavage with CCl₄ for 12 weeks, 2-3 times per week, while control mice received vehicle (olive oil). 3D FLASH images (TR/TE/FA=15 ms/2 ms/30°, 0.25 mm³ spatial resolution) were acquired on a 4.7 T Bruker Biospec prior to and dynamically for 40 min p.i. of 100 μmol/kg Mn-15. Liver to muscle contrast to noise was calculated as CNR=(SI_{Liver}−SI_{Muscle})/SD_{Air} where SI=signal intensity and SD=standard deviation in SI.

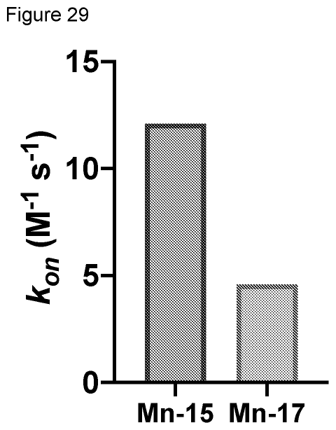

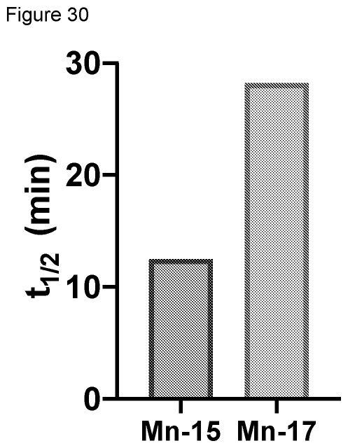

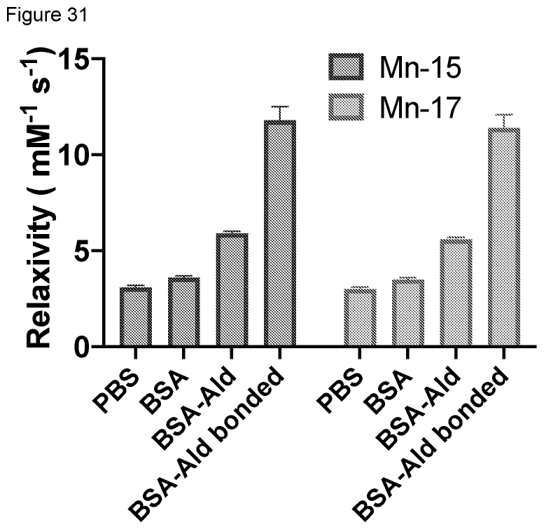

Two macrocyclic Mn(II) chelates with pendant hydrazine moieties for aldehyde targeting were synthesized. The a-carboxylate moiety in Mn-15 results in a 3-fold higher rate constant for hydrazone formation (FIG. 29) compared to Mn-17, and is one of the highest rate constants reported to date. However, the α-carboxylate also catalyzes hydrolysis with the half-life of Mn-15-hydrazone about half that of the Mn-17-hydrazone (FIG. 30). Relaxivities (FIG. 31) of Mn-15 and Mn-17 were similar in PBS (3 mM⁻¹s⁻¹) consistent with the presence of one coordinated water ligand. Relaxivity was not enhanced in BSA solution indicating little nonspecific protein binding, but relaxivity is increased 90% in the presence of allysine modified BSA. The relaxivity of the protein-bound form of Mn-15 and Mn-17 was almost 3 times higher than the unbound form.

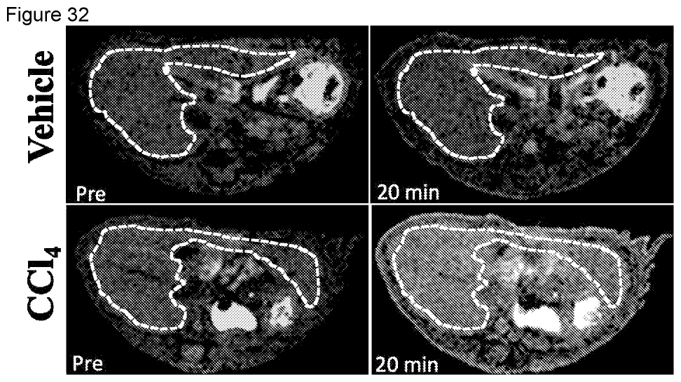

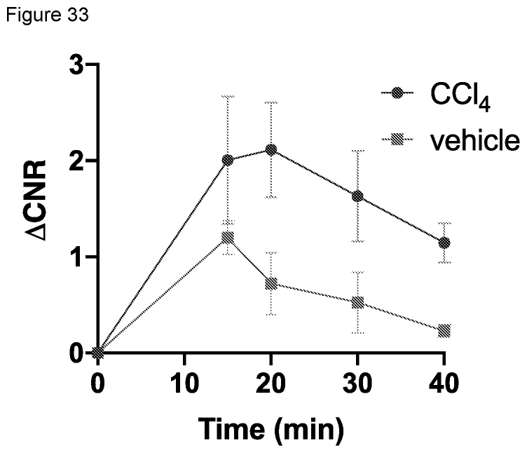

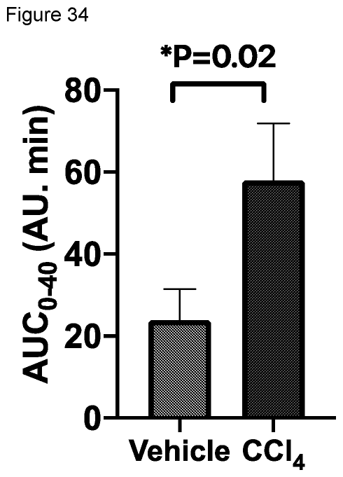

Mn-15 administration produced significant liver signal enhancement in CCl₄ injured mice but not in controls (FIG. 32). DCNR showed persistent liver signal enhancement in CCl₄ treated mice, while the liver only transiently enhanced in mice treated with vehicle (FIG. 33). DCNR was 3-fold higher at 20 minutes post injection, and the area under the ΔCNR curve was significantly larger in CCl₄ treated mice than in vehicle treated mice, demonstrating the potential for imaging liver fibrogenesis.

Mn-15 is a novel macrocyclic Mn(II) MR probe with a pendant hydrazine carboxylate moiety that enables rapid reaction with aldehydes, a 270% turn on in relaxivity at 1.4T, low nonspecific liver enhancement in healthy mice, but markedly higher liver signal enhancement in mice with ongoing liver fibrosis and shows potential for noninvasive imaging of liver fibrosis.

### REFERENCE

Akam, E. A.; Abston, E.; Rotile, N. J.; Slattery, H. R.; Zhou, I. Y.; Lanuti, M.; Caravan, P. Improving the reactivity of hydrazine-bearing MRI probes for in vivo imaging of lung fibrogenesis. Chemical science 2020, 11 (1), 224.

### Example F. Evaluation of New Allysine-Targeting ⁶⁸Ga-7 Probe in Preclinical Model of Pulmonary Fibrosis

Idiopathic pulmonary fibrosis (IPF) is a chronic and progressive lung disease resulting in scarring that impedes oxygen transport, and is ultimately fatal. Measuring disease activity in IPF would improve prognostication and assess whether drug treatment is effective. Allysine, an amino acid residue formed on extracellular proteins during active fibrosis, has been proposed as a disease activity marker. The goal of this work was to develop an allysine targeted PET probe with high specific uptake in fibrotic lungs and low background signal in adjacent tissues of heart, liver, muscle, and blood.

Probe ⁶⁸Ga-7 was synthesized in 5 steps. Male C57BL/6 mice were intratracheally instilled with bleomycin (1.2 U/kg) and imaged after 14 days when allysine levels peak in the lung. Bleomycin injured mice or age-matched controls were placed in a micro-PET/MRI scanner and administered ⁶⁸Ga-7 as a bolus via the lateral tail vein and dynamically imaged for 60 minute. At 90 min p.i. animals were euthanized, and their organs were harvested for gamma counting. After gamma counting, lungs were flash-frozen and later analyzed for hydroxyproline (total collagen, fibrosis measure) and allysine content.

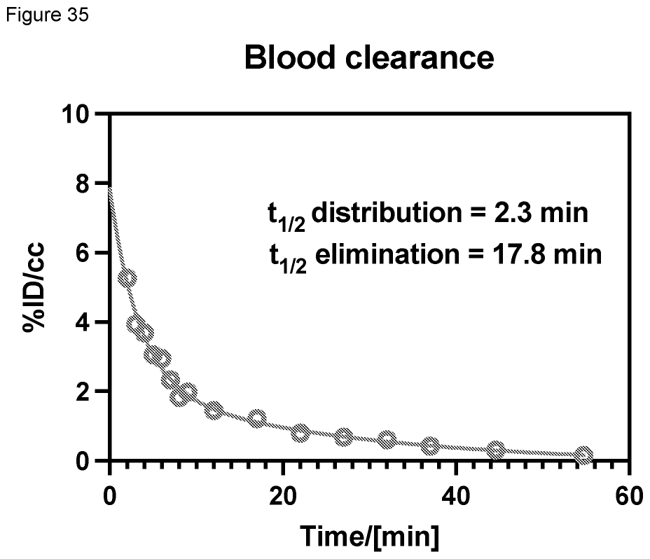

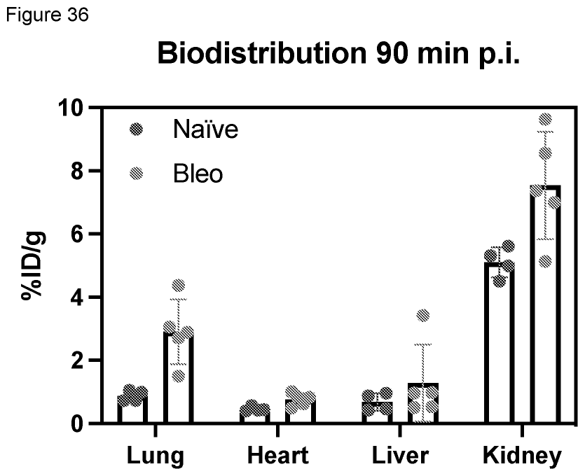

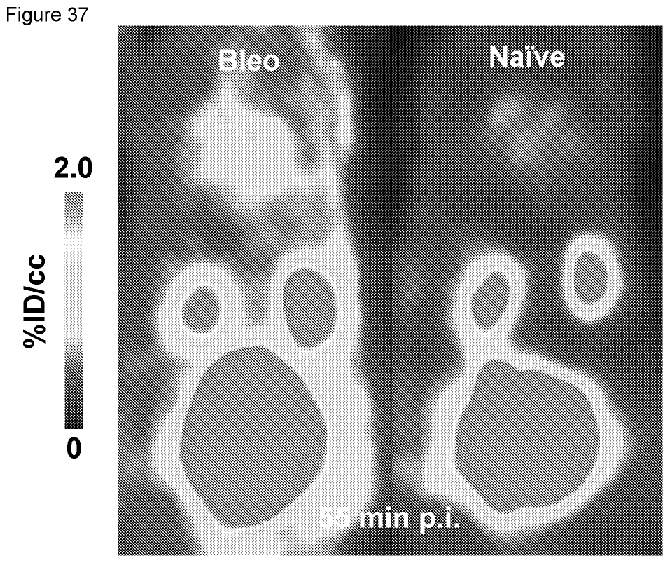

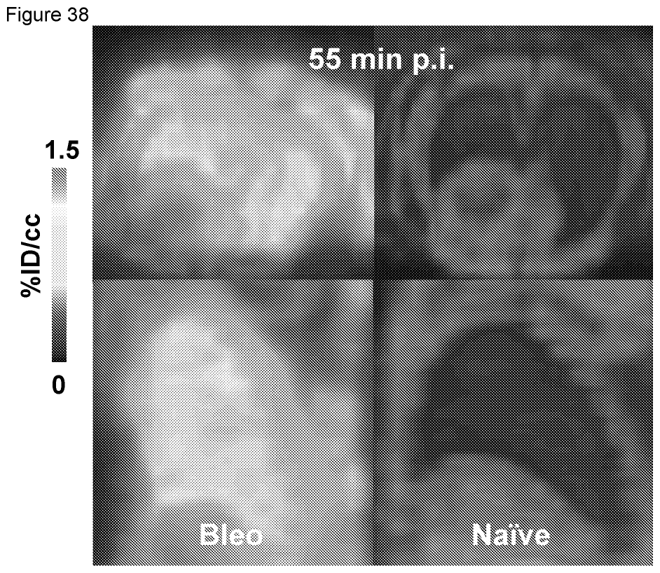

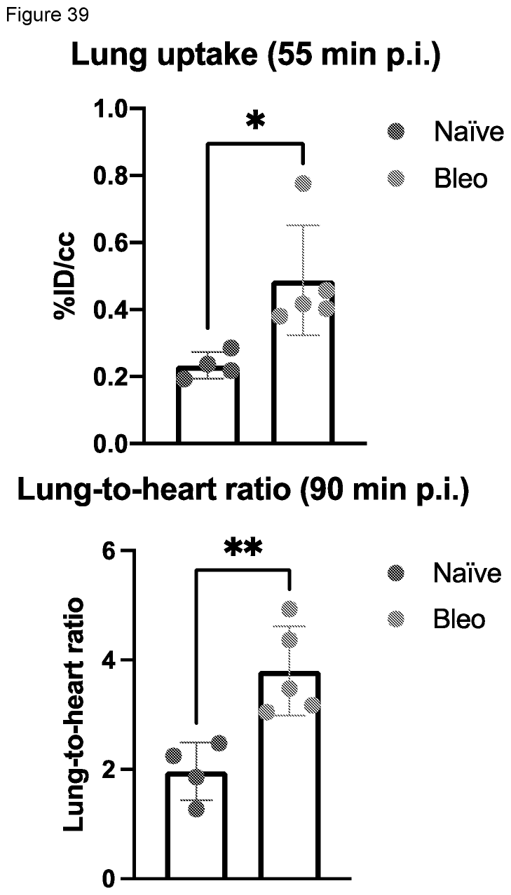

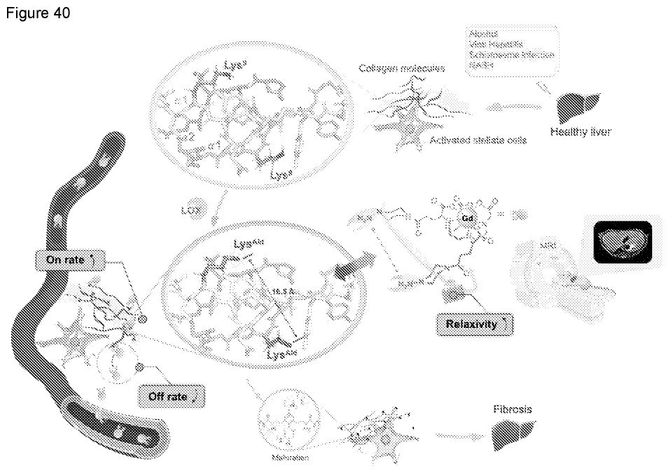

⁶⁸Ga-7 was selected from a library of PET tracers screened to select for high specificity towards aldehydes, rapid renal clearance and low non-specific uptake. ⁶⁸Ga-7 showed rapid blood clearance (FIG. 35) with elimination exclusively through the kidneys (FIG. 37). Very low signal is observed in the liver, heart, muscle, bone, or healthy lungs (FIG. 36 and FIG. 37). Significantly higher probe uptake was observed in the lungs of bleomycin-injured animals than in naïve controls, and lung to background (e.g. heart) ratios were also significantly higher in bleomycin injured mice (FIG. 37, FIG. 38, and FIG. 39).

⁶⁸Ga-7 is a hydrophilic, aldehyde-reactive PET probe with extracellular distribution and rapid renal clearance. Compared to the previously reported ⁶⁸Ga-NODAGA-indole, which employed an indole oxyamine moiety for Pictet-Spengler ligation to aldehydes. ⁶⁸Ga-7 showed twice the uptake in bleomycin injured lung and no hepatobiliary clearance resulting in greater lung-to-background contrast, important for delineating disease activity in the lower lung where injury is more prevalent in IPE

### Example G. Gd-9 MRI can Detect Early Onset of Liver Fibrogenesis and Measure Early Response to Treatment in a Mouse Model of NASH

It was next examined how early in the fibrotic process could Gd-9 enhanced MRI detect disease and whether Gd-9 enhanced MRI would be sensitive to early reduction in fibrogenesis associated with therapeutic intervention. The natural history of the choline-deficient, L-amino acid-defined, high fat diet (CDAHFD) mouse model which carries many of the human NASH phenotypes including liver inflammation and steatosis followed by progressive fibrosis was imaged. Disease can be reversed in this model by switching to normal chow.

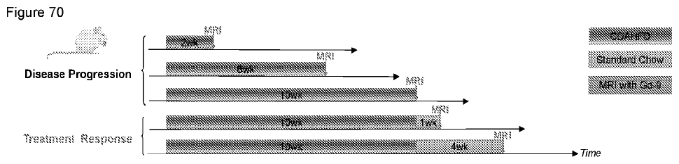

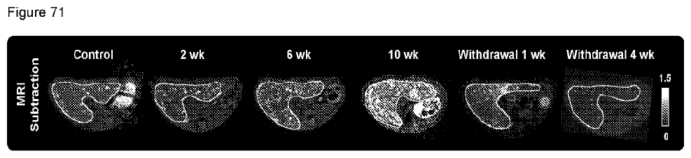

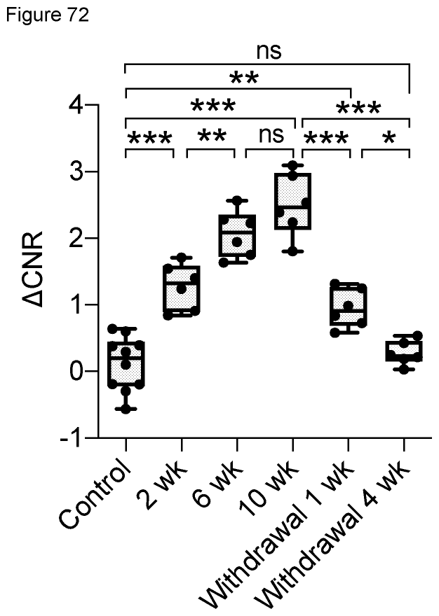

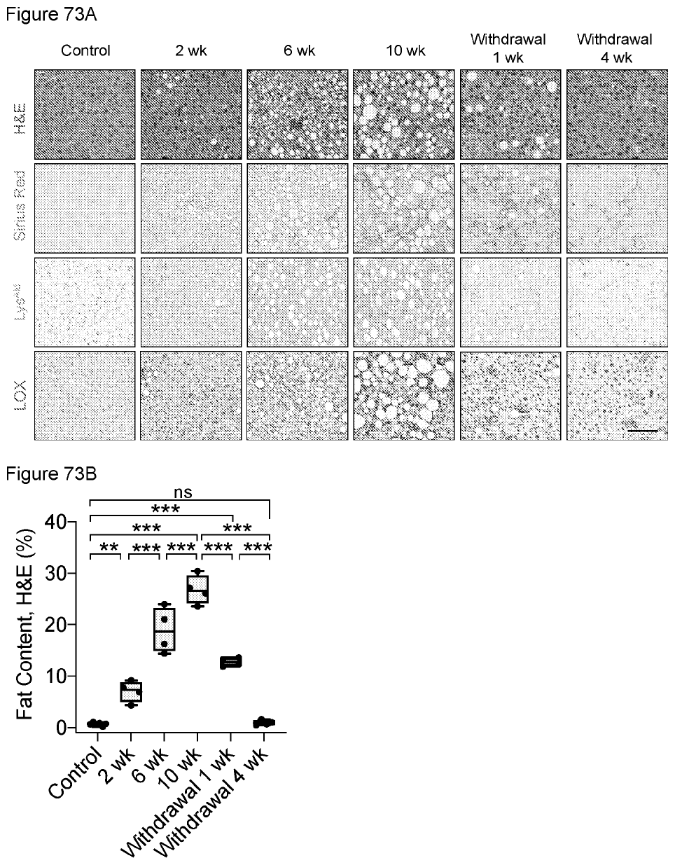

Mice were fed with CDAHFD or standard chow for 2, 6, or 10 weeks; two additional groups of mice received CDAHFD for 10 weeks and then standard chow for 1 or 4 weeks (FIG. 70; 2 wk, 6 wk, and 10 wk bars correspond to CDAHFD; 1 wk and 4 wk bars correspond to standard chow; “MRI” indicates MRI with Gd-9). In vivo 20 min post-Gd-9—pre-injection T1-weighted subtraction images in control and CDAHFD groups are presented in FIG. 71. Gd-9 enhanced MRI showed progressively higher ΔCNR with longer duration on CDAHFD. Importantly, compared to controls (0.1±0.4), significantly greater ΔCNR was seen in the livers of CDAHFD mice as early as 2 weeks on CDAHFD (1.3±0.4, FIG. 72). Switching to standard chow for 1 week after 10 weeks of CDAHFD resulted in significantly lower ΔCNR (10 wk CDAHFD: 2.5±0.5, switch 1 wk: 0.9±0.3), which further reduced after 4 weeks of diet reversal (0.3±0.2).

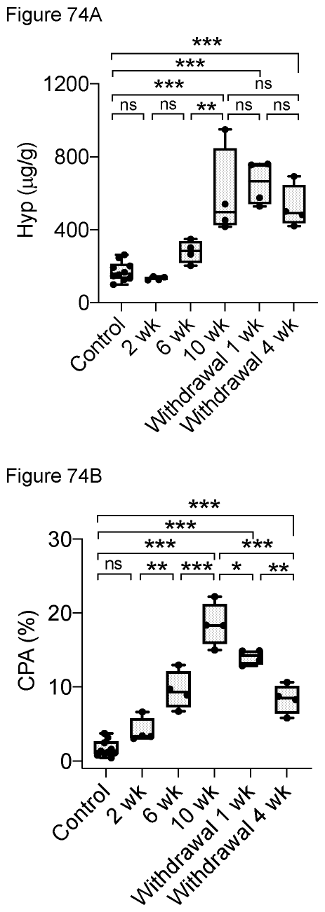

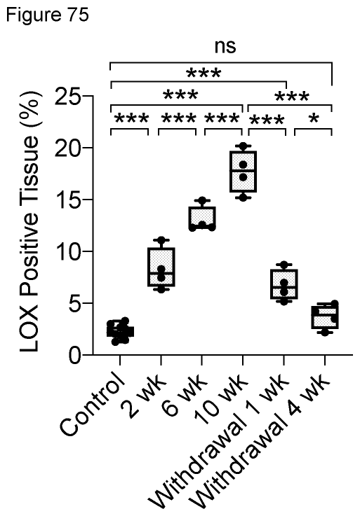

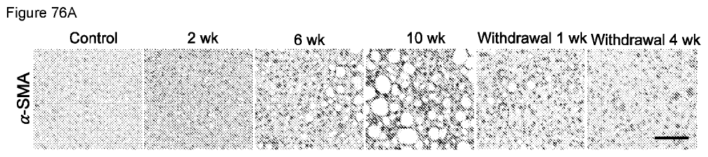

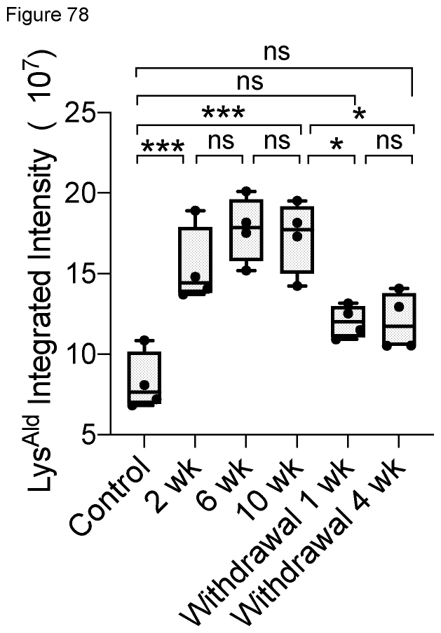

Histological analyses (FIGS. 73A and 73B) showed that steatosis, apparent as lipid droplets, is present after 2 weeks on CDAHFD and increases with time on diet. Fibrosis quantified by hydroxyproline and SR showed that compared to controls, neither was significantly increased after 2 weeks on CDAHFD but were significantly elevated after 6 or 10 weeks on CDAHFD (FIGS. 74A and 74B). However, IHC assessment of fibrogenesis markers LOX, Lys^{Ald} and α-SMA were significantly elevated compared to control livers even at 2 weeks of CDAHFD. The percentage of LOX positive tissue increased from 2.5±0.6% in the control group to 8.3±2.0%, 13.0±1.3%, and 17.7±2.1% after 2, 6, and 10 weeks of CDAHFD, respectively (FIG. 75). The average integrated intensity of Lys^{Ald} increased 86%, 115%, 109% at 2, 6, 10 weeks of CDAHFD compared to control group (FIG. 78).

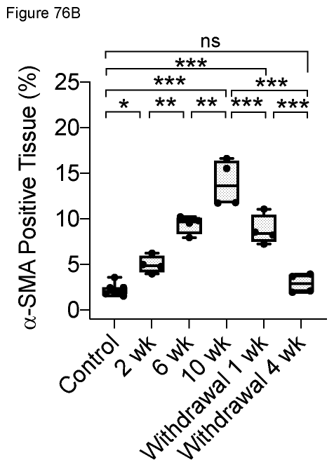

Animals fed CDAHFD for 10 weeks and then switched to standard chow for one week still showed prominent fibrosis (Hyp, FIG. 74A) but marked reductions in LOX (FIG. 75), Lys^{Ald} and α-SMA (FIGS. 76A and 76B) compared to 10 wk. These biomarkers of fibrogenesis (LOX, Lys^{Ald}, α-SMA) change quickly with the treatment of diet reversal, while Hyp as a biomarker of fibrosis does not show a significant decrease even 4 weeks after CDAHFD withdrawal when disease activity was further resolved (FIG. 73A).

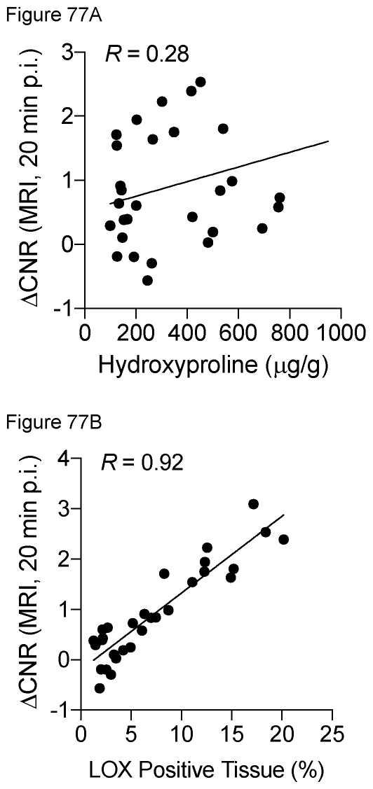

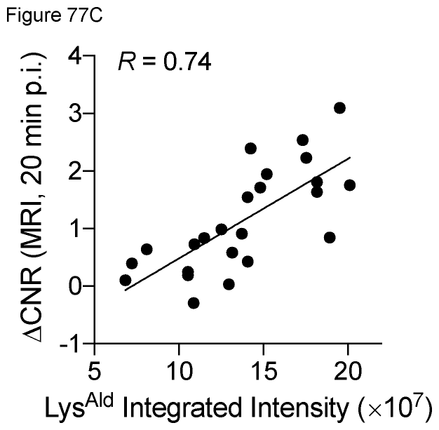

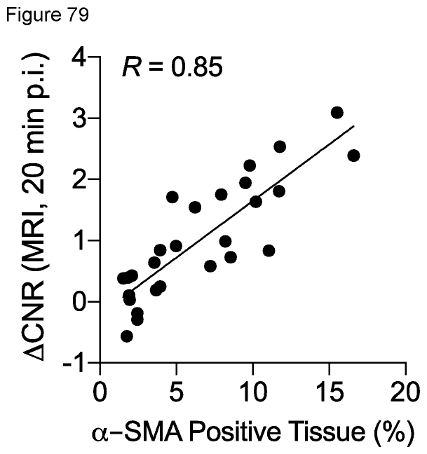

Gd-9 ΔCNR did not correlate with total liver collagen content as assessed biochemically by Hyp (FIG. 77A, R=0.24, P=0.24), but tracked well with measures of fibrogenesis like expression of LOX (FIG. 77B, R=0.92, P<0.0001), Lys Ald (FIG. 77D, R=0.74, P<0.0001) and α-SMA (R=0.85, P<0.0001, FIG. 79).

### Example H. Gd-9 MRI Detects Liver Fibrogenesis in a Rat Model of Obstructive Cholestatic Disease

### Data Analysis

Longitudinal relaxation rate (R1) maps were generated from the T1 mapping images using a custom written MATLAB (Mathworks, Natick, MA) program for voxel wise fitting of the inversion recovery signal intensities as a function of the inversion time. A region of interest (ROI) was manually traced encompassing the liver parenchyma while avoiding major blood vessels. ΔR1 was calculated by subtracting the R1_{Pre} from R1_{Post}, eq S3. ΔCNR were analyzed from 3D T1 weighted FLASH images, the same as that in mouse.

$$Δ R 1 = R {1}_{p o s t} - R {1}_{p r e} \qquad ( S3 )$$

### Results

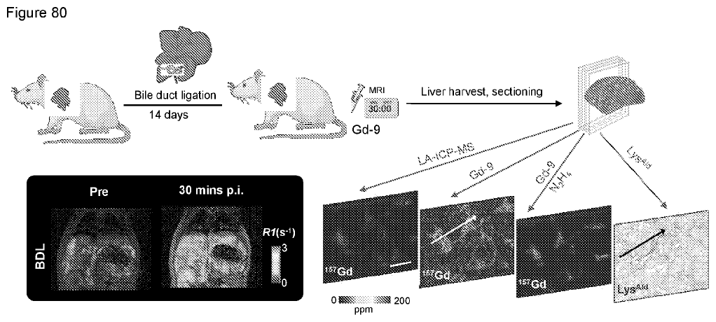

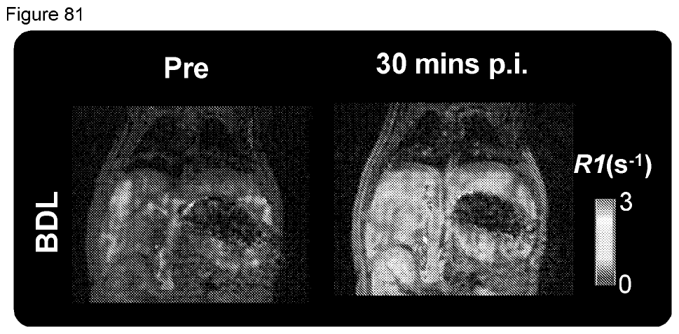

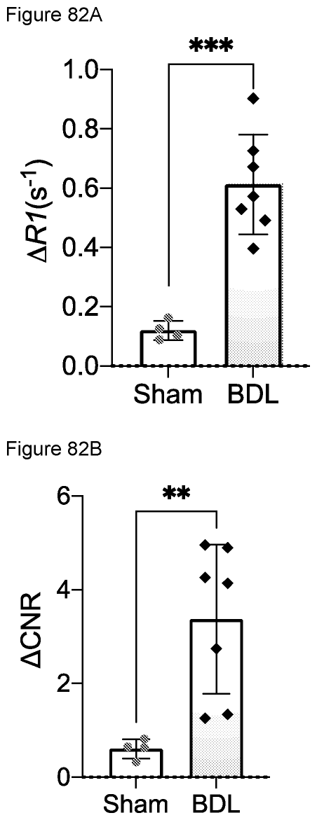

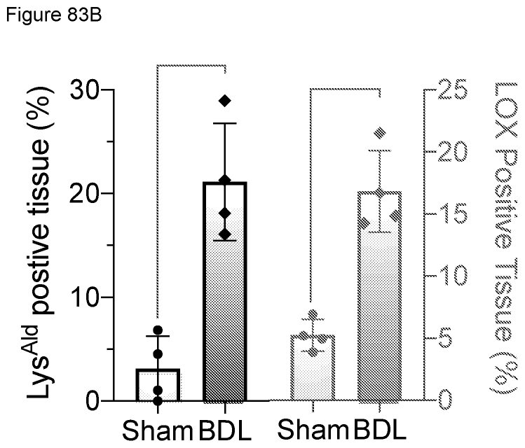

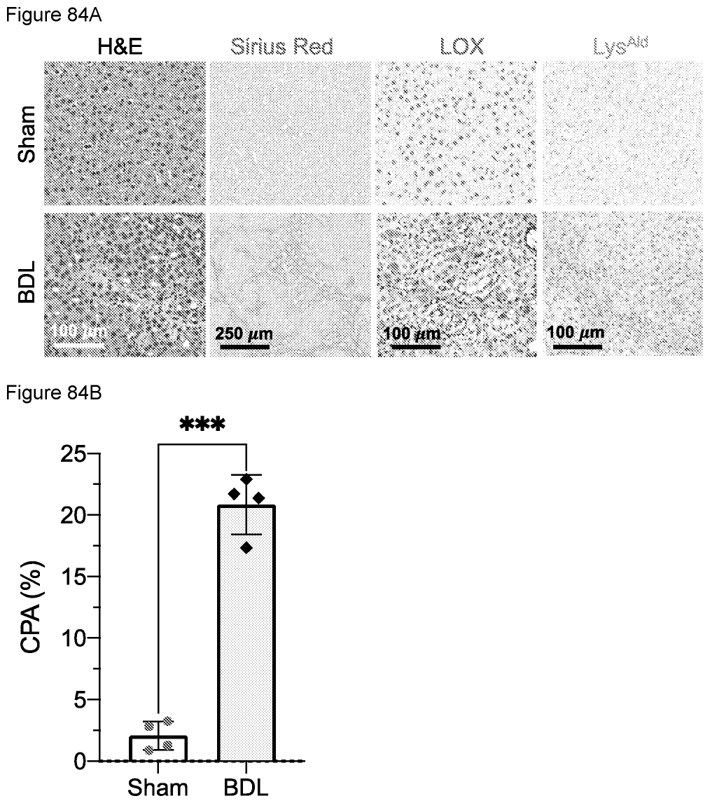

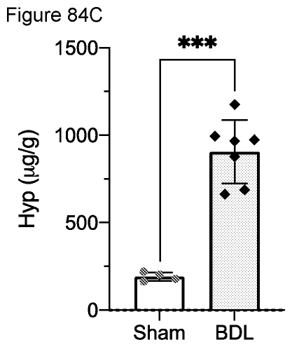

Bile duct ligation (BDL) induced cholestatic liver disease is a model that progresses into liver injury following liver inflammation and fibrosis. Ten days after BDL surgery, rats were imaged before and after 100 μmol/kg i.v. Gd-9 to detect hepatic fibrogenesis in a second species (FIG. 80). Gd-9 enhanced MRI showed significantly higher liver signal in BDL rats than in sham-operated rats at 30 min p.i. (FIG. 81). Both the change of liver longitudinal relaxation rate ΔR1 (0.6±0.2) and ΔCNR (3.4±1.6) were significantly enhanced in the BDL animals (FIGS. 82A and 82B), compared to sham rats (ΔR1=0.1±0.03, ΔCNR=0.6±0.2). Liver fibrosis/fibrogenesis in the BDL rats was confirmed by the presence of elevated CPA, LOX, Lys^{Ald} and hydroxyproline content (FIGS. 83, 84A, 84B, and 84C).

### Example I. Ex Vivo Elemental Gd Imaging of Fibrotic Rodent Livers Corresponds with In Vivo Binding

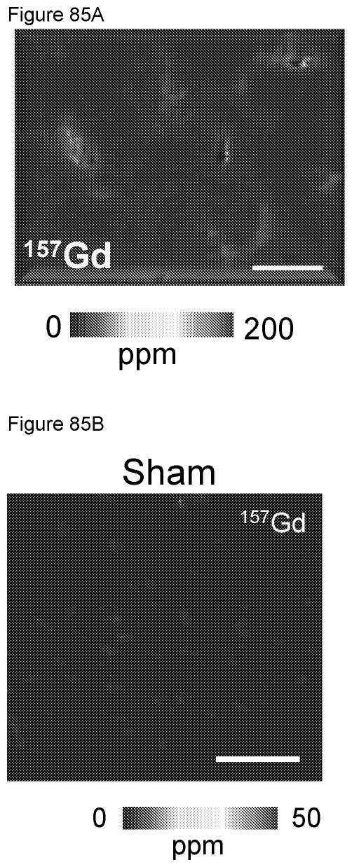

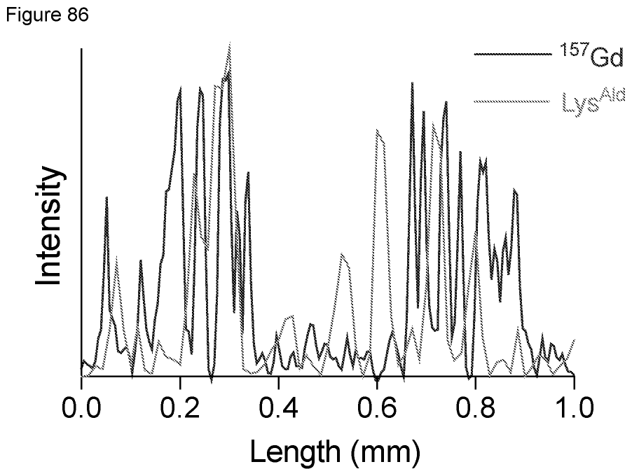

Rat livers were imaged ex vivo using laser ablation inductively coupled plasma mass spectrometry (LA-ICP-MS). Since Gd is not an endogenous element, imaging Gd represents a direct measure of Gd-9 in tissue. Liver tissue from BDL and sham rats imaged with Gd-9 was harvested 30 min p.i.. LA-ICP-MS showed higher liver Gd concentration in BDL rats (FIG. 85A) compared to sham rats (FIG. 85B), with specific accumulation of Gd (˜50 ppm) in fibrotic septa colocalizing with the presence of Lys^{Ald} (FIG. 86). On an adjacent liver slice additional Gd-9 and NaBH₃CN was incubated to make an irreversible linkage which resulted in further increased Gd concentration in fibrotic septa; on another adjacent slice Gd-9 was co-incubated with a 100-fold excess of hydrazine which blocked further binding of Gd-9 and demonstrated the specificity of the probe for tissue aldehyde (FIG. 85A).

### Example J. Gd-9 Binds to Human Fibrotic Liver Tissues

The applicability of Gd-9 to assess fibrogenesis in human liver specimens was investigated. Resected human fibrotic/cirrhotic liver associated with NASH (n=5) and normal liver tissue (n=4) (Table C) were sectioned and stained for Lys^{Ald}.

TABLE C
Clinical characteristics of human liver specimens.

| Patient | Gender | Age | Liver specimen |
|---|---|---|---|
| 1 | Male | 69 | NASH cirrhosis |
| 2 | Male | 57 | NASH cirrhosis |
| 3 | Female | 54 | NASH cirrhosis |
| 4 | Male | 74 | NASH with fibrosis |
| 5 | Male | 57 | NASH with fibrosis |
| 6 | Male | 67 | Normal |
| 7 | Male | 50 | Normal |
| 8 | Male | 38 | Normal |
| 9 | Male | 63 | Normal |

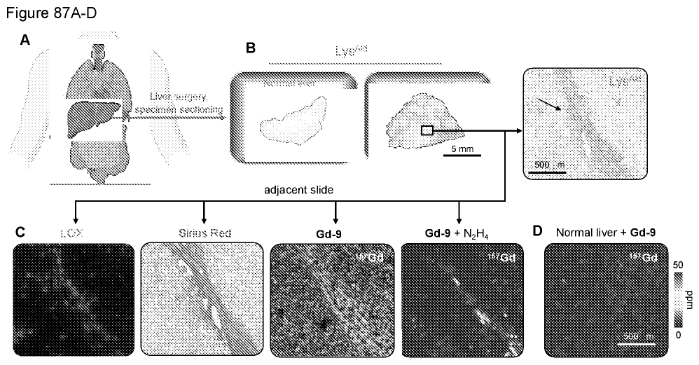

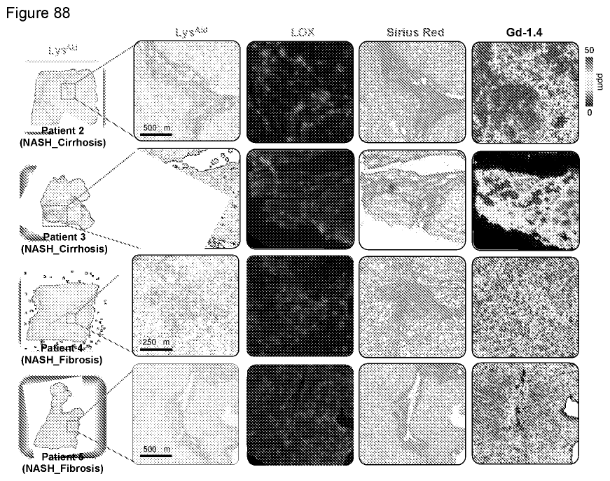

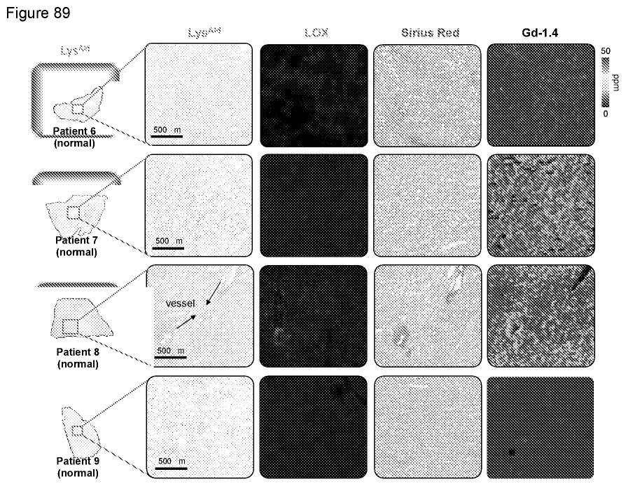

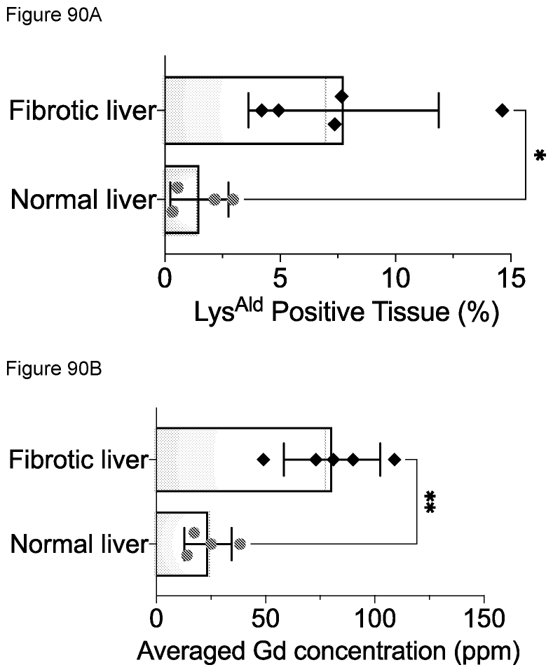

Significantly increased Lys^{Ald} levels were observed in fibrotic regions of liver, while there was negligible staining in normal liver (FIGS. 87A and 87B). Positive staining of collagen and LOX were also observed in the fibrotic septa (FIG. 87C). Adjacent slices from the fibrotic liver were incubated with Gd-9 or Gd-9 with excess N₂H₄. LA-ICP-MS imaging showed that the average Gd concentration in Lys^{Ald} positive areas (30-200 ppm Gd) was significantly higher than in regions with low Lys^{Ald} (<30 ppm Gd, FIGS. 87C and 87D), but excess N₂H₄ blocks Gd-9 binding, demonstrating the specificity of the probe to fibrotic human liver. Across all samples (FIGS. 88 and 89), the average Gd concentration in fibrotic human liver was 80.4±15.5 ppm, compared to 23.7±10.8 ppm in normal liver (FIGS. 90A and 90B).

### Discussion

Liver fibrosis is a progressive form of most CLD that can progress to cirrhosis, hepatocellular carcinoma, liver failure, and death. No noninvasive methods exist to assess disease activity, to sensitively detect early onset of disease, or to detect early response to treatment. Without wishing to be bound by theory, it is believed that Lys^{Ald} pairs transiently produced in the ECM can act as a general fibrogenesis marker. Rational design and systematic probe screening gave extracellular MR probe Gd-9 with two hydrazine moieties that can precisely form reversible covalent hydrazone bonds with Lys^{Ald} pairs on collagen telopeptides. The dual binding approach results in faster on-rate (600%), slower off-rate (50%), higher protein-bound relaxivity (50%) compared to a monobinder, and leads to markedly superior performance (10-fold higher ΔCNR) in vivo for measuring liver fibrogenesis. Gd-9 could specifically detect liver fibrogenesis in toxin- and dietary-induced mouse models, and a cholestasis rat model of liver fibrogenesis. The Gd-9 enhanced MRI signal was reflective of LOX mediated Lys^{Ald} cross-linking related disease activity.

In a mouse model of NASH, Gd-9 molecular MRI could detect the early onset of liver fibrosis (prior to significant increases in liver hydroxyproline) and was very sensitive to a reduction in fibrogenesis following a therapeutic intervention. The change in molecular MRI signal preceded the presence or resolution of fibrosis assessed biochemically and histologically which depend on the change of collagen concentration, highlighting this method for early disease detection and as an early readout of response to effective therapy.

The potential of Gd-9 for clinical translation is very high. Gd-9 is prepared in 5 synthetic steps with high overall yield (>50%) and is based on the inert Gd-DOTA chelate, which itself has been administered millions of times to humans with no unconfounded gadolinium associated toxicity. Gd-9 shows no nonspecific protein binding, nor does it accumulate in normal tissue. The MR signal change observed with Gd-9 is robust across different models and species. Ex vivo analysis of human liver specimens shows absence of extracellular aldehyde in normal liver, but high concentrations in fibrotic regions. Incubating Gd-9 with human fibrotic liver reveals Gd concentrations similar to those observed in rat model, where ΔR₁=0.6 s⁻¹ was observed with in vivo imaging, strongly suggesting that robust MR signal changes will be seen in patients with CLD.

Gd-9 enhanced MRI noninvasively measures hepatic fibrogenesis, but does not report on fibrosis stage. However, Gd-9 MRI may be readily combined with existing techniques like elastography or serum tests. For example, a positive Gd-9 MRI combined with a negative elastography exam could be indicative of disease with F1/F2 fibrosis, while negative Gd-9 MRI and negative elastography could indicate true absence of fibrosis.

In conclusion, it was determined that extracellular Lys^{Ald} pairs formed during active fibrosis serve as a specific biomarker of fibrogenesis that is quantifiable by a dual hydrazine equipped MR probe. The dual binding approach boosts on-rate, lowers off-rate, and increases MR signal upon binding. Gd-9 MRI was highly sensitive to early onset of liver fibrogenesis and could robustly detect treatment response prior to changes in liver collagen concentration.

What is claimed is:

1. A compound of Formula (I) 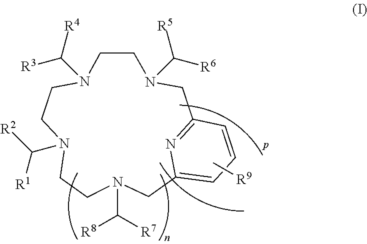
or a pharmaceutically acceptable salt thereof, wherein:
each R¹, R³, R⁵, and R⁷ are independently hydrogen or —C(═O)OH;
each R², R⁴, R⁶, and R⁸ are independently hydrogen or C₃₋₂₅ alkyl, wherein the C₃₋₂₅ alkyl is optionally substituted with 1-2 substituents independently selected from the group consisting of —NR^{A}R^{B}, —OH, halogen, C₁₋₆ alkoxy, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of halogen, —NR^{A}R^{B}, —OH, C₁₋₆ alkyl, and —C₁₋₆ alkyl-(NR^{A}R^{B}); and one or more non-adjacent carbon atoms of the C₃₋₂₅ alkyl are optionally replaced by O, N, NH, or N(CH₃);
R⁹ is H, halogen, —NR^{A}R^{B}, —OH, C₁₋₆ alkyl, or —C₁₋₆ alkyl-(NR^{A}R^{B}),
each R^{A} and R^{B} are independently hydrogen or C₁₋₆ alkyl;
n is 0 or 1; and
p is 0 or 1;
wherein if n is 0 at least two of R¹, R³, and R⁵ are —C(═O)OH and if n is 1 at least two of R¹, R³, R⁵, and R⁷ are —C(═O)OH.

2. The compound of claim 1, or a pharmaceutically acceptable salt thereof, wherein n is 0.

3. The compound of claim 1 or 2, or a pharmaceutically acceptable salt thereof, wherein R², R⁴, and R⁶ are independently selected from the group consisting of:
Hydrogen;

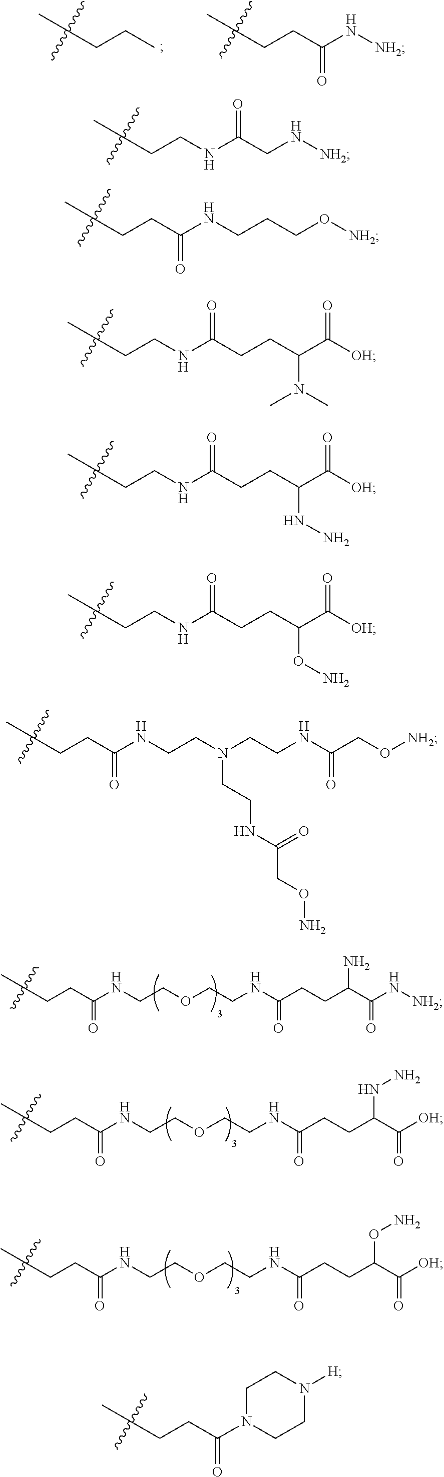

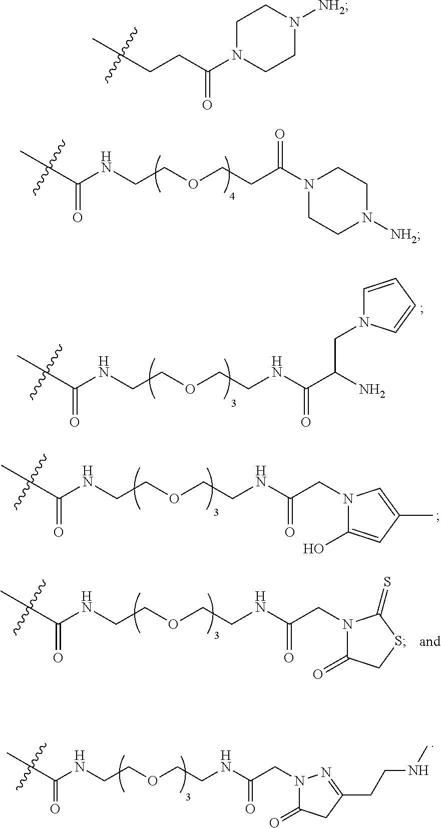

4. The compound of claim 1 or 2, or a pharmaceutically acceptable salt thereof, wherein R², R⁴, and R⁶ are all hydrogen.

5. The compound of claim 1 or 2, or a pharmaceutically acceptable salt thereof, wherein R², R⁴, and R⁶ are all C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

6. The compound of claim 1 or 2, or a pharmaceutically acceptable salt thereof, wherein R² and R⁴ are both hydrogen and R⁶ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

7. The compound of claim 1 or 2, or a pharmaceutically acceptable salt thereof, wherein R² and R⁶ are both hydrogen and R⁴ is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

8. The compound of claim 1 or 2, or a pharmaceutically acceptable salt thereof, wherein R⁶ and R⁴ are both hydrogen and R² is C₃₋₂₅ alkyl optionally substituted with 1-2 substituents independently selected from the group consisting of —NR^{A}R^{B}, —OH, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of —NR^{A}R^{B}, —OH, C₁₋₆ alkyl, and —C₁₋₆ alkyl-(NR^{A}R^{B}); and one to six non-adjacent carbon atoms of the C₃₋₂₅ alkyl are optionally replaced by O, N, NH, or N(CH₃).

9. The compound of any one of claims 1-8, or a pharmaceutically acceptable salt thereof, wherein R¹, R³, and R⁵ are all —C(═O)OH; or
wherein R¹ and R³ are both —C(═O)OH and R⁵ is hydrogen; or
wherein R¹ and R⁵ are both —C(═O)OH and R³ is hydrogen; or
wherein R⁵ and R³ are both —C(═O)OH and R¹ is hydrogen; or
wherein R¹ is —C(═O)OH and R² is hydrogen.

10. The compound of any one of claims 1-8, or a pharmaceutically acceptable salt thereof, wherein R³ is —C(═O)OH and R⁴ is hydrogen.

11. The compound of claim 1, or a pharmaceutically acceptable salt thereof, wherein R⁵ is —C(═O)OH and R⁶ is hydrogen.

12. The compound of claim 1, or a pharmaceutically acceptable salt thereof, wherein n is 1.

13. The compound of any one of claims 20 and 47-51, or a pharmaceutically acceptable salt thereof, wherein R⁷ is —C(═O)OH and R⁸ is hydrogen.

14. The compound of claim 1, or a pharmaceutically acceptable salt thereof, wherein p is 1.

15. The compound of any one of claims 1-14, or a pharmaceutically acceptable salt thereof, wherein R⁹ is H, halogen, or —OH.

16. The compound of any one of claims 1-13, or a pharmaceutically acceptable salt thereof, wherein p is 0.

17. The compound of claim 1, wherein the compound of Formula (I) is a compound of Formula (IA) 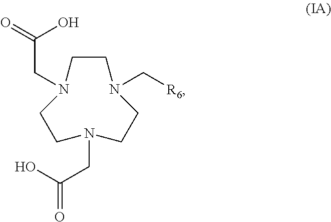
or a pharmaceutically acceptable salt thereof.

18. The compound of claim 17, or a pharmaceutically acceptable salt thereof, wherein R⁶ is selected from the group consisting of:
C₃₋₂₅ alkyl substituted with 4-10 membered heterocyclyl substituted —NR^{A}R^{B} wherein one non-adjacent carbon atom of the C₃₋₂₅ alkyl is replaced by NH and four non-adjacent carbon atom of the C₃₋₂₅ alkyl are replaced by O;
C₃₋₂₅ alkyl substituted with 5-10 membered heteroaryl and —NR^{A}R^{B}, wherein two non-adjacent carbon atoms of the C₃₋₂₅ alkyl are replaced by NH and three non-adjacent carbon atoms of the C₃₋₂₅ alkyl are replaced by 0;
C₃₋₂₅ alkyl substituted with 5-10 membered heteroaryl substituted with —OH and C₁₋₆ alkyl, wherein two non-adjacent carbon atoms of the C₃₋₂₅ alkyl are replaced by NH and three non-adjacent carbon atoms of the C₃₋₂₅ alkyl are replaced by 0;
C₃₋₂₅ alkyl substituted with 4-10 membered heterocyclyl, wherein two non-adjacent carbon atoms of the C₃₋₂₅ alkyl are replaced by NH and three non-adjacent carbon atoms of the C₃₋₂₅ alkyl are replaced by 0;
C₃₋₂₅ alkyl substituted with two —NR^{A}R^{B}, wherein three non-adjacent carbon atoms of the C₃₋₂₅ alkyl are replaced by NH and three non-adjacent carbon atoms of the C₃₋₂₅ alkyl are replaced by 0;
C₃₋₂₅ alkyl substituted with 4-10 membered heterocyclyl substituted with —C₁₋₆ alkyl-(NR^{A}R^{B}), wherein two non-adjacent carbon atom of the C₃₋₂₅ alkyl are replaced by NH and three non-adjacent carbon atom of the C₃₋₂₅ alkyl are replaced by 0;
C₃₋₂₅ alkyl substituted with —NR^{A}R^{B} and —OH, wherein three non-adjacent carbon atoms of the C₃₋₂₅ alkyl are replaced by NH and three non-adjacent carbon atoms of the C₃₋₂₅ alkyl are replaced by O; and
C₃₋₂₅ alkyl substituted with —NR^{A}R^{B} and —OH, wherein two non-adjacent carbon atoms of the C₃₋₂₅ alkyl are replaced by NH and four non-adjacent carbon atoms of the C₃₋₂₅ alkyl are replaced by O.

19. The compound of claim 1, wherein the compound of Formula (I) is a compound of Formula (IB) 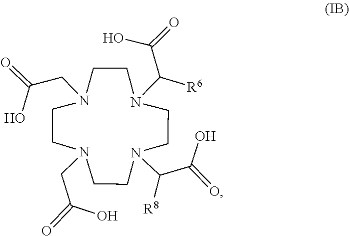
or a pharmaceutically acceptable salt thereof.

20. The compound of claim 18, or a pharmaceutically acceptable salt thereof, wherein R⁶ is selected from the group consisting of:
C₃₋₂₅ alkyl substituted with 4-10 membered heterocyclyl substituted with —NR^{A}R^{B}; and
C₃₋₂₅ alkyl substituted with —NR^{A}R^{B}, wherein one non-adjacent carbon atom of the C₃₋₂₅ alkyl is replaced by NH.

21. The compound of claim 18 or 19, or a pharmaceutically acceptable salt thereof, wherein R⁸ is selected from the group consisting of:
C₃₋₂₅ alkyl substituted with 4-10 membered heterocyclyl substituted with —NR^{A}R^{B};
C₃₋₂₅ alkyl substituted with 4-10 membered heterocyclyl;
C₃₋₂₅ alkyl; and
C₃₋₂₅ alkyl substituted with —NR^{A}R^{B}, wherein one non-adjacent carbon atom of the C₃₋₂₅ alkyl is replaced by NH.

22. The compound of claim 1, wherein the compound of Formula (I) is a compound of Formula (IC) 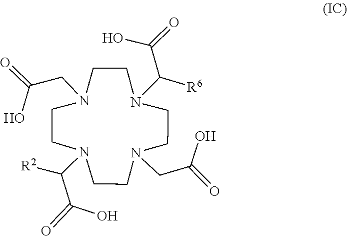
or a pharmaceutically acceptable salt thereof.

23. The compound of claim 22, or a pharmaceutically acceptable salt thereof, wherein R² is selected from the group consisting of:
Hydrogen; and
C₃₋₂₅ alkyl substituted with 4-10 membered heterocyclyl substituted with —NR^{A}R^{B};

24. The compound of claim 23 or 23, or a pharmaceutically acceptable salt thereof, wherein R⁶ is selected from the group consisting of:
C₃₋₂₅ alkyl substituted with two —NR^{A}R^{B}, wherein one non-adjacent carbon atoms of the C₃₋₂₅ alkyl are replaced by N, three non-adjacent carbon atoms of the C₃₋₂₅ alkyl are replaced by NH, and two non-adjacent carbon atoms of the C₃₋₂₅ alkyl are replaced by O; and
C₃₋₂₅ alkyl substituted with 4-10 membered heterocyclyl substituted with —NR^{A}R^{B};

25. The compound of claim 1, wherein the compound of Formula (I) is a compound of Formula (ID) 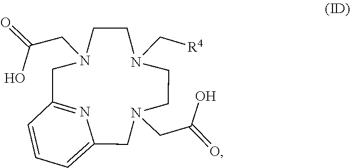
or a pharmaceutically acceptable salt thereof.

26. The compound of claim 25, or a pharmaceutically acceptable salt thereof, wherein R⁴ is selected form the group consisting of:
C₃₋₂₅ alkyl substituted with —NR^{A}R^{B} and —OH, wherein two non-adjacent carbon atoms of the C₃₋₂₅ alkyl are replaced by NH;
C₃₋₂₅ alkyl substituted with —NR^{A}R^{B} and —OH, wherein one non-adjacent carbon atom of the C₃₋₂₅ alkyl is replaced by NH and one non-adjacent carbon atom of the C₃₋₂₅ alkyl is replaced by 0;
C₃₋₂₅ alkyl substituted with —NR^{A}R^{B}, wherein two non-adjacent carbon atoms of the C₃₋₂₅ alkyl are replaced by NH; and
C₃₋₂₅ alkyl substituted with OH, wherein one non-adjacent carbon atom of the C₃₋₂₅ alkyl is replaced by NH and one non-adjacent carbon atom of the C₃₋₂₅ alkyl is replaced by N(CH₃).

27. The compound of claim 1, wherein the compound of Formula (I) is selected from: 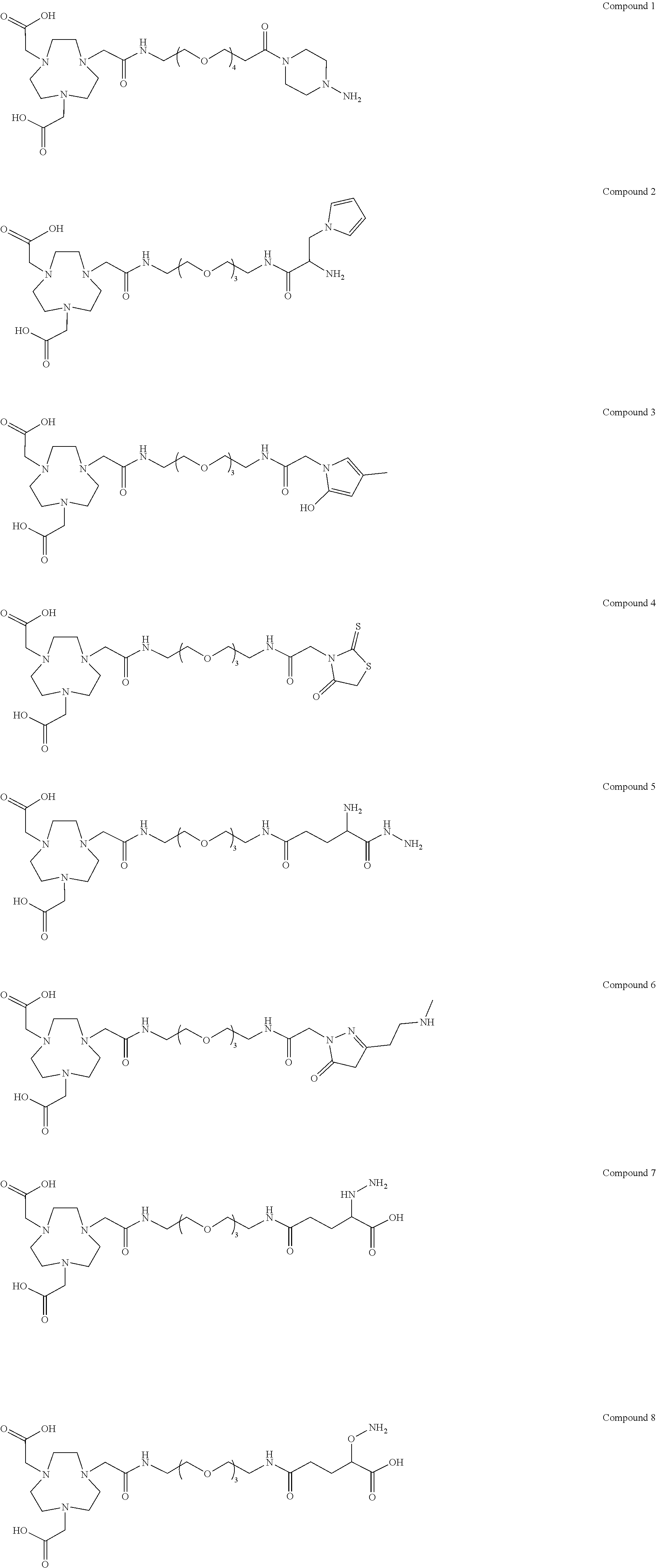 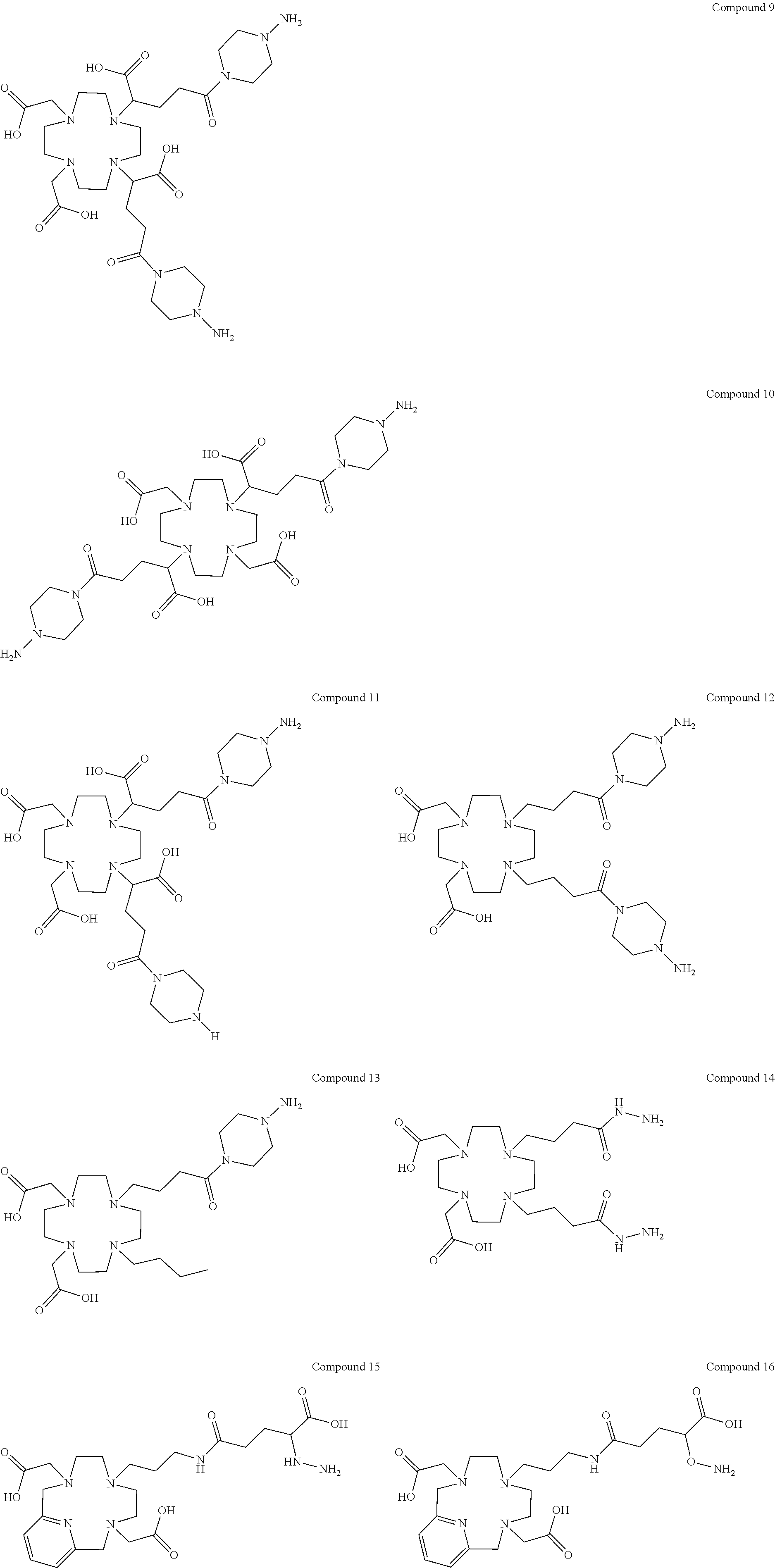 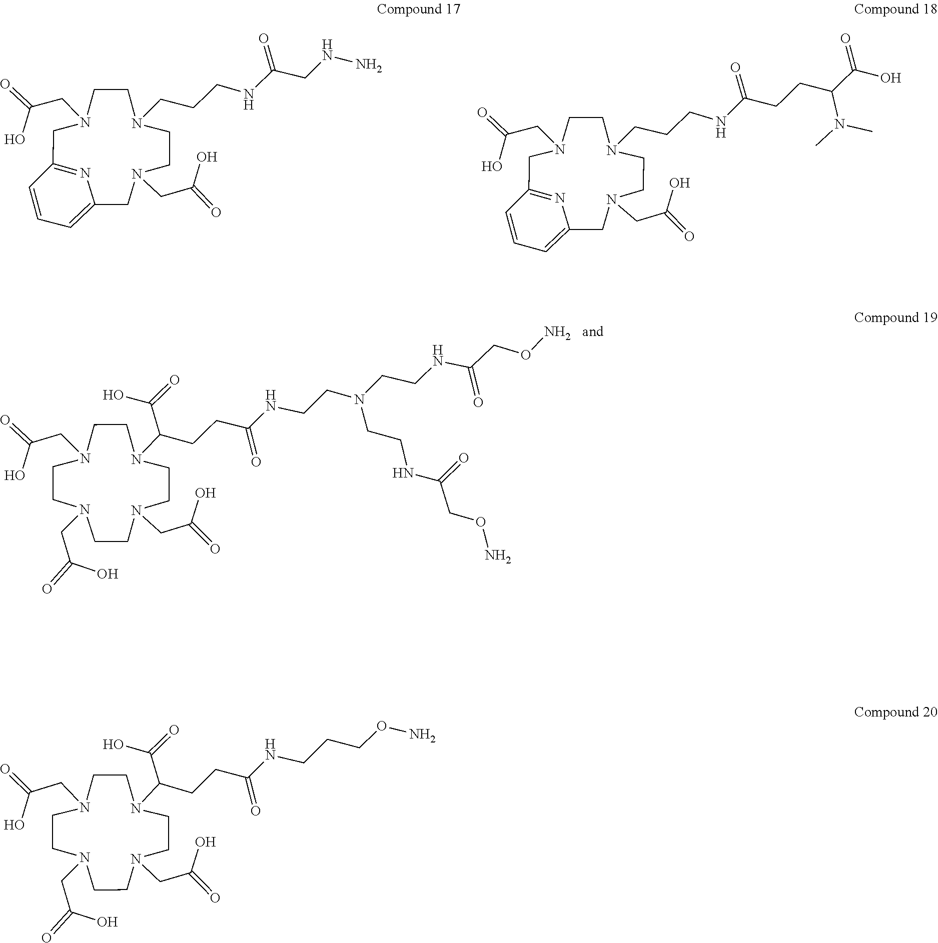 [SMILES: NNCC(=O)NCCCN1CCN(CC(=O)O)Cc2cccc(n2)CN(CC(=O)O)CC1 ; CN(C)C(CCC(=O)NCCCN1CCN(CC(=O)O)Cc2cccc(n2)CN(CC(=O)O)CC1)C(=O)O ; NOCC(=O)NCCN(CCNC(=O)CCC(C(=O)O)N1CCN(CC(=O)O)CCN(CC(=O)O)CCN(CC(=O)O)CC1)CCNC(=O)CON ; "and" ; NOCCCNC(=O)CCC(C(=O)O)N1CCN(CC(=O)O)CCN(CC(=O)O)CCN(CC(=O)O)CC1]
or a pharmaceutically acceptable salt thereof.

28. The compound of any one of claims 1-27, or a pharmaceutically acceptable salt thereof, wherein the compound further comprises a complexed metal cation.

29. The compound of claim 28, or a pharmaceutically acceptable salt thereof, wherein the metal cation is a Zn, Ga, Gd, Cu, Yb, Mn, Tc, or In cation.

30. The compound of claim 27 or 28, or a pharmaceutically acceptable salt thereof, wherein the metal cation is Zn²⁺, Ga³⁺, Gd³⁺, Cu²⁺, Yb³⁺, or Mn²⁺.

31. The compound of claim 28, wherein the compound of Formula (I) is 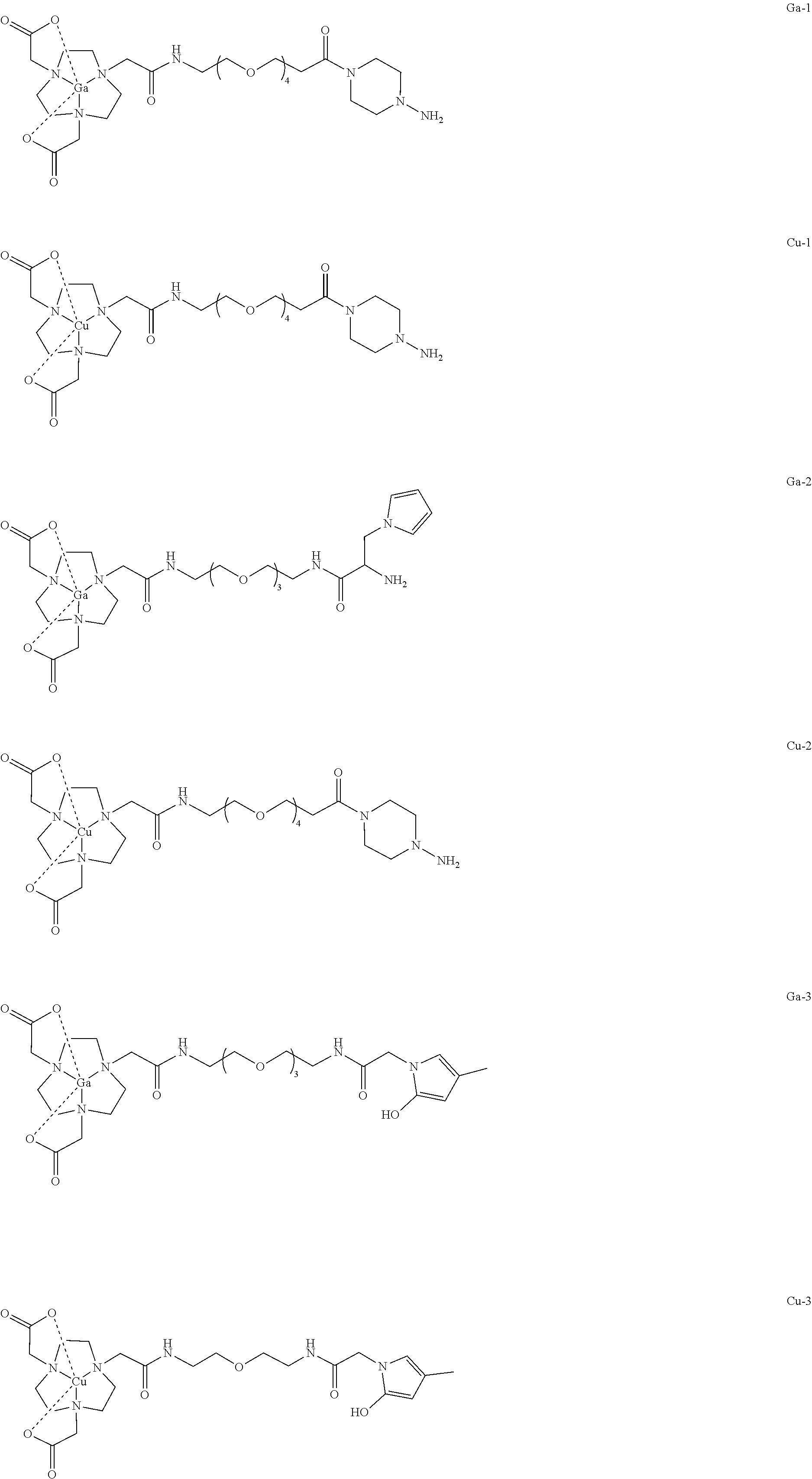 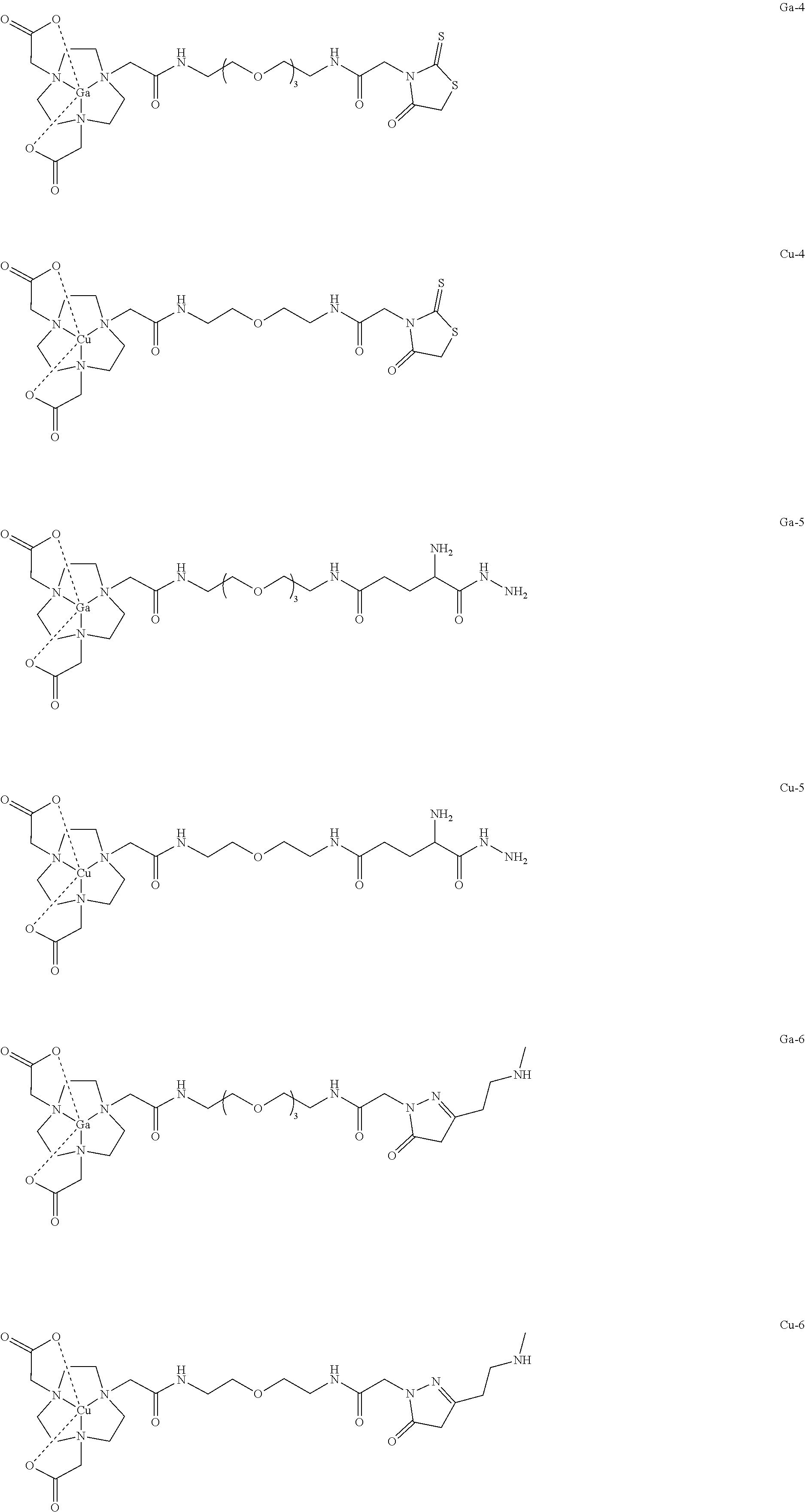 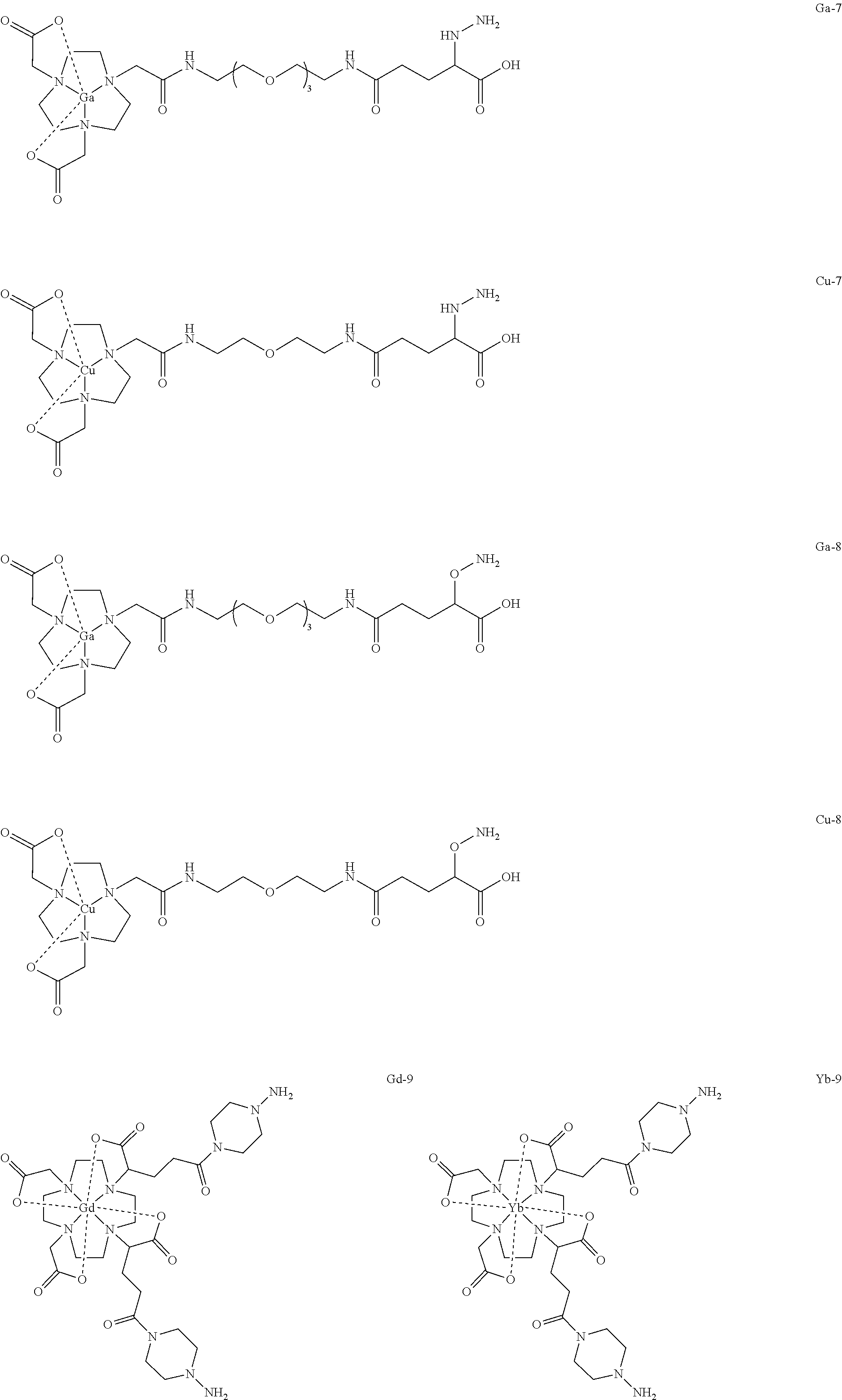 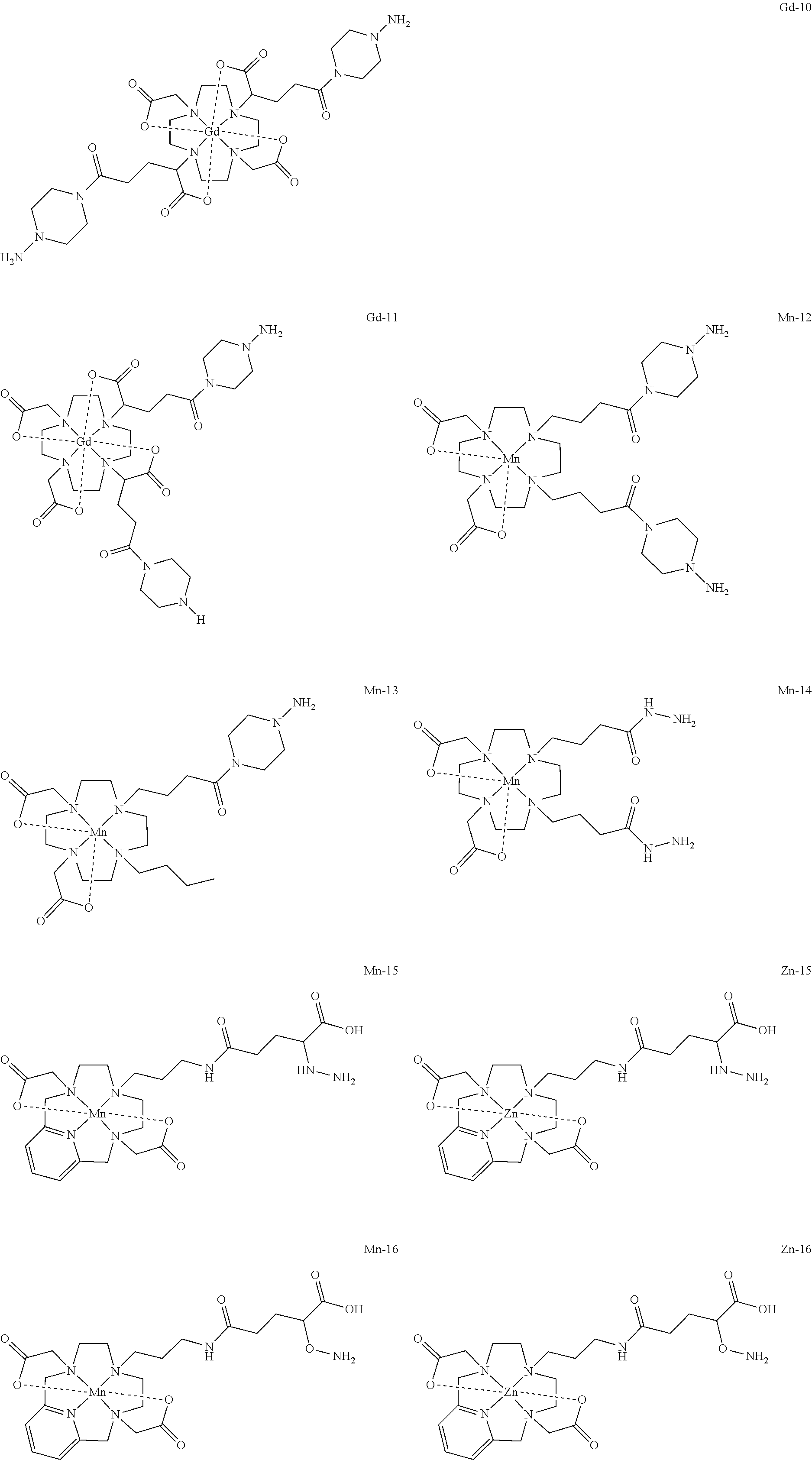 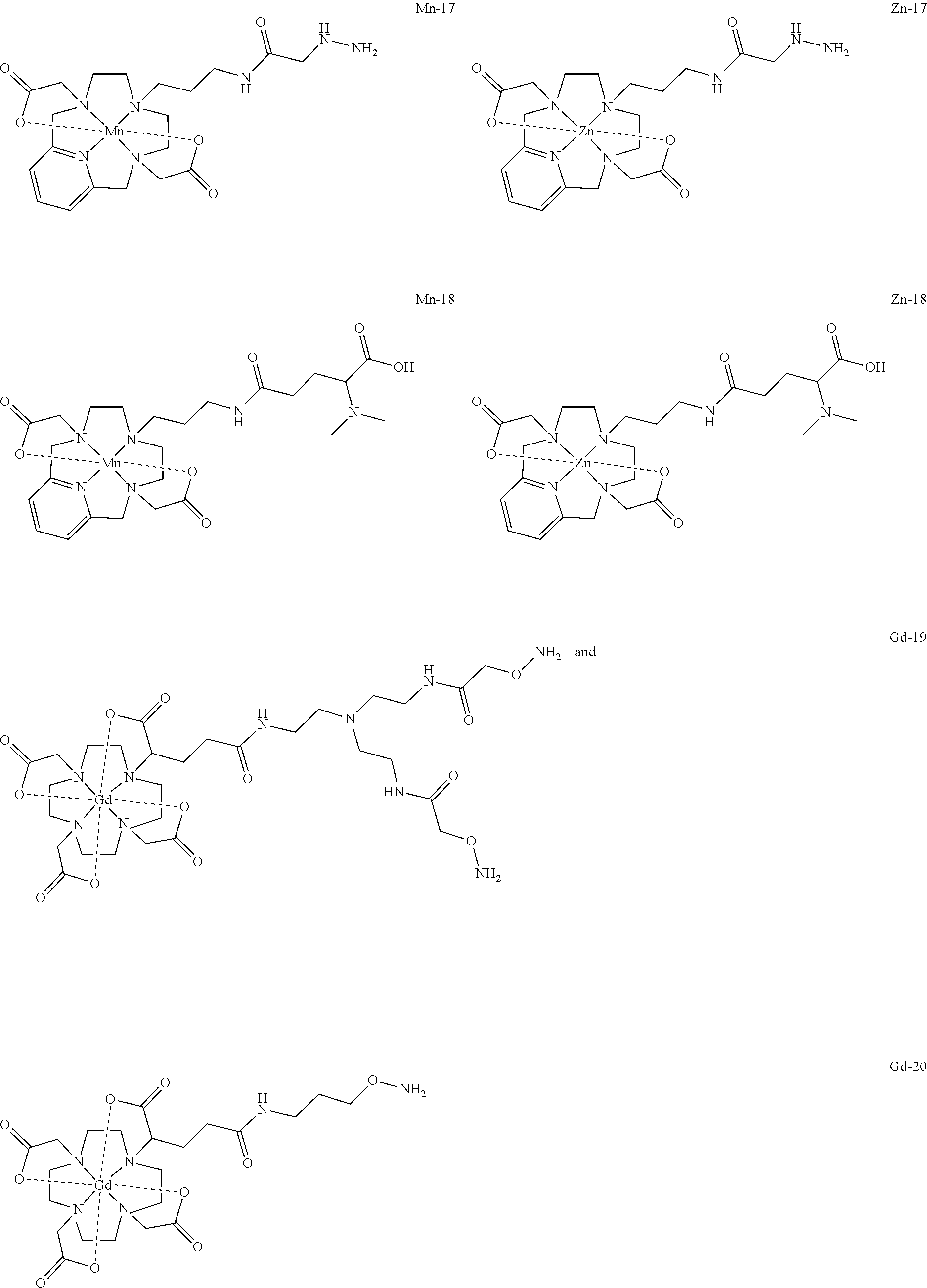
or a pharmaceutically acceptable salt thereof.

32. A composition comprising a compound of any one of claims 1-31, or a pharmaceutically acceptable salt thereof, and a pharmaceutically acceptable carrier.

33. A method of magnetic resonance (MR) imaging a subject comprising:
(a) obtaining a first magnetic resonance image of the subject;
(b) administering to a subject a compound of any one of claims 1-31 or a composition of claim 32;
(c) obtaining a second magnetic resonance image of the subject after a period of time; and
(d) comparing the first magnetic resonance image of the subject and the second magnetic resonance image of the subject.

34. A method for imaging liver fibrogenesis in a subject comprising:
(a) administering to a subject a compound of any one of claims 1-31 or a composition of claim 32; and
(b) obtaining a magnetic resonance image of the liver of the subject after a period of time.

35. A method of measuring liver fibrogenesis in a subject comprising:
(a) administering to a subject a compound of any one of claims 1-31 or a composition of claim 32;
(b) obtaining a first magnetic resonance image of the subject after a period of time;
(c) administering to a subject a compound of any one of claims 1-31 or a composition of claim 32 after a second period of time;
(d) obtaining a second magnetic resonance image of the subject after a period of time; and
(e) comparing the first magnetic resonance image of the subject and the second magnetic resonance image of the subject, thereby measuring liver fibrogenesis in the subject.

36. A method for detecting liver fibrogenesis in a subject comprising:
(a) administering to a subject a compound of any one of claims 1-31 or a composition of claim 32; and
(b) obtaining a magnetic resonance image of the subject after a period of time, thereby detecting the presence or absence of liver fibrogenesis in the subject.

37. A method of detecting liver fibrogenesis in a subject comprising:
(a) administering to a subject a compound of any one of claims 1-31 or a composition of claim 32;
(b) obtaining a first magnetic resonance image of the subject after a period of time;
(c) administering to a subject a compound of any one of claims 1-31 or a composition of claim 32 after a second period of time;
(d) obtaining a second magnetic resonance image of the subject after a period of time; and
(e) comparing the first magnetic resonance image of the subject and the second magnetic resonance image of the subject, thereby detecting the presence or absence of liver fibrogenesis in the subject.

38. A method for detecting liver fibrogenesis in a subject comprising obtaining a magnetic resonance image of the subject within a period of time after the subject has been administered subject a compound of any one of claims 1-31 or a composition of claim 32.

39. A method of positron emission tomography (PET) imaging a subject comprising:
(a) administering to a subject a compound of any one of claims 1-31 or a composition of claim 32; and
(b) obtaining a positron emission tomography image of the subject after a period of time.

40. A compound of Formula (II) 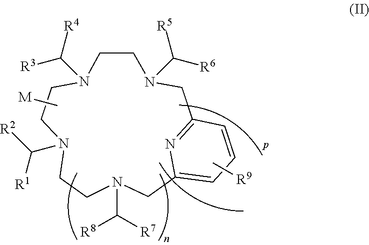
or a pharmaceutically acceptable salt thereof, wherein:
M is a metal cation;
each R¹, R³, R⁵, and R⁷ are independently hydrogen or —C(═O)OH;
each R², R⁴, R⁶, and R⁸ are independently hydrogen or C₃₋₂₅ alkyl, wherein the C₃₋₂₅ alkyl is optionally substituted with 1-2 substituents independently selected from the group consisting of —NR^{A}R^{B}, —OH, halogen, C₁₋₆ alkoxy, 5-10 membered heteroaryl, and 4-10 membered heterocyclyl, wherein the 5-10 membered heteroaryl and 4-10 membered heterocyclyl are each optionally substituted with 1-2 substituents independently selected from the group consisting of halogen, —NR^{A}R^{B}, —OH, C₁₋₆ alkyl, and —C₁₋₆ alkyl-(NR^{A}R^{B}); and one or more non-adjacent carbon atoms of the C₃₋₂₅ alkyl are optionally replaced by O, N, NH, or N(CH₃);
R⁹ is H, halogen, —NR^{A}R^{B}, —OH, C₁₋₆ alkyl, or —C₁₋₆ alkyl-(NR^{A}R^{B});
each R^{A} and R^{B} are independently hydrogen or C₁₋₆ alkyl;
n is 0 or 1; and
p is 0 or 1;
wherein if n is 0 at least two of R¹, R³, and R⁵ are —C(═O)OH and if n is 1 at least two of R¹, R³, R⁵, and R⁷ are —C(═O)OH.
